(19) United States
(12) Patent Application Publication
(10) Pub. No.: US 2026/0151482 A1
(43) Pub. Date: Jun. 4, 2026

(54) CHIMERIC ANTIGEN RECEPTORS SPECIFIC FOR B-CELL MATURATION ANTIGEN FOR USE IN TREATING MYELOMA

(71) Applicant: Celgene Corporation, Summit, NJ (US)

(72) Inventors: Timothy CAMPBELL, San Francisco, CA (US); Nathan Thomas MARTIN, Seattle, WA (US); Shari KAISER, Seattle, WA (US); Kristen HEGE, Burlingame, CA (US); Everton DeC ROWE, Hackensack, NJ (US)

(73) Assignee: Celgene Corporation, Summit, NJ (US)

(21) Appl. No.: 18/707,136

(22) PCT Filed: Nov. 2, 2022
(86) PCT No.: PCT/US2022/079190

§ 371 Date: May 2, 2024

Related U.S. Application Data
(60) Provisional application No. 63/275,414, filed on Nov. 3, 2021.
(60) Provisional application No. 63/287,904, filed on Dec. 9, 2021.

(51) Int. Cl. A61K 40/42; A61K 40/11; A61K 40/31; A61P 35/00

(52) U.S. Cl. CPC A61K 40/4215; A61K 40/11; A61K 40/31; A61P 35/00

(57) ABSTRACT

Provided herein are adoptive cell therapy methods involving the administration of genetically engineered cells for treating disease and conditions, including certain plasma cell malignancy. The cells generally express recombinant receptors such as chimeric antigen receptors (CARs) specific to B-cell maturation antigen (BCMA). In some embodiments, the methods are for selecting and treating subjects with multiple myeloma (MM).

### CROSS-REFERENCE TO RELATED APPLICATIONS

This application claims priority from U.S. provisional application No. 63/275,414, filed Nov. 3, 2021, entitled “METHODS FOR TREATMENT USING CHIMERIC ANTIGEN RECEPTORS SPECIFIC FOR B-CELL MATURATION ANTIGEN” and U.S. provisional application No. U.S. 63/287,904, filed Dec. 9, 2021, entitled “METHODS FOR TREATMENT USING CHIMERIC ANTIGEN RECEPTORS SPECIFIC FOR B-CELL MATURATION ANTIGEN.” The contents of which are incorporated by reference in their entirety.

### INCORPORATION BY REFERENCE OF SEQUENCE LISTING

The present application is being filed with a Sequence Listing in electronic format. The Sequence Listing is provided as a file entitled 683772001440SeqList.XML, created on Nov. 2, 2022, which is 349,655 bytes in size. The information in electronic format of the Sequence Listing is incorporated by reference in its entirety.

### FIELD

The present disclosure relates in some aspects to adoptive cell therapy involving the administration of genetically engineered cells for treating multiple myeloma (MM). The cells generally express recombinant receptors such as chimeric antigen receptors (CARs) specific to B-cell maturation antigen (BCMA). In some embodiments, the present disclosure also relates to methods for selecting subjects with MM for treatment with CAR-expressing cells, and predicting the response of subjects to treatment with the same.

### BACKGROUND

B cell maturation antigen (BCMA) is a transmembrane type III protein expressed on mature B lymphocytes. Following binding of BCMA to its ligands, B cell activator of the TNF family (BAFF) or a proliferation inducing ligand (APRIL), a pro-survival cell signal is delivered to the B cell which has been found to be required for plasma cell survival. The expression of BCMA has been linked to several diseases including cancer, autoimmune disorders and infectious diseases. Due to the role of BCMA in various diseases and conditions, including cancer, BCMA is a therapeutic target. Various BCMA-binding chimeric antigen receptors (CARs), and cells expressing such CARs, are available. However, there remains a need for improved methods of selecting subjects for, and treating patients with, BCMA-binding CARs and engineered BCMA-CAR expressing targeting cells, such as for use in adoptive cell therapy. Provided herein are embodiments that meet such needs.

### SUMMARY

Provided herein are methods of treating a subject having a multiple myeloma (MM), including: (a) determining that a subject has (i) a serum soluble B cell maturation antigen (sBCMA) level lower than about 600 ng/mL; and/or (ii) an absence of IgG heavy chain disease (HCD); and (b) administering to the subject a T cell therapy containing a dose of genetically engineered T cells expressing a chimeric antigen receptor (CAR) that binds to human BCMA.

Also provided herein are methods of treating a subject having or suspected of having a multiple myeloma (MM), including administering to the subject a T cell therapy containing a dose of genetically engineered T cells expressing a chimeric antigen receptor (CAR) that binds to human B cell maturation antigen (BCMA), wherein the subject was previously determined to have (i) a serum soluble B cell maturation antigen (sBCMA) level lower than about 600 ng/mL; and/or (ii) an absence of IgG heavy chain disease (HCD).

Also provided herein is a method of treating a subject having a multiple myeloma (MM), including: (a) selecting a subject for treatment with a T cell therapy containing a dose of genetically engineered T cells expressing a chimeric antigen receptor (CAR) that binds to human BCMA, wherein the subject was previously determined to have: (i) a serum soluble B cell maturation antigen (sBCMA) level lower than about 600 ng/ml; and/or (ii) an absence of IgG heavy chain disease (HCD); and (b) administering the T cell therapy to the subject.

Also provided herein are methods of selecting a subject having a multiple myeloma (MM) for treatment with a T cell therapy containing a dose of genetically engineered T cells, including determining that a subject has: (i) a serum soluble B cell maturation antigen (sBCMA) level lower than about 600 ng/mL; and/or (ii) an absence of IgG heavy chain disease (HCD), wherein, if the subject is determined to have (i) and/or (ii), the subject is selected for administration with a T cell therapy containing a dose of genetically engineered T cells expressing a chimeric antigen receptor (CAR) that binds to human BCMA.

Also provided herein are methods of predicting the response of a subject having a multiple myeloma (MM) to treatment with a T cell therapy containing a dose of genetically engineered T cells expressing a chimeric antigen receptor (CAR) that binds to human B cell maturation antigen (BCMA), including determining that a subject has (i) a serum soluble B cell maturation antigen (sBCMA) level lower than about 600 ng/mL; and/or (ii) an absence of IgG heavy chain disease (HCD), wherein, if the subject has (i) and/or (ii), the subject is predicted to achieve a complete response (CR) or stringent complete response (sCR), and wherein the treatment includes administration of the dose of genetically engineered T cells to the subject.

Also provided herein are methods of treating a subject having a multiple myeloma (MM), including determining that the subject has: (i) a serum soluble B cell maturation antigen (sBCMA) level higher than about 600 ng/mL; and/or (ii) a presence of IgG heavy chain disease (HCD); (b) debulking the MM; and (c) administering to the subject a T cell therapy containing a dose of genetically engineered T cells expressing a chimeric antigen receptor (CAR) that binds to human BCMA.

Also provided herein are methods of treating a subject having a multiple myeloma (MM), including administering to a subject a T cell therapy containing a dose of genetically engineered T cells expressing a chimeric antigen receptor (CAR) that binds to human B cell maturation antigen (BCMA), wherein (a) the subject was previously determined to have (i) a serum soluble B cell maturation antigen (sBCMA) level higher than about 600 ng/mL; and/or (ii) a presence of IgG heavy chain disease (HCD); and (b) the MM was debulked at a time between (1) the subject being determined to have (i) and/or (ii) and (2) the subject being administered the T cell therapy.

Also provided herein are methods of selecting a subject having a multiple myeloma (MM) for de-bulking, including determining that a subject has (i) a serum soluble B cell maturation antigen (sBCMA) level higher than about 600 ng/mL; and/or (ii) a presence of IgG heavy chain disease (HCD), wherein, if the subject is determined to have (i) and/or (ii), the subject is selected for debulking the MM prior to administration to the subject of a T cell therapy containing a dose of genetically engineered T cells expressing a chimeric antigen receptor (CAR) that binds to human BCMA.

Also provided herein are methods of predicting the response of a subject having a multiple myeloma (MM) to treatment with a T cell therapy containing a dose of genetically engineered cells expressing a chimeric antigen receptor (CAR) that binds to human B cell maturation antigen (BCMA), including determining that a subject has (i) a serum soluble B cell maturation antigen (sBCMA) level higher than about 600 ng/mL; and/or (ii) a presence of IgG heavy chain disease (HCD), wherein, if the subject has (i) and/or (ii), the subject is predicted not to achieve a complete response (CR) or stringent complete response (sCR)), and wherein the treatment includes administration of the dose of genetically engineered T cells to the subject.

Also provided herein are methods of treating a subject having a multiple myeloma (MM), including: (a) determining that a subject has a first serum soluble B cell maturation antigen (sBCMA) level higher than about 600 ng/mL; (b) debulking the MM: (c) determining that the subject has a post-debulking serum sBCMA level lower than about 600 ng/mL; and (d) administering to the subject a T cell therapy containing a dose of genetically engineered T cells expressing a chimeric antigen receptor (CAR) that binds to human BCMA.

In some embodiments, the subject is a human subject.

In some embodiments, the method further includes determining that the subject has (i) a serum sBCMA level or a post-debulking serum sBCMA level higher or lower than about 600 ng/mL; and/or (ii) a presence or absence of IgG HCD. In some embodiments, the method includes determining that the subject has a serum sBCMA level or a post-debulking serum sBCMA level higher or lower than about 600 ng/mL.

In some embodiments, the method includes determining the subject has a serum soluble B cell maturation antigen (sBCMA) level lower than about 600 ng/mL. In some embodiments, the method includes determining the subject has an absence of IgG heavy chain disease (HCD). In some embodiments, the method includes determining the subject has a serum soluble B cell maturation antigen (sBCMA) level lower than about 600 ng/mL and an absence of IgG heavy chain disease (HCD).

In some embodiments, the subject is determined to have a serum soluble B cell maturation antigen (sBCMA) level lower than about 600 ng/mL. In some embodiments, the subject is determined to have an absence of IgG heavy chain disease (HCD). In some embodiments, the subject is determined to have a serum soluble B cell maturation antigen (sBCMA) level lower than about 600 ng/mL and an absence of IgG heavy chain disease (HCD).

In some embodiments, the method includes determining that the subject has a serum sBCMA level higher or lower than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL. In some embodiments, the subject is determined to have a serum sBCMA level higher or lower than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL.

In some embodiments, the method includes determining that the subject has serum sBCMA level higher or lower than about 566 ng/mL. In some embodiments, the subject is determined to have a serum sBCMA level higher or lower than about 566 ng/mL. In some embodiments, determining the subject has a presence of IgG HCD includes detecting IgG in serum of the subject. In some embodiments, determining the subject has a presence of IgG HCD includes detecting IgG in urine of the subject.

In some embodiments, the determination of the serum sBCMA level is carried out about three months before, about two months before, about one month before, about three weeks before, about two weeks before, or about one week before: (i) administration of the T cell therapy to the subject; or (ii) the cells of the dose of genetically engineered CAR T cells are obtained from the subject. In some embodiments, the determination of the serum sBCMA level is carried out about three months before, about two months before, about one month before, about three weeks before, about two weeks before, or about one week before administration of the T cell therapy to the subject. In some embodiments, the determination of the serum sBCMA level is carried out about three months before, about two months before, about one month before, about three weeks before, about two weeks before, or about one week before the cells of the dose of genetically engineered CAR T cells are obtained from the subject.

In some embodiments, the method includes determining that the subject has a presence or absence of IgG HCD. In some embodiments, determining the subject has a presence of IgG HCD includes detecting IgG in serum and/or urine of the subject.

In some embodiments, the determination that the subject has a presence or absence of IgG HCD is carried out between about three months before, about two months before, about one month before, about three weeks before, about two weeks before, or about one week before: (i) administration of the T cell therapy to the subject; or (ii) the cells of the dose of genetically engineered CAR T cells are obtained from the subject. In some embodiments, the determination that the subject has a presence or absence of IgG HCD is carried out between about three months before, about two months before, about one month before, about three weeks before, about two weeks before, or about one week before administration of the T cell therapy to the subject. In some embodiments, the determination that the subject has a presence or absence of IgG HCD is carried out between about three months before, about two months before, about one month before, about three weeks before, about two weeks before, or about one week before the cells of the dose of genetically engineered CAR T cells are obtained from the subject.

In some embodiments, the method includes administering the dose of genetically engineered cells to the subject.

In some embodiments, if the subject is predicted to achieve a CR or a sCR, the method further includes administering to the subject a T cell therapy comprising a dose of genetically engineered T cells expressing a chimeric antigen receptor (CAR) that binds to BCMA.

In some embodiments, if the subject is predicted not to achieve a CR or a sCR, the method further includes selecting the subject for debulking the MM prior to administration to the subject of a dose of genetically engineered T cells expressing a chimeric antigen receptor (CAR) that binds to BCMA.

In some embodiments, following administration of the dose of genetically engineered T cells to the subject, the subject achieves a complete response (CR) or a stringent complete response (sCR). In some embodiments, following administration of the dose of genetically engineered T cells to the subject, the subject achieves a CR. In some embodiments, following administration of the dose of genetically engineered T cells to the subject, the subject achieves a sCR.

In some embodiments, the debulking includes administering chemotherapy, radiation, and/or an immunomodulatory agent to the subject. In some embodiments, the debulking includes administering chemotherapy to the subject. In some embodiments, the debulking includes administering radiation to the subject. In some embodiments, the debulking includes administering an immunomodulatory agent to the subject. In some embodiments, the debulking includes administering chemotherapy and radiation to the subject. In some embodiments, the debulking includes administering chemotherapy and an immunomodulatory agent to the subject. In some embodiments, the debulking includes administering radiation and an immunomodulatory agent to the subject. In some embodiments, the debulking includes administering chemotherapy, radiation, and an immunomodulatory agent to the subject.

In some embodiments, the chemotherapy is or includes melphalan, doxorubicin, or cyclophosphamide chemotherapy. In some embodiments, the chemotherapy is or includes melphalan. In some embodiments, the chemotherapy is or includes doxorubicin. In some embodiments, the chemotherapy is or includes cyclophosphamide chemotherapy. In some embodiments, the immunomodulatory agent is thalidomide, lenalidomide, or pomalidomide. In some embodiments, the immunomodulatory agent is thalidomide. In some embodiments, the immunomodulatory agent is lenalidomide. In some embodiments, the immunomodulatory agent is pomalidomide. In some embodiments, the immunomodulatory agent is a checkpoint inhibitor.

In some embodiments, the debulking is carried out within about three months before, within about two months before, within about one month before, within about three weeks before, within about two weeks before, or within about one week before administration of the T cell therapy to the subject.

In some embodiments, prior to the administration of the dose of genetically engineered T cells to the subject, the subject has received a lymphodepleting therapy comprising the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days. In some embodiments, prior to the administration of the dose of genetically engineered T cells to the subject, the subject has received a lymphodepleting therapy comprising the administration of fludarabine at or about 30 mg/m² body surface area of the subject, daily, and cyclophosphamide at or about 300 mg/m² body surface area of the subject, daily, for 3 days.

In some embodiments, the debulking is carried out prior to the lymphodepleting therapy. In some embodiments, the debulking is carried out after the lymphodepleting therapy. In some embodiments, the debulking is carried out prior to the lymphodepleting therapy and after the lymphodepleting therapy. In some embodiments, the subject is treated with a gamma-secretase inhibitor prior to administration of the dose of genetically engineered T cells to the subject.

In some embodiments, the MM is a high-risk MM or a relapsed and/or refractory (r/r) MM. In some embodiments, the MM is a high-risk MM or a relapsed and refractory (r/r) MM. In some embodiments, the MM is a high-risk MM. In some embodiments, the MM is a relapsed and refractory (r/r) MM. In some embodiments, the MM is a relapsed or refractory (r/r) MM. In some embodiments, the MM is a high-risk MM and a relapsed and refractory (r/r) MM.

In some embodiments, the subject is 18 year of age or older. In some embodiments, the subject has previously received three or more prior lines of therapy for the MM. In some embodiments, each of the three or more prior lines of therapy included two consecutive cycles, unless progressive disease was the best response to the line of therapy. In some embodiments, progressive disease is progression within 60 days after the last dose of the line of therapy. In some embodiments, the three or more prior lines of therapy include a proteasome inhibitor, an immunomodulatory agent, and an anti-CD38 antibody. In some embodiments, the subject is refractory to the last of the three or more prior lines of therapy. In some embodiments, the subject has an Eastern Cooperative Oncology Group (ECOG) performance status of 0 or 1. In some embodiments, the subject has an Eastern Cooperative Oncology Group (ECOG) performance status of 0. In some embodiments, the subject has an Eastern Cooperative Oncology Group (ECOG) performance status of 1.

In some embodiments, the subject has measurable disease at the time of the administration of the dose of genetically engineered T cells. In some embodiments, measurable disease includes: (i) serum M-protein greater or equal to 1.0 g/dL: (ii) urine M-protein greater or equal to 200 mg/24 h; and/or (iii) involved serum free light chain (FLC) level greater or equal to 10 mg/dL if serum FLC ratio is abnormal. In some embodiments, measurable disease includes serum M-protein greater or equal to 1.0 g/dL. In some embodiments, measurable disease includes urine M-protein greater or equal to 200 mg/24 h. In some embodiments, measurable disease includes involved serum free light chain (FLC) level greater or equal to 10 mg/dL if serum FLC ratio is abnormal. In some embodiments, measurable disease includes: (i) serum M-protein greater or equal to 1.0 g/dL; and (ii) urine M-protein greater or equal to 200 mg/24 h. In some embodiments, measurable disease includes: (i) serum M-protein greater or equal to 1.0 g/dL; and (ii) involved serum free light chain (FLC) level greater or equal to 10 mg/dL if serum FLC ratio is abnormal. In some embodiments, measurable disease includes: (i) urine M-protein greater or equal to 200 mg/24 h; and (ii) involved scrum free light chain (FLC) level greater or equal to 10 mg/dL if serum FLC ratio is abnormal. In some embodiments, measurable disease includes: (i) serum M-protein greater or equal to 1.0 g/dL; (ii) urine M-protein greater or equal to 200 mg/24 h; and (iii) involved serum free light chain (FLC) level greater or equal to 10 mg/dL if serum FLC ratio is abnormal.

In some embodiments, the subject does not have: (i) central nervous system (CNS) involvement; and/or (ii) a history or presence of clinically relevant CNS pathology. In some embodiments, the subject does not have central nervous system (CNS) involvement. In some embodiments, the subject does not have a history or presence of clinically relevant CNS pathology. In some embodiments, the subject does not have: (i) central nervous system (CNS) involvement; or (ii) a history or presence of clinically relevant CNS pathology. In some embodiments, the subject does not have active or a history of plasma cell leukemia (PCL).

In some embodiments, the CAR contains an extracellular antigen-binding domain that binds to BCMA, a transmembrane domain, and an intracellular signaling region.

In some embodiments, the extracellular antigen-binding domain contains a variable heavy chain (V_{H}) region. In some embodiments, the extracellular antigen-binding domain contains or consists of a single-domain antibody (sdAb). In some embodiments, the sdAb is a variable heavy chain (V_{H}) region. In some embodiments, the extracellular antigen-binding domain contains or consists of two sdAbs. In some embodiments, each of the two sdAbs is a variable heavy chain (V_{H}) region. In some embodiments, each of the two sdAbs binds to a different epitope of BCMA. In some embodiments, each of the two sdAbs bind the same epitope of BCMA.

In some embodiments, the extracellular antigen-binding domain comprises a variable heavy chain (V_{H}) region and a variable light chain (V_{L}) region. In some embodiments, the V_{H} region contains a CDR-H1, a CDR-H2, and a CDR-H3 containing the amino acid sequences set forth in SEQ ID NOS: 189, 190, and 191, respectively; and the V_{L} region contains a CDR-L1, a CDR-L2, and a CDR-L3 containing the amino acid sequences set forth in SEQ ID NOS: 192, 193, and 194, respectively; or the V_{H} region contains a CDR-H1, a CDR-H2, and a CDR-H3 containing the amino acid sequences set forth in SEQ ID NOS: 173, 174 and 175, respectively; and the V_{L} region contains a CDR-L1, a CDR-L2, and a CDR-L3 containing the amino acid sequences set forth in SEQ ID NOS: 183, 184 and 185, respectively. In some embodiments, the V_{H} region contains a CDR-H1, a CDR-H2, and a CDR-H3 containing the amino acid sequences set forth in SEQ ID NOS: 189, 190, and 191, respectively; and the V_{L} region contains a CDR-L1, a CDR-L2, and a CDR-L3 containing the amino acid sequences set forth in SEQ ID NOS: 192, 193, and 194, respectively. In some embodiments, the V_{H} region contains a CDR-H1, a CDR-H2, and a CDR-H3 containing the amino acid sequences set forth in SEQ ID NOS: 173, 174 and 175, respectively; and the V_{L} region contains a CDR-L1, a CDR-L2, and a CDR-L3 containing the amino acid sequences set forth in SEQ ID NOS: 183, 184 and 185, respectively. In some embodiments, the V_{H} region comprises an amino acid sequence set forth in SEQ ID NO: 18, and the V_{L} region comprises the amino acid sequence set forth in SEQ ID NO: 19; or the V_{H} region comprises an amino acid sequence set forth in SEQ ID NO: 24, and the V_{L} region comprises the amino acid sequence set forth in SEQ ID NO: 25. In some embodiments, the V_{H} region comprises an amino acid sequence set forth in SEQ ID NO: 18, and the V_{L} region comprises the amino acid sequence set forth in SEQ ID NO: 19. In some embodiments, the V_{H} region comprises an amino acid sequence set forth in SEQ ID NO: 24, and the V_{L} region comprises the amino acid sequence set forth in SEQ ID NO: 25.

In some embodiments, the extracellular antigen-binding domain is a single chain variable fragment (scFv). In some embodiments, the scFv comprises the amino acid sequence set forth in SEQ ID NO: 213 or SEQ ID NO: 188. In some embodiments, the scFv comprises the amino acid sequence set forth in SEQ ID NO: 213. In some embodiments, the scFv comprises the amino acid sequence set forth in SEQ ID NO: 188. In some embodiments, the scFv comprises the amino acid sequence set forth in any one of SEQ ID NOS: 216-247.

In some embodiments, the intracellular signaling region comprises the cytoplasmic signaling domain of a CD3-zeta (CD3ζ) chain. In some embodiments, the intracellular signaling region further contains a costimulatory signaling domain. In some embodiments, the costimulatory signaling domain comprises an intracellular signaling domain of CD28, 4-1BB, or ICOS, or a signaling portion thereof. In some embodiments, the costimulatory signaling domain comprises an intracellular signaling domain of CD28, or a signaling portion thereof. In some embodiments, the costimulatory signaling domain comprises an intracellular signaling domain of 4-1BB, or a signaling portion thereof. In some embodiments, the costimulatory signaling domain comprises an intracellular signaling domain of ICOS, or a signaling portion thereof.

In some embodiments, the costimulatory signaling domain is between the transmembrane domain and the cytoplasmic signaling domain of a CD3-zeta (CD3ζ) chain. In some embodiments, the transmembrane domain is or contains a transmembrane domain from CD28 or CD8. In some embodiments, the transmembrane domain is or contains a transmembrane domain from CD28. In some embodiments, CD28 is human CD28. In some embodiments, the transmembrane domain is or contains a transmembrane domain from CD8. In some embodiments, CD8 is human CD8.

In some embodiments, the CAR further contains an extracellular spacer between the extracellular antigen-binding domain and the transmembrane domain. In some embodiments, the spacer is from CD8. In some embodiments, the spacer is a CD8a hinge.

In some embodiments, the CAR comprises the amino acid sequence set forth in any one of SEQ ID NOS: 90-141. In some embodiments, the CAR comprises the amino acid sequence set forth in SEQ ID NO: 116 or SEQ ID NO: 124. In some embodiments, the CAR comprises the amino acid sequence set forth in SEQ ID NO: 116. In some embodiments, the CAR comprises the amino acid sequence set forth in SEQ ID NO: 124. In some embodiments, the CAR is encoded by the polynucleotide sequence set forth in SEQ ID NO: 214.

In some embodiments, the dose of genetically engineered T cells contains: idecabtagene vicleucel cells (e.g., such as ABECMA® cells); bb21217 cells; orvacabtagene autoleucel cells; CT103A cells: ciltacabtagene autoleucel cells; KITE585 cells; CT053 cells: BCMA-CS1 cCAR (BC1cCAR) cells; P-BCMA-101 cells; P-BCMA-ALLO1 cells: C-CAR088 cells: Descartes-08 cells; PBCAR269A cells: ALLO-715 cells; PHE885 cells; AUTOS cells; CTX120 cells; CB-011 cells: ALLO-605 (TuboCAR/MM) cells: pCDCAR1 (TriCAR-Z136) cells, or GC012F cells. In some embodiments, the dose of genetically engineered T cells contains idecabtagene vicleucel cells (e.g., such as ABECMA® cells).

In some embodiments, the dose of genetically engineered T cells contains CD3⁺ CAR-expressing T cells. In some embodiments, the dose of genetically engineered T cells contains a combination of CD4⁺ T cells or CD8⁺ T cells. In some embodiments, the dose of genetically engineered T cells contains a combination of CD4⁺ T cells and CD8⁺ T cells. In some embodiments, the dose of genetically engineered T cells contains a combination of CD4⁺ CAR-expressing T cells or CD8⁺ CAR-expressing T cells. In some embodiments, the dose of genetically engineered T cells contains a combination of CD4⁺ CAR-expressing T cells and CD8⁺ CAR-expressing T cells. In some embodiments, the ratio of CD4⁺ CAR-expressing T cells to CD8⁺ CAR-expressing T cells is or is approximately 1:1. In some embodiments, the ratio of CD4⁺ CAR-expressing T cells to CD8⁺ CAR-expressing T cells is or is between at or approximately 1:3 and at or approximately 3:1. In some embodiments, the ratio of CD4⁺ T cells to CD8⁺ T cells is or is approximately 1:1. In some embodiments, the ratio of CD4 T cells to CD8⁺ T cells is or is between at or approximately 1:3 and at or approximately 3:1.

In some embodiments, the percentage of naive-like T cells is greater than or greater than about 60% of the total genetically engineered T cells in the dose, optionally greater than or greater than about 65%, 70%, 80%, 90% or 95%. In some embodiments, the percentage of naive-like T cells is greater than or greater than about 40% of the total CD4+ genetically engineered T cells in the dose, optionally greater than or greater than about 50%, 60%, 70%, 80%, 90% or 95%. In some embodiments, the percentage of naive-like T cells s is greater than or greater than about 40% of the total CD8+ genetically engineered T cells in the dose, optionally greater than or greater than about 50%, 60%, 70%, 80%, 90% or 95%. In some embodiments, the naive-like T cells are CCR7+CD45RA+, CD27+CCR7+, or CD62L-CCR7+. In some embodiments, the naive-like T cells are CCR7+CD45RA+. In some embodiments, the naive-like T cells are CD27+CCR7+. In some embodiments, the naive-like T cells are CD62L-CCR7+.

In some embodiments, the percentage of central memory T cells is greater than or greater than about 60% of the total genetically engineered T cells in the dose, optionally greater than or greater than about 65%, 70%, 80%, 90% or 95%. In some embodiments, the percentage of central memory T cells is greater than or greater than about 40% of the total CD4+ genetically engineered T cells in the dose, optionally greater than or greater than about 50%, 60%, 70%, 80%, 90% or 95%. In some embodiments, the percentage of central memory T cells is greater than or greater than about 40% of the total CD8+ genetically engineered T cells in the dose, optionally greater than or greater than about 50%, 60%, 70%, 80%, 90% or 95%.

In some embodiments, the dose of genetically engineered T cells contains between about 0.5×10⁶ and about 600×10⁶ CAR-positive T cells. In some embodiments, the dose of genetically engineered T cells contains between about 0.5×10⁶ and about 100×10⁶ CAR-positive T cells. In some embodiments, the dose of genetically engineered T cells contains between about 0.5×10⁶ and about 10×10⁶ CAR-positive T cells. In some embodiments, the dose of genetically engineered T cells contains between about 1×10⁶ and about 10×10⁶ CAR-positive T cells. In some embodiments, the dose of genetically engineered T cells contains between about 0.5×10⁶ and about 1×10⁶ CAR-positive T cells. In some embodiments, the dose of genetically engineered T cells contains about 0.5×10⁶ CAR-positive T cells. In some embodiments, the dose of genetically engineered T cells contains about 0.75×10⁶ CAR-positive T cells. In some embodiments, the dose of genetically engineered T cells contains about 1.0×10⁶ CAR-positive T cells. In some embodiments, the dose of genetically engineered T cells contains about 2.5×10⁶ CAR-positive T cells. In some embodiments, the dose of genetically engineered T cells contains about 5.0×10⁶ CAR-positive T cells. In some embodiments, the dose of genetically engineered T cells contains about 7.5×10⁶ CAR-positive T cells. In some embodiments, the dose of genetically engineered T cells contains about 10.0×10⁶ CAR-positive T cells. In some embodiments, the dose of genetically engineered T cells contains between about 50×10⁶ and about 1000×10⁶ CAR-positive T cells. In some embodiments, the dose of genetically engineered T cells contains between about 100×10⁶ and about 600×10⁶ CAR-positive T cells. In some embodiments, the dose of genetically engineered T cells contains between about 150×10⁶ and about 450×10⁶ CAR-positive T cells. In some embodiments, the dose of genetically engineered T cells contains about 150×10⁶, 300×10⁶, or about 450×10⁶ CAR-positive T cells. In some embodiments, the dose of genetically engineered T cells contains about 150×10⁶ CAR-positive T cells. In some embodiments, the dose of genetically engineered T cells contains about 300×10⁶ CAR-positive T cells. In some embodiments, the dose of genetically engineered T cells contains about 450×10⁶ CAR-positive T cells.

In some embodiments, the cells of the dose of genetically engineered CAR T cells were obtained from the subject. In some embodiments, the cells of the dose of genetically engineered CAR T cells are obtained from the subject, such as for genetic engineering. In some embodiments, the dose of genetically engineered T cells are autologous to the subject. In some embodiments, the dose of genetically engineered T cells are allogencic to the subject.

### BRIEF DESCRIPTION OF THE DRAWINGS

FIG. 1 shows the correlation of baseline features with increased likelihood of CR/sCR (right side) or increased likelihood of non-CR/sCR (left side), ranked by importance (each dot represents an individual patient; dark gray: high or present levels; light gray: low or absent levels: CR: complete response; sCR: stringent complete response). ^{a}From patient laboratory measurements.

FIG. 2A shows the association of baseline levels of soluble BCMA (sBCMA) with clinical responses (CR: complete response; sCR stringent complete response; VGPR very good partial response: PR: partial response). Box represents Q1, Q3, and median. Whiskers represent the lowest value greater than Q1−1.5*IQR and the highest value less than Q3+1.5*IQR.

FIG. 2B shows the association of baseline levels of β-2 microglobulin with clinical responses (CR: complete response; sCR: stringent complete response; VGPR: very good partial response; PR: partial response). Box represents Q1, Q3, and median. Whiskers represent the lowest value greater than Q1−1.5*IQR and the highest value less than Q3+1.5*IQR.

FIG. 2C shows the number of patients with and without IgG heavy chain disease who exhibited a CR/sCR or non-CR/sCR (CR: complete response; sCR: stringent complete response).

FIG. 2D shows the association of baseline levels of D-dimer, ferritin, and sodium with clinical responses (CR complete response; sCR: stringent complete response; VGPR: very good partial response; PR: partial response). Box represents Q1, Q3, and median. Whiskers represent the lowest value greater than Q1−1.5*IQR, and the highest value less than Q3+1.5*IQR.

### DETAILED DESCRIPTION

Provided herein are methods of treating a subject having a multiple myeloma (MM) with a T cell therapy, e.g., a T cell therapy targeting BCMA, such as comprising a dose of genetically engineered T cells expressing a chimeric antigen receptor (CAR) that binds to BCMA, wherein the subject is determined to have a serum soluble BCMA (sBCMA) level lower than about 600 ng/ml and/or an absence of IgG heavy chain disease (HCD). In some embodiments, the subject is selected for administration of the T cell therapy.

Also provide herein are methods of treating a subject having a multiple myeloma (MM) with a T cell therapy, e.g., a T cell therapy targeting BCMA, such as comprising a dose of genetically engineered T cells expressing a chimeric antigen receptor (CAR) that binds to BCMA, wherein the subject is determined to have a serum soluble BCMA (sBCMA) level higher than about 600 ng/ml and/or a presence of IgG heavy chain disease (HCD). In some embodiments, the subject is selected for debulking of the MM and administration of the T cell therapy. In some embodiments, the subject is selected for debulking of the MM. In some embodiments, the MM is debulked prior to administration of the T cell therapy.

In some embodiments, the methods of selecting patients for administration of a T cell therapy are based on determining that the subject has a serum level of sBCMA lower than a particular threshold, such as lower than about 600 ng/mL, and/or an absence of IgG heavy chain disease. In some embodiments, the methods of selecting patients for debulking of the MM are based on determining that the subject has a serum level of sBCMA higher than a particular threshold, such as higher than about 600 ng/mL, and/or a presence of IgG heavy chain disease.

Also provided herein are methods of predicting the likelihood of a subject to respond to treatment with a T cell therapy comprising a dose of genetically engineered T cells expressing a chimeric antigen receptor (CAR) that binds to BCMA. In some embodiments, the response is a complete response (CR) or a stringent complete response (sCR). In some embodiments, the likelihood of the subject exhibiting a CR or sCR to treatment with the T cell therapy is based on determining that the subject has a serum level of soluble BCMA lower than a particular threshold, such as lower than about 600 ng/mL, and/or an absence of IgG heavy chain disease. In some embodiments, a subject is predicted to achieve a CR or sCR in response to treatment with the T cell therapy based on determining that the subject has a serum level of soluble BCMA lower than a particular threshold, such as lower than about 600 ng/mL, and/or an absence of IgG heavy chain disease. In some embodiments, a subject is predicted not to achieve a CR or sCR in response to treatment with the T cell therapy based on determining that the subject has a serum level of soluble BCMA higher than a particular threshold, such as higher than about 600 ng/mL, and/or a presence of IgG heavy chain disease.

Among the provided embodiments are methods, compositions, articles of manufacture, methods and uses including those targeting or directed to BCMA and BCMA-expressing cells (i.e. multiple myeloma). It is observed that BCMA is expressed on malignant plasma cells such as from all relapsed or newly diagnosed myeloma patients, for example, with little expression on normal tissues. Among the provided embodiments are approaches useful in the treatment of subjects having a multiple myeloma, and the selection of subjects having a multiple myeloma for treatment, with a T cell therapy comprising genetically engineered T cells expressing a chimeric antigen receptor (CAR) that binds BCMA (BCMA CAR T cells), and compositions and articles of manufacture comprising the same. The BCMA CAR T cells generally include extracellular antigen-binding domains that include antibodies (including antigen-binding antibody fragments, such as heavy chain variable (V_{H}) regions, single domain antibody fragments and single chain fragments (including scFvs), specific for BCMA. In some embodiments, the antigen-binding antibody fragments, including scFvs, include V_{H} and light chain variable (V_{L}) regions. Also provided are cells, such as engineered or recombinant cells expressing such anti-BCMA CARs and/or containing nucleic acids encoding such anti-BCMA CARs, and compositions and articles of manufacture and therapeutic doses containing such cells.

Also provided are methods of selecting subjects having a multiple myeloma for debulking of the MM prior to administration of a T cell therapy comprising BCMA CAR T cells. In some cases, a subject is selecting for debulking if the subject is determined to have a serum soluble BCMA (sBCMA) level above a particular threshold, such as above about 600 ng/mL, and/or a presence of IgG heavy chain disease (HCD).

Also provided are methods of predicting whether a subject having a multiple myeloma will achieve a complete response (CR) or a stringent complete response (sCR) following administration of a T cell therapy comprising BCMA CAR T cells to the subject. In some cases, the methods of predicting are based on the level of serum soluble BCMA (sBCMA), such as in a sample obtained from the patient prior to administration of the T cell therapy, and/or the presence or absence of IgG heavy chain disease (HCD).

Adoptive cell therapies (including those involving the administration of BCMA CAR T cells for treatment of multiple myeloma, as well as other adoptive immune cell and adoptive T cell therapies) can be effective in the treatment of cancer and other diseases and disorders. In certain contexts, however, available approaches to adoptive cell therapy may not always be entirely satisfactory.

In some aspects, available approaches for treatment of multiple myeloma (e.g. relapsed and refractory MM) are complex and may not always be entirely satisfactory. Patients with relapsed or refractory MM have poor outcomes with currently available therapies. Relapsed and refractory MM often does not respond to further treatments and usually progresses within 2 to 4 months. (Chari et al., N Engl J Med (2019) 381:727-38 and Lonial et al., Lancet Oncol (2020) 21:207-21). In some aspects, choosing a treatment regimen can depend on numerous factors including drug availability, response to prior therapy, aggressiveness of the relapse, eligibility for autologous stem cell transplantation (ASCT), and whether the relapse occurred on or off therapy. In some aspects, MM results in relapses and remissions, and existing regimens in some cases can result in relapse and/or toxicity from the treatment. In some cases, subjects with particularly aggressive disease, such as subjects that have persistent or relapsed disease after various therapies, subjects with a high disease burden, such as a high tumor burden, and/or subjects with high risk disease (i.e. high risk cytogenetics), can be particularly difficult to treat, and responses to certain therapies in these subjects can be poor or have a short duration. In some cases, subjects who have been heavily pre-treated, e.g., subjects who have relapsed after several different prior lines of therapy, can exhibit a low response rate and/or high incidence of adverse events.

In particular, outcomes for patients with relapsed and/or refractory multiple myeloma (R/R MM) with previous exposure to immunomodulatory agents, proteasome inhibitors (PIs), and anti-CD38 antibodies are poor. (Chari et al., N Engl J Med (2019) 381:727-38; Lonial et al., Poster presentation at the European Hematology Association (EHA) Virtual Meeting 2021: Abstract EP970; and Richardson et al., J Clin Oncol (2021) 39:757-67). Further, it is currently difficult to predict which patients will achieve deep responses to CAR T cell treatment. In some aspects, the provided embodiments are based on an observation that treatment of subjects or the selection of subjects for treatment according to the provided embodiments results in a high response rate (i.e. CR or sCR), including in subjects with previous exposure to prior lines of therapy (e.g. an immunomodulatory agent, a proteasome inhibitor (PI), and an anti-CD38 antibody). In particular, the provided embodiments are based on an observation that a subject is more likely, or predicted, to achieve a CR or sCR in response to treatment with BCMA CAR T cells if the subject has a serum sBCMA level below a particular threshold (e.g. below 500, 566, or 600 ng/mL) and/or if the subject has an absence of IgG heavy chain disease (HCD). Conversely, it is observed herein that a subject is less likely, or predicted not, to achieve a CR or sCR in response to treatment with BCMA CAR T cells if the subject has a serum sBCMA level above a particular threshold (e.g. above 500, 566, or 600 ng/mL) and/or if the subject has presence of IgG heavy chain disease (HCD). Based on these observations, is it contemplated that a subject having a serum sBCMA level above a particular threshold (e.g. above 500, 566, or 600 ng/mL) and/or a presence of IgG heavy chain disease (HCD) can be selected for and/or subjected to debulking of the MM prior to administration of the T cell therapy. In some cases, the debulking of a MM prior to administration of the T cell therapy results in the subject having a serum sBCMA level lower than a particular threshold (e.g. lower than 500, 566, or 600 ng/mL). In some cases, after the debulking, the subject is administered the T cell therapy.

The provided embodiments, in some contexts, are based on an observation from a clinical study, that administration of BCMA CAR T cells, such as those described herein, results in higher rates of CR or sCR in subjects having lower serum sBCMA levels and/or an absence of IgG HCD, including in relapsed and refractory subjects who have received three or more prior lines of therapy. In some aspects, the provided methods, uses, and cells result in high rates of CR or sCR. In some aspects, such high response may be achieved from selecting subjects for administration of the T cell therapy who are determined to have a serum sBCMA level below a particular threshold (e.g. below 500, 566, or 600 ng/mL) and/or an absence of IgG heavy chain disease (HCD). Further, such high response may be achieved from selecting subjects for debulking of the MM prior to administration of the T cell therapy who are determined to have a serum sBCMA level above a particular threshold (e.g. above 500, 566, or 600 ng/mL) and/or a presence of IgG heavy chain disease (HCD). In some aspects, treatment of subjects with high risk and/or relapsed and refractory MM (e.g., heavily pre-treated subjects, subjects with a high tumor burden and/or subjects with high risk cytogenetics) according to the provided embodiments, was observed to provide effective and durable treatment, such as that resulting is a CR or sCR.

In various aspects, the provided methods allow for the selection and/or treatment of subjects having high risk and/or R/R MM that can overcome or counteract certain limitations that can reduce optimal responses to cell therapy, in such subjects. In some contexts, the provided methods and uses of the engineered cells or compositions comprising the engineered cells, has been observed to provide an advantage in treating subjects, that results in a high CR or sCR rate at various different dose levels tested. Further, the provided methods and uses of the engineered cells or compositions comprising the engineered cells, has been observed to provide an advantage in treating subjects with particularly high risk and/or R/R disease, including subjects who have relapsed and are refractory to numerous different prior treatments for the disease.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS OF ASSESSING BIOMARKERS AND SELECTING SUBJECTS FOR AND PREDICTING RESPONSE TO TREATMENT WITH A T CELL THERAPY

Among the provided methods and uses are methods of selecting a subject for treatment with a T cell therapy, which may include assessing or detecting biomarkers or parameters, such as in a sample from a subject, that are associated with a response outcome (e.g., complete response (CR) or stringent complete response (sCR)) to treatment with the T cell therapy. Also among the provided methods and uses are methods of selecting a subject for debulking treatment, and for predicting the response of a subject to treatment with the T cell therapy, which may include assessing or detecting biomarkers or parameters, such as in a sample from a subject, that are associated with a response outcome (e.g., complete response (CR) or stringent complete response (sCR)) to treatment with the T cell therapy. Also among the provided methods and uses are methods of predicting whether a subject will exhibit a response outcome to treatment with a T cell therapy, which may include assessing or detecting biomarkers or parameters, such as in a sample from a subject, that are associated with the response outcome (e.g., complete response (CR) or stringent complete response (sCR)) to treatment with the T cell therapy.

Complete response (CR) and stringent complete response (sCR) are defined by the International Myeloma Working Group (IMWG) Standard Response Criteria as set forth in Table 1 below (Kumar et al., Lancet Oncol (2016) 17(8):e328-46).

TABLE 1
Standard IMWG Response Criteria for sCR and CR

| | |
|---|---|
| Stringent | Complete response as defined below plus normal FLC ratio** |
| complete | and absence of clonal cells in bone marrow biopsy by |
| response | immunohistochemistry (κ/λ ratio ≤4:1 or ≥1:2 for κ and λ |
| (sCR) | patients, respectively, after counting ≥100 plasma |
| | cells)†† |
| Complete | Negative immunofixation on the serum and urine and |
| response | disappearance of any soft tissue plasmacytomas and <5% |
| (CR) | plasma cells in bone marrow aspirates |

**All recommendations regarding clinical uses relating to serum FLC levels or FLC ratio are based on results obtained with the validated Freelite test (Binding Site, Birmingham, UK).
††Presence/absence of clonal cells on immunohistochemistry is based upon the κ/λ/L ratio. An abnormal κ/λ ratio by immunohistochemistry requires a minimum of 100 plasma cells for analysis. An abnormal ratio reflecting presence of an abnormal clone is κ/λ of >4:1 or <1:2.

In some of any embodiments, the methods involve assessing the serum soluble B cell maturation antigen (sBCMA) level, such as in a sample obtained from a subject. In some embodiments, the sample is a serum sample. In some of any embodiments, the methods involve determining whether a subject has a serum sBCMA level lower than about 600 ng/mL. In some of any embodiments, the methods involve determining whether a subject has a serum sBCMA level higher than about 600 ng/mL. In some of any embodiments, the methods involve determining whether a subject has a serum sBCMA level lower than about 566 ng/mL. In some of any embodiments, the methods involve determining whether a subject has a serum sBCMA level higher than about 566 ng/mL. In some of any embodiments, the methods involve determining whether a subject has a serum sBCMA level lower than about 500 ng/mL. In some of any embodiments, the methods involve determining whether a subject has a serum sBCMA level higher than about 500 ng/mL. In some embodiments, the methods involve comparing, individually, the level, amount or concentration of serum sBCMA from a sample obtained from a subject to a threshold level, thereby determining a likelihood that the subject will achieve a CR or sCR to the T cell therapy. In some aspects, the threshold level is determined based on the mean or median values and values within a range or standard deviation of the mean or median values of the level, amount or concentration of serum sBCMA, in biological samples obtained from a group of subjects prior to receiving a T cell therapy, wherein each of the subjects of the group went on to exhibit a CR or sCR, or did not go on to exhibit a CR or sCR.

In some of any embodiments, the methods involve assessing whether a patient has IgG heavy chain disease (HCD), such as in a sample obtained from a subject. In some embodiments, the sample is a serum sample or a urine sample. In some of any embodiments, the methods involve determining whether a subject has a presence of IgG HCD. In some of any embodiments, the methods involve determining whether a subject has an absence of IgG HCD.

In some of any embodiments, the methods involve assessing the number of copies of vector in the dose of engineered cells. In some embodiments, the vector comprises or encodes the chimeric antigen receptor (CAR). In some embodiments, the methods involve comparing the number of copies of vector in the dose of engineered cells to a threshold level, thereby determining a likelihood that the subject will achieve a CR or sCR to the T cell therapy.

In some of any embodiments, the methods involve assessing the β-2 microglobulin level, such as in a sample obtained from a subject. In some embodiments, the sample is a blood sample. In some embodiments, the sample is a urine sample. In some embodiments, the sample is a cerebrospinal fluid (CSF) sample. In some embodiments, the methods involve comparing, individually, the level, amount or concentration of β-2 microglobulin from a sample obtained from a subject to a threshold level, thereby determining a likelihood that the subject will achieve a CR or sCR to the T cell therapy.

In some of any embodiments, the methods involve assessing the D-dimer level, such as in a sample obtained from a subject. In some embodiments, the sample is a blood sample. In some embodiments, the methods involve comparing, individually, the level, amount or concentration of D-dimer from a sample obtained from a subject to a threshold level, thereby determining a likelihood that the subject will achieve a CR or sCR to the T cell therapy.

In some of any embodiments, the methods involve assessing the ferritin level, such as in a sample obtained from a subject. In some embodiments, the sample is a blood sample. In some embodiments, the methods involve comparing, individually, the level, amount or concentration of ferritin from a sample obtained from a subject to a threshold level, thereby determining a likelihood that the subject will achieve a CR or sCR to the T cell therapy.

In some of any embodiments, the methods involve assessing the sodium level, such as in a sample obtained from a subject. In some embodiments, the sample is a blood sample. In some embodiments, the methods involve comparing, individually, the level, amount or concentration of sodium from a sample obtained from a subject to a threshold level, thereby determining a likelihood that the subject will achieve a CR or sCR to the T cell therapy.

In some of any embodiments, the methods involve assessing the prothrombin time-international normalized ratio (PT-INR), such as in a sample obtained from a subject. In some embodiments, the sample is a blood sample. In some embodiments, the methods involve comparing PT-INR from a sample obtained from a subject to a threshold PT-INR, thereby determining a likelihood that the subject will achieve a CR or sCR to the T cell therapy.

In some embodiments, the T cell therapy comprises a dose of engineered cells expressing a chimeric antigen receptor (CAR) that binds to BCMA (i.e. BCMA CAR T cells). In some embodiments, the level of serum sBCMA and/or absence or presence of IgG HCD is assessed from a subject or in a sample obtained from a subject, that has a multiple myeloma, such as a relapsed and refractory (RR) multiple myeloma. In some embodiments, the subject is a candidate for treatment with a T cell therapy, and/or has received treatment with a T cell therapy. In some embodiments, the provided methods can be used to identify or select subjects that are likely to respond to a T cell therapy; and/or select subjects for a particular treatment prior to administration of the T cell therapy, such as debulking.

In In some aspects, the methods involve further monitoring the subject for possible response, based on the likelihood of response as determined in accordance with the provided embodiments, e.g. by assessment of serum sBCMA and/or IgG HCD.

In some embodiments, the methods involve assessing or detecting the presence or absence of IgG HCD and/or the concentration, amount, or level of serum sBCMA. In some cases, the methods can include comparing the concentration, amount, or level of serum sBCMA to a particular reference value, such as a threshold level, e.g., that associated with a particular response, such as CR and/or sCR. In some embodiments, the methods also involve selecting subjects for treatment with a T cell therapy based on the assessment of the presence or absence of IgG HCD and/or comparison of the serum sBCMA to a reference value or threshold level of serum sBCMA.

In some aspects, a biological sample, e.g., a serum or urine sample from the subject, can be obtained for detecting the presence or absence of IgG HCD. In some aspects, a biological sample, e.g., a blood or serum sample from the subject, can be obtained for detecting the concentration, amount, or level of serum sBCMA.

In some embodiments, the IgG HCD and/or serum sBCMA level is an objectively measurable characteristic or a molecule expressed by or in a biological sample, including cells, which can be indicative of or associated with a particular state or phenomenon, such as a therapeutic outcome or a disease state. In some aspects, the IgG HCD and/or serum sBCMA can be measured or detected. For example, the presence or absence of IgG HCD can be detected. In some aspects, the parameters such as concentration, amount, or level of serum sBCMA can be measured or detected. In some embodiments, the presence or absence of IgG HCD and/or the concentration, amount, or level of serum sBCMA can be associated with, correlated to, indicative of and/or predictive of particular states, such as particular therapeutic outcomes or state of the subject. In some aspects, the presence or absence of IgG HCD and/or the concentration, amount, or level of serum sBCMA can be used to assess the likelihood of a particular outcome or state, such as a particular therapeutic outcome, including response outcome. In some embodiments, the response outcome is complete response (CR) or stringent complete response (sCR). In some embodiments, the response outcome is CR. In some embodiments, the response outcome is sCR. Thus, in some embodiments, the presence or absence of IgG HCD and/or the concentration, amount, or level of serum sBCMA can be used to assess the likelihood a subject will exhibit a CR or a sCR following administration of the T cell therapy.

In some embodiments, the absence or presence of IgG HCD can be used singly or in combination with the concentration, amount, or level of serum sBCMA. In some embodiments, the concentration, amount, or level of serum sBCMA can be used singly or in combination with the absence or presence of IgG HCD.

In some embodiments, the presence or absence of IgG HCD and/or the level of serum sBCMA is determined from a biological sample. In some aspects, the biological sample is a bodily fluid or a tissue. In some such embodiments, the biological sample, e.g., bodily fluid, is or contains whole blood, serum or plasma. In some such embodiments, the biological sample, e.g., bodily fluid, is or contains serum. In some such embodiments, the biological sample, e.g., bodily fluid, is or contains urine.

In some embodiments, the presence or absence of IgG HCD and/or the level of serum sBCMA is determined prior to administration of the T cell therapy (e.g., pre-infusion), e.g., obtained up to 1 day, up to 2 days, up to 7 days, up to 14 days, up to 21 days, up to 28 days, up to 35 days, up to 40 days, up to 2 months, or up to 3 months prior to initiation of the administration of the T cell therapy.

In some embodiments, the presence or absence of IgG HCD is determined prior to administration of the T cell therapy (e.g., pre-infusion), e.g., obtained up to 1 day, up to 2 days, up to 7 days, up to 14 days, up to 21 days, up to 28 days, up to 35 days, up to 40 days, up to 2 months, or up to 3 months prior to initiation of the administration of the T cell therapy. In some embodiments, determining the subject has a presence or absence of IgG HCD is carried out between about three months before, about two months before, about one month before, about three weeks before, about two weeks before, or about one week before administration of the T cell therapy to the subject. In some embodiments, determining the subject has a presence or absence of IgG HCD is carried out between about three months before administration of the T cell therapy to the subject. In some embodiments, determining the subject has a presence or absence of IgG HCD is carried out between about two months before administration of the T cell therapy to the subject. In some embodiments, determining the subject has a presence or absence of IgG HCD is carried out between about three weeks before administration of the T cell therapy to the subject. In some embodiments, determining the subject has a presence or absence of IgG HCD is carried out between about two weeks before administration of the T cell therapy to the subject. In some embodiments, determining the subject has a presence or absence of IgG HCD is carried out between about one week before administration of the T cell therapy to the subject. In some embodiments, determining the subject has a presence or absence of IgG HCD is carried out prior to leukapheresis. In some embodiments, determining the subject has a presence or absence of IgG HCD is carried out prior to administration of a lymphodepleting therapy to the subject.

In some embodiments, the level of serum sBCMA is determined prior to administration of the T cell therapy (e.g., pre-infusion), e.g., obtained up to 1 day, up to 2 days, up to 7 days, up to 14 days, up to 21 days, up to 28 days, up to 35 days, up to 40 days, up to 2 months, or up to 3 months prior to initiation of the administration of the T cell therapy. In some embodiments, the first level of serum sBCMA is determined prior to administration of the T cell therapy (e.g., pre-infusion), e.g., obtained up to 1 day, up to 2 days, up to 7 days, up to 14 days, up to 21 days, up to 28 days, up to 35 days, up to 40 days, up to 2 months, or up to 3 months prior to initiation of the administration of the T cell therapy. In some embodiments, the post-debulking level of serum sBCMA is determined prior to administration of the T cell therapy (e.g., pre-infusion), e.g., obtained up to 1 day, up to 2 days, up to 7 days, up to 14 days, up to 21 days, up to 28 days, up to 35 days, up to 40 days, up to 2 months, or up to 3 months prior to initiation of the administration of the T cell therapy.

In some embodiments, determining the serum sBCMA level is carried out about three months before, about two months before, about one month before, about three weeks before, about two weeks before, or about one week before administration of the T cell therapy to the subject. In some embodiments, determining the serum sBCMA level is carried out about three months before administration of the T cell therapy to the subject. In some embodiments, determining the serum sBCMA level is carried out about two months before administration of the T cell therapy to the subject. In some embodiments, determining the serum sBCMA level is carried out about one month before administration of the T cell therapy to the subject. In some embodiments, determining the serum sBCMA level is carried out about three weeks before administration of the T cell therapy to the subject. In some embodiments, determining the serum sBCMA level is carried out about two weeks before administration of the T cell therapy to the subject. In some embodiments, determining the serum sBCMA level is carried out about one week before administration of the T cell therapy to the subject. In some embodiments, determining the serum sBCMA level is carried out prior to leukapheresis. In some embodiments, determining the serum sBCMA level is carried out prior to administration of a lymphodepleting therapy to the subject.

In some embodiments, the biological sample is obtained from the subject prior to administration of the cell therapy (e.g., pre-infusion), e.g., obtained up to up to 1 day, up to 2 days, up to 7 days, up to 14 days, up to 21 days, up to 28 days, up to 35 days, up to 40 days, up to 2 months, or up to 3 months prior to initiation of the administration of the T cell therapy. In some embodiments, the biological sample is obtained from the subject prior to leukapheresis. In some embodiments, the biological sample is obtained from the subject prior to administration of a lymphodepleting therapy to the subject.

In some embodiments, the level of serum sBCMA is determined in a biological sample. In some embodiments, the biological sample is a blood, serum or plasma sample. In some embodiments, the biological sample is a blood sample. In some embodiments, the biological sample is an apheresis or leukapheresis sample. In some embodiments, the biological sample is a serum sample. In some embodiments, the level of serum sBCMA is determined in a serum sample from the subject.

In some embodiments, the presence or absence of IgG HCD is determined in a biological sample. In some embodiments, the biological sample is a blood sample, a serum sample, a plasma sample, or a urine sample. In some embodiments, the biological sample is a blood sample. In some embodiments, the biological sample is an apheresis or leukapheresis sample. In some embodiments, the biological sample is a serum sample. In some embodiments, the presence or absence of IgG HCD is determined in a serum sample from the subject. In some embodiments, the biological sample is a urine sample. In some embodiments, the presence or absence of IgG HCD is determined in a urine sample from the subject.

In some embodiments, reagents can be used prior to the administration of the T cell therapy or after the administration of the T cell therapy, for diagnostic purposes, to identify subjects and/or to assess treatment outcomes.

In some embodiments, determining the presence or absence of IgG HCD and/or the level of serum sBCMA comprises performing an in vitro assay. In some embodiments, determining the presence or absence of IgG HCD comprises performing an in vitro assay. In some embodiments, determining the level of serum sBCMA comprises performing an in vitro assay. In some aspects, the in vitro assay is an immunoassay, an aptamer-based assay, a histological or cytological assay, or an mRNA expression level assay. In some embodiments, the determining comprises measuring by an enzyme linked immunosorbent assay (ELISA), immunoblotting, immunoprecipitation, radioimmunoassay (RIA), immunostaining, a flow cytometry assay, surface plasmon resonance (SPR), a chemiluminescence assay, a lateral flow immunoassay, an inhibition assay or an avidity assay. In some cases, the determining comprises using a binding reagent that specifically binds to IgG. In some cases, the determining comprises using a binding reagent that specifically binds to BCMA, e.g. sBCMA. In some aspects, the binding reagent is an antibody or antigen-binding fragment thereof, an aptamer or a nucleic acid probe.

In some embodiments, tumor-associated BCMA expression is assessed from bone marrow biopsies. In some embodiments, tumor-associated BCMA expression is assessed immunohistochemically using an antibody, such as a monoclonal antibody directed against an intracellular BCMA epitope. In some embodiments, tumor cell surface BCMA expression is quantified on fresh bone marrow aspirates by flow cytometry, such as using Quantibrite™ beads. In some embodiments, soluble BCMA is assessed in serum, such as using a Luminex® immunoassay. In some embodiments, soluble BCMA is assessed in serum longitudinally (such as on consecutive days, or at regular intervals). Methods for measuring soluble BCMA, including serum BCMA, are known in the art, and may include any methods described in Munshi et al., N Engl J Med (2021) 384:705-16. sBCMA levels may also be determined with the ONCOtracker assay (oncotracker.com/sbcma-biomarker/).

In some embodiments, IgG heavy chain disease is determined by any methods known in the art, including serum or urine immunofixation.

### A. Serum Soluble BCMA

i. Selection for Treatment and/or Debulking

In some embodiments, the methods comprise determining the level of serum soluble B cell maturation antigen (sBCMA) in a sample obtained from a subject having a MM. In some embodiments, based on the level of serum sBCMA, the subject is selected for administration of a T cell therapy and/or debulking of the MM. In some embodiments, based on the level of serum sBCMA, the subject is selected for administration of a T cell therapy. In some embodiments, based on the level of serum sBCMA, the subject is selected for debulking of the MM. In some embodiments, based on the level of serum sBCMA, the subject is selected for administration of a T cell therapy and debulking of the MM.

In some embodiments, the methods comprise determining whether a subject has a serum sBCMA level lower than about 600 ng/mL, such as lower than about 566 ng/mL or 500 ng/mL. In some embodiments, if the subject is determined to have a serum sBCMA level lower than about 600 ng/mL, such as lower than about 566 ng/mL or 500 ng/mL, the subject is administered a T cell therapy comprising a dose of genetically engineered T cells expressing a chimeric antigen receptor (CAR) that binds to BCMA (i.e. BCMA CAR T cells). In some embodiments, if the subject is determined to have a serum sBCMA level lower than about 600 ng/mL, such as lower than about 566 ng/mL or 500 ng/mL, the subject is selected for administration of a T cell therapy comprising a dose of genetically engineered T cells expressing a chimeric antigen receptor (CAR) that binds to BCMA (i.e. BCMA CAR T cells).

In some embodiments, the methods comprise determining whether a subject has a scrum sBCMA level higher than about 600 ng/mL, such as higher than about 500 ng/mL or 566 ng/mL. In some embodiments, if the subject is determined to have a serum sBCMA level higher than about 600 ng/mL, such as higher than about 500 ng/mL or 566 ng/mL, the subject is not administered a T cell therapy comprising a dose of genetically engineered T cells expressing a chimeric antigen receptor (CAR) that binds to BCMA (i.e. BCMA CAR T cells).

Thus, in some embodiments, the methods comprise (a) determining that a subject has a serum soluble B cell maturation antigen (sBCMA) level lower than about 600 ng/ml; and (b) administering to the subject a T cell therapy comprising a dose of genetically engineered T cells expressing a chimeric antigen receptor (CAR) that binds to BCMA. In some embodiments, the methods comprise administering to the subject a T cell therapy comprising a dose of genetically engineered T cells expressing a chimeric antigen receptor (CAR) that binds to B cell maturation antigen (BCMA), wherein the subject was previously determined to have a serum soluble B cell maturation antigen (sBCMA) level lower than about 600 ng/ml. In some embodiments, the methods comprise selecting a subject having a multiple myeloma (MM) for treatment with a T cell therapy comprising a dose of genetically engineering T cells, comprising determining that a subject has a serum soluble B cell maturation antigen (sBCMA) level lower than about 600 ng/ml, wherein, if the subject is determined to have a serum sBCMA level lower than about 600 ng/ml, the subject is selected for administration with the T cell therapy.

In some embodiments, the methods comprise determining whether a subject has a serum sBCMA level higher than about 600 ng/mL, such as higher than about 500 ng/mL or 566 ng/mL. In some embodiments, if the subject is determined to have a serum sBCMA level higher than about 600 ng/mL, such as higher than about 500 ng/mL or 566 ng/mL, the multiple myeloma is debulked prior to administration of a T cell therapy comprising a dose of genetically engineered T cells expressing a chimeric antigen receptor (CAR) that binds to BCMA (i.e. BCMA CAR T cells). In some embodiments, the methods comprise determining whether a subject has a serum sBCMA level higher than about 600 ng/mL, such as higher than about 500 ng/mL or 566 ng/mL. In some embodiments, if the subject is determined to have a serum sBCMA level higher than about 600 ng/mL, such as higher than about 500 ng/mL or 566 ng/mL, the subject is selected for debulking of the multiple myeloma prior to administration of a T cell therapy comprising a dose of genetically engineered T cells expressing a chimeric antigen receptor (CAR) that binds to BCMA (i.e. BCMA CAR T cells).

Thus, in some embodiments, the methods comprise (a) determining that the subject has a serum soluble B cell maturation antigen (sBCMA) level higher than about 600 ng/ml; (b) debulking the MM; and (c) administering to the subject a T cell therapy comprising a dose of genetically engineered T cells expressing a chimeric antigen receptor (CAR) that binds to BCMA. In some embodiments, the methods comprise administering to a subject a T cell therapy comprising a dose of genetically engineered T cells expressing a chimeric antigen receptor (CAR) that binds to BCMA, wherein: (a) the subject was previously determined to have a serum soluble B cell maturation antigen (sBCMA) level higher than about 600 ng/ml; and (b) the MM was previously debulked at a time between (1) the subject being determined to have a serum sBCMA level higher than about 600 ng/mL and (2) the subject being administered the T cell therapy. In some embodiments, the methods comprise determining that a subject has a serum soluble B cell maturation antigen (sBCMA) level higher than about 600 ng/ml, wherein, if the subject is determined to have a serum sBCMA level higher than about 600 ng/mL, the subject is selected for debulking the MM prior to administration to the subject of a T cell therapy comprising a dose of genetically engineered T cells expressing a chimeric antigen receptor (CAR) that binds to BCMA. In some embodiments, the method comprises (a) determining that a subject has a first serum soluble B cell maturation antigen (sBCMA) level higher than about 600 ng/ml; (b) debulking the MM: (c) determining that the subject has a post-debulking serum sBCMA level lower than about 600 ng/ml; and (d) administering to the subject a T cell therapy comprising a dose of genetically engineered T cells expressing a chimeric antigen receptor (CAR) that binds to BCMA.

In some embodiments, the debulking is carried out as described in Section II.B.

In some embodiments, debulking comprises administration of chemotherapy, radiation, or an immunomodulatory agent to the subject. In some embodiments, debulking comprises administration of chemotherapy to the subject. In some embodiments, the chemotherapy comprises melphalan, doxorubicin, or cyclophosphamide chemotherapy. In some embodiments, comprises administration of radiation to the subject. In some embodiments, the immunomodulatory agent is thalidomide, lenalidomide, or pomalidomide. In some embodiments, comprises administration of an immunomodulatory agent to the subject. In some embodiments, the immunomodulatory agent is a checkpoint inhibitor.

In some embodiments, the debulking is carried out within about three months before, within about two months before, within about one month before, within about three weeks before, within about two weeks before, or within about one week before administration of the T cell therapy to the subject. In some embodiments, the debulking is carried out within about three months before administration of the T cell therapy to the subject. In some embodiments, the debulking is carried out within about two months before administration of the T cell therapy to the subject. In some embodiments, the debulking is carried out within about one month before administration of the T cell therapy to the subject. In some embodiments, the debulking is carried out within about three weeks before administration of the T cell therapy to the subject. In some embodiments, the debulking is carried out within about two weeks before administration of the T cell therapy to the subject. In some embodiments, the debulking is carried out within about one week before administration of the T cell therapy to the subject. In some embodiments, the debulking is carried out prior to administration of a lymphodepleting therapy to the subject. In some embodiments, the debulking is carried out after the administration of a lymphodepleting therapy to a subject. In some embodiments, the subject is treated with a gamma-secretase inhibitor prior to administration of the T cell therapy to the subject.

In some embodiments, the method comprises determining that the subject has a serum sBCMA level higher or lower than about 590 ng/ml, about 580 ng/ml, about 570 ng/ml, about 560 ng/ml, about 550 ng/ml, about 540 ng/ml, about 530 ng/ml, about 520 ng/ml, about 510 ng/ml, or about 500 ng/ml. In some embodiments, the method comprises determining that the subject has a serum sBCMA level higher or lower than about 590 ng/ml. In some embodiments, the method comprises determining that the subject has a serum sBCMA level higher or lower than about 580 ng/ml. In some embodiments, the method comprises determining that the subject has a serum sBCMA level higher or lower than about 570 ng/ml. In some embodiments, the method comprises determining that the subject has a serum sBCMA level higher or lower than about 566 ng/ml. In some embodiments, the method comprises determining that the subject has a serum sBCMA level higher or lower than about 560 ng/ml. In some embodiments, the method comprises determining that the subject has a serum sBCMA level higher or lower than about 550 ng/ml. In some embodiments, the method comprises determining that the subject has a serum sBCMA level higher or lower than about 540 ng/ml. In some embodiments, the method comprises determining that the subject has a serum sBCMA level higher or lower than about 530 ng/ml. In some embodiments, the method comprises determining that the subject has a serum sBCMA level higher or lower than about 520 ng/ml. In some embodiments, the method comprises determining that the subject has a serum sBCMA level higher or lower than about 510 ng/ml. In some embodiments, the method comprises determining that the subject has a serum sBCMA level higher or lower than about 500 ng/ml.

In some embodiments, the subject is determined to have a serum sBCMA level higher or lower than about 590 ng/ml, about 580 ng/ml, about 570 ng/ml, about 560 ng/ml, about 550 ng/ml, about 540 ng/ml, about 530 ng/ml, about 520 ng/ml, about 510 ng/ml, or about 500 ng/ml. In some embodiments, the subject is determined to have a serum sBCMA level higher or lower than about 590 ng/ml. In some embodiments, the subject is determined to have a serum sBCMA level higher or lower than about 580 ng/ml. In some embodiments, the subject is determined to have a serum sBCMA level higher or lower than about 570 ng/ml. In some embodiments, the subject is determined to have a serum sBCMA level higher or lower than about 566 ng/ml. In some embodiments, the subject is determined to have a serum sBCMA level higher or lower than about 560 ng/ml. In some embodiments, the subject is determined to have a serum sBCMA level higher or lower than about 550 ng/ml. In some embodiments, the subject is determined to have a serum sBCMA level higher or lower than about 540 ng/ml. In some embodiments, the subject is determined to have a serum sBCMA level higher or lower than about 530 ng/ml. In some embodiments, the subject is determined to have a serum sBCMA level higher or lower than about 520 ng/ml. In some embodiments, the subject is determined to have a serum sBCMA level higher or lower than about 510 ng/ml. In some embodiments, the subject is determined to have a serum sBCMA level higher or lower than about 500 ng/ml.

ii. Response

In some embodiments, the methods comprise determining whether a subject has a serum soluble B cell maturation antigen (sBCMA) level higher or lower than about 600 ng/mL. In some embodiments, the methods comprise determining the level of serum sBCMA in a sample obtained from a subject. In some embodiments, based on the level of serum sBCMA being lower than a particular threshold, such as lower than 566 or 600 ng/mL, the subject is predicted to achieve a CR or sCR following administration of the T cell therapy.

In some embodiments, the methods comprise determining whether a subject has a serum sBCMA level lower than about 600 ng/mL, such as lower than about 566 ng/mL or 500 ng/mL. In some embodiments, if the subject is determined to have a serum sBCMA level lower than about 600 ng/mL, such as lower than about 566 ng/mL or 500 ng/mL, the subject is predicted to exhibit a CR or sCR following administration of a T cell therapy comprising a dose of genetically engineered T cells expressing a chimeric antigen receptor (CAR) that binds to BCMA (i.e. BCMA CAR T cells). In some embodiments, if the subject is determined to have a serum sBCMA level lower than about 600 ng/mL, the subject is about one time, two times, three times, four times, five times, six times, seven times, eight times, nine times, or about ten times more likely to achieve a CR or sCR following administration of the T cell therapy, as compared to a subject having a serum sBCMA level higher than about 600 ng/mL and administered the T cell therapy. In some embodiments, if the subject is determined to have a serum sBCMA level lower than about 600 ng/mL, the subject is about 10%, 20%, 30%, 40%, 50%, 60%, 70%, 80%, 90%, or 100% more likely to achieve a CR or sCR following administration of the T cell therapy, as compared to a subject having a serum sBCMA level higher than about 600 ng/mL and administered the T cell therapy. In some embodiments, if the subject is determined to have a serum sBCMA level lower than about 566 ng/mL, the subject is about one time, two times, three times, four times, five times, six times, seven times, eight times, nine times, or about ten times more likely to achieve a CR or sCR following administration of the T cell therapy, as compared to a subject having a serum sBCMA level higher than about 566 ng/mL and administered the T cell therapy. In some embodiments, if the subject is determined to have a serum sBCMA level lower than about 566 ng/mL, the subject is about 10%, 20%, 30%, 40%, 50%, 60%, 70%, 80%, 90%, or 100% more likely to achieve a CR or sCR following administration of the T cell therapy, as compared to a subject having a serum sBCMA level higher than about 566 ng/mL and administered the T cell therapy. In some embodiments, if the subject is determined to have a serum sBCMA level lower than about 500 ng/mL, the subject is about one time, two times, three times, four times, five times, six times, seven times, eight times, nine times, or about ten times more likely to achieve a CR or sCR following administration of the T cell therapy, as compared to a subject having a serum sBCMA level higher than about 500 ng/mL and administered the T cell therapy.

In some embodiments, the methods comprise determining whether a subject has a serum sBCMA level higher than about 600 ng/mL, such as higher than about 500 ng/mL or 566 ng/mL In some embodiments, if the subject is determined to have a serum sBCMA level higher than about 600 ng/mL, such as higher than about 500 ng/mL or 566 ng/mL, the subject is predicted not to exhibit a CR or sCR following administration of a T cell therapy comprising a dose of genetically engineered T cells expressing a chimeric antigen receptor (CAR) that binds to BCMA (i.e. BCMA CAR T cells). In some embodiments, if the subject is determined to have a serum sBCMA level higher than about 600 ng/mL, the subject is about one time, two times, three times, four times, five times, six times, seven times, eight times, nine times, or about ten times more likely not to achieve a CR or sCR following administration of the T cell therapy, as compared to a subject having a serum sBCMA level lower than about 600 ng/mL and administered the T cell therapy. In some embodiments, if the subject is determined to have a serum sBCMA level higher than about 600 ng/mL, the subject is about 10%, 20%, 30%, 40%, 50%, 60%, 70%, 80%, 90%, or 100% more likely not to achieve a CR or sCR following administration of the T cell therapy, as compared to a subject having a serum sBCMA level lower than about 600 ng/mL and administered the T cell therapy. In some embodiments, if the subject is determined to have a serum sBCMA level higher than about 566 ng/mL, the subject is about one time, two times, three times, four times, five times, six times, seven times, eight times, nine times, or about ten times more likely not to achieve a CR or sCR following administration of the T cell therapy, as compared to a subject having a serum sBCMA level lower than about 566 ng/mL and administered the T cell therapy. In some embodiments, if the subject is determined to have a serum sBCMA level higher than about 566 ng/mL, the subject is about 10%, 20%, 30%, 40%, 50%, 60%, 70%, 80%, 90%, or 100% more likely not to achieve a CR or sCR following administration of the T cell therapy, as compared to a subject having a serum sBCMA level lower than about 566 ng/mL and administered the T cell therapy. In some embodiments, if the subject is determined to have a serum sBCMA level higher than about 500 ng/mL, the subject is about one time, two times, three times, four times, five times, six times, seven times, eight times, nine times, or about ten times more likely not to achieve a CR or sCR following administration of the T cell therapy, as compared to a subject having a serum sBCMA level lower than about 500 ng/mL and administered the T cell therapy. In some embodiments, if the subject is determined to have a serum sBCMA level higher than about 500 ng/mL, the subject is about 10%, 20%, 30%, 40%, 50%, 60%, 70%, 80%, 90%, or 100% more likely not to achieve a CR or sCR following administration of the T cell therapy, as compared to a subject having a serum sBCMA level lower than about 500 ng/mL and administered the T cell therapy.

Thus, in some embodiments, the methods comprise predicting the response of a subject having a multiple myeloma (MM) to treatment with a T cell therapy comprising a dose of genetically engineered T cells, comprising determining that a subject has a serum soluble B cell maturation antigen (sBCMA) level lower than about 600 ng/ml, wherein, if the subject has a serum sBCMA level lower than about 600 ng/mL, the subject is predicted to achieve a complete response (CR) or stringent complete response (sCR).

In some embodiments, the method comprises determining that the subject has a serum sBCMA level higher or lower than about 590 ng/ml, about 580 ng/ml, about 570 ng/ml, about 560 ng/ml, about 550 ng/ml, about 540 ng/ml, about 530 ng/ml, about 520 ng/ml, about 510 ng/ml, or about 500 ng/ml. In some embodiments, the method comprises determining that the subject has a serum sBCMA level higher or lower than about 590 ng/ml. In some embodiments, the method comprises determining that the subject has a serum sBCMA level higher or lower than about 580 ng/ml. In some embodiments, the method comprises determining that the subject has a serum sBCMA level higher or lower than about 570 ng/ml. In some embodiments, the method comprises determining that the subject has a serum sBCMA level higher or lower than about 566 ng/ml. In some embodiments, the method comprises determining that the subject has a serum sBCMA level higher or lower than about 560 ng/ml. In some embodiments, the method comprises determining that the subject has a serum sBCMA level higher or lower than about 550 ng/ml. In some embodiments, the method comprises determining that the subject has a serum sBCMA level higher or lower than about 540 ng/ml. In some embodiments, the method comprises determining that the subject has a serum sBCMA level higher or lower than about 530 ng/ml. In some embodiments, the method comprises determining that the subject has a serum sBCMA level higher or lower than about 520 ng/ml. In some embodiments, the method comprises determining that the subject has a serum sBCMA level higher or lower than about 510 ng/ml. In some embodiments, the method comprises determining that the subject has a serum sBCMA level higher or lower than about 500 ng/ml.

In some embodiments, the subject is determined to have a serum sBCMA level higher or lower than about 590 ng/ml, about 580 ng/ml, about 570 ng/ml, about 560 ng/ml, about 550 ng/ml, about 540 ng/ml, about 530 ng/ml, about 520 ng/ml, about 510 ng/ml, or about 500 ng/ml. In some embodiments, the subject is determined to have a serum sBCMA level higher or lower than about 590 ng/ml. In some embodiments, the subject is determined to have a serum sBCMA level higher or lower than about 580 ng/ml. In some embodiments, the subject is determined to have a serum sBCMA level higher or lower than about 570 ng/ml. In some embodiments, the subject is determined to have a serum sBCMA level higher or lower than about 566 ng/ml. In some embodiments, the subject is determined to have a serum sBCMA level higher or lower than about 560 ng/ml. In some embodiments, the subject is determined to have a serum sBCMA level higher or lower than about 550 ng/ml. In some embodiments, the subject is determined to have a serum sBCMA level higher or lower than about 540 ng/ml. In some embodiments, the subject is determined to have a serum sBCMA level higher or lower than about 530 ng/ml. In some embodiments, the subject is determined to have a serum sBCMA level higher or lower than about 520 ng/ml. In some embodiments, the subject is determined to have a serum sBCMA level higher or lower than about 510 ng/ml. In some embodiments, the subject is determined to have a serum sBCMA level higher or lower than about 500 ng/ml.

### B. Heavy Chain Disease

i. Selection for Treatment and/or Debulking

In some embodiments, the methods comprise determining the presence of absence of IgG heavy chain disease (HCD) in a sample obtained from a subject. In some embodiments, based on the presence or absence of IgG HCD, the subject is selected for administration of a T cell therapy and/or debulking of the MM. In some embodiments, based on the presence or absence of IgG HCD, the subject is selected for administration of a T cell therapy. In some embodiments, based on the presence or absence of IgG HCD, the subject is selected for debulking of the MM. In some embodiments, based on the presence or absence of IgG HCD, the subject is selected for administration of a T cell therapy and debulking of the MM.

In some embodiments, the methods comprise determining whether a subject has an absence of IgG HCD. In some embodiments, if the subject is determined to have an absence of heavy chain disease, the subject is administered a T cell therapy comprising a dose of genetically engineered T cells expressing a chimeric antigen receptor (CAR) that binds to BCMA (i.e. BCMA CAR T cells). In some embodiments, if the subject is determined to have an absence of heavy chain disease, the subject is selected for administration of a T cell therapy comprising a dose of genetically engineered T cells expressing a chimeric antigen receptor (CAR) that binds to BCMA (i.e. BCMA CAR T cells).

In some embodiments, the methods comprise determining whether a subject has a presence of IgG HCD. In some embodiments, if the subject is determined to have a presence of IgG HCD, the subject is not administered a T cell therapy comprising a dose of genetically engineered T cells expressing a chimeric antigen receptor (CAR) that binds to BCMA (i.e. BCMA CAR T cells).

Thus in some embodiments, the methods comprise (a) determining that a subject has an absence of IgG heavy chain disease (HCD); and (b) administering to the subject a T cell therapy comprising a dose of genetically engineered T cells expressing a chimeric antigen receptor (CAR) that binds to BCMA. In some embodiments, the methods comprise administering to the subject a T cell therapy comprising a dose of genetically engineered T cells expressing a chimeric antigen receptor (CAR) that binds to B cell maturation antigen (BCMA), wherein the subject was previously determined to have an absence of IgG heavy chain disease (HCD). In some embodiments, the methods comprise selecting a subject having a multiple myeloma (MM) for treatment with a T cell therapy comprising a dose of genetically engineered T cells, comprising determining that a subject has an absence of IgG heavy chain disease (HCD), wherein, if the subject is determined to have an absence of IgG HCD, the subject is selected for administration with the T cell therapy.

In some embodiments, the methods comprise determining whether a subject has a presence of IgG HCD. In some embodiments, if the subject is determined to have a presence of IgG HCD, the multiple myeloma is debulked prior to administration of a T cell therapy comprising a dose of genetically engineered T cells expressing a chimeric antigen receptor (CAR) that binds to BCMA (i.e. BCMA CAR T cells). In some embodiments, if the subject is determined to have a presence of IgG HCD, the subject is selected for debulking of the multiple myeloma prior to administration of a T cell therapy comprising a dose of genetically engineered T cells expressing a chimeric antigen receptor (CAR) that binds to BCMA (i.e. BCMA CAR T cells). In some embodiments, the debulking is carried out as described in the section above or Section II.B.

In some embodiments, debulking comprises administration of chemotherapy, radiation, or an immunomodulatory agent to the subject. In some embodiments, debulking comprises administration of chemotherapy to the subject. In some embodiments, the chemotherapy comprises melphalan, doxorubicin, or cyclophosphamide chemotherapy. In some embodiments, comprises administration of radiation to the subject. In some embodiments, the immunomodulatory agent is thalidomide, lenalidomide, or pomalidomide. In some embodiments, comprises administration of an immunomodulatory agent to the subject. In some embodiments, the immunomodulatory agent is a checkpoint inhibitor.

In some embodiments, the debulking is carried out within about three months before, within about two months before, within about one month before, within about three weeks before, within about two weeks before, or within about one week before administration of the T cell therapy to the subject. In some embodiments, the debulking is carried out within about three months before administration of the T cell therapy to the subject. In some embodiments, the debulking is carried out within about two months before administration of the T cell therapy to the subject. In some embodiments, the debulking is carried out within about one month before administration of the T cell therapy to the subject. In some embodiments, the debulking is carried out within about three weeks before administration of the T cell therapy to the subject. In some embodiments, the debulking is carried out within about two weeks before administration of the T cell therapy to the subject. In some embodiments, the debulking is carried out within about one week before administration of the T cell therapy to the subject. In some embodiments, the debulking is carried out prior to administration of a lymphodepleting therapy to the subject. In some embodiments, the debulking is carried out after the administration of a lymphodepleting therapy to a subject. In some embodiments, the subject is treated with a gamma-secretase inhibitor prior to administration of the T cell therapy to the subject.

ii. Response

In some embodiments, the methods comprise determining is a subject has an absence of IgG heavy chain disease (HCD). In some embodiments, the methods comprise determining the presence of absence of IgG heavy chain disease (HCD) in a sample obtained from a subject. In some embodiments, based on the absence of IgG HCD, the subject is predicted to achieve a CR or sCR following administration of the T cell therapy. Conversely, in some embodiments, based on the presence of IgG HCD, the subject is predicted not to achieve a CR or sCR following administration of the T cell therapy.

In some embodiments, the methods comprise determining whether a subject has an absence of IgG HCD. In some embodiments, if the subject is determined to have an absence of IgG HCD, the subject is predicted to exhibit a CR or sCR following administration of a T cell therapy comprising a dose of genetically engineered T cells expressing a chimeric antigen receptor (CAR) that binds to BCMA (i.e. BCMA CAR T cells). In some embodiments, if the subject is determined to have an absence of IgG HCD, the subject is about one time, two times, three times, four times, five times, six times, seven times, eight times, nine times, or about ten times more likely to achieve a CR or sCR following administration of the T cell therapy, as compared to a subject having a presence of IgG HCD and administered the T cell therapy.

In some embodiments, if the subject is determined to have an absence of IgG HCD, the subject is about eight times, nine times, ten times, eleven times, or twelve times more likely to achieve a CR or sCR following administration of the T cell therapy, as compared to a subject having a presence of IgG HCD and administered the T cell therapy. In some embodiments, the methods comprise determining whether a subject has a presence of IgG HCD. In some embodiments, if the subject is determined to have a presence of IgG HCD, the subject is predicted not to exhibit a CR or sCR following administration of a T cell therapy comprising a dose of genetically engineered T cells expressing a chimeric antigen receptor (CAR) that binds to BCMA (i.e. BCMA CAR T cells). In some embodiments, if the subject is determined to have a presence of IgG HCD, the subject is about one time, two times, three times, four times, five times, six times, seven times, eight times, nine times, or about ten times more likely not to achieve a CR or sCR following administration of the T cell therapy, as compared to a subject having an absence of IgG HCD and administered the T cell therapy. In some embodiments, if the subject is determined to have a presence of IgG HCD, the subject is about eight times, nine times, ten times, eleven times, or twelve times more likely not to achieve a CR or sCR following administration of the T cell therapy, as compared to a subject having an absence of IgG HCD and administered the T cell therapy.

Thus, in some embodiments, the methods comprise predicting the response of a subject having a multiple myeloma (MM) to treatment with a T cell therapy comprising a dose of genetically engineered cells, comprising determining that a subject has an absence of IgG heavy chain disease (HCD), wherein, if the subject has an absence of IgG HCD the subject is predicted to achieve a complete response (CR) or stringent complete response (sCR). In some embodiments, the methods comprise predicting the response of a subject having a multiple myeloma (MM) to treatment with a T cell therapy comprising a dose of genetically engineered cells, comprising determining that a subject has a presence of IgG heavy chain disease (HCD), wherein, if the subject has a presence of IgG HCD the subject is predicted not to achieve a complete response (CR) or stringent complete response (sCR).

### C. Other Biomarkers

In some of any embodiments, the methods involve assessing the number of copies of vector in the dose of engineered cells. In some embodiments, based on the number of copies of vector in the dose of engineered cells, the subject is predicted to achieve a CR or sCR following administration of the T cell therapy or is predicted not to achieve a CR or sCR following administration of the T cell therapy.

In some embodiments, the methods involve comparing the number of copies of vector in the dose of engineered cells to a threshold level, thereby determining a likelihood that the subject will achieve a CR or sCR to the T cell therapy. In some aspects, the threshold level is determined based on the mean or median values and values within a range or standard deviation of the mean or median values of the number of copies of vector in the dose of engineered cells provided to a group of subjects, wherein each of the subjects of the group went on to exhibit a CR or sCR, or did not go on to exhibit a CR or sCR after receiving the dose of engineered cells. In some embodiments, if the number of copies of vector in the dose of engineered cells to be provided to the subject is lower than the threshold level, the subject is predicted not to exhibit a CR or sCR to the T cell therapy. In some embodiments, if the number of copies of vector in the dose of engineered cells to be provided to the subject is higher than the threshold level, the subject is predicted to exhibit a CR or sCR to the T cell therapy.

In some of any embodiments, the methods involve assessing the β-2 microglobulin level, such as in a sample obtained from a subject. In some embodiments, based on the β-2 microglobulin level in a sample obtained from a subject, the subject is predicted to achieve a CR or sCR following administration of the T cell therapy or is predicted not to achieve a CR or sCR following administration of the T cell therapy.

In some embodiments, the methods involve comparing, individually, the level, amount or concentration of β-2 microglobulin from a sample obtained from a subject to a threshold level, thereby determining a likelihood that the subject will achieve a CR or sCR to the T cell therapy. In some aspects, the threshold level is determined based on the mean or median values and values within a range or standard deviation of the mean or median values of the level, amount or concentration of β-2 microglobulin, in biological samples obtained from a group of subjects prior to receiving a T cell therapy, wherein each of the subjects of the group went on to exhibit a CR or sCR, or did not go on to exhibit a CR or sCR. In some embodiments, if the level, amount or concentration of β-2 microglobulin from the sample obtained from a subject is higher than the threshold level, the subject is predicted not to exhibit a CR or sCR to the T cell therapy. In some embodiments, the threshold level is about 5.5 mg/mL. In some embodiments, if the level, amount or concentration of β-2 microglobulin from the sample obtained from a subject is higher than about 5.5 mg/mL, the subject is predicted not to exhibit a CR or sCR to the T cell therapy. In some embodiments, if the level, amount or concentration of β-2 microglobulin from the sample obtained from a subject is lower than the threshold level, the subject is predicted to exhibit a CR or sCR to the T cell therapy. In some embodiments, the threshold level is about 3.5 mg/mL. In some embodiments, if the level, amount or concentration of β-2 microglobulin from the sample obtained from a subject is lower than about 3.5 mg/mL, the subject is predicted to exhibit a CR or sCR to the T cell therapy.

In some of any embodiments, the methods involve assessing the D-dimer level, such as in a sample obtained from a subject. In some embodiments, the sample is a blood sample. In some embodiments, the methods involve comparing, individually, the level, amount or concentration of D-dimer from a sample obtained from a subject to a threshold level, thereby determining a likelihood that the subject will achieve a CR or sCR to the T cell therapy. In some aspects, the threshold level is determined based on the mean or median values and values within a range or standard deviation of the mean or median values of the level, amount or concentration of D-dimer, in biological samples obtained from a group of subjects prior to receiving a T cell therapy, wherein each of the subjects of the group went on to exhibit a CR or sCR, or did not go on to exhibit a CR or sCR. In some embodiments, if the level, amount or concentration of D-dimer from the sample obtained from a subject is higher than the threshold level, the subject is predicted not to exhibit a CR or sCR to the T cell therapy. In some embodiments, the threshold level is about 0.8 mg/L, about 0.9 mg/L, about 1.0 mg/L, about 1.1 mg/L, about 1.2 mg/L, or about 1.3 mg/L. In some embodiments, if the level, amount or concentration of D-dimer from the sample obtained from a subject is higher than 0.8 mg/L, about 0.9 mg/L, about 1.0 mg/L, about 1.1 mg/L, about 1.2 mg/L, or about 1.3 mg/L, the subject is predicted not to exhibit a CR or sCR to the T cell therapy. In some embodiments, if the level, amount or concentration of D-dimer from the sample obtained from a subject is lower than the threshold level, the subject is predicted to exhibit a CR or sCR to the T cell therapy. In some embodiments, the threshold level is about 0.8 mg/L, about 0.7 mg/L, about 0.6 mg/L, about 0.5 mg/L, about 0.4 mg/L, or about 0.3 mg/L. In some embodiments, if the level, amount or concentration of D-dimer from the sample obtained from a subject is lower than about 0.8 mg/L, about 0.7 mg/L, about 0.6 mg/L, about 0.5 mg/L, about 0.4 mg/L, or about 0.3 mg/L, the subject is predicted to exhibit a CR or sCR to the T cell therapy.

In some of any embodiments, the methods involve assessing the ferritin level, such as in a sample obtained from a subject. In some embodiments, based on the ferritin level in a sample from a subject, the subject is predicted to achieve a CR or sCR following administration of the T cell therapy or is predicted not to achieve a CR or sCR following administration of the T cell therapy.

In some embodiments, the methods involve comparing, individually, the level, amount or concentration of ferritin from a sample obtained from a subject to a threshold level, thereby determining a likelihood that the subject will achieve a CR or sCR to the T cell therapy. In some aspects, the threshold level is determined based on the mean or median values and values within a range or standard deviation of the mean or median values of the level, amount or concentration of ferritin, in biological samples obtained from a group of subjects prior to receiving a T cell therapy, wherein each of the subjects of the group went on to exhibit a CR or sCR, or did not go on to exhibit a CR or sCR. In some embodiments, if the level, amount or concentration of ferritin from the sample obtained from a subject is higher than the threshold level, the subject is predicted not to exhibit a CR or sCR to the T cell therapy. In some embodiments, the threshold level is about 350 μg/L, about 400 μg/L, about 450 μg/L, about 500 μg/L, about 550 μg/L, or about 600 μg/L. In some embodiments, if the level, amount or concentration of ferritin from the sample obtained from a subject is higher than about 350 μg/L, about 400 μg/L, about 450 μg/L, about 500 μg/L, about 550 μg/L, or about 600 μg/L, the subject is predicted not to exhibit a CR or sCR to the T cell therapy. In some embodiments, if the level, amount or concentration of ferritin from the sample obtained from a subject is lower than the threshold level, the subject is predicted to exhibit a CR or sCR to the T cell therapy. In some embodiments, the threshold level is about 300 μg/L, about 250 μg/L, about 200 μg/L, about 150 μg/L, about 100 μg/L, or about 50 μg/L. In some embodiments, if the level, amount or concentration of ferritin from the sample obtained from a subject is lower than about 300 μg/L, about 250 μg/L, about 200 μg/L, about 150 μg/L, about 100 μg/L, or about 50 μg/L, the subject is predicted to exhibit a CR or sCR to the T cell therapy.

In some of any embodiments, the methods involve assessing the sodium level, such as in a sample obtained from a subject. In some embodiments, based on the sodium level in a sample from a subject, the subject is predicted to achieve a CR or sCR following administration of the T cell therapy or is predicted not to achieve a CR or sCR following administration of the T cell therapy.

In some embodiments, the methods involve comparing, individually, the level, amount or concentration of sodium from a sample obtained from a subject to a threshold level, thereby determining a likelihood that the subject will achieve a CR or sCR to the T cell therapy. In some aspects, the threshold level is determined based on the mean or median values and values within a range or standard deviation of the mean or median values of the level, amount or concentration of sodium, in biological samples obtained from a group of subjects prior to receiving a T cell therapy, wherein each of the subjects of the group went on to exhibit a CR or sCR, or did not go on to exhibit a CR or sCR. In some embodiments, if the level, amount or concentration of sodium from the sample obtained from a subject is lower than the threshold level, the subject is predicted not to exhibit a CR or sCR to the T cell therapy. In some embodiments, the threshold level is about 140 mmol/L, about 139 mmol/L, about 138 mmol/L, about 137 mmol/L, about 136 mmol/L, or about 135 mmol/L. In some embodiments, if the level, amount or concentration of sodium from the sample obtained from a subject is lower than about 140 mmol/L, about 139 mmol/L, about 138 mmol/L, about 137 mmol/L, about 136 mmol/L, or about 135 mmol/L, the subject is predicted not to exhibit a CR or sCR to the T cell therapy. In some embodiments, if the level, amount or concentration of sodium from the sample obtained from a subject is higher than the threshold level, the subject is predicted to exhibit a CR or sCR to the T cell therapy. In some embodiments, the threshold level is about 140 mmol/L, about 141 mmol/L, about 142 mmol/L, about 143 mmol/L, about 144 mmol/L, or about 145 mmol/L. In some embodiments, if the level, amount or concentration of sodium from the sample obtained from a subject is higher than about 140 mmol/L, about 141 mmol/L, about 142 mmol/L, about 143 mmol/L, about 144 mmol/L, or about 145 mmol/L, the subject is predicted to exhibit a CR or sCR to the T cell therapy. In some of any embodiments, the methods involve assessing the prothrombin time-international normalized ratio (PT-INR), such as in a sample obtained from a subject. In some embodiments, the sample is a blood sample. In some embodiments, the methods involve comparing PT-INR from a sample obtained from a subject to a threshold PT-INR, thereby determining a likelihood that the subject will achieve a CR or sCR to the T cell therapy. In some aspects, the threshold PT-INR is determined based on the mean or median values and values within a range or standard deviation of the mean or median values of the PT-INR, in biological samples obtained from a group of subjects prior to receiving a T cell therapy, wherein each of the subjects of the group went on to exhibit a CR or sCR, or did not go on to exhibit a CR or sCR. In some embodiments, if the PT-INR of the sample obtained from a subject is higher than the threshold PT-INR, the subject is predicted not to exhibit a CR or sCR to the T cell therapy. In some embodiments, if the PT-INR of the sample obtained from a subject is higher than the threshold PT-INR, the subject is about 100 times, 150 times, 200 times, 250 times, or 300 times less likely to exhibit a CR or sCR to the T cell therapy, e.g. as compared to a subject for whom a sample has a PT-INR at or lower than the threshold PT-INR. In some embodiments, if the PT-INR of the sample obtained from a subject is lower than the threshold level, the subject is predicted to exhibit a CR or sCR to the T cell therapy. In some embodiments, if the PT-INR of the sample obtained from a subject is lower than the threshold PT-INR, the subject is about 100 times, 150 times, 200 times, 250 times, or 300 times more likely to exhibit a CR or sCR to the T cell therapy, e.g. as compared to a subject for whom a sample has a PT-1NR at or higher than the threshold PT-1NR.

### II. METHODS OF TREATMENT AND USES

Also provided herein are methods of using and uses of the BCMA-binding molecules, recombinant receptors, engineered cells, and pharmaceutical compositions and formulations thereof, such as in the treatment of multiple myeloma, and/or detection, selection, diagnostic, and prognostic methods. Among such methods, such as methods of treatment and uses, are those that involve administering to a subject engineered cells, such as a plurality of engineered cells, expressing the provided anti-BCMA recombinant receptors (e.g., BCMA CAR T cells).

### A. Subjects and Methods of Use

Also provided are methods of administering and uses, such as therapeutic uses, of the anti-BCMA recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., recombinant receptors), cells (e.g., engineered cells), or compositions containing the same, to a subject having a multiple myeloma (MM). In some embodiments, the molecule, cell, and/or composition is/arc administered in an effective amount to effect treatment of the MM. Provided herein are uses of the recombinant receptors (e.g., CARs), and cells (e.g., engineered cells) in such methods and treatments, and in the preparation of a medicament in order to carry out such therapeutic methods. In some embodiments, the methods are carried out by administering the binding molecules or cells, or compositions comprising the same, to the subject having the MM. In some embodiments, the methods thereby treat the MM in the subject. Also provided herein are of use of any of the compositions, such as pharmaceutical compositions provided herein, for the treatment of a multiple myeloma (MM), such as use in a treatment regimen.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or subject being treated. As sufficient or significant delay can, in effect, encompass prevention, in that the subject does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, binding molecule, antibody, cells, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, antibodies, cells, and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As used herein, a “subject” or an “individual” is a human.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al: U.S. Pat. No. 4,690,915 to Rosenberg: Rosenberg (2011) Nat Rev Clin Oncol. 8(10):577-85). See, e.g., Themeli et al. (2013) Nat Biotechnol. 31(10): 928-933: Tsukahara et al. (2013) Biochem Biophys Res Commun 438(1): 84-9; Davila et al. (2013) PLoS ONE 8(4): e61338.

Among the diseases to be treated is multiple myeloma (MM), which is associated with BCMA expression. Vee Coquery et al., Crit Rev Immunol., 2012, 32(4):287-305 for a review of BCMA. Since BCMA has been implicated in mediating tumor cell survival, it is a potential target for cancer therapy. Chimeric antigen receptors containing mouse anti-human BCMA antibodies and cells expressing such chimeric receptors have been previously described. See Carpenter et al., Clin Cancer Res., 2013, 19(8):2048-2060.

In some embodiments the multiple myeloma (MM) is a high risk MM or a relapsed and/or refractory multiple myeloma. In some embodiments the multiple myeloma (MM) is a high risk MM. In some embodiments, a high risk MM comprises IMWG high risk cytogenetics. In some of any embodiments, at the time of administration of the cell therapy, the subject has IMWG high risk cytogenetics. In some embodiments, high risk cytogenetics comprise del(17p), t(4:14) and t(14;16). In some embodiments the multiple myeloma (MM) is a relapsed and/or refractory multiple myeloma. In some embodiments the multiple myeloma (MM) is a relapsed and refractory multiple myeloma (r/r MM). In some of any embodiments, at the time of administration, the subject has a R/R MM. In some embodiments, the methods may identify a subject who has, is suspected to have, or is at risk for developing a multiple myeloma. Hence, provided are methods for identifying subjects with multiple myeloma and selecting them for treatment with and/or administering to them any of the BCMA-binding recombinant receptors (e.g., CARs) described herein, or engineered cells expressing the same.

In some embodiments, the subject has a serum soluble B cell maturation antigen (sBCMA) level lower than about 600 ng/mL. In some embodiments, the methods include determining that a subject has a serum sBCMA level lower than about 600 ng/mL. In some embodiments, the subject has a serum soluble B cell maturation antigen (sBCMA) level lower than about 566 ng/mL. In some embodiments, the methods include determining that a subject has a serum sBCMA level lower than about 566 ng/mL. In some embodiments, the subject has a serum soluble B cell maturation antigen (sBCMA) level lower than about 500 ng/mL. In some embodiments, the methods include determining that a subject has a serum sBCMA level lower than about 500 ng/mL. In some embodiments, the serum sBCMA level is determined prior to administration of a lymphodepleting therapy to the subject, prior to leukapheresis, and/or prior to administration of a T cell therapy to the subject. In some embodiments, the serum sBCMA level is determined prior to administration of a lymphodepleting therapy to the subject. In some embodiments, the serum sBCMA level is determined prior to leukapheresis. In some embodiments, the serum sBCMA is determined prior to administration of a T cell therapy to the subject. In some embodiments, the provide methods and for selecting subjects for treatment with a T cell therapy. In some embodiments, if a subject is determined to have a serum sBCMA level lower than about 600 ng/mL, the subject is selected for treatment. In some embodiments, if a subject is determined to have a serum sBCMA level higher than about 600 ng/mL, the subject is not selected for treatment. In some embodiments, if a subject is determined to have a serum sBCMA level lower than about 566 ng/mL, the subject is selected for treatment. In some embodiments, if a subject is determined to have a serum sBCMA level higher than about 566 ng/mL, the subject is not selected for treatment. In some embodiments, if a subject is determined to have a serum sBCMA level lower than about 500 ng/mL, the subject is selected for treatment. In some embodiments, if a subject is determined to have a serum sBCMA level higher than about 500 ng/mL, the subject is not selected for treatment. In some embodiments, if a subject is determined to have a serum sBCMA level higher than about 600 ng/mL, the subject is selected for debulking the multiple myeloma prior to administration of the T cell therapy to the subject. In some embodiments, if a subject is determined to have a serum sBCMA level higher than about 566 ng/mL, the subject is selected for debulking the multiple myeloma prior to administration of the T cell therapy to the subject. In some embodiments, if a subject is determined to have a serum sBCMA level higher than about 500 ng/mL, the subject is selected for debulking the multiple myeloma prior to administration of the T cell therapy to the subject.

In some embodiments, the subject has an absence of IgG heavy chain disease (HCD). In some embodiments, the methods include determining that a subject has an absence of IgG HCD. In some embodiments, the absence of IgG HCD is determined prior to administration of a lymphodepleting therapy to the subject, prior to leukapheresis, and/or prior to administration of a T cell therapy to the subject. In some embodiments, the absence of IgG HCD is determined prior to administration of a lymphodepleting therapy to the subject. In some embodiments, the absence of IgG HCD is determined prior to leukapheresis. In some embodiments, the absence of IgG HCD is determined prior to administration of a T cell therapy to the subject. In some embodiments, the provide methods and for selecting subjects for treatment with a T cell therapy. In some embodiments, if a subject is determined to have an absence of IgG HCD, the subject is selected for treatment. In some embodiments, if a subject is determined to have a presence of IgG HCD, the subject is not selected for treatment. In some embodiments, if a subject is determined to have a presence of IgG HCD, the subject is selected for debulking the multiple myeloma prior to administration of the T cell therapy to the subject.

In some embodiments, the subject has persistent or relapsed disease, e.g., following treatment with a prior line of therapy. In some embodiments, prior to the administration of the T cell therapy, the subject has received one or more prior therapies. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies. In some embodiments, the subject has received 3 or more prior therapies.

In some embodiments, each of the three or more prior lines of therapy comprised two consecutive cycles. In some embodiments, each of the three or more prior lines of therapy comprised two consecutive cycles, unless progressive disease was the best response to the line of therapy. In some embodiments, progressive disease is progression within 60 days after the last dose of the line of therapy. In some embodiments, the subject is refractory to the last of the three or more prior lines of therapy. In some embodiments, the three or more prior lines of therapy comprise a proteasome inhibitor (PI), an immunomodulatory agent, and an anti-CD38 antibody.

In some of any embodiments, the immunomodulatory agent is selected from among thalidomide, lenalidomide and pomalidomide. In some of any embodiments, the proteasome inhibitor is selected from among bortezomib, carfilzomib and ixazomib. In some of any embodiments, the anti-CD38 antibody is or comprises daratumumab.

In some embodiments, measurable disease criteria for multiple myeloma can include (1) serum M-protein 1 g/dL or greater; (2) Urine M-protein 200 mg or greater/24 hour; (3) involved serum free light chain (sFLC) level 10 mg/dL or greater, with abnormal κ to λ ratio. In some cases, light chain disease is acceptable only for subjects without measurable disease in the serum or urine. In some embodiments, the subject has measurable disease at the time of the administration of the T cell therapy. In some embodiments, the measurable disease comprises (i) serum M-protein greater or equal to 1.0 g/dL; (ii) urine M-protein greater or equal to 200 mg/24 h; and/or (iii) involved serum free light chain (FLC) level greater or equal to 10 mg/dL if serum FLC ratio is abnormal. In some embodiments, the measurable disease comprises serum M-protein greater or equal to 1.0 g/dL. In some embodiments, the measurable disease comprises urine M-protein greater or equal to 200 mg/24 h. In some embodiments, the measurable disease comprises involved serum free light chain (FLC) level greater or equal to 10 mg/dL if serum FLC ratio is abnormal. In some embodiments, the measurable disease comprises (i) serum M-protein greater or equal to 1.0 g/dL; (ii) urine M-protein greater or equal to 200 mg/24 h; and (iii) involved serum free light chain (FLC) level greater or equal to 10 mg/dL if serum FLC ratio is abnormal. See Kumar et al., Lancet Oncol (2016) 17(8):e328-46.

In some embodiments, the subject has adequate organ function.

In some embodiments, the subject is 18 years of age or older.

In some embodiments, the method can involve including or excluding particular subjects for treatment with the T cell therapy, based on particular criteria, diagnosis or indication. In some embodiments, at the time of administration of the T cell therapy or pre-treatment lymphodepleting chemotherapy, the subject does not have known central nervous system (CNS) involvement with myeloma. In some embodiments, at the time of administration of the T cell therapy or pre-treatment lymphodepleting chemotherapy, the subject does not have a history or presence of clinically relevant CNS pathology. In some embodiments, at the time of administration of the T cell therapy or pre-treatment lymphodepleting chemotherapy, the subject has not had active or history of plasma cell leukemia (PCL). In some embodiments, at the time of administration of the T cell therapy or pre-treatment lymphodepleting chemotherapy, the subject does not have solitary plasmacytomas or non-secretory myeloma without other evidence of measurable disease. In some embodiments, at the time of administration of the T cell therapy or pre-treatment lymphodepleting chemotherapy, the subject does not have history of an allogeneic hematopoietic stem cell transplantation. In some embodiments, at the time of administration of the T cell therapy or pre-treatment lymphodepleting chemotherapy, the subject does not have history of treatment with any gene therapy-based therapeutic for cancer. In some embodiments, at the time of administration of the T cell therapy or pre-treatment lymphodepleting chemotherapy, the subject does not have history of treatment with an investigational cellular therapy for cancer. In some embodiments, at the time of administration of the T cell therapy or pre-treatment lymphodepleting chemotherapy, the subject does not have a history of treatment with a BCMA targeted therapy.

In some embodiments, the assessment for the criteria, diagnosis or indication can be performed at the time of screening the subjects for eligibility or suitability of treatment according to the provided methods, at various steps of the treatment regimen, at the time of receiving lymphodepleting therapy, and/or at or immediately prior to the initiation of administration of the engineered cells or composition thereof.

Thus, the provided methods and uses include methods and uses for adoptive cell therapy. In some embodiments, the methods include administration of the cells or a composition containing the cells to a subject, tissue, or cell, such as one having, at risk for, or suspected of having a multiple myeloma. In some embodiments, the cells, populations, and compositions are administered to a subject having a multiple myeloma, e.g., via adoptive cell therapy, such as adoptive T cell therapy. In some embodiments, the cells or compositions are administered to the subject, such as a subject having or at risk for a multiple myeloma. In some aspects, the methods thereby treat, e.g., ameliorate one or more symptom of a multiple myeloma, such as by lessening tumor burden.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the T cell therapy, e.g., adoptive cell therapy, e.g., adoptive T cell therapy, is carried out by autologous transfer, in which the cells are isolated and/or otherwise prepared from the subject who is to receive the T cell therapy, or from a sample derived from such a subject. Thus, in some aspects, the cells are derived from a subject, e.g., patient, in need of a treatment and the cells, following isolation and processing are administered to the same subject.

In some embodiments, the T cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The subject can be male or female and can be any suitable age, including infant, juvenile, adolescent, adult, and geriatric subjects. In some embodiments, the subject is an adult (i.e. 18 years of age or older).

In some embodiments, the dose and/or frequency of administration is determined based on efficacy and/or response. In some embodiments, efficacy is determined by evaluating disease status. Exemplary methods for assessing disease status include: measurement of M protein in biological fluids, such as blood and/or urine, by electrophoresis and immunofixation: quantification of sFLC (κ and λ) in blood; skeletal survey; and imaging by positron emission tomography (PET)/computed tomography (CT) in subjects with extramedullary disease. In some embodiments, disease status can be evaluated by bone marrow examination. In some examples, dose and/or frequency of administration is determined by the expansion and persistence of the recombinant receptor or cell in the blood and/or bone marrow. In some embodiments, dose and/or frequency of administration is determined based on the antitumor activity of the recombinant receptor or engineered cell. In some embodiments antitumor activity is determined by the overall response rate (ORR) and/or International Myeloma Working Group (IMWG) Uniform Response Criteria (see Kumar et al. (2016) Lancet Oncol 17(8):e328-346). In some embodiments, response is evaluated using minimal residual disease (MRD) assessment. In some embodiments. MRD can be assessed by methods such as flow cytometry and high-throughput sequencing, e.g., deep sequencing. In some embodiments, response is evaluated based on the duration of response following administration of the recombinant receptor or cells. In some examples, dose and/or frequency of administration can be based on toxicity. In some embodiments, dose and/or frequency can be determined based on health-related quality of life (HRQoL) of the subject to which the recombinant receptor and/or cells is/are administered. In some embodiments, dose and/or frequency of administration can be changed, i.e., increased or decreased, based on any of the above criteria.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sorensen et al., (1993) Br J Cancer 67(4) 773-775. In some embodiments, the subjects that are to be administered according to the methods or treatment regimen provided herein include those with an ECOG performance status of 0 or 1. In some embodiments, the subject has an Eastern Cooperative Oncology Group (ECOG) performance status of 0 or 1. In some embodiments, the subject has an Eastern Cooperative Oncology Group (ECOG) performance status of 0. In some embodiments, the subject has an Eastern Cooperative Oncology Group (ECOG) performance status of 1.

In some embodiments, the administration can treat the subject despite the subject having become resistant to another therapy. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, when administered to subjects according to the embodiments described herein, the dose or the composition is capable of achieving stringent complete response (sCR) at least 20%, 30%, 40% 50%, 60% or 70% of subjects that were administered. In some embodiments, when administered to subjects according to the embodiments described herein, the dose or the composition is capable of achieving complete response (CR) at least 20%, 30%, 40% 50%, 60% or 70% of subjects that were administered. In some aspects, particular response to the treatment, e.g., according to the methods provided herein, can be assessed based on the International Myeloma Working Group (IMWG) Uniform Response Criteria (see Kumar et al. (2016) Lancet Oncol 17(8):e328-346).

In some embodiments, toxicity and/or side-effects of treatment can be monitored and used to adjust dose and/or frequency of administration of the recombinant receptor, e.g., CAR, cells, and or compositions. For example, adverse events and laboratory abnormalities can be monitored and used to adjust dose and/or frequency of administration. Adverse events include infusion reactions, cytokine release syndrome (CRS), neurotoxicity, macrophage activation syndrome, and tumor lysis syndrome (TLS). Any of such events can establish dose-limiting toxicities and warrant decrease in dose and/or a termination of treatment. Other side effects or adverse events which can be used as a guideline for establishing dose and/or frequency of administration include non-hematologic adverse events, which include but are not limited to fatigue, fever or febrile neutropenia, increase in transaminases for a set duration (e.g., less than or equal to 2 weeks or less than or equal to 7 days), headache, bone pain, hypotension, hypoxia, chills, diarrhea, nausea/vomiting, neurotoxicity (e.g., confusion, aphasia, seizures, convulsions, lethargy, and/or altered mental status), disseminated intravascular coagulation, other asymptomatic non-hematological clinical laboratory abnormalities. such as electrolyte abnormalities. Other side effects or adverse events which can be used as a guideline for establishing dose and/or frequency of administration include hematologic adverse events, which include but are not limited to neutropenia, leukopenia, thrombocytopenia, animal, and/or B-cell aplasia and hypogammaglobinemia.

In some embodiments, treatment according to the provided methods can result in a lower rate and/or lower degree of toxicity, toxic outcome or symptom, toxicity-promoting profile, factor, or property, such as a symptom or outcome associated with or indicative of cytokine release syndrome (CRS) or neurotoxicity, such as severe CRS or severe neurotoxicity, for example, compared to administration of other therapies.

In some embodiments, the subject may receive a bridging therapy after leukapheresis and before lymphodepleting chemotherapy. A treating physician can determine if bridging therapy is necessary, for example for disease control, during manufacturing of the provided composition or cells. In some embodiments, bridging therapies are discontinued prior to initiation of lymphodepletion. In some embodiments, bridging therapies are discontinued 1 day, 2 days 3 days, 4 days, 5 days, 7 days, 10 days, 14 days, 21 days, 28 days, 45 days, or 60 days before lymphodepletion.

Once the cells are administered to a mammal (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR expressed by the cells in some embodiments is conjugated either directly or indirectly through a linker to a targeting moiety. The practice of conjugating compounds, e.g., the CAR, to targeting moieties is known in the art. See, for instance, Wadwa et al., J. Drug Targeting, 3(2):111 (1995), and U.S. Pat. No. 5,087,616.

### B. Tumor Debulking

In some embodiments of the methods provided herein, the methods include debulking the multiple myeloma (MM) prior to administration of the T cell therapy to the subject. In some embodiments of the methods provided herein, the methods include selecting the subject for debulking the MM prior to administration of the T cell therapy to the subject.

In some embodiments, the methods comprise determining that the subject has a presence of IgG heavy chain disease (HCD) and/or a serum soluble B cell maturation antigen (sBCMA) level higher than about 600 ng/mL. In some embodiments, the methods comprise determining that the subject has a presence of IgG heavy chain disease (HCD) and/or a serum soluble B cell maturation antigen (sBCMA) level higher than about 566 ng/mL. In some embodiments, the methods comprise determining that the subject has a presence of IgG heavy chain disease (HCD) and/or a serum soluble B cell maturation antigen (sBCMA) level higher than about 500 ng/mL. In some embodiments, the subject has been determined to have a presence of IgG HCD and/or serum sBCMA level higher than about 600 ng/mL. In some embodiments, the subject has been determined to have a presence of IgG HCD and/or serum sBCMA level higher than about 566 ng/mL. In some embodiments, the subject has been determined to have a presence of IgG HCD and/or serum sBCMA level higher than about 500 ng/mL.

In some embodiments, the method comprises determining that the subject has a serum sBCMA level higher than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL. In some embodiments, the subject is determined to have a serum sBCMA level higher than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL. In some embodiments, the method comprises determining that the subject has serum sBCMA level higher than about 566 ng/mL. In some embodiments, the subject is determined to have a serum sBCMA level higher than about 566 ng/mL.

In some embodiments, if the subject is determined to have a presence of IgG HCD and/or serum sBCMA level higher than about 600 ng/mL, the method comprises debulking the MM. In some embodiments, if the subject is determined to have a presence of IgG HCD and/or serum sBCMA level higher than about 566 ng/mL, the method comprises debulking the MM. In some embodiments, if the subject is determined to have a presence of IgG HCD and/or serum sBCMA level higher than about 500 ng/mL, the method comprises debulking the MM. In some embodiments, if the subject is determined to have a presence of IgG HCD and/or serum sBCMA level higher than about 600 ng/mL, the MM is debulked. In some embodiments, if the subject is determined to have a presence of IgG HCD and/or serum sBCMA level higher than about 566 ng/mL, the MM is debulked. In some embodiments, if the subject is determined to have a presence of IgG HCD and/or serum sBCMA level higher than about 500 ng/mL, the MM is debulked. In some embodiments, if the subject is determined to have a presence of IgG HCD and/or serum sBCMA level higher than about 600 ng/mL, the subject is selected for debulking. In some embodiments, if the subject is determined to have a presence of IgG HCD and/or serum sBCMA level higher than about 566 ng/mL, the subject is selected for debulking. In some embodiments, if the subject is determined to have a presence of IgG HCD and/or serum sBCMA level higher than about 500 ng/mL, the subject is selected for debulking.

In some embodiments, the methods comprise determining that the subject has a serum sBCMA level higher than about 600 ng/mL. In some embodiments, the methods comprise determining that the subject has a serum sBCMA level higher than about 566 ng/mL. In some embodiments, the methods comprise determining that the subject has a serum sBCMA level higher than about 500 ng/mL. In some embodiments, the subject has been determined to have a serum sBCMA level higher than about 600 ng/mL. In some embodiments, the subject has been determined to have a serum sBCMA level higher than about 566 ng/mL. In some embodiments, the subject has been determined to have a serum sBCMA level higher than about 500 ng/mL. In some embodiments, if the subject is determined to have a serum sBCMA level higher than about 600 ng/mL, the method comprises debulking the MM. In some embodiments, if the subject is determined to have a serum sBCMA level higher than about 566 ng/mL, the method comprises debulking the MM. In some embodiments, if the subject is determined to have a serum sBCMA level higher than about 500 ng/mL, the method comprises debulking the MM. In some embodiments, if the subject is determined to have a serum sBCMA level higher than about 600 ng/mL, the MM is debulked. In some embodiments, if the subject is determined to have a serum sBCMA level higher than about 566 ng/mL, the MM is debulked. In some embodiments, if the subject is determined to have a serum sBCMA level higher than about 500 ng/mL, the MM is debulked. In some embodiments, if the subject is determined to have a serum sBCMA level higher than about 600 ng/mL, the subject is selected for debulking. In some embodiments, if the subject is determined to have a serum sBCMA level higher than about 566 ng/mL, the subject is selected for debulking. In some embodiments, if the subject is determined to have a serum sBCMA level higher than about 500 ng/mL, the subject is selected for debulking.

In some embodiments, the methods comprise determining that the subject has a presence of IgG HCD. In some embodiments, the subject has been determined to have a presence of IgG HCD. In some embodiments, if the subject is determined to have a presence of IgG HCD, the method comprises debulking the MM. In some embodiments, if the subject is determined to have a presence of IgG HCD, the MM is debulked. In some embodiments, if the subject is determined to have a presence of IgG HCD, the subject is selected for debulking.

In some embodiments, the methods comprise determining that the subject has a presence of IgG HCD and a scrum sBCMA level higher than about 600 ng/mL. In some embodiments, the methods comprise determining that the subject has a presence of IgG HCD and a serum sBCMA level higher than about 566 ng/mL. In some embodiments, the methods comprise determining that the subject has a presence of IgG HCD and a serum sBCMA level higher than about 500 ng/mL. In some embodiments, the subject has been determined to have a presence of IgG HCD and serum sBCMA level higher than about 600 ng/mL. In some embodiments, the subject has been determined to have a presence of IgG HCD and serum sBCMA level higher than about 566 ng/mL. In some embodiments, the subject has been determined to have a presence of IgG HCD and serum sBCMA level higher than about 500 ng/mL. In some embodiments, if the subject is determined to have a presence of IgG HCD and serum sBCMA level higher than about 600 ng/mL, the method comprises debulking the MM. In some embodiments, if the subject is determined to have a presence of IgG HCD and serum sBCMA level higher than about 566 ng/mL, the method comprises debulking the MM. In some embodiments, if the subject is determined to have a presence of IgG HCD and serum sBCMA level higher than about 500 ng/mL, the method comprises debulking the MM. In some embodiments, if the subject is determined to have a presence of IgG HCD and serum sBCMA level higher than about 600 ng/mL, the MM is debulked. In some embodiments, if the subject is determined to have a presence of IgG HCD and serum sBCMA level higher than about 566 ng/mL, the MM is debulked. In some embodiments, if the subject is determined to have a presence of IgG HCD and serum sBCMA level higher than about 500 ng/mL, the MM is debulked. In some embodiments, if the subject is determined to have a presence of IgG HCD and serum sBCMA level higher than about 600 ng/mL, the subject is selected for debulking. In some embodiments, if the subject is determined to have a presence of IgG HCD and serum sBCMA level higher than about 566 ng/mL, the subject is selected for debulking. In some embodiments, if the subject is determined to have a presence of IgG HCD and serum sBCMA level higher than about 500 ng/mL, the subject is selected for debulking.

In some embodiments, the methods comprise: (a) determining that the subject has: (i) a serum soluble B cell maturation antigen (sBCMA) level higher than about 600 ng/mL; and/or (ii) a presence of IgG heavy chain disease (HCD); (b) debulking the MM; and (c) administering to the subject a T cell therapy comprising a dose of genetically engineered T cells expressing a chimeric antigen receptor (CAR) that binds to BCMA. In some embodiments, the methods comprise: (a) determining that the subject has a serum soluble B cell maturation antigen (sBCMA) level higher than about 600 ng/mL; and (c) administering to the subject a T cell therapy comprising a dose of genetically engineered T cells expressing a chimeric antigen receptor (CAR) that binds to BCMA. In some embodiments, the methods comprise: (a) determining that the subject has a presence of IgG heavy chain disease (HCD); (b) debulking the MM; and (c) administering to the subject a T cell therapy comprising a dose of genetically engineered T cells expressing a chimeric antigen receptor (CAR) that binds to BCMA. In some embodiments, the methods comprise: (a) determining that the subject has: (i) a serum soluble B cell maturation antigen (sBCMA) level higher than about 600 ng/mL; and (ii) a presence of IgG heavy chain disease (HCD); (b) debulking the MM; and (c) administering to the subject a T cell therapy comprising a dose of genetically engineered T cells expressing a chimeric antigen receptor (CAR) that binds to BCMA.

In some embodiments, the methods comprise: (a) determining that the subject has: (i) a serum soluble B cell maturation antigen (sBCMA) level higher than about 566 ng/mL; and/or (ii) a presence of IgG heavy chain disease (HCD); (b) debulking the MM; and (c) administering to the subject a T cell therapy comprising a dose of genetically engineered T cells expressing a chimeric antigen receptor (CAR) that binds to BCMA. In some embodiments, the methods comprise: (a) determining that the subject has a serum soluble B cell maturation antigen (sBCMA) level higher than about 566 ng/mL; and (c) administering to the subject a T cell therapy comprising a dose of genetically engineered T cells expressing a chimeric antigen receptor (CAR) that binds to BCMA. In some embodiments, the methods comprise: (a) determining that the subject has a presence of IgG heavy chain disease (HCD); (b) debulking the MM; and (c) administering to the subject a T cell therapy comprising a dose of genetically engineered T cells expressing a chimeric antigen receptor (CAR) that binds to BCMA. In some embodiments, the methods comprise: (a) determining that the subject has: (i) a serum soluble B cell maturation antigen (sBCMA) level higher than about 566 ng/mL; and (ii) a presence of IgG heavy chain disease (HCD); (b) debulking the MM; and (c) administering to the subject a T cell therapy comprising a dose of genetically engineered T cells expressing a chimeric antigen receptor (CAR) that binds to BCMA.

In some embodiments, the methods comprise: (a) determining that the subject has: (i) a serum soluble B cell maturation antigen (sBCMA) level higher than about 500 ng/mL; and/or (ii) a presence of IgG heavy chain disease (HCD); (b) debulking the MM; and (c) administering to the subject a T cell therapy comprising a dose of genetically engineered T cells expressing a chimeric antigen receptor (CAR) that binds to BCMA. In some embodiments, the methods comprise: (a) determining that the subject has a serum soluble B cell maturation antigen (sBCMA) level higher than about 500 ng/mL; and (c) administering to the subject a T cell therapy comprising a dose of genetically engineered T cells expressing a chimeric antigen receptor (CAR) that binds to BCMA. In some embodiments, the methods comprise: (a) determining that the subject has a presence of IgG heavy chain disease (HCD); (b) debulking the MM; and (c) administering to the subject a T cell therapy comprising a dose of genetically engineered T cells expressing a chimeric antigen receptor (CAR) that binds to BCMA. In some embodiments, the methods comprise: (a) determining that the subject has: (i) a serum soluble B cell maturation antigen (sBCMA) level higher than about 500 ng/mL; and (ii) a presence of IgG heavy chain disease (HCD); (b) debulking the MM; and (c) administering to the subject a T cell therapy comprising a dose of genetically engineered T cells expressing a chimeric antigen receptor (CAR) that binds to BCMA.

In some embodiments, the methods comprise administering to a subject a T cell therapy comprising a dose of genetically engineered T cells expressing a chimeric antigen receptor (CAR) that binds to BCMA, wherein: (a) the subject was previously determined to have: (i) a serum soluble B cell maturation antigen (sBCMA) level higher than about 600 ng/mL; and/or (ii) a presence of IgG heavy chain disease (HCD); and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have a serum soluble B cell maturation antigen (sBCMA) level higher than about 600 ng/mL; and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have a presence of IgG heavy chain disease (HCD); and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have: (i) a serum soluble B cell maturation antigen (sBCMA) level higher than about 600 ng/mL; and (ii) a presence of IgG heavy chain disease (HCD); and (b) the MM was debulked at a time between (1) the subject being determined to have (i) and/or (ii) and (2) the subject being administered the T cell therapy.

In some embodiments, the methods comprise administering to a subject a T cell therapy comprising a dose of genetically engineered T cells expressing a chimeric antigen receptor (CAR) that binds to BCMA, wherein: (a) the subject was previously determined to have: (i) a serum soluble B cell maturation antigen (sBCMA) level higher than about 566 ng/mL; and/or (ii) a presence of IgG heavy chain disease (HCD); and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have a serum soluble B cell maturation antigen (sBCMA) level higher than about 566 ng/mL; and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have a presence of IgG heavy chain disease (HCD); and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have: (i) a serum soluble B cell maturation antigen (sBCMA) level higher than about 566 ng/mL; and (ii) a presence of IgG heavy chain disease (HCD); and (b) the MM was debulked at a time between (1) the subject being determined to have (i) and/or (ii) and (2) the subject being administered the T cell therapy.

In some embodiments, the methods comprise administering to a subject a T cell therapy comprising a dose of genetically engineered T cells expressing a chimeric antigen receptor (CAR) that binds to BCMA, wherein: (a) the subject was previously determined to have: (i) a serum soluble B cell maturation antigen (sBCMA) level higher than about 500 ng/mL; and/or (ii) a presence of IgG heavy chain disease (HCD); and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have a serum soluble B cell maturation antigen (sBCMA) level higher than about 500 ng/mL; and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have a presence of IgG heavy chain disease (HCD); and (b) the MM was debulked at a time between (1) the subject being determined to have (i) and/or (ii) and (2) the subject being administered the T cell therapy. In some embodiments, the methods comprise administering to a subject a T cell therapy comprising a dose of genetically engineered T cells expressing a chimeric antigen receptor (CAR) that binds to BCMA, wherein: (a) the subject was previously determined to have: (i) a serum soluble B cell maturation antigen (sBCMA) level higher than about 500 ng/mL; and (ii) a presence of IgG heavy chain disease (HCD); and (b) the MM was debulked at a time between (1) the subject being determined to have (i) and/or (ii) and (2) the subject being administered the T cell therapy.

In some embodiments, the methods comprise determining that the subject has a first serum soluble B cell maturation antigen (sBCMA) level higher than about 600 ng/mL. In some embodiments, the methods comprise determining that the subject has a first serum soluble B cell maturation antigen (sBCMA) level higher than about 566 ng/mL. In some embodiments, the methods comprise determining that the subject has a first serum soluble B cell maturation antigen (sBCMA) level higher than about 500 ng/mL. In some embodiments, the subject has been determined to have a first serum sBCMA level higher than about 600 ng/mL. In some embodiments, the subject has been determined to have a first serum sBCMA level higher than about 566 ng/mL. In some embodiments, the subject has been determined to have a first serum sBCMA level higher than about 500 ng/mL. In some embodiments, if the subject is determined to have a first serum sBCMA level higher than about 600 ng/mL, the method comprises debulking the MM. In some embodiments, if the subject is determined to have a first serum sBCMA level higher than about 566 ng/mL, the method comprises debulking the MM. In some embodiments, if the subject is determined to have a first serum sBCMA level higher than about 500 ng/mL, the method comprises debulking the MM. In some embodiments, if the subject is determined to have a first serum sBCMA level higher than about 600 ng/mL, the MM is debulked. In some embodiments, if the subject is determined to have a first serum sBCMA level higher than about 566 ng/mL, the MM is debulked. In some embodiments, if the subject is determined to have a first serum sBCMA level higher than about 500 ng/mL, the MM is debulked. In some embodiments, if the subject is determined to have a first serum sBCMA level higher than about 600 ng/mL, the subject is selected for debulking. In some embodiments, if the subject is determined to have a first serum sBCMA level higher than about 566 ng/mL, the subject is selected for debulking. In some embodiments, if the subject is determined to have a first serum sBCMA level higher than about 500 ng/mL, the subject is selected for debulking.

In some embodiments, following the debulking, the methods comprise determining that the subject has a post-debulking serum soluble B cell maturation antigen (sBCMA) level lower than about 600 ng/mL. In some embodiments, following the debulking, the methods comprise determining that the subject has a post-debulking serum soluble B cell maturation antigen (sBCMA) level lower than about 566 ng/mL. In some embodiments, following the debulking, the methods comprise determining that the subject has a post-debulking serum soluble B cell maturation antigen (sBCMA) level lower than about 500 ng/mL. In some embodiments, following the debulking, the subject has been determined to have a post-debulking serum sBCMA level lower than about 600 ng/mL. In some embodiments, following the debulking, the subject has been determined to have a post-debulking serum sBCMA level lower than about 566 ng/mL. In some embodiments, following the debulking, the subject has been determined to have a post-debulking serum sBCMA level lower than about 500 ng/mL. In some embodiments, if the subject is determined to have a post-debulking serum sBCMA level lower than about 600 ng/mL, the method comprises administering the T cell therapy to the subject. In some embodiments, if the subject is determined to have a post-debulking serum sBCMA level lower than about 566 ng/mL, the method comprises administering the T cell therapy to the subject. In some embodiments, if the subject is determined to have a post-debulking serum sBCMA level lower than about 500 ng/mL, the method comprises administering the T cell therapy to the subject. In some embodiments, if the subject is determined to have a post-debulking serum sBCMA level lower than about 600 ng/mL, the T cell therapy is administered to the subject. In some embodiments, if the subject is determined to have a post-debulking serum sBCMA level lower than about 566 ng/mL, the T cell therapy is administered to the subject. In some embodiments, if the subject is determined to have a post-debulking serum sBCMA level lower than about 500 ng/mL, the T cell therapy is administered to the subject.

In some embodiments, the methods comprise: (a) determining that a subject has a first serum soluble B cell maturation antigen (sBCMA) level higher than about 600 ng/mL; (b) debulking the MM; (c) determining that the subject has a post-debulking serum sBCMA level lower than about 600 ng/mL; and (d) administering to the subject a T cell therapy comprising a dose of genetically engineered T cells expressing a chimeric antigen receptor (CAR) that binds to BCMA. In some embodiments, if the subject is determined to have a post de-bulking serum sBCMA level higher than about 600 ng/mL, the T cell therapy is not administered to the subject.

In some embodiments, the methods comprise: (a) determining that a subject has a first serum soluble B cell maturation antigen (sBCMA) level higher than about 566 ng/mL; (b) debulking the MM; (c) determining that the subject has a post-debulking serum sBCMA level lower than about 566 ng/mL; and (d) administering to the subject a T cell therapy comprising a dose of genetically engineered T cells expressing a chimeric antigen receptor (CAR) that binds to BCMA. In some embodiments, if the subject is determined to have a post de-bulking serum sBCMA level higher than about 566 ng/mL, the T cell therapy is not administered to the subject.

In some embodiments, the methods comprise: (a) determining that a subject has a first serum soluble B cell maturation antigen (sBCMA) level higher than about 500 ng/mL; (b) debulking the MM; (c) determining that the subject has a post-debulking serum sBCMA level lower than about 500 ng/mL; and (d) administering to the subject a T cell therapy comprising a dose of genetically engineered T cells expressing a chimeric antigen receptor (CAR) that binds to BCMA. In some embodiments, if the subject is determined to have a post de-bulking serum sBCMA level higher than about 500 ng/mL, the T cell therapy is not administered to the subject.

In some embodiments, if the subject has (i) a serum sBCMA lower than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL; and/or (ii) an absence of IgG HCD, the MM is not debulked. In some embodiments, if the subject has a serum sBCMA lower than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL, the MM is not debulked. In some embodiments, if the subject has (i) a serum sBCMA lower than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL; and/or (ii) an absence of IgG HCD, the subject is not selected for debulking. In some embodiments, if the subject has a serum sBCMA lower than about 590 ng/mL, about 580 ng/mL, about 570 ng/mL, about 560 ng/mL, about 550 ng/mL, about 540 ng/mL, about 530 ng/mL, about 520 ng/mL, about 510 ng/mL, or about 500 ng/mL, the subject is not selected for debulking. In some embodiments, if the subject has serum sBCMA level lower than about 600 ng/mL, the MM is not debulked prior to administration of the T cell therapy to the subject. In some embodiments, if the subject has serum sBCMA level lower than about 600 ng/mL, the subject is not selected for debulking prior to administration of the T cell therapy to the subject. In some embodiments, if the subject has serum sBCMA level lower than about 566 ng/mL, the MM is not debulked prior to administration of the T cell therapy to the subject. In some embodiments, if the subject has serum sBCMA level lower than about 566 ng/mL, the subject is not selected for debulking prior to administration of the T cell therapy to the subject.

In some embodiments, debulking comprises administration of chemotherapy, radiation, or an immunomodulatory agent to the subject. In some embodiments, debulking comprises administration of chemotherapy to the subject. In some embodiments, the chemotherapy comprises melphalan, doxorubicin, or cyclophosphamide chemotherapy. In some embodiments, comprises administration of radiation to the subject. In some embodiments, the immunomodulatory agent is thalidomide, lenalidomide, or pomalidomide. In some embodiments, comprises administration of an immunomodulatory agent to the subject. In some embodiments, the immunomodulatory agent is a checkpoint inhibitor.

In some of any of the provided embodiments, the debulking is carried out prior to the administration of the T cell therapy to the subject.

In some embodiments, the debulking the MM is carried out within about three months before, within about two months before, within about one month before, within about three weeks before, within about two weeks before, or within about one week before administration of the T cell therapy to the subject. In some embodiments, the debulking the MM is carried out within about three months before administration of the T cell therapy to the subject. In some embodiments, the debulking the MM is carried out within about two months before administration of the T cell therapy to the subject. In some embodiments, the debulking the MM is carried out within about one month before administration of the T cell therapy to the subject. In some embodiments, the debulking the MM is carried out within about three weeks before administration of the T cell therapy to the subject. In some embodiments, the debulking the MM is carried out within about two weeks before administration of the T cell therapy to the subject. In some embodiments, the debulking the MM is carried out within about one week before administration of the T cell therapy to the subject.

In some embodiments, the debulking the MM is carried out at about three months before, at about two months before, at about one month before, at about three weeks before, at about two weeks before, or at about one week before administration of the T cell therapy to the subject. In some embodiments, the debulking the MM is carried out at about three months before administration of the T cell therapy to the subject. In some embodiments, the debulking the MM is carried out at about two months before administration of the T cell therapy to the subject. In some embodiments, the debulking the MM is carried out at about one month before administration of the T cell therapy to the subject. In some embodiments, the debulking the MM is carried out at about three weeks before administration of the T cell therapy to the subject. In some embodiments, the debulking the MM is carried out at about two weeks before administration of the T cell therapy to the subject. In some embodiments, the debulking the MM is carried out at about one week before administration of the T cell therapy to the subject. In some embodiments, the debulking is carried out prior to administration of a lymphodepleting therapy to the subject. In some embodiments, the debulking is carried out after the administration of a lymphodepleting therapy to a subject. In some embodiments, the subject is treated with a gamma-secretase inhibitor prior to administration of the T cell therapy to the subject.

### C. Dosage and Administration of T Cell Therapy

In some embodiments of the methods, compositions, combinations, kits and uses provided herein, the treatment includes administering to a subject a T cell therapy (e.g. CAR-expressing T cells). For example, the T cell therapy is an anti-BCMA CAR T cell therapy.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

In some embodiments, the cell-based therapy is or comprises administration of cells, such as immune cells, for example T cell or NK cells, that target a molecule expressed on the surface of a lesion, such as a tumor or a cancer. In some embodiments, the cells express a recombinant receptor, e.g. CAR, that contains an extracellular ligand-binding domain that specifically binds to an antigen. In some embodiments, the recombinant receptor is a CAR that contains an extracellular antigen-recognition domain that specifically binds to BCMA. In some embodiments, the immune cells express a recombinant receptor, such as a chimeric antigen receptor (CAR). In some embodiments, the T cell therapy includes administering T cells engineered to express a chimeric antigen receptor (CAR). In particular embodiments, the cell therapy, e.g. anti-BCMA CAR T cell therapy, is for treating a multiple myeloma, such as a relapsed and refractory (R/R multiple myeloma). In some embodiments, the cells are autologous to the subject. In some embodiments, the cells are allogeneic to the subject. Exemplary engineered cells for administering as a cell therapy in the provided methods are described in Section IV.

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4+ vs CD8+ T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, the dose includes fewer than about 5×10⁸ total recombinant receptor (e.g., CAR)-expressing cells, T cells, or peripheral blood mononuclear cells (PBMCs), e.g., in the range of about 1×10⁸ to 5×10⁸ such cells, such as 1.5×10⁸, 3×10⁸, or 4.5×10⁸ or total such cells, or the range between any two of the foregoing values.

The cells can be administered by any suitable means. The cells are administered in a dosing regimen to achieve a therapeutic effect, such as a reduction in tumor burden. Dosing and administration may depend in part on the schedule of administration of the debulking, which is carried out prior to initiation of administration of the T cell therapy. Various dosing schedules of the T cell therapy include but are not limited to single or multiple administrations over various time-points, bolus administration, and pulse infusion.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is administered a preconditioning agent (lymphodepleting treatment) as described in Section II.D.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In some embodiments, a dose of cells is administered to subjects in accord with the provided T cell therapy methods. In some embodiments, the size or timing of the doses is determined as a function of the particular disease or condition in the subject. One may empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells: in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the dose of cells is a flat dose of cells or fixed dose of cells such that the dose of cells is not tied to or based on the body surface area or weight of a subject.

In some embodiments, the cell therapy comprises administration of a dose comprising a number of cell from or from about 1×10⁵¹ to 2×10⁹ total recombinant receptor-expressing cells, total T cells, or total peripheral blood mononuclear cells (PBMCs), from or from about 5×10⁵ to 1×10⁹ total recombinant receptor-expressing cells, total T cells, or total peripheral blood mononuclear cells (PBMCs) or from or from about 1×10⁶ to 1×10⁹ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸ at least or about at least 1×10⁹ of such cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 1.0×10⁷ to at or about 1.2×10⁹ such cells, such as at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ total such cells, or the range between any two of the foregoing values. In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1.5×10⁸, 3×10⁸, 4.5×10⁸, 6×10⁸, 8×10⁸ or 1.2×10⁹ total such cells, or the range between any two of the foregoing values. In some embodiments, for example, where the subject is a human, the dose includes at or about 1.0×10⁷ total recombinant receptor (e.g., CAR)-expressing cells, T cells, or peripheral blood mononuclear cells (PBMCs). In some embodiments, for example, where the subject is a human, the dose includes at or about 1.5×10⁷ total recombinant receptor (e.g., CAR)-expressing cells, T cells, or peripheral blood mononuclear cells (PBMCs). In some embodiments, for example, where the subject is a human, the dose includes at or about 2.0×10⁷ total recombinant receptor (e.g., CAR)-expressing cells, T cells, or peripheral blood mononuclear cells (PBMCs). In some embodiments, for example, where the subject is a human, the dose includes at or about 2.5×10⁷ total recombinant receptor (e.g., CAR)-expressing cells, T cells, or peripheral blood mononuclear cells (PBMCs). In some embodiments, for example, where the subject is a human, the dose includes at or about 5×10⁷ total recombinant receptor (e.g., CAR)-expressing cells, T cells, or peripheral blood mononuclear cells (PBMCs). In some embodiments, for example, where the subject is a human, the dose includes at or about 1.5×10⁸ total recombinant receptor (e.g., CAR)-expressing cells, T cells, or peripheral blood mononuclear cells (PBMCs). In some embodiments, for example, where the subject is a human, the dose includes at or about 3×10⁸ total recombinant receptor (e.g., CAR)-expressing cells, T cells, or peripheral blood mononuclear cells (PBMCs). In some embodiments, for example, where the subject is a human, the dose includes at or about 4.5×10⁸ total recombinant receptor (e.g., CAR)-expressing cells, T cells, or peripheral blood mononuclear cells (PBMCs). In some embodiments, for example, where the subject is a human, the dose includes at or about 6×10⁸ total recombinant receptor (e.g., CAR)-expressing cells, T cells, or peripheral blood mononuclear cells (PBMCs). In some embodiments, for example, where the subject is a human, the dose includes at or about 8×10⁸ total recombinant receptor (e.g., CAR)-expressing cells, T cells, or peripheral blood mononuclear cells (PBMCs). In some embodiments, for example, where the subject is a human, the dose includes at or about 1.2×10⁹ total recombinant receptor (e.g., CAR)-expressing cells, T cells, or peripheral blood mononuclear cells (PBMCs).

In some embodiments, the dose of genetically engineered cells comprises from at or about 1×10⁵ to at or about 2×10⁹ total CAR-expressing (CAR+) T cells, from at or about 1×10⁵ to at or about 5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells. In some embodiments, the dose of genetically engineered cells comprises from at or about 1.0×10⁷ to at or about 8×10⁸ total CAR-expressing (CAR+) T cells, from at or about 1.0×10⁷ to at or about 6.5×10⁸ total CAR+ T cells, from at or about 1.5×10⁷ to at or about 6.5×10⁸ total CAR+ T cells, from at or about 1.5×10⁷ to at or about 6.0×10⁸ total CAR+ T cells, from at or about 2.5×10⁷ to at or about 6.0×10⁸ total CAR+ T cells, or from at or about 5.0×10⁷ to at or about 6.0×10⁸ total CAR+ T cells.

In some embodiments, the dose of genetically engineered cells comprises between at or about 2.5×10⁷ CAR-expressing (CAR+) T cells, total T cells, or total peripheral blood mononuclear cells (PBMCs) and at or about 1.2×10⁹ CAR-expressing T cells, total T cells, or total PBMCs, between at or about 5.0×10⁷ CAR-expressing T cells, total T cells, or total peripheral blood mononuclear cells (PBMCs) and at or about 6.0×10⁸ CAR-expressing T cells, total T cells, or total PBMCs, between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive.

In some embodiments, the dose is at or about 1.0×10⁷ CD3+ CAR-expressing cells. In some embodiments, the dose is at or about 1.5×10⁷ CD3+ CAR-expressing cells. In some embodiments, the dose is at or about 2.0×10⁷ CD3+ CAR-expressing cells. In some embodiments, the dose is at or about 2.5×10⁷ CD3+ CAR-expressing cells. In some embodiments, the dose is at or about 5×10⁷ CD3+ CAR-expressing cells. In some embodiments, the dose is at or about 1.5×10⁸ CD3+ CAR-expressing cells. In some embodiments, the dose is at or about 3×10⁸ CD3+ CAR-expressing cells. In some embodiments, the dose is at or about 4.5×10⁸ CD3+ CAR-expressing cells. In some embodiments, the dose is at or about 6×10⁸ CD3+ CAR-expressing cells. In some embodiments, the dose is at or about 8×10⁸ CD3+ CAR-expressing cells. In some embodiments, the dose is at or about 1.2×10⁹ CD3+ CAR-expressing cells.

In some embodiments, the dose of genetically engineered cells is with reference to the total number of CD3+ CAR-expressing (CAR+) or CD4+/CD8+ CAR-expressing (CAR+) cells. In some embodiments, the dose comprises a number of genetically engineered cells from or from about 1.0×10⁷ to or to about 1.2×10⁹ CD3+ or CD4+/CD8+ total T cells or CD3+ CAR-expressing or CD4+/CD8+ CAR-expressing cells, from or from about 1.5×10⁷ to or to about 1.2×10⁹ CD3+ or CD4+/CD8+ total T cells or CD3+ CAR-expressing or CD4+/CD8+ CAR-expressing cells, from or from about 2.0×10⁷ to or to about 1.2×10⁹ CD3+ or CD4+/CD8+ total T cells or CD3+ CAR-expressing or CD4+/CD8+ CAR-expressing cells, from or from about 2.5×10⁷ to or to about 1.2×10⁹ CD3+ or CD4+/CD8+ total T cells or CD3+ CAR-expressing or CD4+/CD8+ CAR-expressing cells, from or from about 5.0×10⁷ to or to about 6.0×10⁸ CD3+ or CD4+/CD8+ total T cells or CD3+ CAR-expressing or CD4+/CD8+ CAR-expressing cells, from or from about 5.0×10⁷ to or to about 4.5×10⁸ CD3+ or CD4+/CD8+ total T cells or CD3+ CAR-expressing or CD4+/CD8+ CAR-expressing cells, or from or from about 1.5×10⁸ to or to about 3.0×10⁸ CD3+ or CD4+/CD8+ total T cells or CD3+ CAR-expressing or CD4+/CD8+ CAR-expressing cells, each inclusive. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ CD3+ or CD4+/CD8+ total T cells or CD3+ CAR-expressing or CD4+/CD8+ CAR-expressing cells. In some embodiments, the dose comprises at or about 2.5×10⁷, 5×10⁷, 1.5×10⁸, 3×10⁸, 4.5×10⁸, 6×10⁸, 8×10⁸ or 1.2×10⁹ CD3+ CAR-expressing cells. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ CD4+/CD8+ CAR-expressing cells.

In some embodiments, the dose is at or about 1.0×10⁷ CD4+/CD8+ CAR-expressing cells. In some embodiments, the dose is at or about 1.5×10⁷ CD4+/CD8+ CAR-expressing cells. In some embodiments, the dose is at or about 2.0×10⁷ CD4+/CD8+ CAR-expressing cells. In some embodiments, the dose is at or about 2.5×10⁷ CD4+/CD8+ CAR-expressing cells. In some embodiments, the dose is at or about 5×10⁷ CD4+/CD8+ CAR-expressing cells. In some embodiments, the dose is at or about 1.5×10⁸ CD4+/CD8+ CAR-expressing cells. In some embodiments, the dose is at or about 3×10⁸ CD4+/CD8+ CAR-expressing cells. In some embodiments, the dose is at or about 4.5×10⁸ CD4+/CD8+ CAR-expressing cells. In some embodiments, the dose is at or about 6×10⁸ CD4+/CD8+ CAR-expressing cells. In some embodiments, the dose is at or about 8×10⁸ CD4+/CD8+ CAR-expressing cells. In some embodiments, the dose is at or about 1.2×10⁹ CD4+/CD8+ CAR-expressing cells. In some embodiments, the dose is at or about 2.5×10⁷ CD4+ or CD8+ CAR-expressing cells. In some embodiments, the dose is at or about 5×10⁷ CD4+ or CD8+ CAR-expressing cells. In some embodiments, the dose is at or about 1.5×10⁸ CD4+ or CD8+ CAR-expressing cells. In some embodiments, the dose is at or about 3×10⁸ CD4+ or CD8+ CAR-expressing cells. In some embodiments, the dose is at or about 4.5×10⁸ CD4+ or CD8+ CAR-expressing cells. In some embodiments, the dose is at or about 6×10⁸ CD4+ or CD8+ CAR-expressing cells. In some embodiments, the dose is at or about 6.5×10⁸ CD4+ or CD8+ CAR-expressing cells. In some embodiments, the dose is at or about 8×10⁸ CD4+ or CD8+ CAR-expressing cells. In some embodiments, the dose is at or about 1.2×10⁹ CD4+ or CD8+ CAR-expressing cells.

In some embodiments, the T cells of the dose include CD4+ T cells, CD8+ T cells or CD4+ and CD8+ T cells. In some embodiments, the T cells of the dose include CD4+ T cells. In some embodiments, the T cells of the dose include CD8+ T cells. In some embodiments, the T cells of the dose include CD4+ T cells or CD8+ T cells. In some embodiments, the T cells of the dose include CD4+ and CD8+ T cells.

In some embodiments, for example, where the subject is human, the total of CD4+ T cells and CD8+ T cells of the dose includes between at or about 1×10⁶ and at or about 2×10⁹ total CAR-expressing CD4+ cells and CAR-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 1.0×10⁷, at or about 2.5×10⁷, at or about 2.0×10⁷, at or about 2.5×10⁷, at or about 5×10⁷, at or about 1.5×10⁸, at or about 3×10⁸, at or about 4.5×10⁸, at or about 6×10⁸, at or about 6.5×10⁸, at or about 8×10⁸, or at or about 1.2×10⁹ total such cells, or the range between any two of the foregoing values. In some embodiments, for example, where the subject is human, the CD8+ T cells of the dose, including in a dose including CD4+ T cells and CD8+ T cells, includes between at or about 1×10⁶ and at or about 2×10⁹ total recombinant receptor (e.g., CAR)-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 2.5×10⁷, at or about 5×10⁷, at or about 1.5×10⁸, at or about 3×10⁸, at or about 4.5×10⁸, at or about 6×10⁸, at or about 8×10⁸, or at or about 1.2×10⁹ total such cells, or the range between any two of the foregoing values.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values. In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as Caspase 3. In some aspects, activation of Caspase-3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active Caspase 3. In some embodiments, the marker of apoptosis is a reagent that detects a feature in a cell that is associated with apoptosis. In certain embodiments, the reagent is an Annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%. 3%, 2% or 1% of the CAR-expressing T cells in the dose of engineered T cells express a marker of apoptosis, optionally Annexin V or active Caspase 3. In some of any embodiments, less than 5%, 4%. 3%, 2% or 1% of the CAR-expressing T cells in the dose of engineered T cells express Annexin V or active Caspase 3.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1 or is between approximately 1:3 and approximately 3:1, such as approximately 1:1. In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1. In some embodiments, the dose or composition of cells includes a defined or target ratio of CD4+ cells expressing a recombinant receptor to CD8+ cells expressing a recombinant receptor and/or of CD4+ cells to CD8+ cells that is between approximately 1:3 and approximately 3:1.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, for example, the dose contains between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷ recombinant-receptor expressing cells. In some embodiments, the dose of cells contains a number of cells, that is about 1.5×10⁸ recombinant-receptor expressing cells, about 3.0×10⁸ recombinant-receptor expressing cells, or about 4.5×10⁸ recombinant-receptor expressing cells, such as recombinant-receptor expressing cells that are CD3+. In some embodiments, the dose of cells contains a number of cells, that is between at least or at least about 5×10⁶, 6×10⁶, 7×10⁶, 8×10⁶, 9×10⁶, 10×10⁶ and about 15×10⁶ recombinant-receptor expressing cells, such as recombinant-receptor expressing cells that are CD8+. In some embodiments, such dose, such as such target number of cells refers to the total recombinant-receptor expressing cells in the administered composition.

In some embodiments, for example, the lower dose contains less than about 5×10⁶ cells, recombinant receptor (e.g. CAR)-expressing cells, T cells, and/or PBMCs per kilogram body weight of the subject, such as less than about 4.5×10⁶, 4×10⁶, 3.5×10⁶, 3×10⁶, 2.5×10⁶, 2×10⁶, 1.5×10⁶, 1×10⁶, 5×10⁵, 2.5×10⁵, or 1×10⁵ such cells per kilogram body weight of the subject. In some embodiments, the lower dose contains less than about 1×10⁵, 2×10⁵, 5×10⁵, or 1×10⁶ of such cells per kilogram body weight of the subject, or a value within the range between any two of the foregoing values. In some embodiments, such values refer to numbers of recombinant receptor-expressing cells; in other embodiments, they refer to number of T cells or PBMCs or total cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses. In some embodiments, one or more subsequent dose of cells can be administered to the subject. In some embodiments, the subsequent dose of cells is administered greater than or greater than about 7 days. 14 days, 21 days. 28 days or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated.

### D. Lymphodepleting Treatment

In some aspects, the provided methods can further include administering one or more lymphodepleting therapies, such as prior to initiation of administration of the T cell therapy. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See. e.g., Dudley et al., Science, 298, 850-54 (2002); Rosenberg et al., Cin Cancer Res, 17(13):4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011): Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system: impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every, other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy. 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject.

### III. EXEMPLARY TREATMENT OUTCOMES AND METHODS FOR ASSESSING SAME

In some embodiments of the methods, uses, kits and articles of manufacture provided herein, the provided T cell therapy results in one or more treatment outcomes, such as a feature associated with any one or more of the parameters associated with the therapy or treatment, as described below. In some embodiments, the method includes assessment of the cytotoxicity of the T cells toward cancer cells, e.g., T cells administered for the T cell based therapy. In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before, during, or after administering the T cell therapy provided herein.

In some embodiments, the T cell therapy can further include one or more screening steps to identify subjects for treatment with the T cell therapy and/or continuing the T cell therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the T cell therapy provided herein.

In some embodiments, any of the screening steps and/or assessment of treatment of outcomes described herein can be used prior to, during, during the course of, or subsequent to administration of one or more steps of the provided T cell therapy (e.g. anti-BCMA CAR T cells). In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of one or more steps of the provided T cell therapy, for example, to screen and identify patients suitable and/or susceptible to receive the T cell therapy. In some embodiments, the assessment is performed during, during the course of, or subsequent to administration of one or more steps of the provided T cell therapy, for example, to assess the intermediate or final treatment outcome, e.g., to determine the efficacy of the treatment and/or to determine whether to continue or repeat the treatments and/or to determine whether to administer the remaining steps of the T cell therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known in the art, and can be performed one or more times, e.g., prior to, during, during the course of, or subsequently to administration of one or more steps of the T cell therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the cytotoxicity of recombinant receptor-expressing, e.g., CAR-expressing, cells in the subject following administration of the dose of cells in the method with debulking, is greater as compared to that achieved via a method without debulking. In some embodiments, the cytotoxicity of recombinant receptor-expressing, e.g., CAR-expressing, cells in the subject following administration of the dose of cells in the method wherein a subject is selected for treatment as having a serum sBCMA level lower than about 600 ng/mL and/or an absence of IgG heavy chain disease (HCD), is greater as compared to that achieved via a method without selecting the subject. In some embodiments, cytotoxicity in the subject of the administered T cell therapy, e.g., CAR-expressing T cells is assessed as compared to a method in which the T cell therapy is administered to a subject who is not selected for treatment. In some embodiments, the methods result in the administered T cells exhibiting increased or prolonged cytotoxicity in the subject as compared to a method in which the T cell therapy is administered to a subject who is not selected for treatment.

In some embodiments, the debulking of a tumor prior to treatment with the T cell therapy decreases disease burden, e.g., tumor burden, in the subject as compared to a method in which the tumor is not debulked prior to treatment. In some embodiments, the selecting of a subject for treatment decreases disease burden, e.g., tumor burden, in the subject, as compared to a method in which the subject is not selected for treatment. In some embodiments, the debulking of a tumor prior to treatment with the T cell therapy results in improved clinical outcomes, e.g., objective response rate (ORR), progression-free survival (PFS) and/or overall survival (OS), compared to a method in which the tumor is not debulked prior to treatment. In some embodiments, the selecting of a subject for treatment with the T cell therapy results in improved clinical outcomes, e.g., objective response rate (ORR), progression-free survival (PFS) and/or overall survival (OS), compared to a method in which the subject is not selected for treatment.

In some embodiments, the subject can be screened prior to the administration of one or more steps of the T cell therapy. For example, the subject can be screened for characteristics of the disease and/or disease burden, e.g., tumor burden, prior to administration of the T cell therapy, to determine suitability, responsiveness and/or susceptibility to administering the T cell therapy. For example, the subject can be screened for characteristics of the disease prior to administration of the T cell therapy, to determine suitability, responsiveness and/or susceptibility to administering the T cell therapy. In some embodiments, the screening step and/or assessment of treatment outcomes can be used to determine the dose, frequency, duration, timing and/or order of the T cell therapy provided herein.

In some embodiments, the subject can be screened after administration of one of the steps of the T cell therapy, to determine and identify subjects to receive the remaining steps of the T cell therapy and/or to monitor efficacy of the therapy. In some embodiments, the number, level or amount of administered T cells and/or proliferation and/or activity of the administered T cells is assessed after administration of the engineered T cells.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

Assessment and determination of parameters associated with T cell health, function, activity, and/or outcomes, such as response, efficacy and/or toxicity outcomes, can be assessed at various time points. In some aspects, the assessment can be performed multiple times, e.g., prior to, during, and/or after manufacturing of the cells, prior to, during, and/or after the initiation of administration of the T cell therapy.

In some embodiments, functional attributes of the administered cells and/or cell compositions include monitoring pharmacokinetic (PK) and pharmacodynamics parameters, expansion and persistence of the cells, cell functional assays (e.g., any described herein, such as cytotoxicity assay, cytokine secretion assay and in vivo assays), high-dimensional T cell signaling assessment, and assessment of exhaustion phenotypes and/or signatures of the T cells.

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g. CAR-expressing T cells) can reduce or prevent the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

In some embodiments, the provided methods result in a decreased tumor burden in treated subjects compared to alternative methods in which the T cell therapy (e.g. anti-BCMA CAR T cells) is given without debulking the tumor prior to treatment. In some embodiments, the provided methods result in a decreased tumor burden in treated subjects compared to alternative methods in which the T cell therapy (e.g. anti-BCMA CAR T cells) is given to a subject without selecting the subject for treatment.

It is not necessary that the tumor burden actually be reduced in all subjects receiving the T cell therapy, but that tumor burden is reduced on average in subjects treated, such as based on clinical data, in which a majority of subjects treated with such a T cell therapy exhibit a reduced tumor burden, such as at least 50%/6, 60%, 70%, 80%, 90%, 95% or more of subjects treated with the T cell therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/l): renal insufficiency attributable to myeloma; anemia (hemoglobin <10 g/dl); and/or bone lesions (lytic lesions or osteoporosis with compression fractures)).

Exemplary methods for assessing disease status or disease burden include: measurement of M protein in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination. In some examples, efficacy of the T cell therapy following its administration to the subject is determined by the expansion and persistence of the T cells (e.g. BCMA CAR T cells) in the blood and/or bone marrow. In some embodiments, efficacy of the T cell therapy is determined based on the antitumor activity of the administered cells (e.g. BCMA CAR T cells). In some embodiments antitumor activity is determined by the overall response rate (ORR) and/or International Myeloma Working Group (IMWG) Uniform Response Criteria (see Kumar et al. (2016) Lancet Oncol 17(8):e328-346). In some embodiments, response is evaluated using minimal residual disease (MRD) assessment. In some embodiments, MRD can be assessed by methods such as flow cytometry and high-throughput sequencing, e.g., deep sequencing. In some aspects, subjects that have a MRD-negative disease include those exhibiting absence of aberrant clonal plasma cells on bone marrow aspirate, ruled out by an assay with a minimum sensitivity of 1 in 10⁵ nucleated cells or higher (i.e., 10⁻⁵ sensitivity), such as flow cytometry (next-generation flow cytometry: NGF) or high-throughput sequencing. e.g., deep sequencing or next-generation sequencing (NGS).

In some aspects, sustained MRD-negative includes subjects that exhibit MRD negativity in the marrow (NGF or NGS, or both) and by imaging as defined below, confirmed minimum of 1 year apart. Subsequent evaluations can be used to further specify the duration of negativity (e.g., MRD-negative at 5 years). In some aspects, flow MRD-negative includes subjects that exhibit an absence of phenotypically aberrant clonal plasma cells by NGF on bone marrow aspirates using the EuroFlow standard operation procedure for MRD detection in multiple myeloma (or validated equivalent method) with a minimum sensitivity of 1 in 10⁵ nucleated cells or higher. In some aspects, sequencing MRD-negative includes subjects that exhibit an absence of clonal plasma cells by NGS on bone marrow aspirate in which presence of a clone is defined as less than two identical sequencing reads obtained after DNA sequencing of bone marrow aspirates using the LymphoSIGHT platform (or validated equivalent method) with a minimum sensitivity of 1 in 10⁵ nucleated cells or higher. In some aspects, imaging plus MRD-negative includes subjects that exhibit MRD negativity as assessed by NGF or NGS plus disappearance of every area of increased tracer uptake found at baseline or a preceding PET/CT or decrease to less mediastinal blood pool SUV or decrease to less than that of surrounding normal tissue (see Kumar et al. (2016) Lancet Oncol 17(8):e328-346).

In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of tumor burden is specified. In some embodiments, exemplary parameters for determination include particular clinical outcomes indicative of amelioration or improvement in the tumor. Such parameters include: duration of disease control, including objective response (OR), complete response (CR), stringent complete response (sCR), very good partial response (VGPR), partial response (PR), minimal response (MR), Stable disease (SD). Progressive disease (PD) or relapse (see, e.g., International Myeloma Working Group (IMWG) Uniform Response Criteria; see Kumar et al. (2016) Lancet Oncol 17(8):e328-346), objective response rate (ORR), progression-free survival (PFS) and overall survival (OS). In some embodiments, response is evaluated using minimal residual disease (MRD) assessment. In some embodiments, response is evaluated using complete response (CR) or stringent CR (sCR) assessment. In some embodiments, response is evaluated using complete response (CR) assessment. In some embodiments, response is evaluated using stringent CR (sCR) assessment. Specific thresholds for the parameters can be set to determine the efficacy of the methods provided herein. In some embodiments, the disease or disorder to be treated is multiple myeloma. In some embodiments, measurable disease criteria for multiple myeloma can include (1) serum M-protein 1 g/dL or greater; (2) Urine M-protein 200 mg or greater/24 hour; (3) involved serum free light chain (sFLC) level 10 mg/dL or greater, with abnormal κ to λ ratio. In some cases, light chain disease is acceptable only for subjects without measurable disease in the serum or urine.

In some embodiments, response is evaluated based on the duration of response following administration of the T cell therapy, e.g. BCMA CAR T cells. In some aspects, the response to the therapy, e.g., according to the provided embodiments, can be measured at a designated time point after the initiation of administration of the T cell therapy. In some embodiments, the designated time point is at or about 1, 2, 3, 6, 9, 12, 18, 24, 30 or 36 months following initiation of the administration, or within a range defined by any of the foregoing. In some embodiments, the designated time point is 4, 8, 12, 16, 20, 24, 28, 32, 36, 48 or 52 weeks months following initiation of the administration, or within a range defined by any of the foregoing. In some embodiments, the designated time point is at or about 1 month following initiation of the administration. In some embodiments, the designated time point is at or about 3 months following initiation of the administration. In some embodiments, the designated time point is at or about 6 months following initiation of the administration. In some embodiments, the designated time point is at or about 9 months following initiation of the administration. In some embodiments, the designated time point is at or about 12 months following initiation of the administration. In some embodiments, the response is a CR or a sCR. In some embodiments, the response is a CR. In some embodiments, the response is a sCR.

In some embodiments, the response or outcome determined at or about 3, 6, 9 or 12 months after the designated time point is equal to or improved compared to the response or outcome determined at the initial designated time point. For example, in some aspects, if the response or outcome determined at the initial designated time point is stable disease (SD), progressive disease (PD) or relapse, the subject treated according to the provided embodiments can show an equal or improved response or outcome (e.g., exhibiting a better response outcome according to the International Mycloma Working Group (IMWG) Uniform Response Criteria; see Kumar et al. (2016) Lancet Oncol 17(8):e328-346) at a subsequent time point, after at or about 3, 6, 9 or 12 months after the initial designated time point, that is equal to the response or outcome at the initial designated time point, or a response or outcome that is objective response (OR), complete response (CR), stringent complete response (sCR), very good partial response (VGPR) or partial response (PR). In some embodiments, the response is a CR or a sCR. In some embodiments, the response is a CR. In some embodiments, the response is a sCR. In some aspects, subjects treated according to the provided embodiments can show a response or outcome that is improved between two time point of determination. In some aspects, the subject can exhibit a PR or VGPR in the initial designated time point for assessment, e.g., at 4 weeks after the initiation of administration, then exhibit an improved response, such as a CR or an sCR, at a later time point, e.g., at 12 weeks after the initiation of administration. In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response or outcome is durable for greater than at or about 3, 6, 9 or 12 months.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Sorensen et al., (1993) Br J Cancer 67(4) 773-775. In some embodiments, the subject that are to be administered according to the methods or treatment regimen provided herein include those with an ECOG performance status of 0 or 1.

In some embodiments, the methods and/or administration of an a T cell therapy (e.g. BCMA CAR T cells) decrease(s) disease burden as compared with disease burden at a time immediately prior to the administration of the T cell therapy.

In some aspects, administration of the T cell therapy may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives T cell therapy in the absence of debulking the tumor prior to treatment and/or in the absence of the subject being selected for treatment. In some embodiments, disease burden is reduced to a greater extent or for a greater duration following the of administration of the T cell therapy, compared to the reduction that would be effected without debulking the tumor prior to treatment and/or without selecting the subject for treatment.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). In some embodiments, the parameter is CR or sCR. In some embodiments, the parameter is CR. In some embodiments, the parameter is sCR. Specific thresholds for the parameters can be set to determine the efficacy of the method of T cell therapy provided herein.

In some embodiments, the subjects treated according to the method achieve a more durable response. In some cases, a measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months, greater than 6 months, or great than 12 months. In some particular embodiments, the subjects treated according to the method achieve a more durable response after the subject previously relapsed following remission in response to the administration of the genetically engineered cells.

In some aspects, disease burden is measured or detected prior to administration of the debulking, prior to administration of the T cell therapy, and/or following the debulking but prior to administration of the T cell therapy. In the context of multiple administration of one or more steps of the T cell therapy, disease burden in some embodiments may be measured prior to or following administration of any of the steps, doses and/or cycles of administration, or at a time between administration of any of the steps, doses and/or cycles of administration.

In some embodiments, the burden is decreased by or by at least at or about 10, 20, 30, 40, 50, 60, 70, 80, 90, or 100 percent by the provided methods compared to immediately prior to the administration of the T cell therapy. In some embodiments, disease burden, tumor size, tumor volume, tumor mass, and/or tumor load or bulk is reduced following administration of the T cell therapy and the debulking, by at least at or about 10, 20, 30, 40, 50, 60, 70, 80, 90% or more compared to that immediately prior to the administration of the T cell therapy and/or the debulking.

In some embodiments, reduction of disease burden by the method comprises an induction in morphologic complete remission, for example, as assessed at 1 month, 2 months, 3 months, or more than 3 months, after administration of, e.g., initiation of, the T cell therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of T cell therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of T cell therapy, is less than about 80%, less than about 70%, less than about 60%, less than about 50%, less than about 40%, less than about 30%, less than about 20%, or less than about 10%.

In some embodiments, the administration can treat the subject despite the subject having become resistant to another therapy. In some embodiments, when administered to subjects according to the embodiments described herein, the dose or the composition is capable of achieving complete response (CR) or stringent CR (sCR), in at least 50%, at least 60%, at least 70%, at least 80%, at least 90%, or at least 95% of subjects that were administered. In some embodiments, when administered to subjects according to the embodiments described herein, the dose or the composition is capable of achieving complete response (CR), in at least 50%, at least 60%, at least 70%, at least 80%, at least 90%, or at least 95% of subjects that were administered. In some embodiments, when administered to subjects according to the embodiments described herein, the dose or the composition is capable of achieving stringent CR (sCR), in at least 50%, at least 60%, at least 70%, at least 80%, at least 90%, or at least 95% of subjects that were administered. In some embodiments, when administered to subjects according to the embodiments described herein, the dose or the composition is capable of achieving objective response (OR), in at least 50%, at least 60%, at least 70%, at least 80%, at least 90%, or at least 95% of subjects that were administered. In some embodiments, OR includes subjects who achieve stringent complete response (sCR), complete response (CR), very good partial response (VGPR), partial response (PR) and minimal response (MR). In some embodiments, when administered to subjects according to the embodiments described herein, the dose or the composition is capable of achieving stringent complete response (sCR), complete response (CR), very good partial response (VGPR) or partial response (PR), in at least 50%, 60%, 70%, 80%, or 85% of subjects that were administered. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, when administered to subjects according to the embodiments described herein, the dose or the composition is capable of achieving stringent complete response (sCR) at least 20%, 30%, 40% 50%, 60% or 70% of subjects that were administered. In some embodiments, when administered to subjects according to the embodiments described herein, the dose or the composition is capable of achieving complete response (CR) at least 20%, 30%, 40% 50%, 60% or 70% of subjects that were administered. In some embodiments, exemplary doses include about 1.0×10⁷, 1.5×10⁷, 2.0×10⁷, 2.5×10⁷, 5.0×10⁷, 1.5×10⁸, 3.0×10⁸, 4.5×10⁸, 6.0×10⁸ or 8.0×10⁸ CAR-expressing (CAR+) T cells. In some embodiments, exemplary doses include about 1.5×10⁸, 3.0×10⁸, or 4.5×10⁸, CAR-expressing (CAR+) T cells. In some aspects, particular response to the treatment, e.g., according to the methods provided herein, can be assessed based on the International Myeloma Working Group (IMWG) Uniform Response Criteria (see Kumar et al. (2016) Lancet Oncol 17(8):e328-346).

### IV. BCMA-TARGETING THERAPY AND ENGINEERED CELLS

Also provided herein are BCMA-targeting therapies. In some embodiments, the BCMA-targeting therapy is provided to a subject having a multiple myeloma. In some embodiments, the BCMA-targeting therapy is an antibody, an antibody-drug conjugate (ADC), or a T cell engager. In some embodiments, the BCMA-targeting therapy is an antibody. In some embodiments, the BCMA-targeting therapy is an antibody-drug conjugate (ADC). In some embodiments, the BCMA-targeting therapy is a T cell engager (TCE). In some embodiments, the BCMA-targeting therapy is a T cell engaging therapy capable of stimulating activity of T cells. In some embodiments, the BCM-targeting therapy is a bispecific T cell engager (BiTE) therapy. In some embodiments, the BCMA-targeting is a cell therapy selected from among the group consisting of a tumor infiltrating lymphocytic (TIL) therapy, an endogenous T cell therapy, a natural kill (NK) cell therapy, a transgenic TCR therapy, and a recombinant-receptor expressing cell therapy, which optionally is a chimeric antigen receptor (CAR)-expressing cell therapy. In some embodiments, the cell therapy is a recombinant receptor-expressing cell therapy. In some embodiments, the cell therapy is a chimeric antigen receptor (CAR)-expressing cell therapy. In any of the embodiments provided herein, the BCMA-targeting therapy is administered to a subject having a multiple myeloma.

Also provided are cells such as engineered cells that contain a recombinant receptor (e.g., a chimeric antigen receptor) such as one that contains an extracellular domain including an anti-BCMA binding moiety, such as an antibody or fragment as described herein. Also provided are populations of such cells, compositions containing such cells and/or enriched for such cells, such as in which cells expressing the BCMA-binding molecule make up at least 50, 60, 70, 80, 90, 91, 92, 93, 94, 95, 96, 97, 98, 99, or more percent of the total cells in the composition or cells of a certain type such as PBMCs, T cells or CD3+, CD8+ or CD4+ cells.

Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes. e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein

In some embodiments, the T cell therapy for use in accord with the provided methods includes administering engineered T cells expressing recombinant receptors designed to recognize and/or specifically bind to molecules associated with multiple myeloma, for example relapsed and refractory (R/R) multiple myeloma (MM) (e.g., BCMA). In some embodiments, binding to the antigen results in a response, such as an immune response against such molecules upon binding to such molecules. In some embodiments, the cells contain or are engineered to contain an engineered receptor, e.g., an engineered antigen receptor, such as a chimeric antigen receptor (CAR), or a T cell receptor (TCR). The recombinant receptor, such as a CAR, generally includes an extracellular antigen (or ligand) binding domain that is directed against BCMA, linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Recombinant Receptors, e.g. Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

In some embodiments of the provided methods and uses, the engineered cells, such as T cells, express a chimeric receptor, such as a chimeric antigen receptor (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments. Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody, VHH) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof. IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme): Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A. “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme)

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 2, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 2
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IMGT or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993): Clarkson et al., Nature 352:624-628 (1991).

Among the antigen binding domains included in the CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; heavy chain variable (V_{H}) regions, single-chain antibody molecules such as scFvs and single-domain antibodies comprising only the V_{F}, region; and multispecific antibodies formed from antibody fragments. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody. In certain embodiments, a single-domain antibody is a human single-domain antibody. In some embodiments, the CAR comprises an antibody heavy chain domain that specifically binds BCMA.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb), or a single domain antibody (sdAb), such as sdFv, nanobody, V_{H}H and V_{NAR}. In some embodiments, an antigen-binding fragment comprises antibody variable regions joined by a flexible linker.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the CAR comprises a sdAb. In some embodiments, the CAR comprises two sdAbs. In some embodiments, each of the two sdAbs is a V_{H} domain. In some embodiments, the two sdAbs bind to different epitopes of BCMA. In some embodiments, the two sdAbs bind to the same epitope of BCMA. In some embodiments, the antibody or antigen binding fragment is an scFv comprising a heavy chain variable (V_{H}) region and a light chain variable (V_{L}) region.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the anti-BCMA antibodies included in the provided CARs are murine antibodies. A “murine antibody” is an antibody with an amino acid sequence corresponding to that of an antibody produced by a murine or a murine cell, or non-murine source that utilizes murine antibody repertoires or other murine antibody-encoding sequences, including murine antibody libraries.

Also among the anti-BCMA antibodies included in the provided CARs are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the antibodies included in the provided CARs are those that are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

Thus, in some embodiments, the chimeric antigen receptor, including TCR-like CARs, includes an extracellular portion containing an antibody or antibody fragment. In some embodiments, the antibody or fragment includes an scFv. In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{H}) region.

In some embodiments, the antibody is an antigen-binding fragment, such as a scFv, that includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50/o, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO:26) or GGGS (3GS; SEQ ID NO:27), such as between 2, 3, 4, and 5 repeats of such a sequence. Exemplary linkers include those having or consisting of an sequence set forth in SEQ ID NO:28 (GGGGSGGGGSGGGGS), SEQ ID NO:29 (GSTSGSGKPGSGEGSTKG), SEQ ID NO: 30 (SRGGGGSGGGGSGGGGSLEMA), or SEQ ID NO:38 (ASGGGGSGGRASGGGGS). In some embodiments, the linker is or comprises the sequence set forth in SEQ ID NO:29.

In some embodiments, the CAR includes a BCMA-binding portion or portions of the antibody molecule, such as a heavy chain variable (Vu) region and/or light chain variable (V_{L}) region of the antibody. e.g., an scFv antibody fragment. The chimeric receptors, such as CARs, generally include an extracellular antigen binding domain, such as a portion of an antibody molecule, generally a variable heavy (V_{H}) chain region and/or variable light (V_{L}) chain region of the antibody, e.g., an scFv antibody fragment. In some embodiments, the provided BCMA-binding CARs contain an antibody, such as an anti-BCMA antibody, or an antigen-binding fragment thereof that confers the BCMA-binding properties of the provided CAR. In some embodiments, the antibody or antigen-binding domain can be any anti-BCMA antibody described or derived from any anti-BCMA antibody described. See. e.g., Carpenter et al., C/n. Cancer Res., 2013, 19(8):2048-2060; Feng et al., Scand. J. Immunol. (2020) 92:e12910; U.S. Pat. Nos. 9,034,324 9,765,342; U.S. Patent publication No. US2016/0046724, US20170183418; and International published PCT App. No. WO 2016090320, WO2016090327, WO2016094304, WO2016014565, WO2016014789, WO2010104949, WO2017025038, WO2017173256, WO2018085690, or WO2021091978. Any of such anti-BCMA antibodies or antigen-binding fragments can be used in the provided CARs. In some embodiments, the anti-BCMA CAR contains one or more single-domain anti-BCMA antibodies. In some embodiments, the one or more single-domain anti-BCMA antibodies is derived from an antibody described in WO2017025038 or WO2018028647. In some embodiments, the anti-BCMA CAR contains two single-domain anti-BCMA antibodies. In some embodiments, the two single-domain anti-BCMA antibodies are derived from one or more antibodies described in WO2017025038 or WO2018028647 (with or without signal peptide). In some embodiments, the BCMA binding domain comprises or consists of A37353-G4S-A37917 (G4S being a linker between the two binding domains), described in WO2017025038 or WO2018028647, and provided, e.g., in SEQ ID NOs: 300, 301 and 302 of WO2017025038 or WO2018028647. In some embodiments, the anti-BCMA CAR contains an antigen-binding domain that is an scFv containing a variable heavy (V_{H}) and/or a variable light (V_{L}) region. In some embodiments, the scFv containing a variable heavy (V_{H}) and/or a variable light (V_{L}) region is derived from an antibody described in WO2016090320 or WO2016090327. In some embodiments, the scFv containing a variable heavy (V_{H}) and/or a variable light (V_{L}) region is derived from an antibody described in WO 2019/090003. In some embodiments, the scFv containing a variable heavy (V_{H}) and/or a variable light (V_{L}) region is derived from an antibody described in WO2016094304 or WO2021091978. In some embodiments, the scFv containing a variable heavy (V_{H}) and/or a variable light (V_{L}) region is derived from an antibody described in WO2018133877. In some embodiments, the scFv containing a variable heavy (V_{H}) and/or a variable light (V_{L}) region is derived from an antibody described in WO2019149269. In some embodiments, the anti-BCMA CAR is any as described in WO2019173636 or WO2020051374A. In some embodiments, the anti-BCMA CAR is any as described in WO2018102752. In some embodiments, the anti-BCMA CAR is any as described in WO2020112796 or WO2021173630.

In some embodiments, the antibody, e.g., the anti-BCMA antibody or antigen-binding fragment, contains a heavy and/or light chain variable (V_{H} or V_{L}) region sequence as described, or a sufficient antigen-binding portion thereof. In some embodiments, the anti-BCMA antibody, e.g., antigen-binding fragment, contains a V_{H} region sequence or sufficient antigen-binding portion thereof that contains a CDR-H1, CDR-H2 and/or CDR-H3 as described. In some embodiments, the anti-BCMA antibody, e.g., antigen-binding fragment, contains a V_{L} region sequence or sufficient antigen-binding portion that contains a CDR-L1, CDR-L2 and/or CDR-L3 as described. In some embodiments, the anti-BCMA antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, about 91%, about 92%, about 93%, about 94%, about 95%, about 96%, about 97%, about 98%, or about 99% identical to such a sequence.

In some embodiments, the antibody is a single domain antibody (sdAb) comprising only a V_{H} region sequence or a sufficient antigen-binding portion thereof, such as any of the above described V_{H} sequences (e.g., a CDR-H1, a CDR-H2, a CDR-H3 and/or a CDR-H4).

In some embodiments, an antibody provided herein (e.g., an anti-BCMA antibody) or antigen-binding fragment thereof comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19(8):2048-2060, U.S. Pat. No. 9,765,342, WO 2016/090320, WO2016090327, WO2010104949A2, WO2016/0046724, WO2016/014789, WO2016/094304, WO2017/025038, and WO2017173256.

In some embodiments, the anti-BCMA CAR contains an antigen-binding domain, such as an scFv, containing a variable heavy (V_{H}) and/or a variable light (V_{L}) region derived from an antibody described in WO2016094304 or WO2021091978. In some embodiments, the antigen-binding domain is an antibody fragment containing a variable heavy chain (VH) and a variable light chain (VL) region. In some embodiments, the anti-BCMA CAR contains an antigen-binding domain, such as an scFv, containing a variable heavy (V_{H}) and/or a variable light (V_{L}) region derived from an antibody described in WO 2016/090320 or WO2016090327.

In some embodiments, the antigen-binding domain is an antibody fragment containing a variable heavy chain (V_{H}) and a variable light chain (V_{L}) region. In some aspects, the V_{H} region is or includes an amino acid sequence having at least 90%, 91%, 92%, 93%, 94%, 95%, 96%, 97%, 98% or 99% sequence identity to the V_{H} region amino acid sequence set forth in any of SEQ ID NOs: 18, 20, 22, 24, 32, 34, 36, 41, 43, 45, 47, 49, 51, 53, 55, 57, 59, 61, 63, 65, 67, 69, 71, 73, 75, 145, 147, 149 and 151; and/or the V_{L} region is or includes an amino acid sequence having at least 90%, 91%, 92%, 93%, 94%, 95%, 96%, 97%, 98%, or 99% sequence identity to the V_{H} region amino acid sequence set forth in any of SEQ ID NOs: 19, 21, 23, 25, 33, 35, 37, 42, 44, 46, 48, 50, 52, 54, 56, 58, 60, 62, 64, 66, 68, 70, 72, 74, 76, 146, 148, 150 and 152.

In some embodiments, the antigen-binding domain, such as an scFv, contains a V_{H} set forth in SEQ ID NO: 18 and a V_{L} set forth in SEQ ID NO:19. In some embodiments, the antigen-binding domain, such as an scFv, contains a V_{H} set forth in SEQ ID NO: 20 and a V_{L} set forth in SEQ ID NO:21. In some embodiments, the antigen-binding domain, such as an scFv, contains a V_{H} set forth in SEQ ID NO: 22 and a V_{L} set forth in SEQ ID NO:23. In some embodiments, the antigen-binding domain, such as an scFv, contains a V_{H} set forth in SEQ ID NO: 24 and a V_{L} set forth in SEQ ID NO:25. In some embodiment the antigen-binding domain, such as an scFv, contains a V_{H} set forth in SEQ ID NO: 32 and a V_{L} set forth in SEQ ID NO:33. In some embodiments, the antigen-binding domain, such as an scFv, contains a V_{H} set forth in SEQ ID NO:34 and a V_{L} set forth in SEQ ID NO:35. In some embodiments, the antigen-binding domain, such as an scFv, contains a V_{H} set forth in SEQ ID NO: 36 and a V_{L} set forth in SEQ ID NO:37. In some embodiments, the antigen-binding domain, such as an scFv, contains a V_{L} set forth in SEQ ID NO: 41 and a V_{L} set forth in SEQ ID NO: 42. In some embodiments, the antigen-binding domain, such as an scFv, contains a V_{H} set forth in SEQ ID NO: 43 and a V_{L} set forth in SEQ ID NO: 44. In some embodiments, the antigen-binding domain, such as an scFv, contains a V_{H} set forth in SEQ ID NO: 45 and a V_{L} set forth in SEQ ID NO: 46. In some embodiments, the antigen-binding domain, such as an scFv, contains a V_{L} set forth in SEQ ID NO: 47 and a V_{L} set forth in SEQ ID NO: 48. In some embodiments, the antigen-binding domain, such as an scFv, contains a V_{H} set forth in SEQ ID NO: 49 and a V_{L} set forth in SEQ ID NO: 50. In some embodiments, the antigen-binding domain, such as an scFv, contains a V_{H} set forth in SEQ ID NO: 51 and a V_{L} set forth in SEQ ID NO: 52. In some embodiments, the antigen-binding domain, such as an scFv, contains a V_{H} set forth in SEQ ID NO: 53 and a V_{L} set forth in SEQ ID NO: 54. In some embodiments, the antigen-binding domain, such as an scFv, contains a V_{H} set forth in SEQ ID NO: 55 and a V_{L} set forth in SEQ ID NO: 56. In some embodiments, the antigen-binding domain, such as an scFv, contains a V_{H} set forth in SEQ ID NO: 57 and a V_{L} set forth in SEQ ID NO: 58. In some embodiments, the antigen-binding domain, such as an scFv, contains a V_{H} set forth in SEQ ID NO: 59 and a V_{L} set forth in SEQ ID NO: 60. In some embodiments, the antigen-binding domain, such as an scFv, contains a V_{H} set forth in SEQ ID NO: 61 and a V_{L} set forth in SEQ ID NO: 62. In some embodiments, the antigen-binding domain, such as an scFv, contains a V_{H} set forth in SEQ ID NO: 63 and a V_{L} set forth in SEQ ID NO: 64. In some embodiments, the antigen-binding domain, such as an scFv, contains a V_{H} set forth in SEQ ID NO: 65 and a V_{L} set forth in SEQ ID NO: 66. In some embodiments, the antigen-binding domain, such as an scFv, contains a V_{H} set forth in SEQ ID NO: 67 and a V_{L} set forth in SEQ ID NO: 68. In some embodiments, the antigen-binding domain, such as an scFv, contains a V_{H} set forth in SEQ ID NO: 69 and a V_{L} set forth in SEQ ID NO: 70. In some embodiments, the antigen-binding domain, such as an scFv, contains a V_{H} set forth in SEQ ID NO: 71 and a V_{L} set forth in SEQ ID NO: 72. In some embodiments, the antigen-binding domain, such as an scFv, contains a Vu set forth in SEQ ID NO: 73 and a V_{L} set forth in SEQ ID NO: 74. In some embodiments, the antigen-binding domain, such as an scFv, contains a V_{H} set forth in SEQ ID NO: 75 and a V_{L} set forth in SEQ ID NO: 76. In some embodiments, the antigen-binding domain, such as an scFv, contains a V_{H} set forth in SEQ ID NO: 145 and a V_{L} set forth in SEQ ID NO: 146. In some embodiments, the antigen-binding domain, such as an scFv, contains a V_{H} set forth in SEQ ID NO: 147 and a V_{L} set forth in SEQ ID NO: 148. In some embodiments, the antigen-binding domain, such as an scFv, contains a V_{H} set forth in SEQ ID NO: 149 and a V_{L} set forth in SEQ ID NO: 150. In some embodiments, the antigen-binding domain, such as an scFv, contains a V_{H} set forth in SEQ ID NO: 151 and a V_{L} set forth in SEQ ID NO: 152. In some embodiments, the V_{H} or V_{L} has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing V_{H}, or V_{L} sequences, and retains binding to BCMA. In some embodiments, the V_{H} region is amino-terminal to the V_{L} region. In some embodiments, the V_{H} region is carboxy-terminal to the V_{L} region. In some embodiments, the variable heavy and variable light chains are connected by a linker. In some embodiments, the linker is set forth in SEQ ID NO: 28, 29, 30, or 38.

Among a provided anti-BCMA CAR is a CAR in which the antibody or antigen-binding fragment contains a V_{H} region comprising the sequence set forth in SEQ ID NO: 18 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:18; and contains a V_{L} region comprising the sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 19. In some embodiments, the antibody or antigen-binding fragment of the provided CAR contains a V_{L} region that has a CDRH1, a CDRH2 and a CDRH3 comprising the amino acid sequence of SEQ ID NOS: 189, 190, and 191, respectively and a V_{L} region that has a CDRL1, a CDRL2 and a CDRL3 comprising the amino acid sequence of SEQ ID NOS: 192, 193, and 194, respectively. In some embodiments, the antibody or antigen-binding fragment of the provided CAR contains a V_{H} region that has a CDRH1, a CDRH2 and a CDRH3 comprising the amino acid sequence of SEQ ID NOS: 195, 196, and 197, respectively and a V_{L} region that has a CDRL1, a CDRL2 and a CDRL3 comprising the amino acid sequence of SEQ ID NOS: 198, 199, and 200, respectively. In some embodiments, the antibody or antigen-binding fragment of the provided CAR contains a V_{H} region that has a CDRH1, a CDRH2 and a CDRH3 comprising the amino acid sequence of SEQ ID NOS: 201, 202, and 203, respectively and a V_{L} region that has a CDRL1, a CDRL2 and a CDRL3 comprising the amino acid sequence of SEQ ID NOS: 204, 205, and 206, respectively. In some embodiments, the antibody or antigen-binding fragment of the provided CAR contains a V_{H} region that has a CDRH1, a CDRH2 and a CDRH3 comprising the amino acid sequence of SEQ ID NOS: 207, 208, and 209, respectively and a V_{L} region that has a CDRL1, a CDRL2 and a CDRL3 comprising the amino acid sequence of SEQ ID NOS: 210, 211, and 212, respectively. In some embodiments, the V_{H} region comprises the sequence set forth in SEQ ID NO:18 and the V_{L} region comprises the sequence set forth in SEQ ID NO:19. In some embodiments, the antibody or antigen-binding fragment is a single-chain antibody fragment, such as an scFv. In some embodiments, the scFv comprises the sequence of amino acids set forth in SEQ ID NO:213 or a sequence of amino acids at least at or about 90%, at or about 91%, at or about 92%, at or about 93%, at or about 94%, at or about 95/a, at or about 96%, at or about 97%, at or about 98%, or at or about 99% identity to SEQ ID NO:213. In some embodiments, the anti-BCMA CAR has the sequence of amino acids set forth in SEQ NO: 116 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:116. In some embodiments, the anti-BCMA CAR is encoded by the polynucleotide sequence set forth in SEQ NO: 214 or a polynucleotide sequence of at least at or about 90%, at or about 91%, at or about 92%, at or about 93%, at or about 94%, at or about 95%, at or about 96%, at or about 97%, at or about 98%, or at or about 99% identity to SEQ ID NO:214.

Among a provided anti-BCMA CAR is a CAR in which the antibody or antigen-binding fragment contains a V_{H} region comprising the sequence set forth in SEQ ID NO: 24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24; and contains a V_{L} region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25. In some embodiments, the antibody or antigen-binding fragment of the provided CAR contains a V_{H} region that has a CDRH1, a CDRH2 and a CDRH3 comprising the amino acid sequence of SEQ ID NOS: 173, 174 and 175, respectively and a V_{L} region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a V_{H} region that has a CDRH1, a CDRH2 and a CDRH3 comprising the amino acid sequence of SEQ ID NOS: 176, 177 and 175, respectively and a V_{L} region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a V_{H} region that has a CDRH1, a CDRH2 and a CDRH3 comprising the amino acid sequence of SEQ ID NOS: 178, 179 and 175, respectively and a V_{L} region that has a CDR1, a CDRL2 and a CDRL3 comprising the amino acid sequence of SEQ ID NOS: 183, 184 and 185, respectively. In some embodiments, the antibody or antigen-binding fragment of the provided CAR contains a V_{H} region that has a CDRH1, a CDRH2 and a CDRH3 comprising the amino acid sequence of SEQ ID NOS: 180, 181 and 182, respectively and a V_{L} region that has a CDRL1, a CDRL2 and a CDRL3 comprising the amino acid sequence of SEQ ID NOS: 186, 187 and 185, respectively. In some embodiments, the V_{H} region comprises the sequence set forth in SEQ ID NO:24 and the V_{L} region comprises the sequence set forth in SEQ ID NO:25. In some embodiments, the antibody or antigen-binding fragment is a single-chain antibody fragment, such as an scFv. In some embodiments, the scFv comprises the sequence of amino acids set forth in SEQ ID NO:188 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:188. In some embodiments, the anti-BCMA CAR has the sequence of amino acids set forth in SEQ NO: 124 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:124. In some embodiments, the anti-BCMA CAR has the sequence of amino acids set forth in SEQ NO: 125 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:125.

In some embodiments, the scFv comprises the amino acid sequence set forth in any one of SEQ ID NOS: 216-247, or an amino acid sequence having at least 90, 95, 96, 97, 98, 99, or 100% sequence identity to a sequence set forth in any one of SEQ ID NOS: 216-247.

In some embodiments, the antigen-binding domain comprises an sdAb. In some embodiments, the antigen-binding domain contains the sequence set forth by SEQ ID NO:77. In some embodiments, the antigen-binding domain comprises a sequence at least or about 50, 60, 70, 80, 85, 90, 95, 96, 97, 98, 99, or 100% identical to the sequence set forth by SEQ ID NO:77.

In some embodiments, the CAR comprises the amino acid sequence set forth in any one of SEQ ID NOS: 90-141, or an amino acid sequence having at least 90, 95, 96, 97, 98, or 99% sequence identity to a sequence set forth in any one of SEQ ID NOS: 190-141.

In some embodiments, among such antibodies or antigen-binding domains in the provided CARs are antibodies capable of binding BCMA protein, such as human BCMA protein, with at least a certain affinity, as measured by any of a number of known methods. In some embodiments, the affinity is represented by an equilibrium dissociation constant (K_{D}); in some embodiments, the affinity is represented by EC50.

A variety of assays are known for assessing binding affinity and/or determining whether a binding molecule (e.g., an antibody or fragment thereof) specifically binds to a particular ligand (e.g., an antigen, such as a BCMA protein). It is within the level of a skilled artisan to determine the binding affinity of a binding molecule, e.g., an antibody, for an antigen, e.g., BCMA. For example, in some embodiments, a BIAcore® instrument can be used to determine the binding kinetics and constants of a complex between two proteins (e.g., an antibody or fragment thereof, and an antigen, such as a BCMA cell surface protein, soluble BCMA protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In some embodiments, the binding molecule, e.g., antibody or fragment thereof or antigen-binding domain of a CAR, binds, such as specifically binds, to an antigen, e.g., a cell surface BCMA protein or soluble BCMA protein or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 105 M⁻¹. In some embodiments, the antibody or fragment thereof or antigen-binding domain of a CAR exhibits a binding affinity for the peptide epitope with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸ M to 10⁻¹⁰ M, or 10⁻⁹ M to 10⁻¹⁰ M. The on-rate (association rate constant; kₒₙ or kₐ: units of 1/NIs) and the off-rate (dissociation rate constant; k_{off} or k_{d}; units of 1/Ms) can be determined using any of the assay methods known in the art, for example, surface plasmon resonance (SPR).

In some embodiments, the binding affinity (EC50) and/or the dissociation constant of the antibody (e.g. antigen-binding fragment) or antigen-binding domain of a CAR to a BCMA protein, such as human BCMA protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, is from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC50) and/or the equilibrium dissociation constant, K_{D}, of the antibody to a BCMA protein, such as human BCMA protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a BCMA protein, such as human BCMA protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a K_{A} of up to 10‘ M-’, up to 10⁶ M⁻¹, up to 10⁵ M⁻¹. In some cases, a binding molecule (e.g. antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In some embodiments, the binding affinity (EC50) and/or the equilibrium dissociation constant, K_{D}, of the binding molecule, e.g., anti-BCMA antibody or fragment thereof or antigen-binding domain of a CAR, to a BCMA protein, is from or from about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC50) and/or the dissociation constant of the equilibrium dissociation constant, K_{D}, of the binding molecule, e.g., anti-BCMA antibody or fragment thereof or antigen-binding domain of a CAR, to a BCMA protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, II nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody (e.g., anti-BCMA reference antibody).

In some embodiments, the binding affinity of the anti-BCMA antibody or antigen-binding domain of a CAR, for different form or topological type of antigens, e.g., soluble or shed BCMA protein compared to the binding affinity to a membrane-bound BCMA, to determine the preferential binding or relative affinity for a particular form or topological type. For example, in some aspects, an anti-BCMA antibodies or antigen-binding domain of a CAR can exhibit preferential binding to membrane-bound BCMA as compared to soluble or shed BCMA and/or exhibit greater binding affinity for, membrane-bound BCMA compared to soluble or shed BCMA. In some embodiments, the equilibrium dissociation constant, K_{D}, for different form or topological type of BCMA proteins, can be compared to determine preferential binding or relative binding affinity. In some embodiments, the preferential binding or relative affinity to a membrane-bound BCMA compared to soluble or shed BCMA can be high. For example, in some cases, the ratio of K_{D} for soluble or shed BCMA and the K_{D} for membrane-bound BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the ratio of K_{A} for membrane-bound BCMA and the K_{A} for soluble or shed BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the antibody or antigen-binding domain of CAR binds soluble or shed BCMA and membrane-bound BCMA to a similar degree, e.g., the ratio of K_{D} for soluble BCMA and KD for membrane-bound BCMA is or is about 1. In some cases, the antibody or antigen-binding domain of CAR binds soluble or shed BCMA and membrane-bound BCMA to a similar degree, e.g., the ratio of K_{A} for soluble BCMA and K_{A} for membrane-bound BCMA is or is about 1. The degree of preferential binding or relative affinity for membrane-bound BCMA or soluble or shed BCMA can be compared with that of a known antibody, such as a reference antibody (e.g., reference anti-BCMA CAR). In some embodiments, the reference antibody (e.g., reference anti-BCMA CAR) binds to membrane-bound and soluble or shed BCMA protein.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a C_{H}1/C_{L}, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

The spacer can be of a length that provides for increased responsiveness of the cell following antigen binding, as compared to in the absence of the spacer. Exemplary spacers. e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is a spacer having at least a particular length, such as having a length that is at least 100 amino acids, such as at least 110, 125, 130, 135, 140, 145, 150, 160, 170, 180, 190, 200, 210, 220, 230, 240, or 250 amino acids in length. Exemplary spacers include IgG4 hinge alone, IgG4 hinge linked to C_{H}2 and C₁₁3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al., Clin. Cancer Res., 19:3153 (2013), Hudecek et al. (2015) Cancer Immunol Res. 3(2): 125-135, international patent application publication number WO2014031687, U.S. Pat. No. 8,822,647 or published app. No. US2014/0271635. In some embodiments, the spacer includes a sequence of an immunoglobulin hinge region, a C_{H}2 and C_{H}3 region. In some embodiments, one of more of the hinge, C_{H}2 and C_{H}3 is derived all or in part from IgG4 or IgG2. In some cases, the hinge, C_{H}2 and C_{H}3 is derived from IgG4. In some aspects, one or more of the hinge, C_{H}2 and C_{H}3 is chimeric and contains sequence derived from IgG4 and IgG2. In some examples, the spacer contains an IgG4/2 chimeric hinge, an IgG2/4 C_{H}2, and an IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence or an N176Q at position 176, in the C_{H}2 region, of the full-length IgG4 Fc sequence.

In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the encoded spacer is or contains the sequence set forth in SEQ ID NO: 31. In some embodiments, the constant region or portion is of IgD. In some embodiments, the spacer has the sequence set forth in SEQ ID NO: 5. In some embodiments, the spacer has the sequence set forth in SEQ ID NO: 89.

Other exemplary spacer regions include hinge regions derived from CD8a, CD28, CTLA4, PD-1, or FcγRIIIa. In some embodiments, the spacer contains a truncated extracellular domain or hinge region of a CD8a, CD28, CTLA4, PD-1, or FcγRIIIa. In some embodiments, the spacer is a truncated CD28 hinge region. In some embodiments, a short oligo- or polypeptide linker, for example, a linker of between 2 and 10 amino acids in length, such as one containing alanines or alanine and arginine, e.g., alanine triplet (AAA) or RAAA (SEQ ID NO: 144), is present and forms a linkage between the scFv and the spacer region of the CAR. In some embodiments, the spacer has the sequence set forth in SEQ ID NO: 78. In some embodiments, the spacer has the sequence set forth in SEQ ID NO: 80. In some embodiments, the spacer has the sequence set forth in any of SEQ ID NOs: 81-83, In some embodiments, the spacer has the sequence set forth in SEQ ID NO: 84. In some embodiments, the spacer has the sequence set forth in SEQ ID NO: 86. In some embodiments, the spacer has the sequence set forth in SEQ ID NO: 88.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4, 5, 31, 78, 80, 81, 82, 83, 84, 86, 88, or 89.

In some embodiments, the spacer has the sequence set forth in SEQ ID NOS: 157-165. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 157-165.

This antigen recognition domain generally is linked to one or more intracellular signaling components, such as signaling components that mimic stimulation and/or activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the chimeric antigen receptor includes a transmembrane domain linking the extracellular domain and the intracellular signaling domain. In some embodiments, the transmembrane domain is fused to the extracellular domain, such as linked or fused between the extracellular domain (e.g. scFv) and intracellular signaling domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD8a, CD9, CD16, CD22, CD33, CD37, CD64, CD80, CD86, CD134, CD137 (4-1BB), CD154, CTLA-4 or PD-1. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28. Exemplary sequences of transmembrane domains are or comprise the sequences set forth in SEQ ID NOs: 8, 79, 85, 87, 142, or 143 Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR CD3 chain that mediates T-cell stimulation and/or activation and cytotoxicity, e.g., CD3 zeta chain, CD3 gamma, CD3 delta. CD3 epsilon, FcR gamma, FcR beta, CDS, CD22, CD79a, CD79b and CD66d. In some examples of ITAM containing primary cytoplasmic signaling sequences include those derived from CD3 zeta chain, FcR gamma. CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell stimulation and/or activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25 or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell stimulation and/or activation is in some aspects described as being mediated by two classes of cytoplasmic signaling sequences: those that initiate antigen-dependent primary stimulation and/or activation through the TCR (primary cytoplasmic signaling regions, domains or sequences), and those that act in an antigen-independent manner to provide a secondary or co-stimulatory signal (secondary cytoplasmic signaling regions, domains or sequences). In some aspects, the CAR includes one or both of such signaling components.

In some embodiments, the CAR includes a signaling region and/or transmembrane portion of a costimulatory receptor, such as CD28, 4-1BB, OX40 (CD134). CD27, DAP10, DAP12, ICOS and/or other costimulatory receptors. In some aspects, the same CAR includes both the primary cytoplasmic signaling region and costimulatory signaling components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, one or more different recombinant receptors can contain one or more different intracellular signaling region(s) or domain(s). In some embodiments, the primary cytoplasmic signaling region is included within one CAR, whereas the costimulatory component is provided by another receptor, e.g., another CAR recognizing another antigen. In some embodiments, the CARs include activating or stimulatory CARs, and costimulatory CARs, both expressed on the same cell (see WO2014/055668).

In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12. NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS.

In some cases, CARs are referred to as first, second, and/or third generation CARs. In some aspects, a first generation CAR is one that solely provides a CD3-chain induced signal upon antigen binding: in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P10747.1), or CD8a (Accession No. P01732.1), or variant thereof, such as a transmembrane domain that comprises the sequence of amino acids set forth in SEQ ID NO: 8, 79, 142, or 143 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8, 79, 142, or 143. In some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the transmembrane domain is a transmembrane domain from CD8a. In some embodiments, the transmembrane domain is any as described in Milone et al., Mol. Ther. (2009) 12(9):1453-64. In some embodiments, the transmembrane domain is or comprises the sequence set forth in SEQ ID NO:143.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 970, 98%, 99% or more sequence identity to SEQ ID NO: 10 or 11. In some embodiments, the intracellular domain comprises an intracellular costimulatory signaling domain of 4-1BB (e.g. Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular domain comprises an intracellular costimulatory signaling domain of 4-1BB, In some embodiments, the 4-1 BB co-stimulatory molecule is any as described in Milone et al., Mol. Ther. (2009) 12(9):1453-64. In some embodiments, the co-stimulatory molecular has the sequence set forth in SEQ ID NO: 12.

In some embodiments, the intracellular signaling domain of the recombinant receptor, e.g. the CAR, comprises a human CD3 zeta stimulatory signaling domain or functional variant thereof, such as a 112 AA cytoplasmic domain of isoform 3 of human CD3ζ (Accession No. P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95% 96%, 97%, 98%, 99% or more sequence identity to SEQ ID NO: 13, 14 or 15. In some embodiments, the CD3-zeta domain is any as described in Milone et al., Mol. Ther. (2009) 12(9):1453-64. In some embodiments, the CD3-zeta is or comprises the sequence set forth in SEQ ID NO: 13.

In some aspects, the spacer contains only a hinge region of an IgG, such as only a hinge of IgG4 or IgG1, such as the hinge only spacer set forth in SEQ ID NO: 1 or SEQ ID NO: 89. In other embodiments, the spacer is or contains an Ig hinge, e.g., an IgG4-derived hinge, optionally linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 4. In some embodiments, the spacer is an Ig hinge, e.g., an IgG4 hinge, linked to a C_{H}3 domain only, such as set forth in SEQ ID NO: 3. In some embodiments, the spacer is or comprises a glycine-serine rich sequence or other flexible linker such as known flexible linkers. In some embodiments, the spacer is a CD8a hinge, such as set forth in any of SEQ ID NOs: 81-83, an FeγRIIIa hinge, such as set forth in SEQ ID NO: 88, a CTLA4 hinge, such as set forth in SEQ ID NO: 84, or a PD-1 hinge, such as set forth in SEQ ID NO: 86. In some embodiments the spacer is derived from CD8. In some embodiments, the spacer is a CD8a hinge sequence. In some embodiments, the hinge sequence is any as described in Milone et al., Mol. Ther. (2009) 12(9):1453-64. In some embodiments, the hinge is or comprises the sequence set forth in SEQ ID NO:82.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain. In some embodiments, the CAR includes an antibody or fragment, such as scFv, a spacer such as any of the Ig-hinge containing spacers, a CD8-derived transmembrane domain, a 4-1BB-derived intracellular signaling domain, and a CD3 zeta-derived signaling domain.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred. In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence. In some embodiments, such CAR constructs further includes a T2A ribosomal skip element and/or a tEGFR sequence, e.g., downstream of the CAR.

An exemplary polypeptide for a truncated EGFR (e.g. tEGFR) comprises the sequence of amino acids set forth in SEQ ID NO: 7 or 166 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 166. An exemplary T2A linker sequence comprises the sequence of amino acids set forth in SEQ ID NO: 6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7.

In some embodiments, the encoded CAR can sequence can further include a signal sequence or signal peptide that directs or delivers the CAR to the surface of the cell in which the CAR is expressed. In some embodiments, the signal peptide is derived from a transmembrane protein. In some examples the signal peptide is derived from CD8a, CD33, or an IgG. Exemplary signal peptides include the sequences set forth in SEQ ID NOs: 39,40 and 153. In some examples the signal peptide is derived from CD8α. In some embodiments, the signal peptide is the sequence set forth in Accession No. NM_001768. In some embodiments, the signal peptide include the sequences set forth in SEQ ID NO: 39.

In some embodiments, the CAR includes an anti-BCMA antibody or fragment, such as any of the anti-human BCMA antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-BCMA antibody or fragment, such as any of the anti-human BCMA antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition. In some embodiments, the CAR specifically binds to BCMA, such as human BCMA, and includes an anti-human BCMA antibody or fragment as described. Non-limiting exemplary CAR sequences, including anti-BCMA CAR sequences, are set forth in SEQ ID NOs: 90-141. In some embodiments, an anti-BCMA CAR includes the amino acid sequence set forth in any of SEQ ID NOS: 90-141 or an amino acid sequence that exhibits at least at or about 90%, at or about 91%, at or about 92%, at or about 93%, at or about 94%, at or about 95%, at or about 96%, at or about 97%, at or about 96%, at or about 97%, at or about 98%, at or about 99% sequence identity to any one of SEQ ID NOS: 90-141, and wherein the CAR specifically binds BCMA, e.g. human BCMA.

In some embodiments, the dose of genetically engineered T cells comprises idecabtagene vicleucel cells (e.g., such as ABECMA® cells); bb21217 cells; orvacabtagene autoleucel cells; CT103A cells: ciltacabtagene autoleucel cells; KITE585 cells; CT053 cells: BCMA-CS1 cCAR (BC1cCAR) cells; P-BCMA-101 cells: P-BCMA-ALLO1 cells: C-CAR088 cells: Descartes-08 cells; PBCAR269A cells; ALLO-715 cells; PHE885 cells; AUTOS cells; CTX120 cells; CB-011 cells; ALLO-605 (TuboCAR/MM) cells; pCDCAR1 (TriCAR-Z136) cells, or GC012F cells. In some embodiments, the dose of genetically engineered T cells comprises idecabtagene vicleucel cells (e.g., such as ABECMA® cells).

### B. Cells and Preparation of Cells for Genetic Engineering

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells. TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes. B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., Blood. 1:72-82 (2012); Wang et al., J Immunother. 35(9):689-701 (2012). In some embodiments, combining TCM-enriched CD8⁺ T cells and CD4⁺ T cells further enhances efficacy.

In embodiments, memory T cells are present in both CD62L⁺ and CD62L− subsets of CD8-peripheral blood lymphocytes. PBMC can be enriched for or depleted of CD62L-CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T(m cells is carried out by depletion of cells expressing CD4, CD14, CD45RA, and positive selection or enrichment for cells expressing CD62L. In one aspect, enrichment for central memory T (Tc) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4C cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO−, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L− and CD45RO−.

In one example, to enrich for CD4⁺ cells by negative selection, a monoclonal antibody cocktail typically includes antibodies to CD14, CD20, CD11b. CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher C Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in PCT Pub. Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al., J Immunother. 35(9): 651-660 (2012), Terakura et al., Blood. 1:72-82 (2012), and Wang et al., J Immunother. 35(9):689-701 (2012).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., Lab Chip 10, 1567-1573 (2010); and Godin et al., J Biophoton. 1(5):355-376 (2008). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al., J Immunother. 35(9): 651-660 (2012). Terakura et al., Blood. 1:72-82 (2012), and/or Wang et al., J Immunother. 35(9):689-701 (2012).

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### C. Nucleic Acids, Vectors and Methods for Genetic Engineering

In some embodiments, the cells, e.g., T cells, are genetically engineered to express a recombinant receptor. In some embodiments, the engineering is carried out by introducing nucleic acid molecules that encode the recombinant receptor. Also provided are nucleic acid molecules encoding a recombinant receptor, and vectors or constructs containing such nucleic acids and/or nucleic acid molecules.

In some cases, the nucleic acid sequence encoding the recombinant receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some embodiments, the signal peptide is derived from a transmembrane protein. In some examples the signal peptide is derived from CD8a, CD33, or an IgG. Non-limiting exemplary examples of signal peptides include, for example, the CD33 signal peptide set forth in SEQ ID NO: 153, CD8a signal peptide set forth in SEQ ID NO:154, or the signal peptide set forth in SEQ ID NO:39 or modified variant thereof. In some embodiments, the signal peptide is the CD8a signal peptide set forth in Accession No. NM_001768.

In some embodiments, the nucleic acid molecule encoding the recombinant receptor contains at least one promoter that is operatively linked to control expression of the recombinant receptor. In some examples, the nucleic acid molecule contains two, three, or more promoters operatively linked to control expression of the recombinant receptor. In some embodiments, nucleic acid molecule can contain regulatory sequences, such as transcription and translation initiation and termination codons, which are specific to the type of host (e.g., bacterium, fungus, plant, or animal) into which the nucleic acid molecule is to be introduced, as appropriate and taking into consideration whether the nucleic acid molecule is DNA- or RNA-based. In some embodiments, the nucleic acid molecule can contain regulatory/control elements, such as a promoter, an enhancer, an intron, a polyadenylation signal, a Kozak consensus sequence, and splice acceptor or donor. In some embodiments, the nucleic acid molecule can contain a nonnative promoter operably linked to the nucleotide sequence encoding the recombinant receptor and/or one or more additional polypeptide(s). In some embodiments, the promoter is selected from among an RNA pol 1, pol II or pol III promoter. In some embodiments, the promoter is recognized by RNA polymerase II (e.g., a CMV. SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with mycloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the nucleic acid molecule does not include a regulatory element, e.g. promoter.

In some embodiments, the nucleic acid molecule encoding the recombinant receptor, e.g., CAR or other antigen receptor, further includes nucleic acid sequences encoding a marker and/or cells expressing the CAR or other antigen receptor further includes a marker, e.g., a surrogate marker, such as a cell surface marker, which may be used to confirm transduction or engineering of the cell to express the receptor, such as a truncated version of a cell surface receptor, such as truncated EGFR (tEGFR). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the nucleic acid molecule, e.g., a nucleic acid molecule encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same nucleic acid molecule that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 166) or a prostate-specific membrane antigen (PSMA) or modified form thereof, tEGFR may contain an epitope recognized by the antibody cetuximab (Erbitux®)) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some aspects, the marker, e.g. surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:7 or 166. An exemplary T2A linker sequence comprises the sequence of amino acids set forth in SEQ ID NO:6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 166.

In some embodiments, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus (F2A; e.g. SEQ ID NO:172), equine rhinitis A virus (E2A; e.g. SEQ ID NO:170), Thosea asigna virus (T2A, e.g., SEQ ID NO: 6 or 167), and porcine teschovirus-1 (P2A; e.g. SEQ ID NO:168 or 169) as described in U.S. Patent Publication No. 20070116690.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as ‘self’ by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Introduction of the nucleic acid molecules encoding the recombinant receptor in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46: Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), mycloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740, 6,207,453: 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods. Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection: tungsten particle-facilitated microparticle. bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See. e.g., Riddell et al., U.S. Pat. No. 6,040,177, at columns 14-17.

### V. COMPOSITIONS AND FORMULATIONS

Also provided are compositions including the BCMA recombinant receptors, and engineered cells, including pharmaceutical compositions and formulations. Among such compositions are those that include engineered cells, such as a plurality of engineered cells, expressing the provided anti-BCMA recombinant receptors (e.g. CARs). In some aspects, also provided are compositions, e.g., cell compositions for use in the provided methods and uses, e.g., therapeutic methods and uses. In some embodiments, the provided compositions are capable of achieving certain therapeutic outcomes, e.g., response or safety outcomes, when administered to subjects that have a disease or disorder, e.g., multiple myeloma.

Provided are pharmaceutical formulations comprising a BCMA-binding recombinant chimeric antigen receptors or engineered cells expressing said receptors, a plurality of engineered cells expressing said receptors and/or additional agents for combination treatment or therapy. The pharmaceutical compositions and formulations generally include one or more optional pharmaceutically acceptable carrier(s) or excipient(s). In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium: metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy. Lippincott Williams & Wilkins; 21st ed. (May 1, 2005).

Formulations of the antibodies described herein can include lyophilized formulations and aqueous solutions.

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells containing the binding molecules, e.g., CAR, a subject is administered the range of at or about one million to at or about 100 billion cells, such as, e.g., 1 million to at or about 50 billion cells (e.g., at or about 5 million cells, at or about 25 million cells, at or about 50 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 100 million cells, at or about 120 million cells, at or about 150 million cells, at or about 250 million cells, at or about 300 million cells, at or about 350 million cells, at or about 450 million cells, at or about 650 million cells, at or about 800 million cells, at or about 900 million cells, at or about 1.2 billion cells, at or about 3 billion cells, at or about 30 billion cells, at or about 45 billion cells) or any value in between these ranges, and/or such a number of cells per kilogram of body weight of the subject. In some aspects, in the context of genetically engineered cells expressing the binding molecules, e.g., CAR, a composition can contain at least the number of cells for administration for a dose of cell therapy, such as about or at least a number of cells described herein for administration, e.g., in Section II.C.

The may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### VI. ARTICLES OF MANUFACTURE AND KITS

Also provided are articles of manufacture or kit containing the provided recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., multiple myeloma). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the BCMA-expressing or BCMA-associated disease, disorder or condition in an individual. The label or package insert may indicate that the composition is used for treating a multiple myeloma, such as a relapsed and refractory multiple myeloma. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a multiple myeloma, such as a relapsed and refractory multiple myeloma, in an individual. The label or package insert may further indicate that the formulation is useful or intended for subcutaneous, intravenous, or other modes of administration for treating or preventing a multiple myeloma, including relapsed and refractory multiple myeloma.

The container in some embodiments holds a composition which is by itself or combined with another composition effective for treating, preventing and/or diagnosing the multiple myeloma. The article of manufacture or kit may include (a) a first container with a composition contained therein (i.e., first medicament), wherein the composition includes the antibody (e.g., anti-BCMA antibody) or antigen-binding fragment thereof or recombinant receptor (e.g., CAR); and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

Also provided are articles of manufacture or kit containing one or more reagents for determining the level of serum sBCMA and/or the presence of IgG in a sample obtained in a subject. In some embodiments, the kit further comprises instructions for using the one or more reagents to determine the level of serum sBCMA and/or the presence of IgG in the sample.

### VII. DEFINITIONS

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

“Human BCMA” refers to BCMA found in a human subject, and having, e.g., SEQ ID NO: 215.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “Intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-BCMA antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides, e.g., linkers and BCMA-binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, T cell therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### VIII. EXEMPLARY EMBODIMENTS

Among the provided embodiments are:
1. A method of treating a subject having a multiple myeloma (MM), comprising:
   (a) determining that a subject has:
      (i) a scrum soluble B cell maturation antigen (sBCMA) level lower than about 600 ng/ml; and/or
      (ii) an absence of IgG heavy chain disease (HCD); and
   (b) administering to the subject a T cell therapy comprising a dose of genetically engineered T cells expressing a chimeric antigen receptor (CAR) that binds to human BCMA.
2. A method of treating a subject having a multiple myeloma (MM), comprising administering to the subject a T cell therapy comprising a dose of genetically engineered T cells expressing a chimeric antigen receptor (CAR) that binds to human B cell maturation antigen (BCMA), wherein the subject was previously determined to have:
   (i) a serum soluble B cell maturation antigen (sBCMA) level lower than about 600 ng/ml; and/or
   (ii) an absence of IgG heavy chain disease (HCD).
3. A method of treating a subject having a multiple myeloma (MM), comprising:
   (a) selecting a subject for treatment with a T cell therapy comprising a dose of genetically engineered T cells expressing a chimeric antigen receptor (CAR) that binds to human BCMA, wherein the subject was previously determined to have:
      (i) a serum soluble B cell maturation antigen (sBCMA) level lower than about 600 ng/ml; and/or
      (ii) an absence of IgG heavy chain disease (HCD); and
   (b) administering the T cell therapy to the subject.
4. A method of selecting a subject having a multiple myeloma (MM) for treatment with a T cell therapy comprising a dose of genetically engineered T cells, comprising determining that a subject has:
   (i) a serum soluble B cell maturation antigen (sBCMA) level lower than about 600 ng/ml; and/or
   (ii) an absence of IgG heavy chain disease (HCD),
   wherein, if the subject is determined to have (i) and/or (ii), the subject is selected for administration with a T cell therapy comprising a dose of genetically engineered T cells expressing a chimeric antigen receptor (CAR) that binds to human BCMA.
5. A method of predicting the response of a subject having a multiple myeloma (MM) to treatment with a T cell therapy comprising a dose of genetically engineered T cells expressing a chimeric antigen receptor (CAR) that binds to human B cell maturation antigen (BCMA), comprising determining that a subject has:
   (i) a serum soluble B cell maturation antigen (sBCMA) level lower than about 600 ng/ml; and/or
   (ii) an absence of IgG heavy chain disease (HCD),
   wherein, if the subject has (i) and/or (ii), the subject is predicted to achieve a complete response (CR) or a stringent complete response (sCR),
   and wherein the treatment comprises administration of the dose of genetically engineered T cells to the subject.
6. A method of treating a subject having a multiple myeloma (MM), comprising:
   (a) determining that the subject has:
      (i) a serum soluble B cell maturation antigen (sBCMA) level higher than about 600 ng/ml; and/or
      (ii) a presence of IgG heavy chain disease (HCD);
   (b) debulking the MM; and
   (c) administering to the subject a T cell therapy comprising a dose of genetically engineered T cells expressing a chimeric antigen receptor (CAR) that binds to human BCMA.
7. A method of treating a subject having a multiple myeloma (MM), comprising administering to a subject a T cell therapy comprising a dose of genetically engineered T cells expressing a chimeric antigen receptor (CAR) that binds to human B cell maturation antigen (BCMA), wherein:
   (a) the subject was previously determined to have:
      (i) a serum soluble B cell maturation antigen (sBCMA) level higher than about 600 ng/ml; and/or
      (ii) a presence of IgG heavy chain disease (HCD); and
   (b) the MM was debulked at a time between: (1) the subject being determined to have (i) and/or
   (ii); and (2) the subject being administered the T cell therapy.
8. A method of selecting a subject having a multiple myeloma (MM) for de-bulking, comprising determining that a subject has:
   (i) a serum soluble B cell maturation antigen (sBCMA) level higher than about 600 ng/ml; and/or
   (ii) a presence of IgG heavy chain disease (HCD),
   wherein, if the subject is determined to have (i) and/or (ii), the subject is selected for debulking the MM prior to administration to the subject of a T cell therapy comprising a dose of genetically engineered T cells expressing a chimeric antigen receptor (CAR) that binds to human BCMA.
9. A method of predicting the response of a subject having a multiple myeloma (MM) to treatment with a T cell therapy comprising a dose of genetically engineered cells expressing a chimeric antigen receptor (CAR) that binds to human B cell maturation antigen (BCMA), comprising determining that a subject has:
   (i) a serum soluble B cell maturation antigen (sBCMA) level higher than about 600 ng/ml; and/or
   (ii) a presence of IgG heavy chain disease (HCD),
   wherein, if the subject has (i) and/or (ii), the subject is predicted not to achieve a complete response (CR) or stringent complete response (sCR)),
   and wherein the treatment comprises administration of the dose of genetically engineered T cells to the subject.
10. A method of treating a subject having a multiple myeloma (MM), comprising:
   (a) determining that a subject has a first serum soluble B cell maturation antigen (sBCMA) level higher than about 600 ng/ml;
   (b) debulking the MM;
   (c) determining that the subject has a post-debulking serum sBCMA level lower than about 600 ng/ml; and
   (d) administering to the subject a T cell therapy comprising a dose of genetically engineered T cells expressing a chimeric antigen receptor (CAR) that binds to human BCMA.
11. The method of embodiment 2, 3, or 7, further comprising determining that the subject has:
   (i) a serum sBCMA level or a post-debulking serum sBCMA level higher or lower than about 600 ng/ml; and/or
   (ii) a presence or absence of IgG HCD.
12. The method of any one of embodiments 1-11, wherein the method comprises determining that the subject has a serum sBCMA level or a post-debulking serum sBCMA level higher or lower than about 600 ng/ml.
13. The method of any one of embodiments 1-12, wherein:
   the method comprises determining that the subject has a serum sBCMA level higher or lower than about 590 ng/ml, about 580 ng/ml, about 570 ng/ml, about 566 ng/ml, about 560 ng/ml, about 550 ng/ml, about 540 ng/ml, about 530 ng/ml, about 520 ng/ml, about 510 ng/ml, or about 500 ng/ml; and/or
   the subject is determined to have a serum sBCMA level higher or lower than about 590 ng/ml, about 580 ng/ml, about 570 ng/ml, about 566 ng/ml, about 560 ng/ml, about 550 ng/ml, about 540 ng/ml, about 530 ng/ml, about 520 ng/ml, about 510 ng/ml, or about 500 ng/ml.
14. The method of any one of embodiments 1-9 and 11-13, wherein the method comprises determining that the subject has a presence or absence of IgG HCD.
15. The method of any one of embodiments 1-9 and 11-14, wherein determining the subject has a presence of IgG HCD comprises detecting IgG in serum and/or urine of the subject.
16. The method of any one of embodiments 1-15, wherein the determination of the serum sBCMA level is carried out about three months before, about two months before, about one month before, about three weeks before, about two weeks before, or about one week before: (i) administration of the T cell therapy to the subject; or (ii) the cells of the dose of genetically engineered CAR T cells are obtained from the subject.
17. The method of any one of embodiments 1-9 and 11-16, wherein the determination that the subject has a presence or absence of IgG HCD is carried out between about three months before, about two months before, about one month before, about three weeks before, about two weeks before, or about one week before: (i) administration of the T cell therapy to the subject; or (ii) the cells of the dose of genetically engineered CAR T cells are obtained from the subject.
18. The method of any one of embodiments 4, 5, 8, 9, and 12-17, further comprising administering the dose of genetically engineered cells to the subject.
19. The method of any one of embodiments 5, 9, and 12-18, wherein:
   (i) if the subject is predicted to achieve a CR or a sCR, the method further comprises administering to the subject a T cell therapy comprising a dose of genetically engineered T cells expressing a chimeric antigen receptor (CAR) that binds to BCMA; or
   (ii) if the subject is predicted not to achieve a CR or a sCR, the method further comprises selecting the subject for debulking the MM prior to administration to the subject of a dose of genetically engineered T cells expressing a chimeric antigen receptor (CAR) that binds to BCMA.
20. The method of any one of embodiments 1-19, wherein, following administration of the dose of genetically engineered T cells to the subject, the subject achieves a complete response (CR) or a stringent complete response (sCR).
21. The method of any one of embodiments 1-20, wherein, following administration of the dose of genetically engineered T cells to the subject, the subject achieves a CR.
22. The method of any one of embodiments 1-20, wherein, following administration of the dose of genetically engineered T cells to the subject, the subject achieves a sCR.
23. The method of any one of embodiments 6-8, 10, and 12-22, wherein the debulking comprises administering chemotherapy, radiation, and/or an immunomodulatory agent to the subject.
24. The method of embodiment 23 wherein the chemotherapy comprises melphalan, doxorubicin, or cyclophosphamide chemotherapy.
25. The method of embodiment 23 or embodiment 24 wherein the immunomodulatory agent is thalidomide, lenalidomide, or pomalidomide.
26. The method of any one of embodiments 23-25, wherein the immunomodulatory agent is a checkpoint inhibitor.
27. The method of any one of embodiments 6-8, 10, and 12-26, wherein the debulking is carried out within about three months before, within about two months before, within about one month before, within about three weeks before, within about two weeks before, or within about one week before administration of the T cell therapy to the subject.
28. The method of any one of embodiments 1-27, wherein prior to the administration of the dose of genetically engineered T cells to the subject, the subject has received a lymphodepleting therapy comprising the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.
29. The method of any one of embodiments 1-28, wherein prior to the administration of the dose of genetically engineered T cells to the subject, the subject has received a lymphodepleting therapy comprising the administration of fludarabine at or about 30 mg/m² body surface area of the subject, daily, and cyclophosphamide at or about 300 mg/m² body surface area of the subject, daily, for 3 days.
30. The method of embodiment 28 or embodiment 29 wherein the debulking is carried out prior to the lymphodepleting therapy.
31. The method of any of embodiments 28-30, wherein the debulking is carried out after the lymphodepleting therapy.
32. The method of any one of embodiments 1-31, wherein the MM is a high-risk MM and/or a relapsed and/or refractory (r/r) MM.
33. The method of any one of embodiments 1-32, wherein the MM is a high-risk MM.
34. The method of any one of embodiments 1-33, wherein the MM is a relapsed and/or refractory (r/r) MM.
35. The method of any one of embodiments 1-34, wherein the subject is 18 year of age or older.
36. The method of any one of embodiments 1-35, wherein the subject has previously received three or more prior lines of therapy for the MM.
37. The method of embodiment 36, wherein each of the three or more prior lines of therapy comprises two consecutive cycles, unless progressive disease (PD; progression within 60 days after last dose) was the best response to the line of therapy.
38. The method of embodiment 36 or embodiment 37, wherein the three or more prior lines of therapy comprise a proteasome inhibitor, an immunomodulatory agent, and an anti-CD38 antibody.
39. The method of any one of embodiments 36-38, wherein the subject is refractory to the last of the three or more prior lines of therapy.
40. The method of any one of embodiments 1-39, wherein the subject has an Eastern Cooperative Oncology Group (ECOG) performance status of 0 or 1.
41. The method of any one of embodiments 1-40, wherein the subject has measurable disease at the time of the administration of the dose of genetically engineered T cells.
42. The method embodiment 41, wherein measurable disease comprises:
   (i) serum M-protein greater or equal to 1.0 g/dL;
   (ii) urine M-protein greater or equal to 200 mg/24 h; and/or
   (iii) involved serum free light chain (FLC) level greater or equal to 10 mg/dL if serum FLC ratio is abnormal.
43. The method of any one of embodiments 1-42, wherein the subject does not have:
   (i) central nervous system (CNS) involvement; and/or
   (ii) a history or presence of clinically relevant CNS pathology.
44. The method of any one of embodiments 1-43, wherein the subject does not have active or a history of plasma cell leukemia (PCL).
45. The method of any one of embodiments 1-44, wherein the CAR comprises an extracellular antigen-binding domain that binds to BCMA, a transmembrane domain, and an intracellular signaling region.
46. The method of embodiment 45, wherein the extracellular antigen-binding domain comprises a variable heavy chain (V_{H}) region and, optionally, a variable light chain (V_{L}) region.
47. The method of embodiment 46, wherein:
   the V_{H} region comprises a CDR-H1, a CDR-H2, and a CDR-H3 comprising the amino acid sequences set forth in SEQ ID NOS: 189, 190, and 191, respectively; and the V_{L} region comprises a CDR-L1, a CDR-L2, and a CDR-L3 comprising the amino acid sequences set forth in SEQ ID NOS: 192, 193, and 194, respectively; or
   the V_{H} region comprises a CDR-H1, a CDR-H2, and a CDR-H3 comprising the amino acid sequences set forth in SEQ ID NOS: 173, 174 and 175, respectively; and the V_{L} region comprises a CDR-L1, a CDR-L2, and a CDR-L3 comprising the amino acid sequences set forth in SEQ ID NOS: 183, 184 and 185, respectively.
48. The method of embodiment 46 or embodiment 47, wherein:
   the V_{H} region comprises an amino acid sequence set forth in SEQ ID NO: 18 and the V_{L} region comprises the amino acid sequence set forth in SEQ ID NO: 19;
   or the V_{H} region comprises an amino acid sequence set forth in SEQ ID NO: 24, and the V_{L} region comprises the amino acid sequence set forth in SEQ ID NO: 25.
49. The method of any one of embodiments 45-48, wherein the extracellular antigen-binding domain is a single chain variable fragment (scFv).
50. The method of embodiment 49, wherein the scFv comprises the amino acid sequence set forth in SEQ ID NO: 213 or SEQ ID NO: 188.
51. The method of any one of embodiments 45-50, wherein the intracellular signaling region further comprises a costimulatory signaling domain.
52. The method of embodiment 51, wherein the costimulatory signaling domain comprises an intracellular signaling domain of CD28, 4-1BB, or ICOS, or a signaling portion thereof.
53. The method of embodiment 51 or embodiment 52, wherein the costimulatory signaling domain is between the transmembrane domain and the cytoplasmic signaling domain of a CD3-zeta (CD3ζ) chain.
54. The method of any one of embodiments 45-53, wherein the transmembrane domain is or comprises a transmembrane domain from CD28 or CD8, optionally human CD28 or CD8.
55. The method of any one of embodiments 45-54, wherein the CAR further comprises an extracellular spacer between the antigen binding domain and the transmembrane domain.
56. The method of embodiment 55, wherein the spacer is from CD8, optionally wherein the spacer is a CD8a hinge.
57. The method of embodiment 55 or embodiment 56, wherein the transmembrane domain and the spacer are from CD8.
58. The method of any one of embodiments 45-57, wherein the CAR comprises the amino acid sequence set forth in SEQ ID NO: 116 or SEQ ID NO: 124.
59. The method of any one of embodiments 45-58, wherein the CAR is encoded by the polynucleotide sequence set forth in SEQ ID NO: 214.
60. The method of any one of embodiments 1-59, wherein the CAR is ABECMA®.
61. The method of any one of embodiments 1-60, wherein the dose of genetically engineered T cells comprises: idecabtagene vicleucel cells; bb21217 cells; orvacabtagene autoleucel cells; CT103A cells; ciltacabtagene autoleucel cells: KITE585 cells; CT053 cells: BCMA-CS1 cCAR (BCIcCAR) cells; P-BCMA-101 cells; P-BCMA-ALLO1 cells; C-CAR088 cells; Descartes-08 cells; PBCAR269A cells; ALLO-715 cells: PHE885 cells; AUTO8 cells; CTX120 cells; CB-011 cells; ALLO-605 (TuboCAR/MM) cells; pCDCAR1 (TriCAR-Z136) cells, or GC012F cells.
62. The method of any one of embodiments 1-61, wherein the dose of genetically engineered T cells comprises idecabtagene vicleucel cells.
63. The method of any of embodiments 1-62, wherein the dose of genetically engineered T cells comprises CD3⁺ CAR-expressing T cells.
64. The method of any of embodiments 1-63, wherein the dose of genetically engineered T cells comprises a combination of CD4⁺ T cells and CD8⁺ T cells and/or a combination of CD4⁺ CAR-expressing T cells and CD8⁺ CAR-expressing T cells.
65. The method of embodiment 64, wherein the ratio of CD4⁺ CAR-expressing T cells to CD8⁺ CAR-expressing T cells and/or of CD4⁺ T cells to CD8⁺ T cells, is or is approximately 1:1 or is between at or approximately 1:3 and at or approximately 3:1.
66. The method of any of embodiments 1-65, wherein:
   the percentage of naive-like T cells and/or central memory T cells is greater than or greater than about 60% of the total genetically engineered T cells in the dose, optionally greater than or greater than about 65%, 70%, 80%, 90% or 95%;
   the percentage of naive-like T cells and/or central memory T cells is greater than or greater than about 40% of the total CD4+ genetically engineered T cells in the dose, optionally greater than or greater than about 50%, 60%, 70%, 80%, 90% or 95%; or
   the percentage of naive-like T cells and/or central memory T cells is greater than or greater than about 40% of the total CD8+ genetically engineered T cells in the dose, optionally greater than or greater than about 50%, 60%, 70%, 80%, 90% or 95%.
67. The method of embodiment 66, wherein the naive-like T cells are CCR7+CD45RA+, CD27+CCR7+, or CD62L-CCR7+.
68. The method of any one of embodiments 1-67, wherein the dose of genetically engineered T cells comprises between about 0.5×10⁶ and about 600×10⁶ CAR-positive T cells.
69. The method of any one of embodiments 1-68, wherein the dose of genetically engineered T cells comprises between about 100×10⁶ and about 600×10⁶ CAR-positive T cells.
70. The method of any one of embodiments 1-69, wherein the dose of genetically engineered T cells comprises between about 150×10⁶ and about 450×10⁶ CAR-positive T cells.
71. The method of any one of embodiments 1-70, wherein the dose of genetically engineered T cells comprises about 150×10⁶, 300×10⁶, or about 450×10⁶ CAR-positive T cells.
72. The method of any one of embodiments 1-68, wherein the dose of genetically engineered T cells comprises between about 0.5×10⁶ and about 10×10⁶ CAR-positive T cells.
73. The method of any one of embodiments 1-72, wherein the cells of the dose of genetically engineered CAR T cells were obtained from the subject.
74. The method of any one of embodiments 1-73, wherein the dose of genetically engineered T cells are autologous to the subject.
75. The method of any one of embodiments 1-72, wherein the dose of genetically engineered T cells are allogencic to the subject.

### IX. EXAMPLES

The following example is included for illustrative purposes only and is not intended to limit the scope of the invention.

### Example 1: Response to a BCMA-Directed CAR T Cell Therapy in Patients with Relapsed and Refractory Multiple Myeloma

Chimeric antigen-receptor (CAR)-expressing T cell compositions containing autologous T cells expressing a CAR specific for B-cell maturation antigen (BCMA) were administered to human subjects with relapsed and refractory (R/R) multiple myeloma (MM).

### A. Subjects and Treatment

Compositions containing autologous T cells engineered to express an exemplary anti-BCMA CAR were administered to adult human subjects with R/R MM who had received 3 or more prior lines of therapy (the three or more prior lines of therapy including an immunomodulatory agent, a proteasome inhibitor (PI), and an anti-CD38 antibody) and were disease refractory to the previous line of therapy per International Myeloma Working Group (IMWG) criteria.

The administered T cell compositions were generated by obtaining peripheral-blood mononuclear cells (PBMCs) from leukapheresis samples from individual subjects with MM, stimulating the PBMCs with anti-CD3 and anti-CD28 antibodies, transducing the cells with a lentiviral vector containing the exemplary anti-BCMA CAR, and expanding the cells for 10 days prior to cryopreservation. The exemplary CAR contained an anti-BCMA scFv, a hinge and transmembrane domain from a CD8a, and a CD137 (4-1BB) co-stimulatory domain followed by the intracellular signaling domain of a CD3 chain. The polynucleotide sequence of the exemplary BCMA CAR is set forth in SEQ ID NO: 214, and the polypeptide sequence of the exemplary BCMA CAR is set forth in SEQ ID NO: 116.

Subjects received a lymphodepleting chemotherapy (LDC) with fludarabine (flu, 30 mg/m²/day) and cyclophosphamide (cy, 300 mg/m²/day) for 3 consecutive days, the LDC being completed 2 days before CAR T cell infusion. Bridging therapy was allowed during CAR T cell manufacturing, but was stopped at least 14 days before LDC. The cryopreserved cell compositions were thawed at bedside prior to intravenous administration, and subjects were administered a dose of 150-450×10⁶ (e.g., 150, 300, or 450×10⁶) total CAR-expressing T cells.

### B. Outcomes and Analysis

The primary endpoint was overall response rate (ORR), and complete response/stringent complete response (CR/sCR) rate was a key secondary endpoint. Complete response was defined as no more myeloma protein in blood and urine and <5% myeloma cells in bone marrow. Other secondary endpoints included safety, duration of response (DOR), progression free survival (PFS), overall survival (OS), pharmacokinetics (PK), minimum residual disease (MRD; as assessed by next-generation sequencing), quality of life (QOL), and health economics outcomes research (HEOR). Baseline characteristics were collected prior to lymphodepletion and, for select biomarkers, on day 1. Univariate and multivariate logistic regression models were used to identify baseline characteristics that correlated with the likelihood of achieving a CR/sCR.

In total, 128 of 140 patients had received CAR T cell infusions at the time of results depicted in this example. 42 patients achieved a CR/sCR and 86 exhibited non-CR/sCR (very good partial response [VGPR], partial response [PR], or no response). Among subjects with CR/sCR, 32 (76%) were negative for minimal residual disease (MRD) at a sensitivity level of <10-5 nucleated cells and 19 of these patients maintained MRD negativity at the 12-month follow-up (Table E1).

TABLE E1
Proportion of patients with CR/sCR and MRD negative status

| | CR/SCR (n = 42) |
|---|---|
| Overall MRD negativity^{a}, n (%) | 32 (76.2) |
| Month 6 | 19 (45.2) |
| Month 12 | 19 (45.2) |
| Month 18 | 12 (28.6) |
| Month 24 | 1 (2.4) |

The negative MRD value after infusion is considered. Only a negative MRD value within 3 months prior to achieving CR or above until time of progression/death (exclusive) is considered. The number of patients with CR/sCR is used as the denominator, which includes patients with positive, indeterminate, or missing MRD values. The number of patients evaluated for MRD decreased with time.
^{a}MRD negativity defined as <10⁻⁵ nucleated cells.

Baseline demographics and disease characteristics were generally balanced between patients with CR/sCR and non-CR/sCR. Notable exceptions included revised International Staging System (ISS) stage III disease, presence of immunoglobulin G (IgG) heavy chain type disease, CD138+ plasma cell percentage, and β-2-microglobulin levels (Table E2).

TABLE E2
Baseline characteristics in patients with CR/sCR and non-CR/sCR

| | CR/sCR | | Non-CR/sCR | |
|---|---|---|---|---|
| Characteristic | (n = 42) | | (n = 86) | |
| Age, median (range), years | 59.5 | (38-78) | 61.0 | (33-77) |
| Female, n (%) | 19 | (45.2) | 33 | (38.4) |
| ECOG performance status, n (%) | | | | |
| 0 | 18 | (42.9) | 39 | (45.3) |
| 1 | 24 | (57.1) | 44 | (51.2) |
| 2 | 0 | | 3 | (3.5) |
| Revised ISS Stage III (derived), n (%) | 2 | (4.8) | 19 | (22.1) |
| High risk cytogenetics,^{a} n (%) | 15 | (35.7) | 30 | (34.9) |
| Number of prior regimens, median (range) | 6 | (3-13) | 6 | (3-16) |
| Triple refractory, n (%) | 35 | (83.3) | 73 | (84.9) |
| Bridging therapy, n (%) | 37 | (88.1) | 75 | (87.2) |
| Heavy chain disease, n (%) | 27 | (64.3) | 77 | (89.5) |
| IgG | 14 | (33.3) | 65 | (75.6) |
| Bone marrow biopsy CD138+ plasma cells, | (n = 40) | | (n = 82) | |
| median (range), % | 35 (0-95) | | 60.0 (0-100) | |
| Tumor BCMA expression ≥50%^{b}, n (%) | 37 | (88.1) | 72 | (83.7) |
| β-2-microglobulin, median (range), mg/L | 3.1 | (1.3-23.0) | 4.1 | (1.6-32.0) |
| Hemoglobin, median (range), g/L | 106.5 | (71.0-137.0) | 97.0 | (67.0-148.0) |
| Lymphocyte count, median (range), ×10⁹/L | 0.66 | (0.07-3.30) | 0.60 | (0.04-2.17) |
| D-dimer, median (range), mg/L | (n = 41) | | (n = 79) | |
| | 0.53 (0.19-2.74) | | 0.88 (0.22-19.62) | |
| Ferritin, median (range), ug/L | (n = 42) | | (n = 83) | |
| | 168.5 (8.0-2293.0) | | 430.0 (23.0-7883.0) | |
| Sodium, median (range), mmol/L | 141 | (134-144) | 138 | (126-146) |
| Corrected serum calcium, median (range), | (n = 38) | | (n = 83) | |
| mmol/L | 2.4 (2.1-3.5) | | 2.4 (2.0-3.0) | |
| Albumin, median (range), g/dL | 3.7 | (2.8-4.9) | 3.8 | (1.6-5.0) |

^{a}Includes del (17p), t(4:14), and t(14; 16);
^{b}No minimum tumor BCMA expression required for study entry.

Univariate analysis of CR/sCR by baseline characteristics showed that IgG heavy chain vs. other heavy chain types (odds ratio [OR]:0.162, P<0.0001), high soluble BCMA (sBCMA) (OR: 0.646, P=0.0007), β-2-microglobulin (≥5.5 vs <3.5 mg/L, OR: 0.201, P=0.0072), and presence of extramedullary disease (OR: 0.428, P=0.0394) were negatively associated with CR/sCR, whereas high vector copy number in drug product was positively associated with CR/sCR (OR: 1.290, P=0.0287) (Table E3). A multivariate analysis of CR/sCR identified IgG heavy chain vs. other heavy chain types (OR: 0.100, P<0.0001), high sBCMA (OR: 0.637, P=0.0110), and elevated prothrombin time-international normalized ratio (PT-INR) test (OR: 0.005, P=0.0365) as negative correlates of CR/sCR, and high vector copy number in drug product (OR: 1.486, P=0.0168) as a positive correlate of CR/sCR (Table E3). Descriptive analysis demonstrated lower baseline sBCMA in patients with CR/sCR vs. non-CR/sCR.

TABLE E3
Correlates of CR/sCR vs. non-CR/sCR

| | | | |
|---|---|---|---|
| | | | Positive or |
| | | | negative |
| Baseline patient variable or drug product | Odds | P | correlate of |
| characteristic | ratio | value | CR/SCR^{a} |

Univariate correlates of CR/sCR vs. non-CR/sCR

| | | | |
|---|---|---|---|
| Presence of IgG heavy chain disease | 0.162 | <0.0001 | Negative |
| sBCMA (ng/mL)^{b} | 0.646 | 0.0007 | Negative |
| D-dimer (mg/L) | 0.559 | 0.0015 | Negative |
| B-2 microglobulin ≥5.5 | 0.201 | 0.0072 | Negative |
| mg/L (vs <3.5 mg/L) | | | |
| Ferritin (ug/L) | 0.802 | 0.0155 | Negative |
| Revised ISS stage III (vs I/II) | 0.168 | 0.0207 | Negative |
| Presence of extramedullary disease | 0.428 | 0.0394 | Negative |
| Hemoglobin (g/L) | 1.025 | 0.0255 | Positive |
| Vector copy number in drug product | 1.290 | 0.0287 | Positive |

Multivariate correlates of CR/sCR vs. non-CR/sCR

| | | | |
|---|---|---|---|
| Presence of IgG heavy chain disease | 0.100 | <0.0001 | Negative |
| sBCMA (ng/mL)^{b} | 0.637 | 0.0110 | Negative |
| PT-INR^{c} | 0.005 | 0.0365 | Negative |
| Vector copy number in drug product | 1.486 | 0.0168 | Positive |

^{a}For continuous variables, positive correlates of CR/SCR indicate that higher values increased the probability of CR/sCR; negatives correlates indicate that higher values decreased the probability of CR/sCR.
^{b}sBCMA values were reported as integers.
^{c}PT-INR was reported in increments of 0.1.

Across both groups, sBCMA levels increased between screening and baseline (Table E4). As shown in Table E5, which separates the baseline numbers in Table E2 into selective groups, only 5% of subjects exhibiting CR/sCR had a baseline sBCMA level of greater than 556 ng/mL, whereas 31.33% of subjects exhibiting no CRs/CR had a baseline sBCMA level of greater than 556 ng/mL.

TABLE E4
sBCMA levels at screening and baseline
in patients with CR/sCR and non-CR/sCR

sBCMA, median (range), ng/mL

| Response Category | Screening | Baseline |
|---|---|---|
| CR/sCR (n = 42) | n = 41 | n = 40 |
| | 161 (27-689) | 191 (11-909) |
| non-CR/sCR (n = 86) | n = 86 | n = 83 |
| | 302 (24-1490) | 340 (19-2735) |

TABLE E5
Response by sBCMA level at baseline

| | | Response | | |
|---|---|---|---|---|
| sBCMA (ng/mL) | | CR or sCR | No CR or sCR | Total |
| 11-556 | Frequency | 38 | 57 | 95 |
| | Col Pct | 95.00 | 68.67 | |
| >556-<1100 | Frequency | 2 | 20 | 22 |
| | Col Pct | 5.00 | 24.10 | |
| >1101-1645 | Frequency | 0 | 5 | 5 |
| | Col Pct | 0.00 | 6.02 | |
| >2190-2735 | Frequency | 0 | 1 | 1 |
| | Col Pct | 0.00 | 1.20 | |
| Total | Frequency | 40 | 83 | 123 |

To identify additional correlates of CR/sCR, multivariate XGBoost modeling and SHAP analysis were conducted. An integrated data set of 98 patient-level and laboratory variables, as well as biomarker and drug product characteristics, from 128 patients were compiled. This data set was further pre-processed, transformed, and highly correlated features were removed before a multivariate XGBoost model was trained on the final data se with 60 variables. Cross-validation and hyper-parameter tuning was used to select the best model, and the full model was trained with the best parameter. The most predicted features and their contribution to CR/sCR prediction were identified used Shapley additive explanations (SHAP) analysis, then evaluated for consistency and association with the dependent variable individually. Features without univariate differences in median between CR/sCR and non-CR/sCR groups could be identified in the model based on their difference in distributions and interactions with other features.

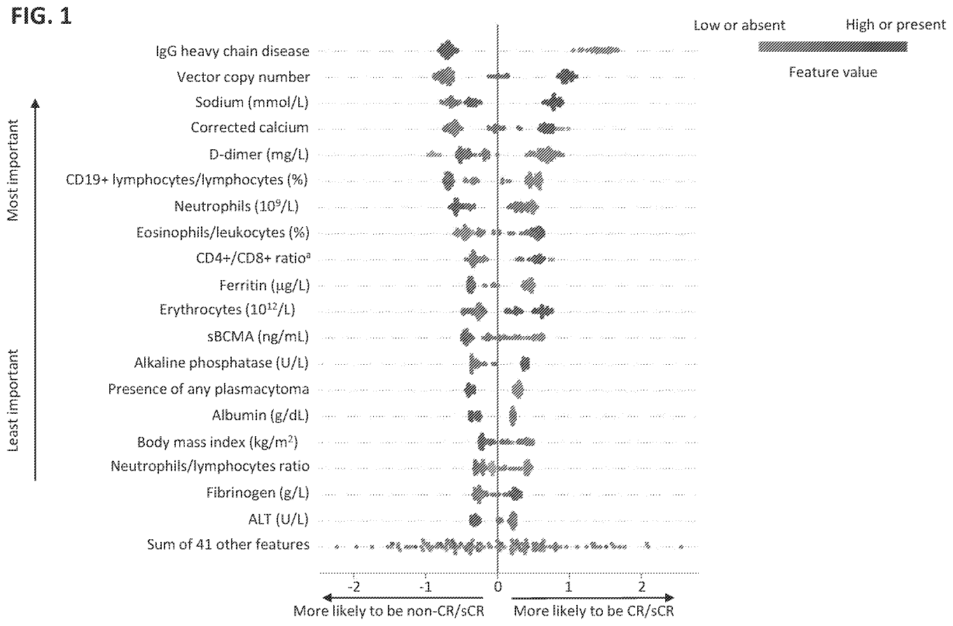

FIG. 1 shows baseline features ranked by importance, with dark gray indicating high or present levels and light gray indicating low or absent levels. Features correlating with an increased likelihood of CR/sCR are shown on the right side of the graph, and features correlating with an increased likelihood of non-CR/sCR are shown on the left side of the graph. Consistent with the more narrow multivariate regression model, presence of IgG heavy chain disease and higher sBCMA were found to negatively correlate with CR/sCR, and higher vector copy number was found to positively correlate with CR/sCR. Additional features found to negatively correlate with CR/sCR, as identified by the XGBoost model, included higher D-dimer, higher ferritin, and lower sodium.

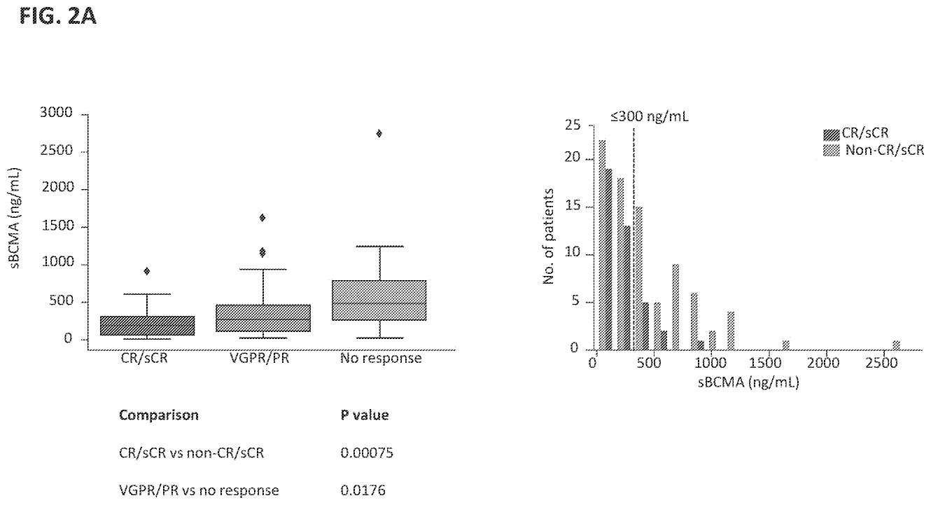

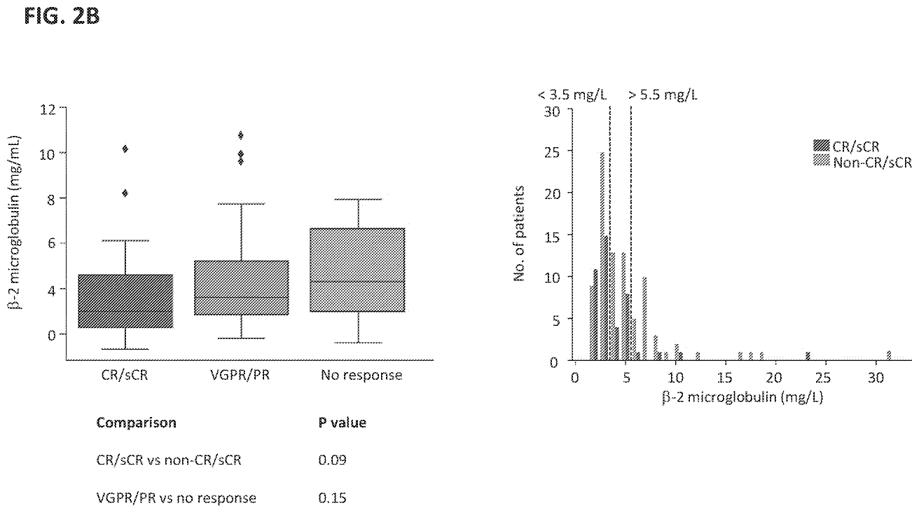

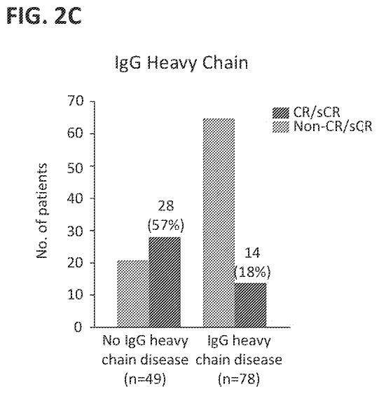

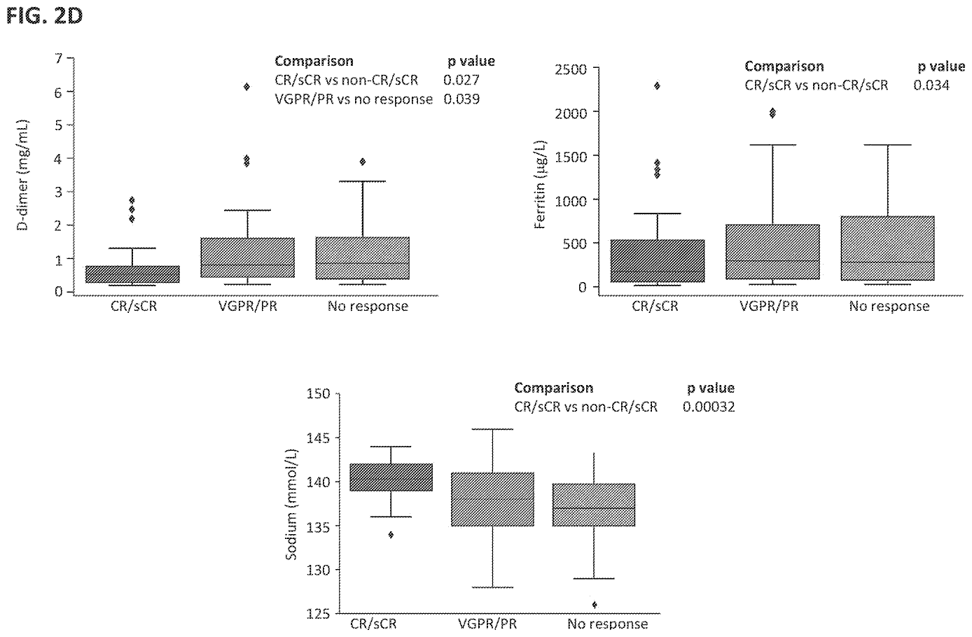

Based on the results of the XGBoost model, additional select univariate analyses were conducted to identify baseline factors that correlated with CR/sCR, VGPR/PR, or no response. Lower baseline levels of sBCMA were associated with deeper clinical responses (FIG. 2A), and higher β-2-microglobulin levels suggested a correlation with lack of response (FIG. 2B). Baseline sBCMA levels of ≤300 ng/mL distinguished most patients with CR/sCR from those with other responses.

The absence of IgG heavy chain disease at baseline was also observed to correlate with CR/sCR (FIG. 2C); only 18% of patients with IgG heavy chain disease exhibited CR/sCR vs. 57% of patients with another chain type of heavy chain disease or undetected heavy chain disease. The median PFS was 8.1 months (95% CI, 4.9-11.0) in patients with IgG heavy chain disease vs. 11.9 months (95% CI, 4.9-16.2) in patients with another chain type of heavy chain disease or undetected heavy chain disease. This difference in CR/sCR could be confounded by the longer half-life reported for IgG antibodies versus non-IgG heavy chains or light chains, leading to a longer time to reach CR/sCR.

Measurements of clinical baseline labs, including indicators of inflammation, showed that D-dimer and ferritin levels were significantly lower in patients with CR/sCR vs. non-CR/sCR, whereas sodium levels were significantly lower in patients with non-CR/sCR vs. CR/sCR (FIG. 2D).

To further assess serum sBCMA levels as a correlate of response, sBCMA levels at screening and baseline were assessed by bridging therapy in patients with CR/sCR vs. non-CR/sCR. Among 112 patients who received bridging therapy, 37 exhibited CR/sCR, and 75 exhibited non-CR/sCR. Baseline sBCMA levels were lower among patients with CR/sCR than patients with non-CR/sCR, regardless of whether bridging therapy was received by the patient (Table E6). In addition, greater increases in serum BCMA levels were observed between screening and just prior to CAR T cell infusion among patients who did not achieve a CR/sCR.

TABLE E6
sBCMA levels at screening and baseline by bridging
therapy in patients with CR/sCR and non-CR/sCR

| | Screening | | | Baseline | | |
|---|---|---|---|---|---|---|
| | | sBCMA, median | | | sBCMA, median | |
| | n | (range), ng/mL | | n | (range), ng/mL | |
| CR/sCR (n = 42) | 41 | 161 | (27-689) | 40 | 191 | (11-909) |
| Bridging | 36 | 169 | (44-332) | 35 | 202 | (55-311) |
| therapy (n = 37) | | | | | | |
| No bridging | 5 | 57 | (31-225) | 5 | 82 | (11-298) |
| therapy (n = 5) | | | | | | |
| Non-CR/sCR (n = 86) | 86 | 302 | (24-1490) | 83 | 340 | (19-2735) |
| Bridging | 75 | 343 | (35-1490) | 73 | 356 | (19-2735) |
| therapy (n = 75) | | | | | | |
| No bridging | 11 | 72 | (24-448) | 10 | 159 | (32-594) |
| therapy (n = 11) | | | | | | |

Together, the results identified IgG, sBCMA, and PT-INR test as negative correlates of CR/sCR, and vector copy number in drug product as a positive correlate of CR/sCR. As sBCMA is an indicator of tumor burden and can affect therapies targeting BCMA, selecting for patients with low tumor burden and controlling tumor burden during manufacturing or bridging therapy may be important in achieving CR/sCR.

### Example 2: Treatment with Anti-BCMA CAR-T Cells in Patients with Clinical High-Risk Multiple Myeloma Due to Inadequate Response to Frontline Autologous Stem Cell Transplantation

Chimeric antigen-receptor (CAR)-expressing T cell compositions containing autologous T cells expressing a CAR specific for B-cell maturation antigen (BCMA) were administered to human subjects with multiple myeloma (MM) who had an inadequate response after frontline therapy with autologous stem cell transplant (ASCT).

The administered T cell compositions were generated by obtaining peripheral-blood mononuclear cells (PBMCs) from leukapheresis samples from individual subjects to be treated, stimulating the PBMCs with anti-CD3 and anti-CD28 antibodies, transducing the cells with a lentiviral vector containing the exemplary anti-BCMA CAR, and expanding the cells for 10 days prior to cryopreservation. The exemplary CAR contained an anti-BCMA scFv, a hinge and transmembrane domain from a CD8a, and a CD137 (4-1BB) co-stimulatory domain followed by the intracellular signaling domain of a CD3ζ chain. The polynucleotide sequence encoding the exemplary BCMA CAR is set forth in SEQ ID NO: 214, and the polypeptide sequence of the exemplary BCMA CAR is set forth in SEQ ID NO: 116.

### A. Eligible Subjects and Treatment

Eligible subjects were adult human subjects with MM who had received ≥3 cycles of induction therapy (including a proteasome inhibitor, immunomodulatory agent, and dexamethasone), and ASCT (single or tandem) but who exhibited an inadequate response to the ASCT defined as less than very good partial response (VGPR) at 70-110 days after last ASCT, without use of consolidation or maintenance therapy. Patients had Eastern Cooperative Oncology Group (ECOG) performance status (PS)≤1.

Eligible subjects received a lymphodepleting chemotherapy (LDC) with fludarabine (flu. 30 mg/m²/day) and cyclophosphamide (cy, 300 mg/m²/day) for 3 consecutive days. Patients received a single infusion of anti-BCMA CAR-T cells at a dose range of 150-450×10⁶ CAR+ T cells. Certain patients further received maintenance therapy with lenalidomide post anti-BCMA CAR-T cell infusion.

The primary efficacy endpoint was complete response (CR) rate (CRR; CR and stringent CR) per International Myeloma Working Group (IMWG) Uniform Response Criteria. Secondary endpoints included overall response rate (ORR: ≥partial response), very good partial response (VGPR) rate (VGPRR), time to response (TTR), duration of response (DOR), progression-free survival (PFS), overall survival (OS), safety, and pharmacokinetics (PK). Exploratory endpoints included assessment of soluble BCMA (sBCMA) level and minimal residual disease negativity (MRD-) by next-generation sequencing and EuroFlow (<10⁻⁵ nucleated cells) data with interpolation to reduce missing/indeterminate data points. Efficacy and safety were analyzed in all subjects who received CAR-T cells. PK, sBCMA, and MRD were analyzed in evaluable subjects.

Anti-BCMA CAR-T cells were manufactured and infused in 31/32 subjects. The median age of the subjects was 64 years old. 67.7% of the subjects had an ECOG PS of 0. Of subjects who were eligible at study entry, 41.9% of subjects had revised International Staging System (R-ISS) stage I disease, 16.1% of subjects had R-ISS stage II disease, and no subject had R-ISS stage 111 disease; 9.7% of subjects had high-risk cytogenetics; 3.2% had bone marrow biopsy-determined high tumor burden (≥50% bone marrow CD138+ plasma cells); and 6.5% had extramedullary disease. Additionally, subjects have low baseline tumor burden at the time of the anti-BCMA CAR-T cell infusion.

At time of analysis, the median follow-up was 27.5 months (range 22-31). CRR was 74.2% (95% CI 55.4-88.1), and ORR was 87.1% (95% CI 70.2-96.4) (Table E7). Median TR was 1 month (range 0.9-2.8).

Time to event endpoint results are depicted in Table E7. Grade 3-4 adverse events (AEs) on or after infusion of CAR-expressing T cell compositions containing autologous T cells expressing a CAR specific for BCMA were reported in 29 subjects (93.5%), most commonly, neutropenia in 25 (80.6%) subjects, leukopenia in 9 (29.0%), and anemia in 7 (22.6%). No grade 5 AEs were reported.

Grade 1 cytokine release syndrome (CRS) occurred in 14 subjects (45.2%). 4 subjects (12.9/%) hade a Grade 2 event, and 0 subject had a Grade 3 event. Moreover, investigator identified neurotoxicity (NT) occurred in 2 subjects (6.5%). One subject had a grade 1 NT while the other had a grade 3 NT.

TABLE E7
Efficacy and Safety outcomes following
anti-BCMA CAR-T cell infusions

| | |
|---|---|
| Efficacy outcome | N = 31 |

Best overall response

| ORR, n | 27 | |
|---|---|---|
| ORR, % (95% CI¹) | 87.1 | (70.2-96.4) |
| VGPRR, n | 26 | |
| VGPRR, % (95% CI¹) | 83.9 | (66.3-94.5) |
| CRR, n | 23 | |
| CRR, % (95% CI¹) | 74.2 | (55.4-88.1) |
| Stringent CR, n (%) | 15 | (48.4) |
| CR, n (%) | 8 | (25.8) |
| VGPR, n (%) | 3 | (9.7) |
| PR, n (%) | 1 | (3.2) |
| Minimal response, n (%) | 2 | (6.5) |
| Stable disease, n (%) | 2 | (6.5) |

| | |
|---|---|
| Progressive discase, n | 0 |

Time to event rates

| | | |
|---|---|---|
| 12 mo DOR rate, % (SE) | 100.0 | (0.00) |
| 24 mo DOR rate, % (SE) | 92.1 | (5.37) |
| 12 mo PFS rate, % (SE) | 90.1 | (5.43) |
| 24 mo PFS rate, % (SE) | 83.1 | (6.89) |
| 12 mo OS rate, % (SE) | 100.0 | (0.00) |
| 24 mo OS rate, % (SE) | 100.0 | (0.00) |

| | N = 31 | | | |
|---|---|---|---|---|
| AEs of special interest | CRS | | NT | |
| Number of subjects with at least one | 18 | (58.1) | 2 | (6.5) |
| event of any grade, n (%) | | | | |
| Number of subjects with at least one | 0 | | 1 | (3.2) |
| grade 3/4 event, n (%) | | | | |
| Time to first onset, median, days (range) | 2.0 | (1-4) | 10.0 | (2-18) |
| Duration of event, median, days (range) | 3.0 | (1-7) | 3.5 | (2-5) |

CI, confidence interval; CR, complete response; CRR, complete response rate; CRS, cytokine release syndrome; DOR, duration of response; ORR, overall response rate; OS, overall survival; PFS, progression-free survival; PR, partial response; SE, standard error; VGPR, very good partial response; VGPRR, very good partial response rate.
¹Clopper-Pearson confidence interval.

Robust cell expansion was observed in 31 evaluable subjects (depicted in Table E8) despite low tumor burden, as assessed by sBCMA levels (range 6.5-154.0 ng/mL at infusion, n=30). Cellular expansion was higher in subjects who achieved ≥CR vs <CR with anti-BCMA CAR-T infusion (Table E8)

TABLE E8
Summary of pharmacokinetic parameters.¹

| | Evaluable | | Evaluable subjects | | Evaluable subjects | |
|---|---|---|---|---|---|---|
| Pharmacokinetic | subjects | | with ≥CR | | with <CR | |
| parameter | (n = 31) | | (n = 23) | | (n = 8) | |
| Cmax, copies/μg | 233,417 | (115) | 328,671 | (80) | 87,262 | (88) |
| (% geometric CV)² | | | | | | |
| AUC0-28 days, | 2,910,792 | (127) | 4,217,970 | (84) | 1,049,561 | (104) |
| days*copies/μg | | | | | | |
| (% geometric CV)² | | | | | | |
| Tmax, median, days | 11 | (7-27) | 11 | (7-21) | 9 | (7-27) |
| (range) | | | | | | |

AUC_{0-28 days}, area under the curve of the transgene level from time of dose to 28 days post-infusion; Cₘₐₓ, peak serum concentration; CV, coefficient of variation; Tₘₐₓ, time to peak concentration.
¹At 15 Jan. 2022 cutoff date.
²Data are presented as geometric mean.

sBCMA was cleared within 2 months post anti-BCMA CAR-T cell infusion in 24/31 (77.4%) evaluable subject, including in 22 who had ≥CR. Low sBCMA is favorable for achieving complete response for achieving CR for anti-BCMA CAR-T cell treatment. Among 23 subjects with ≥CR, 16 (69.6%; 95% CI, 49.1-84.4) subjects had MRD- at 6 mo post-anti-BCMA CAR-T cell infusion. At 12 months, MRD- was seen in 17/27 (62.9%; 95% CI 44.2-78.5) evaluable subjects, regardless of CR status and in 16/23 (69.6%; 95% CI 49.1-84.4) evaluable subjects who had ≥CR. Of note, of the 16 subjects, 11 sustained MRD- at 24 months, 2 subjects did not have 24 months data available, and data for 3 subjects were indeterminate.

The treatment with the anti-BCMA CAR-T cells demonstrated frequent, deep, and durable responses in subjects who experienced an inadequate response to frontline therapy with ASCT. Early deep clearance of tumor was observed in subjects with ≥CR after anti-BCMA CAR-T cell treatment and was sustained at the two year timepoint. Lower incidence of CRS and NT were seen in these subjects versus those subjects who were treated with anti-BCMA CAR-T cell treatment in later lines of therapy. Taken together, the results support a favorable clinical risk benefit profile of anti-BCMA CAR-T cell treatment in NDMM subjects.

### Example 3: Treatment with Anti-BCMA CAR-T Cells in Patients with Clinical High-Risk Multiple Myeloma Due to Early Relapse after Frontline Autologous Stem Cell Transplantation

Chimeric antigen-receptor (CAR)-expressing T cell compositions containing autologous T cells expressing a CAR specific for B-cell maturation antigen (BCMA) were administered to human subjects with multiple myeloma (MM) who had an early relapse or an inadequate response after frontline therapy with autologous stem cell transplant (ASCT).

The administered T cell compositions were generated by obtaining peripheral-blood mononuclear cells (PBMCs) from leukapheresis samples from individual subjects to be treated, stimulating the PBMCs with anti-CD3 and anti-CD28 antibodies, transducing the cells with a lentiviral vector containing the exemplary anti-BCMA CAR, and expanding the cells for 10 days prior to cryopreservation. The exemplary CAR contained an anti-BCMA scFv, a hinge and transmembrane domain from a CD8a, and a CD137 (4-1BB) co-stimulatory domain followed by the intracellular signaling domain of a CD3 chain. The polynucleotide sequence encoding the exemplary BCMA CAR is set forth in SEQ ID NO: 214, and the polypeptide sequence of the exemplary BCMA CAR is set forth in SEQ ID NO: 116.

### A. Eligible Subjects and Treatment

Eligible subjects were adult human subjects who had received a frontline treatment containing induction (≥3 cycles of induction therapy, including a proteasome inhibitor, immunomodulatory agent, and dexamethasone) and ASCT (single or tandem) and lenalidomide-containing maintenance, but who exhibited early relapse defined as progressive disease (PD)>18 months from starting the frontline therapy.

Eligible subjects received a lymphodepleting chemotherapy (LDC) with fludarabine (flu, 30 mg/m²/day) and cyclophosphamide (cy, 300 mg/m²/day) for 3 consecutive days. Patients received a single infusion of anti-BCMA CAR-T cells at a dose range of 150-450×10⁶ CAR+ T cells. Some patients also received a bridging therapy (corticosteroids, alkylating agents, immunomodulatory agents, proteasome inhibitors [PI], and/or anti-CD38 antibodies) before CAR-T cell infusion.

The primary efficacy endpoint was complete response (CR) rate (CRR; CR and stringent CR) per International Myeloma Working Group (IMWG) Uniform Response Criteria. Secondary endpoints included overall response rate (ORR; ≥partial response), very good partial response (VGPR) rate (VGPRR), time to response (TTR), duration of response (DOR), progression-free survival (PFS), overall survival (OS), safety, and pharmacokinetics (PK). Exploratory endpoints included assessment of soluble BCMA (sBCMA) level and minimal residual disease negativity (MRD-) by next-generation sequencing and EuroFlow (<10⁻⁵ nucleated cells) data with interpolation to reduce missing/indeterminate data points. Efficacy and safety were analyzed in all subjects who received CAR-T cells. PK, sBCMA, and MRD were analyzed in evaluable subjects.

Anti-BCMA CAR-T cells were manufactured and infused in 37/39 subjects. The median age of the subjects was 57 years and median time since diagnosis was 1.6 years. A total of 62.2% of subjects had Eastern Cooperative Oncology Group (ECOG) PS 0, and 70.3% of subjects received bridging therapy for MM. Of subjects who were eligible at study entry, 13.5% of subjects had revised International Staging System (R-ISS) stage 1, 51.4% of subjects had R-ISS stage II and 5.4% of subjects had R-ISS stage 111 disease; 32.4% had high-risk cytogenetics; 18.9% had bone marrow biopsy-determined high tumor burden (>50% bone marrow CD138+ plasma cells); and 8.1% had extramedullary disease. Most patients had disease refractory to an immunomodulatory agent (86.5%) or proteasome inhibitor (PI; 89.2%), with 86.5% exhibiting double refractory disease.

At time of analysis, the median follow-up was 21.5 months (range 2-31). CRR was 45.9% (95% CI 29.5-63.1), and ORR was 83.8% (95% CI 68.0-93.8) (Table E9). At 6 months post-anti-BCMA CAR-T cell infusion, MRD- was observed in 11/13 (85%; 95% CI 57.8-95.7) subjects. At 12 months post-anti-BCMA CAR-T cell infusion, MRD- was observed in 7/10 (70%; 95% CI 39.7-89.2) subjects. Of the 7 subjects, 1 had PD at 20.8 months, 5 sustained MRD- at ≥18 months, and 1 subject had unavailable >12 month data. Median TTR was 1 month (range 0.9-2.9). Median DOR was 15.7 months (95% CI 7.62-19.81). Median PFS was 11.4 months (95% CI 5.55-19.58); median OS was not reached. Time to event endpoint results for DOR, PFS, and OS are shown in Table E9.

Grade (Gr) 3-4 adverse events (AEs) on or after anti-BCMA CAR-T cell infusion occurred in all subjects, most commonly neutropenia in 35 subjects (94.6%), anemia in 17 subjects (45.9%), and thrombocytopenia in 14 subjects (37.8%). Two subjects died due to pneumonia and pseudomonal sepsis. Grade 1/2 cytokine release syndrome (CRS) occurred in 30 subjects (81.1%); 1 subject (2.7%) had a Grade 3 event (Table E1). Grades 1 and 2 investigator-identified neurotoxicity (NT) occurred in 8 subjects (21.6%). No subject had a ≥Gr 3 NT.

TABLE E9
Efficacy and Safety outcomes following
anti-BCMA CAR-T cell infusions

| | |
|---|---|
| Efficacy outcome | N = 37 |

Best overall response

| ORR, n | 31 | |
|---|---|---|
| ORR, % (95% CIa) | 83.8 | (68.0-93.8) |
| CRR, n | 17 | |
| CRR, % (95% CIa) | 45.9 | (29.5-63.1) |
| Stringent CR, n (%) | 14 | (37.8) |
| CR, n (%) | 3 | (8.1) |
| Very good PR, n (%) | 8 | (21.6) |
| PR, n (%) | 6 | (16.2) |
| Minimal response | 2 | (5.4) |
| Stable disease | 4 | (10.8) |

| | |
|---|---|
| Progressive disease | 0 |

Time to event rates

| | | |
|---|---|---|
| 12 mo DOR rate, % (SE) | 54.0 | (9.07) |
| 24 mo DOR rate, % (SE) | 31.3 | (8.83) |
| 12 mo PFS rate, % (SE) | 47.9 | (8.30) |
| 24 mo PFS rate, % (SE) | 26.2 | (7.63) |
| 12 mo OS rate, % (SE) | 88.0 | (5.64) |
| 24 mo OS rate, % (SE) | 84.7 | (6.31) |

| | N = 37 | | | |
|---|---|---|---|---|
| AEs of special interest | CRS | | NT | |
| Number of subjects with at least | 31 | (83.8) | 8 | (21.6) |
| one event of any grade, n (%) | | | | |
| Number of subjects with at least | 1 | (2.7) | 0 | |
| one grade 3/4 event, n (%) | | | | |
| Time to first onset, median, days (range) | 2.0 | (1-15) | 3.0 | (1-12) |
| Duration of event, median, days (range) | 3.0 | (1-11) | 3.5 | (2-7) |

CI, confidence interval; CR, complete response; CRR, complete response rate; CRS, cytokine release syndrome; DOR, duration of response; ORR, overall response rate; OS, overall survival; PFS, progression-free survival; PR, partial response; SE, standard error; VGPR, very good partial response; VGPRR, very good partial response rate.
¹Clopper-Pearson confidence interval.

Robust cell expansion was seen in 36 evaluable subjects (Table E4). Anti-BCMA CAR-T cell cellular expansion levels were higher in subjects who had ≥CR versus those who had <CR with anti-BCMA CAR-T infusion (Table E10), sBCMA (range 15.0-737.0 ng/mL at infusion, n=36) was cleared within 2 months post-anti-BCMA CAR-T cell infusion in 25/37 (67.61%) subjects, including all subjects who had ≥CR.

TABLE E10
Summary of pharmacokinetic parameters.¹

| | Evaluable | | Evaluable subjects | | Evaluable subjects | |
|---|---|---|---|---|---|---|
| Pharmacokinetic | subjects | | with ≥CR | | with <CR | |
| parameter | (n = 36) | | (n = 16) | | (n = 20) | |
| Cmax, copies/μg | 286,462 | (136) | 404,171 | (70) | 217,505 | (178) |
| (% geometric CV)² | | | | | | |
| AUC0-28 days, | 3,360,086 | (170) | 4,745,793 | (102) | 2,549,081 | (219) |
| days*copies/μg | | | | | | |
| (% geometric CV)² | | | | | | |
| Tmax, median, days | 11 | (7-23) | 11 | (9-17) | 11 | (7-23) |
| (range) | | | | | | |

AUC_{0-28 days}, area under the curve of the transgene level from time of dose to 28 days post-infusion; Cₘₐₓ, peak serum concentration; CV, coefficient of variation; Tₘₐₓ, time to peak concentration.
¹At 15 Jan. 2022 cutoff date.
²Data are presented as geometric mean.

The treatment with the anti-BCMA CAR-T cells demonstrated frequent, deep responses in subjects who experienced an early relapse after frontline therapy with ASCT. MRD- was sustained in a subset of subjects >18 months. Without wishing to be bound by theory, the maintenance therapy with lenalidomide after ASCT may potentiate the durability in response of the anti-BCMA CAR-T cells. The incidence of CRS and NT were similar in these subjects vs those treated with anti-BCMA CAR-T cells in later lines of therapy. Taken together, these results support a favorable clinical benefit-risk profile of the anti-BCMA CAR-T cell therapy and its potential use in earlier lines of treatment.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO. | SEQUENCE | DESCRIPTION |
| 1 | ESKYGPPCPPCP | spacer |
| | | (IgG4hinge)(aa) |
| | | Homo sapiens |
| 2 | GAATCTAAGTACGGACCGCCCTGCCCCCCTTGCCCT | spacer |
| | | (IgG4hinge)(nt) |
| | | Homo sapiens |
| 3 | ESKYGPPCPPCPGQPREPQVYTLPPSQEEMTKNQVSLTCLVKGFYPSDIAVEWES | Hinge-CH3 |
| | NGQPENNYKTTPPVLDSDGSFFLYSRLTVDKSRWQEGNVFSCSVMHEALHNHYTQ | spacer |
| | KSLSLSLGK | Homo sapiens |
| 4 | ESKYGPPCPPCPAPEFLGGPSVFLFPPKPKDTLMISRTPEVTCVVVDVSQEDPEV | Hinge-CH2-CH3 |
| | QFNWYVDGVEVHNAKTKPREEQFNSTYRVVSVLTVLHQDWINGKEYKCKVSNKGL | spacer |
| | PSSIEKTISKAKGQPREPQVYTLPPSQEEMTKNQVSLTCLVKGFYPSDIAVEWES | Homo sapiens |
| | NGQPENNYKTTPPVLDSDGSFFLYSRLTVDKSRWQEGNVFSCSVMHEALHNHYTQ | |
| | KSLSLSLGK | |
| 5 | RWPESPKAQASSVPTAQPQAEGSLAKATTAPATTRNTGRGGEEKKKEKEKEEQEE | IgD-hinge-Fc |
| | RETKTPECPSHTQPLGVYLLTPAVQDLWLRDKATFTCFVVGSDLKDAHLTWEVAG | Homo sapiens |
| | KVPTGGVEEGLLERHSNGSQSQHSRLTLPRSLWNAGTSVTCTLNHPSLPPQRLMA | |
| | LREPAAQAPVKLSLNLLASSDPPEAASWLLCEVSGFSPPNILLMWLEDQREVNTS | |
| | GFAPARPPPQPGSTTFWAWSVLRVPAPPSPQPATYTCVVSHEDSRTLLNASRSLE | |
| | VSYVTDH | |
| 6 | LEGGGEGRGSLLTCGDVEENPGPR | T2A |
| | | artificial |
| 7 | RKVCNGIGIGEFKDSLSINATNIKHFKNCTSISGDLHILPVAFRGDSFTHTPPLD | tEGFR |
| | PQELDILKTVKEITGFLLIQAWPENRTDLHAFENLEIIRGRTKQHGQFSLAVVSL | artificial |
| | NITSLGLRSLKEISDGDVIISGNKNLCYANTINWKKLFGTSGQKTKIISNRGENS | |
| | CKATGQVCHALCSPEGCWGPEPRDCVSCRNVSRGRECVDKCNLLEGEPREFVENS | |
| | ECIQCHPECLPQAMNITCTGRGPDNCIQCAHYIDGPHCVKTCPAGVMGENNTLVW | |
| | KYADAGHVCHLCHPNCTYGCTGPGLEGCPTNGPKIPSIATGMVGALLLLLVVALG | |
| | IGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKPFWVLVVVGGVLACYSL | CD28 (amino |
| | LVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to |
| | | GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRGRDPEMGGKPRRKNPQ | CD3 zeta |
| | EGLYNELQKDKMAEAYSEIGMKGERRRGKGHDGLYQGLSTATKDTYDALHMQALP | Homo sapiens |
| | PR | |
| 14 | RVKFSRSAEPPAYQQGQNQLYNELNLGRREEYDVLDKRRGRDPEMGGKPRRKNPQ | CD3 zeta |
| | EGLYNELQKDKMAEAYSEIGMKGERRRGKGHDGLYQGLSTATKDTYDALHMQALP | Homo sapiens |
| | PR | |
| 15 | RVKFSRSADAPAYKQGQNQLYNELNLGRREEYDVLDKRRGRDPEMGGKPRRKNPQ | CD3 zeta |
| | EGLYNELQKDKMAEAYSEIGMKGERRRGKGHDGLYQGLSTATKDTYDALHMQALP | Homo sapiens |
| | PR | |
| 16 | PGGG-(SGGGG)5-P- wherein P is proline, G is glycine and | linker |
| | S is serine | |
| 17 | GSADDAKKDAAKKDGKS | Linker |
| 18 | QIQLVQSGPELKKPGETVKISCKASGYTFTDYSINWVKRAPGKGLKWMGWINTET | Variable heavy |
| | REPAYAYDFRGRFAFSLETSASTAYLQINNLKYEDTATYFCALDYSYAMDYWGQG | (VH) Anti- |
| | TSVTVSS | BCMA |
| 19 | DIVLTQSPPSLAMSLGKRATISCRASESVTILGSHLIHWYQQKPGQPPTLLIQLA | Variable light |
| | SNVQTGVPARFSGSGSRTDFTLTIDPVEEDDVAVYYCLQSRTIPRTFGGGTKLEI | (VL) Anti- |
| | K | BCMA |
| 20 | QIQLVQSGPDLKKPGETVKLSCKASGYTFTNFGMNWVKQAPGKGFKWMAWINTYT | Variable heavy |
| | GESYFADDFKGRFAFSVETSATTAYLQINNLKTEDTATYFCARGEIYYGYDGGFA | (VH) Anti- |
| | YWGQGTLVTVSA | BCMA |
| 21 | DVVMTQSHRFMSTSVGDRVSITCRASQDVNTAVSWYQQKPGQSPKLLIFSASYRY | Variable light |
| | TGVPDRFTGSGSGADFTLTISSVQAEDLAVYYCQQHYSTPWTFGGGTKLDIK | (VL) Anti- |
| | | BCMA |
| 22 | EVQLVQSGAEVKKPGESLKISCKGSGYSFTSYWIGWVRQMPGKGLEWMGIIYPGD | Variable heavy |
| | SDTRYSPSFQGHVTISADKSISTAYLQWSSLKASDTAMYYCARYSGSFDNWGQGT | (VH) Anti- |
| | LVTVSS | BCMA |
| 23 | SYELTQPPSASGTPGQRVTMSCSGTSSNIGSHSVNWYQQLPGTAPKLLIYTNNQR | Variable light |
| | PSGVPDRFSGSKSGTSASLAISGLQSEDEADYYCAAWDGSLNGLVFGGGTKLTVL | (VL) Anti- |
| | G | BCMA |
| 24 | EVQLVQSGAEMKKPGASLKLSCKASGYTFIDYYVYWMRQAPGQGLESMGWINPNS | Variable heavy |
| | GGTNYAQKFQGRVTMTRDTSISTAYMELSRLRSDDTAMYYCARSQRDGYMDYWGQ | (VH) Anti- |
| | GTLVTVSS | BCMA |
| 25 | QSALTQPASVSASPGQSIAISCTGTSSDVGWYQQHPGKAPKLMIYEDSKRPSGVS | Variable light |
| | NRFSGSKSGNTASLTISGLQAEDEADYYCSSNTRSSTLVFGGGTKLTVLG | (VL) Anti-BCMA |
| 26 | GGGGS | Linker |
| 27 | GGGS | Linker |
| 28 | GGGGSGGGGSGGGGS | Linker |
| 29 | GSTSGSGKPGSGEGSTKG | Linker |
| 30 | SRGGGGSGGGGSGGGGSLEMA | Linker |
| 31 | ESKYGPPCPPCPAPPVAGPSVFLFPPKPKDTLMISRTPEVTCVVVDVSQEDPEVQ | Hinge-CH2-CH3 |
| | FNWYVDGVEVHNAKTKPREEQFQSTYRVVSVLTVLHQDWLNGKEYKCKVSNKGLP | spacer |
| | SSIEKTISKAKGQPREPQVYTLPPSQEEMTKNQVSLTCLVKGFYPSDIAVEWESN | Homo sapiens |
| | GQPENNYKTTPPVLDSDGSFFLYSRLTVDKSRWQEGNVFSCSVMHEALHNHYTQK | |
| | SLSLSLGK | |
| 32 | EVQLVQSGAEVKKPGSSVKVSCKASGGTFSSYAISWVRQAPGQGLEWMGRIIPIL | Variable heavy |
| | GIANYAQKFQGRVTMTEDTSTDTAYMELSSLRSEDTAVYYCARSGYSKSIVSYMD | (VH) Anti- |
| | YWGQGTLVTVSS | BCMA |
| 33 | LPVLTQPPSTSGTPGQRVTVSCSGSSSNIGSNVVFWYQQLPGTAPKLVIYRNNQR | Variable light |
| | PSGVPDRFSVSKSGTSASLAISGLRSEDEADYYCAAWDDSLSGYVFGTGTKVTVL | (VL) Anti- |
| | G | BCMA |
| 34 | QVQLVQSGAEVKKPGSSVKVSCKASGGTFSSYAISWVRQAPGQGLEWMGRIIPIL | Variable heavy |
| | GTANYAQKFQGRVTITADESTSTAYMELSSLRSEDTAVYYCARSGYGSYRWEDSW | (VH) Anti- |
| | GQGTLVTVSS | BCMA |
| 35 | QAVLTQPPSASGTPGQRVTISCSGSSSNIGSNYVFWYQQLPGTAPKLLIYSNNQR | Variable light |
| | PSGVPDRFSGSKSGTSASLAISGLRSEDEADYYCAAWDDSLSASYVFGTGTKVTV | (VL) Anti- |
| | LG | BCMA |
| 36 | QVQLVQSGAEVKKPGASVKVSCKASGYTFTDYYMHWVRQAPGQRLEWMGWINPNS | Variable heavy |
| | GGTNYAQKFQDRITVTRDTSSNTGYMELTRLRSDDTAVYYCARSPYSGVLDKWGQ | (VH) Anti- |
| | GTLVTVSS | BCMA |
| 37 | QSVLTQPPSVSGAPGQRVTISCTGSSSNIGAGFDVHWYQQLPGTAPKLLIYGNSN | Variable light |
| | RPSGVPDRFSGSKSGTSASLAITGLQAEDEADYYCQSYDSSLSGYVFGTGTKVTV | (VL) Anti- |
| | LG | BCMA |
| 38 | ASGGGGSGGRASGGGGS | Linker |
| 39 | MALPVTALLLPLALLLHAARP | CD8a signal |
| | | peptide |
| 40 | METDTLLLWVLLLWVPGSTG | signal peptide |
| 41 | EVQLLESGGGLVQPGGSLRLSCAASGFTFSSYAMSWVRQAPGKGLEWVSAISGSG | Variable heavy |
| | GSTYYADSVKGRFTISRDNSKNTLYLQMNSLRAEDTAVYYCARAEMGAVFDIWGQ | (VH) Anti- |
| | GTMVTVSS | BCMA |
| 42 | EIVLTQSPATLSLSPGERATLSCRASQSVSRYLAWYQQKPGQAPRLLIYDASNRA | Variable light |
| | TGIPARFSGSGSGTDFTLTISSLEPEDFAVYYCQQRISWPFTFGGGTKVEIK | (VL) Anti- |
| | | BCMA |
| 43 | QVQLVESGGGVVQPGRSLRLSCAASGFTFSSYGMHWVRQAPGKGLEWVAVISYDG | Variable heavy |
| | SNKYYADSVKGRFTISRDNSKNTLYLQMNSLRAEDTAVYYCARDGTYLGGLWYFD | (VH) Anti- |
| | LWGRGTLVTVSS | BCMA |
| 44 | DIVMTQSPLSLPVTPGEPASISCRSSQSLLHSNGYNYLDWYLQKPGQSPQLLIYL | Variable light |
| | GSNRASGVPDRFSGSGSGTDFTLKISRVEAEDVGVYYCMQGLGLPLTFGGGTKVE | (VL) Anti- |
| | IK | BCMA |
| 45 | QVQLVQSGAEVKKPGASVKVSCKASGYTFTSYYMHWVRQAPGQGLEWMGIINPGG | Variable heavy |
| | GSTSYAQKFQGRVTMTRDTSTSTVYMELSSLRSEDTAVYYCARESWPMDVWGQGT | (VH) Anti- |
| | TVTVSS | BCMA |
| 46 | EIVMTQSPATLSVSPGERATLSCRASQSVSSNLAWYQQKPGQAPRLLIYGASTRA | Variable light |
| | TGIPARFSGSGSGTEFTLTISSLQSEDFAVYYCQQYAAYPTFGGGTKVEIK | (VL) Anti- |
| | | BCMA |
| 47 | QLQLQESGPGLVKPSETLSLTCTVSGGSISSSSYYWGWIRQPPGKGLEWIGSISY | Variable heavy |
| | SGSTYYNPSLKSRVTISVDTSKNQFSLKLSSVTAADTAVYYCARGRGYATSLAFD | (VH) Anti- |
| | IWGQGTMVTVSS | BCMA |
| 48 | EIVLTQSPATLSLSPGERATLSCRASQSVSSYLAWYQQKPGQAPRLLIYDASNRA | Variable light |
| | TGIPARFSGSGSGTDFTLTISSLEPEDFAVYYCQQRHVWPPTFGGGTKVEIK | (VL) Anti- |
| | | BCMA |
| 49 | EVQLVESGGGLVQPGGSLRLSCAASGFTFSSYSMNWVRQAPGKGLEWVSTISSSS | Variable heavy |
| | STIYYADSVKGRFTISRDNAKNSLYLQMNSLRAEDTAVYYCARGSQEHLIFDYWG | (VH) Anti- |
| | QGTLVTVSS | BCMA |
| 50 | EIVLTQSPATLSLSPGERATLSCRASQSVSRYLAWYQQKPGQAPRLLIYDASNRA | Variable light |
| | TGIPARFSGSGSGTDFTLTISSLEPEDFAVYYCQQRFYYPWTFGGGTKVEIK | (VL) Anti- |
| | | BCMA |
| 51 | QVQLVESGGGVVQPGRSLRLSCAASGFTFSSYGMHWVRQAPGKGLEWVAVISYDG | Variable heavy |
| | SNKYYADSVKGRFTISRDNSKNTLYLQMNSLRAEDTAVYYCARTDFWSGSPPGLD | (VH) Anti- |
| | YWGQGTLVTVSS | BCMA |
| 52 | DIQLTQSPSSVSASVGDRVTITCRASQGISSWLAWYQQKPGKAPKLLIYGASSLQ | Variable light |
| | SGVPSRFSGSGSGTDFTLTISSLQPEDFATYYCQQIYTFPFTFGGGTKVEIK | (VL) Anti- |
| | | BCMA |
| 53 | QVQLVQSGAEVKKPGSSVKVSCKASGGTFSSYAISWVRQAPGQGLEWMGGIIPIF | Variable heavy |
| | GTANYAQKFQGRVTITADESTSTAYMELSSLRSEDTAVYYCARTPEYSSSIWHYY | (VH) Anti- |
| | YGMDVWGQGTTVTVSS | BCMA |
| 54 | DIVMTQSPDSLAVSLGERATINCKSSQSVLYSSNNKNYLAWYQQKPGQPPKLLIY | Variable light |
| | WASTRESGVPDRFSGSGSGTDFTLTISSLQAEDVAVYYCQQFAHTPFTFGGGTKV | (VL) Anti- |
| | EIK | BCMA |
| 55 | QVQLVESGGGVVQPGRSLRLSCAASGFTFSSYGMHWVRQAPGKGLEWVAVISYDG | Variable heavy |
| | SNKYYADSVKGRFTISRDNSKNTLYLQMNSLRAEDTAVYYCVKGPLQEPPYDYGM | (VH) Anti- |
| | DVWGQGTTVTVSS | BCMA |
| 56 | EIVMTQSPATLSVSPGERATLSCRASQSVSSNLAWYQQKPGQAPRLLIYSASTRA | Variable light |
| | TGIPARFSGSGSGTEFTLTISSLQSEDFAVYYCQQHHVWPLTFGGGTKVEIK | (VL) Anti- |
| | | BCMA |
| 57 | QVQLVQSGAEVKKPGSSVKVSCKASGGTFSSYAISWVRQAPGQGLEWMGRIIPIL | Variable heavy |
| | GIANYAQKFQGRVTITADKSTSTAYMELSSLRSEDTAVYYCARGGYYSHDMWSED | (VH) Anti- |
| | WGQGTLVTVSS | BCMA |
| 58 | LPVLTQPPSASGTPGQRVTISCSGRSSNIGSNSVNWYRQLPGAAPKLLIYSNNQR | Variable light |
| | PPGVPVRFSGSKSGTSASLAISGLQSEDEATYYCATWDDNLNVHYVFGTGTKVTV | (VL) Anti- |
| | LG | BCMA |
| 59 | QVQLVQSGSELKKPGASVKVSCKASGYTFTDYSINWVRQAPGQGLEWMGWINTET | Variable heavy |
| | REPAYAYDFRGRFVFSLDTSVSTAYLQISSLKAEDTAVYYCARDYSYAMDYWGQG | (VH) Anti- |
| | TLVTVSS | BCMA |
| 60 | DIVLTQSPASLAVSIGERATINCRASESVSVIGAHLIHWYQQKPGQPPKLLIYLA | Variable light |
| | SNLETGVPARFSGSGSGTDFTLTISSLQAEDAAIYYCLQSRIFPRTFGQGTKLEI | (VL) Anti- |
| | K | BCMA |
| 61 | EVQLVESGGGLVQPGGSLRLSCAVSGFALSNHGMSWVRRAPGKGLEWVSGIVYSG | Variable heavy |
| | STYYAASVKGRFTISRDNSRNTLYLQMNSLRPEDTAIYYCSAHGGESDVWGQGTT | (VH) Anti- |
| | VTVSS | BCMA |
| 62 | DIQLTQSPSSLSASVGDRVTITCRASQSISSYLNWYQQKPGKAPKLLIYAASSLQ | Variable light |
| | SGVPSRFSGSGSGTDFTLTISSLQPEDFATYYCQQSYSTPYTFGQGTKVEIK | (VL) Anti- |
| | | BCMA |
| 63 | QVQLVESGGGLVQPGRSLRLSCAASGFTFSNYAMSWVRQAPGKGLGWVSGISRSG | Variable heavy |
| | ENTYYADSVKGRFTISRDNSKNTLYLQMNSLRDEDTAVYYCARSPAHYYGGMDVW | (VH) Anti- |
| | GQGTTVTVSS | BCMA |
| 64 | DIVLTQSPGTLSLSPGERATLSCRASQSISSSFLAWYQQKPGQAPRLLIYGASRR | Variable light |
| | ATGIPDRFSGSGSGTDFTLTISRLEPEDSAVYYCQQYHSSPSWTFGQGTKLEIK | (VL) Anti- |
| | | BCMA |
| 65 | QVQLVESGGGLVQPGGSLRLSCAVSGFALSNHGMSWVRRAPGKGLEWVSGIVYSG | Variable heavy |
| | STYYAASVKGRFTISRDNSRNTLYLQMNSLRPEDTAIYYCSAHGGESDVWGQGTT | (VH) Anti- |
| | VTVSS | BCMA |
| 66 | DIRLTQSPSPLSASVGDRVTITCQASEDINKFLNWYHQTPGKAPKLLIYDASTLQ | Variable light |
| | TGVPSRFSGSGSGTDFTLTINSLQPEDIGTYYCQQYESLPLTFGGGTKVEIK | (VL) Anti- |
| | | BCMA |
| 67 | EVQLVESGGGLVQPGGSLRLSCAVSGFALSNHGMSWVRRAPGKGLEWVSGIVYSG | Variable heavy |
| | STYYAASVKGRFTISRDNSRNTLYLQMNSLRPEDTAIYYCSAHGGESDVWGQGTT | (VH) Anti- |
| | VTVSS | BCMA |
| 68 | EIVLTQSPGTLSLSPGERATLSCRASQSIGSSSLAWYQQKPGQAPRLLMYGASSR | Variable light |
| | ASGIPDRFSGSGSGTDFTLTISRLEPEDFAVYYCQQYAGSPPFTFGQGTKVEIK | (VL) Anti- |
| | | BCMA |
| 69 | QIQLVQSGPELKKPGETVKISCKASGYTFRHYSMNWVKQAPGKGLKWMGRINTES | Variable heavy |
| | GVPIYADDFKGRFAFSVETSASTAYLVINNLKDEDTASYFCSNDYLYSLDFWGQG | (VH) Anti- |
| | TALTVSS | BCMA |
| 70 | DIVLTQSPPSLAMSLGKRATISCRASESVTILGSHLIYWYQQKPGQPPTLLIQLA | Variable light |
| | SNVQTGVPARFSGSGSRTDFTLTIDPVEEDDVAVYYCLQSRTIPRTFGGGTKLEI | (VL) Anti- |
| | K | BCMA |
| 71 | QIQLVQSGPELKKPGETVKISCKASGYTFTHYSMNWVKQAPGKGLKWMGRINTET | Variable heavy |
| | GEPLYADDFKGRFAFSLETSASTAYLVINNLKNEDTATFFCSNDYLYSCDYWGQG | (VH) Anti- |
| | TTLTVSS | BCMA |
| 72 | DIVLTQSPASLAMSLGKRATISCRASESVSVIGAHLIHWYQQKPGQPPKLLIYLA | Variable light |
| | SNLETGVPARFSGSGSGTDFTLTIDPVEEDDVAIYSCLQSRIFPRTFGGGTKLEI | (VL) Anti- |
| | K | BCMA |
| 73 | QVQLVQSGAEVKKPGASVKVSCKASGYSFPDYYINWVRQAPGQGLEWMGWIYFAS | Variable heavy |
| | GNSEYNQKFTGRVTMTRDTSINTAYMELSSLTSEDTAVYFCASLYDYDWYFDVWG | (VH) Anti- |
| | QGTMVTVSS | BCMA |
| 74 | DIVMTQTPLSLSVTPGQPASISCKSSQSLVHSNGNTYLHWYLQKPGQSPQLLIYK | Variable light |
| | VSNRFSGVPDRFSGSGSGTDFTLKISRVEAEDVGIYYCSQSSIYPWTFGQGTKLE | (VL) Anti- |
| | IK | BCMA |
| 75 | QVQLVQSGAEVKKPGASVKVSCKASGYSFPDYYINWVRQAPGQGLEWMGWIYFAS | Variable heavy |
| | GNSEYNQKFTGRVTMTRDTSSSTAYMELSSLRSEDTAVYFCASLYDYDWYFDVWG | (VH) Anti- |
| | QGTMVTVSS | BCMA |
| 76 | DIVMTQTPLSLSVTPGEPASISCKSSQSLVHSNGNTYLHWYLQKPGQSPQLLIYK | Variable light |
| | VSNRFSGVPDRFSGSGSGADFTLKISRVEAEDVGVYYCAETSHVPWTFGQGTKLE | (VL) Anti- |
| | IK | BCMA |
| 77 | QVQLVESGGGLVQPGGSLRLSCEASGFTLDYYAIGWFRQAPGKEREGVICISRSD | Anti-BCMA |
| | GSTYYADSVKGRFTISRDNAKKTVYLQMISLKPEDTAAYYCAAGADCSGYLRDYE | sdAb |
| | FRGQGTQVTVSS | |
| 78 | IEVMYPPPYLDNEKSNGTIIHVKGKHLCPSPLFPGPSKP | CD28 spacer |
| 79 | IYIWAPLAGTCGVLLLSLVITLYCN | CD8a TM |
| 80 | LDNEKSNGTIIHVKGKHLCPSPLFPGPSKP | CD28 spacer |
| | | (truncated) |
| 81 | PTTTPAPRPPTPAPTIASQPLSLRPEACRPAAGGAVHTRGLDFACD | CD8a hinge |
| 82 | TTTPAPRPPTPAPTIASQPLSLRPEACRPAAGGAVHTRGLDFACD | CD8a hinge |
| 83 | FVPVFLPAKPTTTPAPRPPTPAPTIASQPLSLRPEACRPAAGGAVHTRGLDFACD | CD8a hinge |
| 84 | DTGLYICKVELMYPPPYYLGIGNGTQIYVIDPEPCPDSD | CTLA4 hinge |
| 85 | FLLWILAAVSSGLFFYSFLLTAVS | CTLA4 TM |
| 86 | QIKESLRAELRVTERRAEVPTAHPSPSPRPAGQFQTLV | PD-1 hinge |
| 87 | VGVVGGLLGSLVLLVWVLAVI | PD-1 TM |
| 88 | GLAVSTISSFFPPGYQ | Fc(gamma)RIIIa |
| | | hinge |
| 89 | EPKSPDKTHTCPPCPAPPVAGPSVFLFPPKPKDTIMIARTPEVTCVVVDVSHEDP | IgG1 hinge |
| | EVKFNWYVDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMHEALHNHY | |
| | TQKSLSLSPGK | |
| 90 | EVQLLESGGGLVQPGGSLRLSCAASGFTFSSYAMSWVRQAPGKGLEWVSAISGSG | anti-BCMA CAR |
| | GSTYYADSVKGRFTISRDNSKNTLYLQMNSLRAEDTAVYYCARAEMGAVFDIWGQ | |
| | GTMVTVSSGSTSGSGKPGSGEGSTKGEIVLTQSPATLSLSPGERATLSCRASQSV | |
| | SRYLAWYQQKPGQAPRLLIYDASNRATGIPARFSGSGSGTDFTLTISSLEPEDFA | |
| | VYYCQQRISWPFTFGGGTKVEIKRAAALDNEKSNGTIIHVKGKHLCPSPLFPGPS | |
| | KPFWVLVVVGGVLACYSLLVTVAFIIFWVRSKRSRLLHSDYMNMTPRRPGPTRKH | |
| | YQPYAPPRDFAAYRSRVKFSRSADAPAYQQGQNQLYNELNLGRREEYDVLDKRRG | |
| | RDPEMGGKPRRKNPQEGLYNELQKDKMAEAYSEIGMKGERRRGKGHDGLYQGLST | |
| | ATKDTYDALHMQALPPR | |
| 91 | EIVLTQSPATLSLSPGERATLSCRASQSVSRYLAWYQQKPGQAPRLLIYDASNRA | anti-BCMA CAR |
| | TGIPARFSGSGSGTDFTLTISSLEPEDFAVYYCQQRISWPFTFGGGTKVEIKRGS | |
| | TSGSGKPGSGEGSTKGEVQLLESGGGLVQPGGSLRLSCAASGFTFSSYAMSWVRQ | |
| | APGKGLEWVSAISGSGGSTYYADSVKGRFTISRDNSKNTLYLQMNSLRAEDTAVY | |
| | YCARAEMGAVFDIWGQGTMVTVSSAAALDNEKSNGTIIHVKGKHLCPSPLFPGPS | |
| | KPFWVLVVVGGVLACYSLLVTVAFIIFWVRSKRSRLLHSDYMNMTPRRPGPTRKH | |
| | YQPYAPPRDFAAYRSRVKFSRSADAPAYQQGQNQLYNELNLGRREEYDVLDKRRG | |
| | RDPEMGGKPRRKNPQEGLYNELQKDKMAEAYSEIGMKGERRRGKGHDGLYQGLST | |
| | ATKDTYDALHMQALPPR | |
| 92 | QVQLVESGGGVVQPGRSLRLSCAASGFTFSSYGMHWVRQAPGKGLEWVAVISYDG | anti-BCMA CAR |
| | SNKYYADSVKGRFTISRDNSKNTLYLQMNSLRAEDTAVYYCARDGTYLGGLWYFD | |
| | LWGRGTLVTVSSGSTSGSGKPGSGEGSTKGDIVMTQSPLSLPVTPGEPASISCRS | |
| | SQSLLHSNGYNYLDWYLQKPGQSPQLLIYLGSNRASGVPDRFSGSGSGTDFTLKI | |
| | SRVEAEDVGVYYCMQGLGLPLTFGGGTKVEIKRAAALDNEKSNGTIIHVKGKHLC | |
| | PSPLFPGPSKPFWVLVVVGGVLACYSLLVTVAFIIFWVRSKRSRLLHSDYMNMTP | |
| | RRPGPTRKHYQPYAPPRDFAAYRSRVKFSRSADAPAYQQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |
| 93 | DIVMTQSPLSLPVTPGEPASISCRSSQSLLHSNGYNYLDWYLQKPGQSPQLLIYL | anti-BCMA CAR |
| | GSNRASGVPDRFSGSGSGTDFTLKISRVEAEDVGVYYCMQGLGLPLTFGGGTKVE | |
| | IKRGSTSGSGKPGSGEGSTKGQVQLVESGGGVVQPGRSLRLSCAASGFTFSSYGM | |
| | HWVRQAPGKGLEWVAVISYDGSNKYYADSVKGRFTISRDNSKNTLYLQMNSLRAE | |
| | DTAVYYCARDGTYLGGLWYFDLWGRGTLVTVSSAAALDNEKSNGTIIHVKGKHLC | |
| | PSPLFPGPSKPFWVLVVVGGVLACYSLLVTVAFIIFWVRSKRSRLLHSDYMNMTP | |
| | RRPGPTRKHYQPYAPPRDFAAYRSRVKFSRSADAPAYQQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |
| 94 | QVQLVQSGAEVKKPGASVKVSCKASGYTFTSYYMHWVRQAPGQGLEWMGIINPGG | anti-BCMA CAR |
| | GSTSYAQKFQGRVTMTRDTSTSTVYMELSSLRSEDTAVYYCARESWPMDVWGQGT | |
| | TVTVSSGSTSGSGKPGSGEGSTKGEIVMTQSPATLSVSPGERATLSCRASQSVSS | |
| | NLAWYQQKPGQAPRLLIYGASTRATGIPARFSGSGSGTEFTLTISSLQSEDFAVY | |
| | YCQQYAAYPTFGGGTKVEIKRAAALDNEKSNGTIIHVKGKHLCPSPLFPGPSKPF | |
| | WVLVVVGGVLACYSLLVTVAFIIFWVRSKRSRLLHSDYMNMTPRRPGPTRKHYQP | |
| | YAPPRDFAAYRSRVKFSRSADAPAYQQGQNQLYNELNLGRREEYDVLDKRRGRDP | |
| | EMGGKPRRKNPQEGLYNELQKDKMAEAYSEIGMKGERRRGKGHDGLYQGLSTATK | |
| | DTYDALHMQALPPR | |
| 95 | EIVMTQSPATLSVSPGERATLSCRASQSVSSNLAWYQQKPGQAPRLLIYGASTRA | anti-BCMA CAR |
| | TGIPARFSGSGSGTEFTLTISSLQSEDFAVYYCQQYAAYPTFGGGTKVEIKRGST | |
| | SGSGKPGSGEGSTKGQVQLVQSGAEVKKPGASVKVSCKASGYTFTSYYMHWVRQA | |
| | PGQGLEWMGIINPGGGSTSYAQKFQGRVTMTRDTSTSTVYMELSSLRSEDTAVYY | |
| | CARESWPMDVWGQGTTVTVSSAAALDNEKSNGTIIHVKGKHLCPSPLFPGPSKPF | |
| | WVLVVVGGVLACYSLLVTVAFIIFWVRSKRSRLLHSDYMNMTPRRPGPTRKHYQP | |
| | YAPPRDFAAYRSRVKFSRSADAPAYQQGQNQLYNELNLGRREEYDVLDKRRGRDP | |
| | EMGGKPRRKNPQEGLYNELQKDKMAEAYSEIGMKGERRRGKGHDGLYQGLSTATK | |
| | DTYDALHMQALPPR | |
| 96 | QLQLQESGPGLVKPSETLSLTCTVSGGSISSSSYYWGWIRQPPGKGLEWIGSISY | anti-BCMA CAR |
| | SGSTYYNPSLKSRVTISVDTSKNQFSLKLSSVTAADTAVYYCARGRGYATSLAFD | |
| | IWGQGTMVTVSSGSTSGSGKPGSGEGSTKGEIVLTQSPATLSLSPGERATLSCRA | |
| | SQSVSSYLAWYQQKPGQAPRLLIYDASNRATGIPARFSGSGSGTDFTLTISSLEP | |
| | EDFAVYYCQQRHVWPPTFGGGTKVEIKRAAALDNEKSNGTIIHVKGKHLCPSPLF | |
| | PGPSKPFWVLVVVGGVLACYSLLVTVAFIIFWVRSKRSRLLHSDYMNMTPRRPGP | |
| | TRKHYQPYAPPRDFAAYRSRVKFSRSADAPAYQQGQNQLYNELNLGRREEYDVLD | |
| | KRRGRDPEMGGKPRRKNPQEGLYNELQKDKMAEAYSEIGMKGERRRGKGHDGLYQ | |
| | GLSTATKDTYDALHMQALPPR | |
| 97 | EIVLTQSPATLSLSPGERATLSCRASQSVSSYLAWYQQKPGQAPRLLIYDASNRA | anti-BCMA CAR |
| | TGIPARFSGSGSGTDFTLTISSLEPEDFAVYYCQQRHVWPPTFGGGTKVEIKRGS | |
| | TSGSGKPGSGEGSTKGQLQLQESGPGLVKPSETLSLTCTVSGGSISSSSYYWGWI | |
| | RQPPGKGLEWIGSISYSGSTYYNPSLKSRVTISVDTSKNQFSLKLSSVTAADTAV | |
| | YYCARGRGYATSLAFDIWGQGTMVTVSSAAALDNEKSNGTIIHVKGKHLCPSPLF | |
| | PGPSKPFWVLVVVGGVLACYSLLVTVAFIIFWVRSKRSRLLHSDYMNMTPRRPGP | |
| | TRKHYQPYAPPRDFAAYRSRVKFSRSADAPAYQQGQNQLYNELNLGRREEYDVLD | |
| | KRRGRDPEMGGKPRRKNPQEGLYNELQKDKMAEAYSEIGMKGERRRGKGHDGLYQ | |
| | GLSTATKDTYDALHMQALPPR | |
| 98 | EVQLVESGGGLVQPGGSLRLSCAASGFTFSSYSMNWVRQAPGKGLEWVSTISSSS | anti-BCMA CAR |
| | STIYYADSVKGRFTISRDNAKNSLYLQMNSLRAEDTAVYYCARGSQEHLIFDYWG | |
| | QGTLVTVSSGSTSGSGKPGSGEGSTKGEIVLTQSPATLSLSPGERATLSCRASQS | |
| | VSRYLAWYQQKPGQAPRLLIYDASNRATGIPARFSGSGSGTDFTLTISSLEPEDF | |
| | AVYYCQQRFYYPWTFGGGTKVEIKRAAALDNEKSNGTIIHVKGKHLCPSPLFPGP | |
| | SKPFWVLVVVGGVLACYSLLVTVAFIIFWVRSKRSRLLHSDYMNMTPRRPGPTRK | |
| | HYQPYAPPRDFAAYRSRVKFSRSADAPAYQQGQNQLYNELNLGRREEYDVLDKRR | |
| | GRDPEMGGKPRRKNPQEGLYNELQKDKMAEAYSEIGMKGERRRGKGHDGLYQGLS | |
| | TATKDTYDALHMQALPPR | |
| 99 | EIVLTQSPATLSLSPGERATLSCRASQSVSRYLAWYQQKPGQAPRLLIYDASNRA | anti-BCMA CAR |
| | TGIPARFSGSGSGTDFTLTISSLEPEDFAVYYCQQRFYYPWTFGGGTKVEIKRGS | |
| | TSGSGKPGSGEGSTKGEVQLVESGGGLVQPGGSLRLSCAASGFTFSSYSMNWVRQ | |
| | APGKGLEWVSTISSSSSTIYYADSVKGRFTISRDNAKNSLYLQMNSLRAEDTAVY | |
| | YCARGSQEHLIFDYWGQGTLVTVSSAAALDNEKSNGTIIHVKGKHLCPSPLFPGP | |
| | SKPFWVLVVVGGVLACYSLLVTVAFIIFWVRSKRSRLLHSDYMNMTPRRPGPTRK | |
| | HYQPYAPPRDFAAYRSRVKFSRSADAPAYQQGQNQLYNELNLGRREEYDVLDKRR | |
| | GRDPEMGGKPRRKNPQEGLYNELQKDKMAEAYSEIGMKGERRRGKGHDGLYQGLS | |
| | TATKDTYDALHMQALPPR | |
| 100 | QVQLVESGGGVVQPGRSLRLSCAASGFTFSSYGMHWVRQAPGKGLEWVAVISYDG | anti-BCMA CAR |
| | SNKYYADSVKGRFTISRDNSKNTLYLQMNSLRAEDTAVYYCARTDFWSGSPPGLD | |
| | YWGQGTLVTVSSGSTSGSGKPGSGEGSTKGDIQLTQSPSSVSASVGDRVTITCRA | |
| | SQGISSWLAWYQQKPGKAPKLLIYGASSLQSGVPSRFSGSGSGTDFTLTISSLQP | |
| | EDFATYYCQQIYTFPFTFGGGTKVEIKRAAALDNEKSNGTIIHVKGKHLCPSPLF | |
| | PGPSKPFWVLVVVGGVLACYSLLVTVAFIIFWVRSKRSRLLHSDYMNMTPRRPGP | |
| | TRKHYQPYAPPRDFAAYRSRVKFSRSADAPAYQQGQNQLYNELNLGRREEYDVLD | |
| | KRRGRDPEMGGKPRRKNPQEGLYNELQKDKMAEAYSEIGMKGERRRGKGHDGLYQ | |
| | GLSTATKDTYDALHMQALPPR | |
| 101 | DIQLTQSPSSVSASVGDRVTITCRASQGISSWLAWYQQKPGKAPKLLIYGASSLQ | anti-BCMA CAR |
| | SGVPSRFSGSGSGTDFTLTISSLQPEDFATYYCQQIYTFPFTFGGGTKVEIKRGS | |
| | TSGSGKPGSGEGSTKGQVQLVESGGGVVQPGRSLRLSCAASGFTFSSYGMHWVRQ | |
| | APGKGLEWVAVISYDGSNKYYADSVKGRFTISRDNSKNTLYLQMNSLRAEDTAVY | |
| | YCARTDFWSGSPPGLDYWGQGTLVTVSSAAALDNEKSNGTIIHVKGKHLCPSPLF | |
| | PGPSKPFWVLVVVGGVLACYSLLVTVAFIIFWVRSKRSRLLHSDYMNMTPRRPGP | |
| | TRKHYQPYAPPRDFAAYRSRVKFSRSADAPAYQQGQNQLYNELNLGRREEYDVLD | |
| | KRRGRDPEMGGKPRRKNPQEGLYNELQKDKMAEAYSEIGMKGERRRGKGHDGLYQ | |
| | GLSTATKDTYDALHMQALPPR | |
| 102 | QVQLVQSGAEVKKPGSSVKVSCKASGGTFSSYAISWVRQAPGQGLEWMGGIIPIF | anti-BCMA CAR |
| | GTANYAQKFQGRVTITADESTSTAYMELSSLRSEDTAVYYCARTPEYSSSIWHYY | |
| | YGMDVWGQGTTVTVSSGSTSGSGKPGSGEGSTKGDIVMTQSPDSLAVSLGERATI | |
| | NCKSSQSVLYSSNNKNYLAWYQQKPGQPPKLLIYWASTRESGVPDRFSGSGSGTD | |
| | FTLTISSLQAEDVAVYYCQQFAHTPFTFGGGTKVEIKRAAALDNEKSNGTIIHVK | |
| | GKHLCPSPLFPGPSKPFWVLVVVGGVLACYSLLVTVAFIIFWVRSKRSRLLHSDY | |
| | MNMTPRRPGPTRKHYQPYAPPRDFAAYRSRVKFSRSADAPAYQQGQNQLYNELNL | |
| | GRREEYDVLDKRRGRDPEMGGKPRRKNPQEGLYNELQKDKMAEAYSEIGMKGERR | |
| | RGKGHDGLYQGLSTATKDTYDALHMQALPPR | |
| 103 | DIVMTQSPDSLAVSLGERATINCKSSQSVLYSSNNKNYLAWYQQKPGQPPKLLIY | anti-BCMA CAR |
| | WASTRESGVPDRFSGSGSGTDFTLTISSLQAEDVAVYYCQQFAHTPFTFGGGTKV | |
| | EIKRGSTSGSGKPGSGEGSTKGQVQLVQSGAEVKKPGSSVKVSCKASGGTFSSYA | |
| | ISWVRQAPGQGLEWMGGIIPIFGTANYAQKFQGRVTITADESTSTAYMELSSLRS | |
| | EDTAVYYCARTPEYSSSIWHYYYGMDVWGQGTTVTVSSAAALDNEKSNGTIIHVK | |
| | GKHLCPSPLFPGPSKPFWVLVVVGGVLACYSLLVTVAFIIFWVRSKRSRLLHSDY | |
| | MNMTPRRPGPTRKHYQPYAPPRDFAAYRSRVKFSRSADAPAYQQGQNQLYNELNL | |
| | GRREEYDVLDKRRGRDPEMGGKPRRKNPQEGLYNELQKDKMAEAYSEIGMKGERR | |
| | RGKGHDGLYQGLSTATKDTYDALHMQALPPR | |
| 104 | QVQLVESGGGVVQPGRSLRLSCAASGFTFSSYGMHWVRQAPGKGLEWVAVISYDG | anti-BCMA CAR |
| | SNKYYADSVKGRFTISRDNSKNTLYLQMNSLRAEDTAVYYCVKGPLQEPPYDYGM | |
| | DVWGQGTTVTVSSGSTSGSGKPGSGEGSTKGEIVMTQSPATLSVSPGERATLSCR | |
| | ASQSVSSNLAWYQQKPGQAPRLLIYSASTRATGIPARFSGSGSGTEFTLTISSLQ | |
| | SEDFAVYYCQQHHVWPLTFGGGTKVEIKRAAALDNEKSNGTIIHVKGKHLCPSPL | |
| | FPGPSKPFWVLVVVGGVLACYSLLVTVAFIIFWVRSKRSRLLHSDYMNMTPRRPG | |
| | PTRKHYQPYAPPRDFAAYRSRVKFSRSADAPAYQQGQNQLYNELNLGRREEYDVL | |
| | DKRRGRDPEMGGKPRRKNPQEGLYNELQKDKMAEAYSEIGMKGERRRGKGHDGLY | |
| | QGLSTATKDTYDALHMQALPPR | |
| 105 | EIVMTQSPATLSVSPGERATLSCRASQSVSSNLAWYQQKPGQAPRLLIYSASTRA | anti-BCMA CAR |
| | TGIPARFSGSGSGTEFTLTISSLQSEDFAVYYCQQHHVWPLTFGGGTKVEIKRGS | |
| | TSGSGKPGSGEGSTKGQVQLVESGGGVVQPGRSLRLSCAASGFTFSSYGMHWVRQ | |
| | APGKGLEWVAVISYDGSNKYYADSVKGRFTISRDNSKNTLYLQMNSLRAEDTAVY | |
| | YCVKGPLQEPPYDYGMDVWGQGTTVTVSSAAALDNEKSNGTIIHVKGKHLCPSPL | |
| | FPGPSKPFWVLVVVGGVLACYSLLVTVAFIIFWVRSKRSRLLHSDYMNMTPRRPG | |
| | PTRKHYQPYAPPRDFAAYRSRVKFSRSADAPAYQQGQNQLYNELNLGRREEYDVL | |
| | DKRRGRDPEMGGKPRRKNPQEGLYNELQKDKMAEAYSEIGMKGERRRGKGHDGLY | |
| | QGLSTATKDTYDALHMQALPPR | |
| 106 | QSALTQPASVSASPGQSIAISCTGTSSDVGWYQQHPGKAPKLMIYEDSKRPSGVS | anti-BCMA CAR |
| | NRFSGSKSGNTASLTISGLQAEDEADYYCSSNTRSSTLVFGGGTKLTVLGSRGGG | |
| | GSGGGGSGGGGSLEMAEVQLVQSGAEMKKPGASLKLSCKASGYTFIDYYVYWMRQ | |
| | APGQGLESMGWINPNSGGTNYAQKFQGRVTMTRDTSISTAYMELSRLRSDDTAMY | |
| | YCARSQRDGYMDYWGQGTLVTVSSAAAIEVMYPPPYLDNEKSNGTIIHVKGKHLC | |
| | PSPLFPGPSKPFWVLVVVGGVLACYSLLVTVAFIIFWVRSKRSRLLHSDYMNMTP | |
| | RRPGPTRKHYQPYAPPRDFAAYRSRVKFSRSADAPAYQQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |
| 107 | QSVLTQPPSVSGAPGQRVTISCTGSSSNIGAGFDVHWYQQLPGTAPKLLIYGNSN | anti-BCMA CAR |
| | RPSGVPDRFSGSKSGTSASLAITGLQAEDEADYYCQSYDSSLSGYVFGTGTKVTV | |
| | LGSRGGGGSGGGGSGGGGSLEMAQVQLVQSGAEVKKPGASVKVSCKASGYTFTDY | |
| | YMHWVRQAPGQRLEWMGWINPNSGGTNYAQKFQDRITVTRDTSSNTGYMELTRLR | |
| | SDDTAVYYCARSPYSGVLDKWGQGTLVTVSSAAAIEVMYPPPYLDNEKSNGTIIH | |
| | VKGKHLCPSPLFPGPSKPFWVLVVVGGVLACYSLLVTVAFIIFWVRSKRSRLLHS | |
| | DYMNMTPRRPGPTRKHYQPYAPPRDFAAYRSRVKFSRSADAPAYQQGQNQLYNEL | |
| | NLGRREEYDVLDKRRGRDPEMGGKPRRKNPQEGLYNELQKDKMAEAYSEIGMKGE | |
| | RRRGKGHDGLYQGLSTATKDTYDALHMQALPPR | |
| 108 | SYELTQPPSASGTPGQRVTMSCSGTSSNIGSHSVNWYQQLPGTAPKLLIYTNNQR | anti-BCMA CAR |
| | PSGVPDRFSGSKSGTSASLAISGLQSEDEADYYCAAWDGSLNGLVFGGGTKLTVL | |
| | GSRGGGGSGGGGSGGGGSLEMAEVQLVQSGAEVKKPGESLKISCKGSGYSFTSYW | |
| | IGWVRQMPGKGLEWMGIIYPGDSDTRYSPSFQGHVTISADKSISTAYLQWSSLKA | |
| | SDTAMYYCARYSGSFDNWGQGTLVTVSSAAAIEVMYPPPYLDNEKSNGTIIHVKG | |
| | KHLCPSPLFPGPSKPFWVLVVVGGVLACYSLLVTVAFIIFWVRSKRSRLLHSDYM | |
| | NMTPRRPGPTRKHYQPYAPPRDFAAYRSRVKFSRSADAPAYQQGQNQLYNELNLG | |
| | RREEYDVLDKRRGRDPEMGGKPRRKNPQEGLYNELQKDKMAEAYSEIGMKGERRR | |
| | GKGHDGLYQGLSTATKDTYDALHMQALPPR | |
| 109 | LPVLTQPPSASGTPGQRVTISCSGRSSNIGSNSVNWYRQLPGAAPKLLIYSNNQR | anti-BCMA CAR |
| | PPGVPVRFSGSKSGTSASLAISGLQSEDEATYYCATWDDNLNVHYVFGTGTKVTV | |
| | LGSRGGGGSGGGGSGGGGSLEMAQVQLVQSGAEVKKPGSSVKVSCKASGGTFSSY | |
| | AISWVRQAPGQGLEWMGRIIPILGIANYAQKFQGRVTITADKSTSTAYMELSSLR | |
| | SEDTAVYYCARGGYYSHDMWSEDWGQGTLVTVSSAAAIEVMYPPPYLDNEKSNGT | |
| | IIHVKGKHLCPSPLFPGPSKPFWVLVVVGGVLACYSLLVTVAFIIFWVRSKRSRL | |
| | LHSDYMNMTPRRPGPTRKHYQPYAPPRDFAAYRSRVKFSRSADAPAYQQGQNQLY | |
| | NELNLGRREEYDVLDKRRGRDPEMGGKPRRKNPQEGLYNELQKDKMAEAYSEIGM | |
| | KGERRRGKGHDGLYQGLSTATKDTYDALHMQALPPR | |
| 110 | QAVLTQPPSASGTPGQRVTISCSGSSSNIGSNYVFWYQQLPGTAPKLLIYSNNQR | anti-BCMA CAR |
| | PSGVPDRFSGSKSGTSASLAISGLRSEDEADYYCAAWDDSLSASYVFGTGTKVTV | |
| | LGSRGGGGSGGGGSGGGGSLEMAQVQLVQSGAEVKKPGSSVKVSCKASGGTFSSY | |
| | AISWVRQAPGQGLEWMGRIIPILGTANYAQKFQGRVTITADESTSTAYMELSSLR | |
| | SEDTAVYYCARSGYGSYRWEDSWGQGTLVTVSSAAAIEVMYPPPYLDNEKSNGTI | |
| | IHVKGKHLCPSPLFPGPSKPFWVLVVVGGVLACYSLLVTVAFIIFWVRSKRSRLL | |
| | HSDYMNMTPRRPGPTRKHYQPYAPPRDFAAYRSRVKFSRSADAPAYQQGQNQLYN | |
| | ELNLGRREEYDVLDKRRGRDPEMGGKPRRKNPQEGLYNELQKDKMAEAYSEIGMK | |
| | GERRRGKGHDGLYQGLSTATKDTYDALHMQALPPR | |
| 111 | LPVLTQPPSASGTPGQRVTISCSGRSSNIGSNSVNWYRQLPGAAPKLLIYSNNQR | anti-BCMA CAR |
| | PPGVPVRFSGSKSGTSASLAISGLQSEDEATYYCATWDDNLNVHYVFGTGTKVTV | |
| | LGSRGGGGSGGGGSGGGGSLEMAQVQLVQSGAEVKKPGSSVKVSCKASGGTFSSY | |
| | AISWVRQAPGQGLEWMGRIIPILGIANYAQKFQGRVTITADKSTSTAYMELSSLR | |
| | SEDTAVYYCARGGYYSHDMWSEDWGQGTLVTVSSAAAPTTTPAPRPPTPAPTIAS | |
| | QPLSLRPEACRPAAGGAVHTRGLDFACDIYIWAPLAGTCGVLLLSLVITLYCNKR | |
| | GRKKLLYIFKQPFMRPVQTTQEEDGCSCRFPEEEEGGCELRVKFSRSAEPPAYQQ | |
| | GQNQLYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 112 | SYELTQPPSASGTPGQRVTMSCSGTSSNIGSHSVNWYQQLPGTAPKLLIYTNNQR | anti-BCMA CAR |
| | PSGVPDRFSGSKSGTSASLAISGLQSEDEADYYCAAWDGSLNGLVFGGGTKLTVL | |
| | GSRGGGGSGGGGSGGGGSLEMAEVQLVQSGAEVKKPGESLKISCKGSGYSFTSYW | |
| | IGWVRQMPGKGLEWMGIIYPGDSDTRYSPSFQGHVTISADKSISTAYLQWSSLKA | |
| | SDTAMYYCARYSGSFDNWGQGTLVTVSSAAAPTTTPAPRPPTPAPTIASQPLSLR | |
| | PEACRPAAGGAVHTRGLDFACDIYIWAPLAGTCGVLLLSLVITLYCNKRGRKKLL | |
| | YIFKQPFMRPVQTTQEEDGCSCRFPEEEEGGCELRVKFSRSAEPPAYQQGQNQLY | |
| | NELNLGRREEYDVLDKRRGRDPEMGGKPRRKNPQEGLYNELQKDKMAEAYSEIGM | |
| | KGERRRGKGHDGLYQGLSTATKDTYDALHMQALPPR | |
| 113 | QAVLTQPPSASGTPGQRVTISCSGSSSNIGSNYVFWYQQLPGTAPKLLIYSNNQR | anti-BCMA CAR |
| | PSGVPDRFSGSKSGTSASLAISGLRSEDEADYYCAAWDDSLSASYVFGTGTKVTV | |
| | LGSRGGGGSGGGGSGGGGSLEMAQVQLVQSGAEVKKPGSSVKVSCKASGGTFSSY | |
| | AISWVRQAPGQGLEWMGRIIPILGTANYAQKFQGRVTITADESTSTAYMELSSLR | |
| | SEDTAVYYCARSGYGSYRWEDSWGQGTLVTVSSAAAPTTTPAPRPPTPAPTIASQ | |
| | PLSLRPEACRPAAGGAVHTRGLDFACDIYIWAPLAGTCGVLLLSLVITLYCNKRG | |
| | RKKLLYIFKQPFMRPVQTTQEEDGCSCRFPEEEEGGCELRVKFSRSAEPPAYQQG | |
| | QNQLYNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| 114 | QSVLTQPPSVSGAPGQRVTISCTGSSSNIGAGFDVHWYQQLPGTAPKLLIYGNSN | anti-BCMA CAR |
| | RPSGVPDRFSGSKSGTSASLAITGLQAEDEADYYCQSYDSSLSGYVFGTGTKVTV | |
| | LGSRGGGGSGGGGSGGGGSLEMAQVQLVQSGAEVKKPGASVKVSCKASGYTFTDY | |
| | YMHWVRQAPGQRLEWMGWINPNSGGTNYAQKFQDRITVTRDTSSNTGYMELTRLR | |
| | SDDTAVYYCARSPYSGVLDKWGQGTLVTVSSAAAPTTTPAPRPPTPAPTIASQPL | |
| | SLRPEACRPAAGGAVHTRGLDFACDIYIWAPLAGTCGVLLLSLVITLYCNKRGRK | |
| | KLLYIFKQPFMRPVQTTQEEDGCSCRFPEEEEGGCELRVKFSRSAEPPAYQQGQN | |
| | QLYNELNLGRREEYDVLDKRRGRDPEMGGKPRRKNPQEGLYNELQKDKMAEAYSE | |
| | IGMKGERRRGKGHDGLYQGLSTATKDTYDALHMQALPPR | |
| 115 | QSALTQPASVSASPGQSIAISCTGTSSDVGWYQQHPGKAPKLMIYEDSKRPSGVS | anti-BCMA CAR |
| | NRFSGSKSGNTASLTISGLQAEDEADYYCSSNTRSSTLVFGGGTKLTVLGSRGGG | |
| | GSGGGGSGGGGSLEMAEVQLVQSGAEMKKPGASLKLSCKASGYTFIDYYVYWMRQ | |
| | APGQGLESMGWINPNSGGTNYAQKFQGRVTMTRDTSISTAYMELSRLRSDDTAMY | |
| | YCARSQRDGYMDYWGQGTLVTVSSAAAPTTTPAPRPPTPAPTIASQPLSLRPEAC | |
| | RPAAGGAVHTRGLDFACDIYIWAPLAGTCGVLLLSLVITLYCNKRGRKKLLYIFK | |
| | QPFMRPVQTTQEEDGCSCRFPEEEEGGCELRVKFSRSAEPPAYQQGQNQLYNELN | |
| | LGRREEYDVLDKRRGRDPEMGGKPRRKNPQEGLYNELQKDKMAEAYSEIGMKGER | |
| | RRGKGHDGLYQGLSTATKDTYDALHMQALPPR | |
| 116 | DIVLTQSPPSLAMSLGKRATISCRASESVTILGSHLIHWYQQKPGQPPTLLIQLA | anti-BCMA CAR |
| | SNVQTGVPARFSGSGSRTDFTLTIDPVEEDDVAVYYCLQSRTIPRTFGGGTKLEI | |
| | KGSTSGSGKPGSGEGSTKGQIQLVQSGPELKKPGETVKISCKASGYTFTDYSINW | |
| | VKRAPGKGLKWMGWINTETREPAYAYDERGRFAFSLETSASTAYLQINNLKYEDT | |
| | ATYFCALDYSYAMDYWGQGTSVTVSSAAATTTPAPRPPTPAPTIASQPLSLRPEA | |
| | CRPAAGGAVHTRGLDFACDIYIWAPLAGTCGVLLLSLVITLYCKRGRKKLLYIFK | |
| | QPFMRPVQTTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAYSEIGMKGER | |
| | RRGKGHDGLYQGLSTATKDTYDALHMQALPPR | |
| 117 | DIVLTQSPASLAVSLGERATINCRASESVSVIGAHLIHWYQQKPGQPPKLLIYLA | anti-BCMA CAR |
| | SNLETGVPARFSGSGSGTDFTLTISSLQAEDAAIYYCLQSRIFPRTFGQGTKLEI | |
| | KGSTSGSGKPGSGEGSTKGQVQLVQSGSELKKPGASVKVSCKASGYTFTDYSINW | |
| | VRQAPGQGLEWMGWINTETREPAYAYDFRGRFVFSLDTSVSTAYLQISSLKAEDT | |
| | AVYYCARDYSYAMDYWGQGTLVTVSSAAATTTPAPRPPTPAPTIASQPLSLRPEA | |
| | CRPAAGGAVHTRGLDFACDIYIWAPLAGTCGVLLLSLVITLYCKRGRKKLLYIFK | |
| | QPFMRPVQTTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAYSEIGMKGER | |
| | RRGKGHDGLYQGLSTATKDTYDALHMQALPPR | |
| 118 | DIVLTQSPASLAVSLGERATINCRASESVSVIGAHLIHWYQQKPGQPPKLLIYLA | anti-BCMA CAR |
| | SNLETGVPARFSGSGSGTDFTLTISSLQAEDAAIYYCLQSRIFPRTFGQGTKLEI | |
| | KGSTSGSGKPGSGEGSTKGQVQLVQSGSELKKPGASVKVSCKASGYTFTDYSINW | |
| | VRQAPGQGLEWMGWINTETREPAYAYDFRGRFVFSLDTSVSTAYLQISSLKAEDT | |
| | AVYYCARDYSYAMDYWGQGTLVTVSSAAADTGLYICKVELMYPPPYYLGIGNGTQ | |
| | IYVIDPEPCPDSDFLLWILAAVSSGLFFYSFLLTAVSKRGRKKLLYIFKQPFMRP | |
| | VQTTQEEDGCSCRFPEEEEGGCELRVKFSRSADAPAYQQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |
| 119 | DIVLTQSPASLAVSIGERATINCRASESVSVIGAHLIHWYQQKPGQPPKLLIYLA | anti-BCMA CAR |
| | SNLETGVPARFSGSGSGTDFTLTISSLQAEDAAIYYCLQSRIFPRTFGQGTKLEI | |
| | KGSTSGSGKPGSGEGSTKGQVQLVQSGSELKKPGASVKVSCKASGYTFTDYSINW | |
| | VRQAPGQGLEWMGWINTETREPAYAYDFRGRFVFSLDTSVSTAYLQISSLKAEDT | |
| | AVYYCARDYSYAMDYWGQGTLVTVSSAAAQIKESLRAELRVTERRAEVPTAHPSP | |
| | SPRPAGQFQTLVVGVVGGLLGSLVLLVWVLAVICSKRGRKKLLYIFKQPFMRPVQ | |
| | TTQEEDGCSCRFPEEEEGGCELRVKFSRSADAPAYQQGQNQLYNELNLGRREEYD | |
| | VLDKRRGRDPEMGGKPRRKNPQEGLYNELQKDKMAEAYSEIGMKGERRRGKGHDG | |
| | LYQGLSTATKDTYDALHMQALPPR | |
| 120 | EVQLVESGGGLVKPGGSLRLSCAASGFTFSDYYMSWIRQAPGKGLEWVSYISSSG | anti-BCMA CAR |
| | STIYYADSVKGRFTISRDNAKNSLYLQMNSLRAEDTAVYYCAKVDGDYTEDYWGQ | |
| | GTLVTVSSGGGGSGGGGSGGGGSQSALTQPASVSGSPGQSITISCTGSSSDVGKY | |
| | NLVSWYQQPPGKAPKLIIYDVNKRPSGVSNRFSGSKSGNTATLTISGLQGDDEAD | |
| | YYCSSYGGSRSYVFGTGTKVTVLESKYGPPCPPCPAPPVAGPSVFLFPPKPKDTL | |
| | MISRTPEVTCVVVDVSQEDPEVQFNWYVDGVEVHNAKTKPREEQFQSTYRVVSVL | |
| | TVLHQDWLNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSRLTVDKSR | |
| | WQEGNVFSCSVMHEALHNHYTQKSLSLSLGKMFWVLVVVGGVLACYSLLVTVAFI | |
| | IFWVKRGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQEGLYNELQK | |
| | DKMAEAYSEIGMKGERRRGKGHDGLYQGLSTATKDTYDALHMQALPPR | |
| 121 | EVQLVQSGGGLVQPGRSLRLSCTASGFTFGDYAMSWFKQAPGKGLEWVGFIRSKA | anti-BCMA CAR |
| | YGGTTEYAASVKGRFTISRDDSKSIAYLQMNSLKTEDTAVYYCAAWSAPTDYWGQ | |
| | GTLVTVSSGGGGSGGGGSGGGGSDIQMTQSPAFLSASVGDRVTVTCRASQGISNY | |
| | LAWYQQKPGNAPRLLIYSASTLQSGVPSRFRGTGYGTEFSLTIDSLQPEDFATYY | |
| | CQQSYTSRQTFGPGTRLDIKESKYGPPCPPCPAPPVAGPSVFLFPPKPKDTLMIS | |
| | RTPEVTCVVVDVSQEDPEVQFNWYVDGVEVHNAKTKPREEQFQSTYRVVSVLTVL | |
| | HQDWLNGKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVDKSRWQE | |
| | GNVFSCSVMHEALHNHYTQKSLSLSLGKMFWVLVVVGGVLACYSLLVTVAFIIFW | |
| | VKRGRKKLLYIFKQPFMRPVQTTQEEDGCSCRFPEEEEGGCELRVKFSRSADAPA | |
| | YQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 122 | EVQLVESGGGLVKPGGSLRLSCAASGFTFSDYYMSWIRQAPGKGLEWVSYISSSG | anti-BCMA CAR |
| | STIYYADSVKGRFTISRDNAKNSLYLQMNSLRAEDTAVYYCAKVDGPPSFDIWGQ | |
| | GTMVTVSSGGGGSGGGGSGGGGSSYVLTQPPSVSVAPGQTARITCGANNIGSKSV | |
| | HWYQQKPGQAPMLVVYDDDDRPSGIPERFSGSNSGNTATLTISGVEAGDEADYFC | |
| | HLWDRSRDHYVFGTGTKLTVLESKYGPPCPPCPAPPVAGPSVFLFPPKPKDTLMI | |
| | SRTPEVTCVVVDVSQEDPEVQFNWYVDGVEVHNAKTKPREEQFQSTYRVVSVLTV | |
| | LHQDWLNGKEYKCKVSNKGLPSSIEKTISKAKGQPREPQVYTLPPSQEEMTKNQV | |
| | SLTCLVKGFYPSDIAVEWESNGQPENNYKTTPPVLDSDGSFFLYSRLTVDKSRWQ | |
| | EGNVFSCSVMHEALHNHYTQKSLSLSLGKMFWVLVVVGGVLACYSLLVTVAFIIF | |
| | WVKRGRKKLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLYNELQKDK | |
| | MAEAYSEIGMKGERRRGKGHDGLYQGLSTATKDTYDALHMQALPPR | |
| 123 | SYELTQPPSASGTPGQRVTMSCSGTSSNIGSHSVNWYQQLPGTAPKLLIYTNNQR | anti-BCMA CAR |
| | PSGVPDRFSGSKSGTSASLAISGLQSEDEADYYCAAWDGSLNGLVFGGGTKLTVL | |
| | GSRGGGGSGGGGSGGGGSLEMAEVQLVQSGAEVKKPGESLKISCKGSGYSFTSYW | |
| | IGWVRQMPGKGLEWMGIIYPGDSDTRYSPSFQGHVTISADKSISTAYLQWSSLKA | |
| | SDTAMYYCARYSGSFDNWGQGTLVTVSSESKYGPPCPPCPAPPVAGPSVFLFPPK | |
| | PKDTLMISRTPEVTCVVVDVSQEDPEVQFNWYVDGVEVHNAKTKPREEQFQSTYR | |
| | VVSVLTVLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFFLYSRLT | |
| | VDKSRWQEGNVFSCSVMHEALHNHYTQKSLSLSLGKMFWVLVVVGGVLACYSLLV | |
| | TVAFIIFWVKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| 124 | QSALTQPASVSASPGQSIAISCTGTSSDVGWYQQHPGKAPKLMIYEDSKRPSGVS | anti-BCMA CAR |
| | NRFSGSKSGNTASLTISGLQAEDEADYYCSSNTRSSTLVFGGGTKLTVLGSRGGG | |
| | GSGGGGSGGGGSLEMAEVQLVQSGAEMKKPGASLKLSCKASGYTFIDYYVYWMRQ | |
| | APGQGLESMGWINPNSGGTNYAQKFQGRVTMTRDTSISTAYMELSRLRSDDTAMY | |
| | YCARSQRDGYMDYWGQGTLVTVSSESKYGPPCPPCPAPPVAGPSVFLFPPKPKDT | |
| | LMISRTPEVTCVVVDVSQEDPEVQFNWYVDGVEVHNAKTKPREEQFQSTYRVVSV | |
| | LTVLHQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLTVDKS | |
| | RWQEGNVFSCSVMHEALHNHYTQKSLSLSLGKMFWVLVVVGGVLACYSLLVTVAF | |
| | IIFWVKRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALPPR | |
| 125 | QSALTQPASVSASPGQSIAISCTGTSSDVGWYQQHPGKAPKLMIYEDSKRPSGVS | anti-BCMA CAR |
| | NRFSGSKSGNTASLTISGLQAEDEADYYCSSNTRSSTLVFGGGTKLTVLGSRGGG | |
| | GSGGGGSGGGGSLEMAEVQLVQSGAEMKKPGASLKLSCKASGYTFIDYYVYWMRQ | |
| | APGQGLESMGWINPNSGGTNYAQKFQGRVTMTRDTSISTAYMELSRLRSDDTAMY | |
| | YCARSQRDGYMDYWGQGTLVTVSSESKYGPPCPPCPAPPVAGPSVFLFPPKPKDT | |
| | LMISRTPEVTCVVVDVSQEDPEVQFNWYVDGVEVHNAKTKPREEQFQSTYRVVSV | |
| | LTVLHQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLTVDKS | |
| | RWQEGNVFSCSVMHEALHNHYTQKSLSLSLGKMFWVLVVVGGVLACYSLLVTVAF | |
| | IIFWVRSKRSRLLHSDYMNMTPRRPGPTRKHYQPYAPPRDFAAYRSRVKFSRSAD | |
| | APAYQQGQNQLYNELNLGRREEYDVLDKRRGRDPEMGGKPRRKNPQEGLYNELQK | |
| | DKMAEAYSEIGMKGERRRGKGHDGLYQGLSTATKDTYDALHMQALPPR | |
| 126 | EVQLVESGGGLVQPGGSLRLSCAVSGFALSNHGMSWVRRAPGKGLEWVSGIVYSG | anti-BCMA CAR |
| | STYYAASVKGRFTISRDNSRNTLYLQMNSLRPEDTAIYYCSAHGGESDVWGQGTT | |
| | VTVSSASGGGGSGGRASGGGGSDIQLTQSPSSLSASVGDRVTITCRASQSISSYL | |
| | NWYQQKPGKAPKLLIYAASSLQSGVPSRFSGSGSGTDFTLTISSLQPEDFATYYC | |
| | QQSYSTPYTFGQGTKVEIKTTTPAPRPPTPAPTIASQPLSLRPEACRPAAGGAVH | |
| | TRGLDFACDIYIWAPLAGTCGVLLLSLVITLYCKRGRKKLLYIFKQPFMRPVQTT | |
| | QEEDGCSCRFPEEEEGGCELRVKFSRSADAPAYKQGQNQLYNELNLGRREEYDVL | |
| | DKRRGRDPEMGGKPRRKNPQEGLYNELQKDKMAEAYSEIGMKGERRRGKGHDGLY | |
| | QGLSTATKDTYDALHMQALPPR | |
| 127 | QVQLVESGGGLVQPGRSLRLSCAASGFTFSNYAMSWVRQAPGKGLGWVSGISRSG | anti-BCMA CAR |
| | ENTYYADSVKGRFTISRDNSKNTLYLQMNSLRDEDTAVYYCARSPAHYYGGMDVW | |
| | GQGTTVTVSSASGGGGSGGRASGGGGSDIVLTQSPGTLSLSPGERATLSCRASQS | |
| | ISSSFLAWYQQKPGQAPRLLIYGASRRATGIPDRFSGSGSGTDFTLTISRLEPED | |
| | SAVYYCQQYHSSPSWTFGQGTKLEIKTTTPAPRPPTPAPTIASQPLSLRPEACRP | |
| | AAGGAVHTRGLDFACDIYIWAPLAGTCGVLLLSLVITLYCKRGRKKLLYIFKQPF | |
| | MRPVQTTQEEDGCSCRFPEEEEGGCELRVKFSRSADAPAYKQGQNQLYNELNLGR | |
| | REEYDVLDKRRGRDPEMGGKPRRKNPQEGLYNELQKDKMAEAYSEIGMKGERRRG | |
| | KGHDGLYQGLSTATKDTYDALHMQALPPR | |
| 128 | QVQLVESGGGLVQPGGSLRLSCAVSGFALSNHGMSWVRRAPGKGLEWVSGIVYSG | anti-BCMA CAR |
| | STYYAASVKGRFTISRDNSRNTLYLQMNSLRPEDTAIYYCSAHGGESDVWGQGTT | |
| | VTVSSASGGGGSGGRASGGGGSDIRLTQSPSPLSASVGDRVTITCQASEDINKFL | |
| | NWYHQTPGKAPKLLIYDASTLQTGVPSRFSGSGSGTDFTLTINSLQPEDIGTYYC | |
| | QQYESLPLTFGGGTKVEIKTTTPAPRPPTPAPTIASQPLSLRPEACRPAAGGAVH | |
| | TRGLDFACDIYIWAPLAGTCGVLLLSLVITLYCKRGRKKLLYIFKQPFMRPVQTT | |
| | QEEDGCSCRFPEEEEGGCELRVKFSRSADAPAYKQGQNQLYNELNLGRREEYDVL | |
| | DKRRGRDPEMGGKPRRKNPQEGLYNELQKDKMAEAYSEIGMKGERRRGKGHDGLY | |
| | QGLSTATKDTYDALHMQALPPR | |
| 129 | EVQLVESGGGLVQPGGSLRLSCAVSGFALSNHGMSWVRRAPGKGLEWVSGIVYSG | anti-BCMA CAR |
| | STYYAASVKGRFTISRDNSRNTLYLQMNSLRPEDTAIYYCSAHGGESDVWGQGTT | |
| | VTVSSASGGGGSGGRASGGGGSEIVLTQSPGTLSLSPGERATLSCRASQSIGSSS | |
| | LAWYQQKPGQAPRLLMYGASSRASGIPDRFSGSGSGTDFTLTISRLEPEDFAVYY | |
| | CQQYAGSPPFTFGQGTKVEIKTTTPAPRPPTPAPTIASQPLSLRPEACRPAAGGA | |
| | VHTRGLDFACDIYIWAPLAGTCGVLLLSLVITLYCKRGRKKLLYIFKQPFMRPVQ | |
| | TTQEEDGCSCRFPEEEEGGCELRVKFSRSADAPAYKQGQNQLYNELNLGRREEYD | |
| | VLDKRRGRDPEMGGKPRRKNPQEGLYNELQKDKMAEAYSEIGMKGERRRGKGHDG | |
| | LYQGLSTATKDTYDALHMQALPPR | |
| 130 | QIQLVQSGPDLKKPGETVKLSCKASGYTFTNFGMNWVKQAPGKGFKWMAWINTYT | anti-BCMA CAR |
| | GESYFADDFKGRFAFSVETSATTAYLQINNLKTEDTATYFCARGEIYYGYDGGFA | |
| | YWGQGTLVTVSAGGGGSGGGGSGGGGSDVVMTQSHRFMSTSVGDRVSITCRASQD | |
| | VNTAVSWYQQKPGQSPKLLIFSASYRYTGVPDRFTGSGSGADFTLTISSVQAEDL | |
| | AVYYCQQHYSTPWTFGGGTKLDIKTTTPAPRPPTPAPTIASQPLSLRPEACRPAA | |
| | GGAVHTRGLDFACDIYIWAPLAGTCGVLLLSLVITLYCKRGRKKLLYIFKQPFMR | |
| | PVQTTQEEDGCSCRFPEEEEGGCELRVKFSRSADAPAYKQGQNQLYNELNLGRRE | |
| | EYDVLDKRRGRDPEMGGKPRRKNPQEGLYNELQKDKMAEAYSEIGMKGERRRGKG | |
| | HDGLYQGLSTATKDTYDALHMQALPPR | |
| 131 | QIQLVQSGPELKKPGETVKISCKASGYTFTDYSINWVKRAPGKGLKWMGWINTET | anti-BCMA CAR |
| | REPAYAYDFRGRFAFSLETSASTAYLQINNLKYEDTATYFCALDYSYAMDYWGQG | |
| | TSVTVSSGGGGSGGGGSGGGGSQIQLVQSGPELKKPGETVKISCKASGYTFTDYS | |
| | INWVKRAPGKGLKWMGWINTETREPAYAYDFRGRFAFSLETSASTAYLQINNLKY | |
| | EDTATYFCALDYSYAMDYWGQGTSVTVSSTTTPAPRPPTPAPTIASQPLSLRPEA | |
| | CRPAAGGAVHTRGLDFACDIYIWAPLAGTCGVLLLSLVITLYCKRGRKKLLYIFK | |
| | QPFMRPVQTTQEEDGCSCRFPEEEEGGCELRVKFSRSADAPAYKQGQNQLYNELN | |
| | LGRREEYDVLDKRRGRDPEMGGKPRRKNPQEGLYNELQKDKMAEAYSEIGMKGER | |
| | RRGKGHDGLYQGLSTATKDTYDALHMQALPPR | |
| 132 | QIQLVQSGPELKKPGETVKISCKASGYTFTDYSINWVKRAPGKGLKWMGWINTET | anti-BCMA CAR |
| | REPAYAYDFRGRFAFSLETSASTAYLQINNLKYEDTATYFCALDYSYAMDYWGQG | |
| | TSVTVSSGGGGSGGGGSGGGGSDIVLTQSPASLAMSLGKRATISCRASESVSVIG | |
| | AHLIHWYQQKPGQPPKLLIYLASNLETGVPARFSGSGSGTDFTLTIDPVEEDDVA | |
| | IYSCLQSRIFPRTFGGGTKLEIKTTTPAPRPPTPAPTIASQPLSLRPEACRPAAG | |
| | GAVHTRGLDFACDIYIWAPLAGTCGVLLLSLVITLYCKRGRKKLLYIFKQPFMRP | |
| | VQTTQEEDGCSCRFPEEEEGGCELRVKFSRSADAPAYKQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |
| 133 | QIQLVQSGPELKKPGETVKISCKASGYTFRHYSMNWVKQAPGKGLKWMGRINTES | anti-BCMA CAR |
| | GVPIYADDFKGRFAFSVETSASTAYLVINNLKDEDTASYFCSNDYLYSLDFWGQG | |
| | TALTVSSGGGGSGGGGSGGGGSDIVLTQSPPSLAMSLGKRATISCRASESVTILG | |
| | SHLIYWYQQKPGQPPTLLIQLASNVQTGVPARFSGSGSRTDFTLTIDPVEEDDVA | |
| | VYYCLQSRTIPRTFGGGTKLEIKTTTPAPRPPTPAPTIASQPLSLRPEACRPAAG | |
| | GAVHTRGLDFACDIYIWAPLAGTCGVLLLSLVITLYCKRGRKKLLYIFKQPFMRP | |
| | VQTTQEEDGCSCRFPEEEEGGCELRVKFSRSADAPAYKQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |
| 134 | QIQLVQSGPELKKPGETVKISCKASGYTFTHYSMNWVKQAPGKGLKWMGRINTET | anti-BCMA CAR |
| | GEPLYADDFKGRFAFSLETSASTAYLVINNLKNEDTATFFCSNDYLYSCDYWGQG | |
| | TTLTVSSGGGGSGGGGSGGGGSDIVLTQSPPSLAMSLGKRATISCRASESVTILG | |
| | SHLIYWYQQKPGQPPTLLIQLASNVQTGVPARFSGSGSRTDFTLTIDPVEEDDVA | |
| | VYYCLQSRTIPRTFGGGTKLEIKTTTPAPRPPTPAPTIASQPLSLRPEACRPAAG | |
| | GAVHTRGLDFACDIYIWAPLAGTCGVLLLSLVITLYCKRGRKKLLYIFKQPFMRP | |
| | VQTTQEEDGCSCRFPEEEEGGCELRVKFSRSADAPAYKQGQNQLYNELNLGRREE | |
| | YDVLDKRRGRDPEMGGKPRRKNPQEGLYNELQKDKMAEAYSEIGMKGERRRGKGH | |
| | DGLYQGLSTATKDTYDALHMQALPPR | |
| 135 | DIVLTQSPPSLAMSIGKRATISCRASESVTILGSHLIHWYQQKPGQPPTLLIQLA | anti-BCMA CAR |
| | SNVQTGVPARFSGSGSRTDFTLTIDPVEEDDVAVYYCLQSRTIPRTFGGGTKLEI | |
| | KGSTSGSGKPGSGEGSTKGQIQLVQSGPELKKPGETVKISCKASGYTFTDYSINW | |
| | VKRAPGKGLKWMGWINTETREPAYAYDFRGRFAFSLETSASTAYLQINNLKYEDT | |
| | ATYFCALDYSYAMDYWGQGTSVTVSSFVPVFLPAKPTTTPAPRPPTPAPTIASQP | |
| | LSLRPEACRPAAGGAVHTRGLDFACDIYIWAPLAGTCGVLLLSLVITLYCNHRNR | |
| | SKRSRLLHSDYMNMTPRRPGPTRKHYQPYAPPRDFAAYRSRVKFSRSADAPAYQQ | |
| | GQNQLYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 136 | QVQLVQSGAEVKKPGASVKVSCKASGYSFPDYYINWVRQAPGQGLEWMGWIYFAS | anti-BCMA CAR |
| | GNSEYNQKFTGRVTMTRDTSINTAYMELSSLTSEDTAVYFCASLYDYDWYFDVWG | |
| | QGTMVTVSSGGGGSGGGGSGGGGSDIVMTQTPLSLSVTPGQPASISCKSSQSLVH | |
| | SNGNTYLHWYLQKPGQSPQLLIYKVSNRFSGVPDRFSGSGSGTDFTLKISRVEAE | |
| | DVGIYYCSQSSIYPWTFGQGTKLEIKGLAVSTISSFFPPGYQIYIWAPLAGTCGV | |
| | LLLSLVITLYCKRGRKKLLYIFKQPFMRPVQTTQEEDGCSCRFPEEEEGGCELRV | |
| | KFSRSADAPAYQQGQNQLYNELNLGRREEYDVLDKRRGRDPEMGGKPRRKNPQEG | |
| | LYNELQKDKMAEAYSEIGMKGERRRGKGHDGLYQGLSTATKDTYDALHMQALPPR | |
| 137 | QVQLVQSGAEVKKPGASVKVSCKASGYSFPDYYINWVRQAPGQGLEWMGWIYFAS | anti-BCMA CAR |
| | GNSEYNQKFTGRVTMTRDTSINTAYMELSSLTSEDTAVYFCASLYDYDWYFDVWG | |
| | QGTMVTVSSGGGGSGGGGSGGGGSDIVMTQTPLSLSVTPGQPASISCKSSQSLVH | |
| | SNGNTYLHWYLQKPGQSPQLLIYKVSNRFSGVPDRFSGSGSGTDFTLKISRVEAE | |
| | DVGIYYCSQSSIYPWTFGQGTKLEIKTTTPAPRPPTPAPTIASQPLSLRPEACRP | |
| | AAGGAVHTRGLDFACDIYIWAPLAGTCGVLLLSLVITLYCKRGRKKLLYIFKQPF | |
| | MRPVQTTQEEDGCSCRFPEEEEGGCELRVKFSRSADAPAYQQGQNQLYNELNLGR | |
| | REEYDVLDKRRGRDPEMGGKPRRKNPQEGLYNELQKDKMAEAYSEIGMKGERRRG | |
| | KGHDGLYQGLSTATKDTYDALHMQALPPR | |
| 138 | QVQLVQSGAEVKKPGASVKVSCKASGYSFPDYYINWVRQAPGQGLEWMGWIYFAS | anti-BCMA CAR |
| | GNSEYNQKFTGRVTMTRDTSINTAYMELSSLTSEDTAVYFCASLYDYDWYFDVWG | |
| | QGTMVTVSSGGGGSGGGGSGGGGSDIVMTQTPLSLSVTPGQPASISCKSSQSLVH | |
| | SNGNTYLHWYLQKPGQSPQLLIYKVSNRFSGVPDRFSGSGSGTDFTLKISRVEAE | |
| | DVGIYYCSQSSIYPWTFGQGTKLEIKEPKSPDKTHTCPPCPAPPVAGPSVFLFPP | |
| | KPKDTLMIARTPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFLYSKL | |
| | TVDKSRWQQGNVFSCSVMHEALHNHYTQKSLSLSPGKIYIWAPLAGTCGVLLLSL | |
| | VITLYCKRGRKKLLYIFKQPFMRPVQTTQEEDGCSCRFPEEEEGGCELRVKFSRS | |
| | ADAPAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| 139 | QVQLVQSGAEVKKPGASVKVSCKASGYSFPDYYINWVRQAPGQGLEWMGWIYFAS | anti-BCMA CAR |
| | GNSEYNQKFTGRVTMTRDTSSSTAYMELSSLRSEDTAVYFCASLYDYDWYFDVWG | |
| | QGTMVTVSSGGGGSGGGGSGGGGSDIVMTQTPLSLSVTPGEPASISCKSSQSLVH | |
| | SNGNTYLHWYLQKPGQSPQLLIYKVSNRFSGVPDRFSGSGSGADFTLKISRVEAE | |
| | DVGVYYCAETSHVPWTFGQGTKLEIKGLAVSTISSFFPPGYQIYIWAPLAGTCGV | |
| | LLLSLVITLYCKRGRKKLLYIFKQPFMRPVQTTQEEDGCSCRFPEEEEGGCELRV | |
| | KFSRSADAPAYQQGQNQLYNELNLGRREEYDVLDKRRGRDPEMGGKPRRKNPQEG | |
| | LYNELQKDKMAEAYSEIGMKGERRRGKGHDGLYQGLSTATKDTYDALHMQALPPR | |
| 140 | QVQLVQSGAEVKKPGASVKVSCKASGYSFPDYYINWVRQAPGQGLEWMGWIYFAS | anti-BCMA CAR |
| | GNSEYNQKFTGRVTMTRDTSSSTAYMELSSLRSEDTAVYFCASLYDYDWYFDVWG | |
| | QGTMVTVSSGGGGSGGGGSGGGGSDIVMTQTPLSLSVTPGEPASISCKSSQSLVH | |
| | SNGNTYLHWYLQKPGQSPQLLIYKVSNRFSGVPDRFSGSGSGADFTLKISRVEAE | |
| | DVGVYYCAETSHVPWTFGQGTKLEIKTTTPAPRPPTPAPTIASQPLSLRPEACRP | |
| | AAGGAVHTRGLDFACDIYIWAPLAGTCGVLLLSLVITLYCKRGRKKLLYIFKQPF | |
| | MRPVQTTQEEDGCSCRFPEEEEGGCELRVKFSRSADAPAYQQGQNQLYNELNLGR | |
| | REEYDVLDKRRGRDPEMGGKPRRKNPQEGLYNELQKDKMAEAYSEIGMKGERRRG | |
| | KGHDGLYQGLSTATKDTYDALHMQALPPR | |
| 141 | QVQLVQSGAEVKKPGASVKVSCKASGYSFPDYYINWVRQAPGQGLEWMGWIYFAS | anti-BCMA CAR |
| | GNSEYNQKFTGRVTMTRDTSSSTAYMELSSLRSEDTAVYFCASLYDYDWYFDVWG | |
| | QGTMVTVSSGGGGSGGGGSGGGGSDIVMTQTPLSLSVTPGEPASISCKSSQSLVH | |
| | SNGNTYLHWYLQKPGQSPQLLIYKVSNRFSGVPDRFSGSGSGADFTLKISRVEAE | |
| | DVGVYYCAETSHVPWTFGQGTKLEIKEPKSPDKTHTCPPCPAPPVAGPSVFLFPP | |
| | KPKDTLMIARTPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFLYSKL | |
| | TVDKSRWQQGNVFSCSVMHEALHNHYTQKSLSLSPGKIYIWAPLAGTCGVLLLSL | |
| | VITLYCKRGRKKLLYIFKQPFMRPVQTTQEEDGCSCRFPEEEEGGCELRVKFSRS | |
| | ADAPAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| 142 | IYIWAPLAGTCGVLLLSLVITLYCNHRN | CD8a TM |
| 143 | IYIWAPLAGTCGVLLLSLVIT | CD8a TM |
| 144 | RAAA | linking peptide |
| 145 | EVQLVESGGGLVKPGGSLRLSCAASGFTFSDYYMSWIRQAPGKGLEWVSYISSSG | Variable heavy |
| | STIYYADSVKGRFTISRDNAKNSLYLQMNSLRAEDTAVYYCAKVDGDYTEDYWGQ | (VH) Anti- |
| | GTLVTVSS | BCMA |
| 146 | QSALTQPASVSGSPGQSITISCTGSSSDVGKYNLVSWYQQPPGKAPKLIIYDVNK | Variable light |
| | RPSGVSNRFSGSKSGNTATLTISGLQGDDEADYYCSSYGGSRSYVFGTGTKVTVL | (VL) Anti- |
| | | BCMA |
| 147 | EVQLVQSGGGLVQPGRSLRLSCTASGFTFGDYAMSWFRQAPGKGLEWVGFIRSKA | Variable heavy |
| | YGGTTEYAASVKGRFTISRDDSKSIAYLQMNSLKTEDTAVYYCAAWSAPTDYWGQ | (VH) Anti- |
| | GTLVTVSS | BCMA |
| 148 | DIQMTQSPAFLSASVGDRVTVTCRASQGISNYLAWYQQKPGNAPRLLIYSASTLQ | Variable light |
| | SGVPSRFRGTGYGTEFSLTIDSLQPEDFATYYCQQSYTSRQTFGPGTRLDIK | (VL) Anti- |
| | | BCMA |
| 149 | EVQLVESGGGLVKPGGSLRLSCAASGFTFSDYYMSWIRQAPGKGLEWVSYISSSG | Variable heavy |
| | STIYYADSVKGRFTISRDNAKNSLYLQMNSLRAEDTAVYYCAKVDGPPSFDIWGQ | (VH) Anti- |
| | GTMVTVSS | BCMA |
| 150 | SYVLTQPPSVSVAPGQTARITCGANNIGSKSVHWYQQKPGQAPMLVVYDDDDRPS | Variable light |
| | GIPERFSGSNSGNTATLTISGVEAGDEADYFCHLWDRSRDHYVFGTGTKLTVL | (VL) Anti- |
| | | BCMA |
| 151 | EVQLVQSGAEVKKPGSSVKVSCKASGGTFSSYAISWVRQAPGQGLEWMGRIIPIL | Variable heavy |
| | GIANYAQKFQGRVTMTEDTSTDTAYMELSSLRSEDTAVYYCARSGYSKSIVSYMD | (VH) Anti- |
| | YWGQGTLVTVSS | BCMA |
| 152 | LPVLTQPPSTSGTPGQRVTVSCSGSSSNIGSNVVFWYQQLPGTAPKLVIYRNNQR | Variable light |
| | PSGVPDRFSVSKSGTSASLAISGLRSEDEADYYCAAWDDSLSGYVFGTGTKVTVL | (VL) Anti- |
| | G | BCMA |
| 153 | MPLLLLLPLLWAGALA | CD33 Signal |
| | | peptide |
| 154 | MALPVTALLLPLALLLHA | CD8 alpha signal |
| | | peptide |
| 155 | atgcttctcctggtgacaagccttctgctctgtgagttaccacacccagcattcc | GMCSFR alpha |
| | tcctgatccca | chain signal |
| | | sequence |
| 156 | MLLLVTSLLLCELPHPAFLLIP | GMCSFR alpha |
| | | chain signal |
| | | sequence |
| 157 | Glu Val Val Val Lys Tyr Gly Pro Pro Cys Pro Pro Cys Pro | Exemplary IgG |
| | | Hinge |
| 158 | X1PPX2P | Exemplary IgG |
| | X1 is glycine, cysteine or arginine | Hinge |
| | X2 is cysteine or threonine | |
| 159 | Glu Pro Lys Ser Cys Asp Lys Thr His Thr Cys Pro Pro Cys | Exemplary IgG |
| | Pro | Hinge |
| 160 | Glu Arg Lys Cys Cys Val Glu Cys Pro Pro Cys Pro | Exemplary IgG |
| | | Hinge |
| 161 | ELKTPLGDTHTCPRCPEPKSCDTPPPCPRCPEPKSCDTPPPCPRCPEPKSCDTPP | Exemplary IgG |
| | PCPRCP | Hinge |
| 162 | Glu Ser Lys Tyr Gly Pro Pro Cys Pro Ser Cys Pro | Exemplary IgG |
| | | Hinge |
| 163 | Glu Ser Lys Tyr Gly Pro Pro Cys Pro Pro Cys Pro | Exemplary IgG |
| | | Hinge |
| 164 | Tyr Gly Pro Pro Cys Pro Pro Cys Pro | Exemplary IgG |
| | | Hinge |
| 165 | Lys Tyr Gly Pro Pro Cys Pro Pro Cys Pro | Exemplary IgG |
| | | Hinge |
| 166 | MLLLVTSLLLCELPHPAFLLIPRKVCNGIGIGEFKDSLSINATNIKHFKNCTSIS | tEGFR |
| | GDLHILPVAFRGDSFTHTPPLDPQELDILKTVKEITGFLLIQAWPENRTDLHAFE | artificial |
| | NLEIIRGRTKQHGQFSLAVVSLNITSLGLRSLKEISDGDVIISGNKNLCYANTIN | |
| | WKKLFGTSGQKTKIISNRGENSCKATGQVCHALCSPEGCWGPEPRDCVSCRNVSR | |
| | GRECVDKCNLLEGEPREFVENSECIQCHPECLPQAMNITCTGRGPDNCIQCAHYI | |
| | DGPHCVKTCPAGVMGENNTLVWKYADAGHVCHLCHPNCTYGCTGPGLEGCPTNGP | |
| | KIPSIATGMVGALLLLLVVALGIGLFM | |
| 167 | EGRGSLLTCGDVEENPGP | T2A artificial |
| 168 | GSGATNFSLLKQAGDVEENPGP | P2A |
| 169 | ATNFSLLKQAGDVEENPGP | P2A |
| 170 | QCTNYALLKLAGDVESNPGP | E2A |
| 171 | VKQTLNFDLLKLAGDVESNPGP | F2A |
| 172 | MLQMAGQCSQNEYFDSLLHACIPCQLRCSSNTPPLTCQRYCNASVTNSVKGTNAG | BCMA-Fc fusion |
| | GGGSPKSSDKTHTCPPCPAPEAEGAPSVFLFPPKPKDTIMISRTPEVTCVVVDVS | polypeptide |
| | HEDPEVKFNWYVDGVEVHNAKTKPREEQYNSTYRVVSVLTVLHQDWLNGKEYKCK | |
| | VSNKALPSSIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSCSVMHEAL | |
| | HNHYTQKSLSLSPGK | |
| 173 | DYYVY | CDR-H1 |
| 174 | WINPNSGGTNYAQKFQG | CDR-H2 |
| 175 | SQRDGYMDY | CDR-H3 |
| 176 | GYTFIDY | CDR-H1 |
| 177 | NPNSGG | CDR-H2 |
| 178 | GYTFIDYYVY | CDR-H1 |
| 179 | WINPNSGGTN | CDR-H2 |
| 180 | GYTFIDYY | CDR-H1 |
| 181 | INPNSGGT | CDR-H2 |
| 182 | ARSQRDGYMDY | CDR-H3 |
| 183 | TGTSSDVG | CDR-L1 |
| 184 | EDSKRPS | CDR-L2 |
| 185 | SSNTRSSTLV | CDR-L3 |
| 186 | ISCTGTSSD | CDR-L1 |
| 187 | EDS | CDR-L2 |
| 188 | QSALTQPASVSASPGQSIAISCTGTSSDVGWYQQHPGKAPKLMIYEDSKRPSGVS | anti-BCMA scFv |
| | NRFSGSKSGNTASLTISGLQAEDEADYYCSSNTRSSTLVFGGGTKLTVLGSRGGG | |
| | GSGGGGSGGGGSLEMAEVQLVQSGAEMKKPGASLKLSCKASGYTFIDYYVYWMRQ | |
| | APGQGLESMGWINPNSGGTNYAQKFQGRVTMTRDTSISTAYMELSRLRSDDTAMY | |
| | YCARSQRDGYMDYWGQGTLVTVSS | |
| 189 | DYSIN | CDR-H1 |
| 190 | WINTETREPAYAYDFRG | CDR-H2 |
| 191 | DYSYAMDY | CDR-H3 |
| 192 | RASESVTILGSHLIH | CDR-L1 |
| 193 | LASNVQT | CDR-L2 |
| 194 | LQSRTIPRT | CDR-L3 |
| 195 | GYTFTDY | CDR-H1 |
| 196 | NTETPE | CDR-H2 |
| 197 | DYSYAMDY | CDR-H3 |
| 198 | RASESVTILGSHLIH | CDR-L1 |
| 199 | LASNVQT | CDR-L2 |
| 200 | LQSRTIPRT | CDR-L3 |
| 201 | GYTFTDYSIN | CDR-H1 |
| 202 | WINTETREPA | CDR-H2 |
| 203 | DYSYAMDY | CDR-H3 |
| 204 | RASESVTILGSHLIH | CDR-L1 |
| 205 | LASNVQT | CDR-L2 |
| 206 | LQSRTIPRT | CDR-L3 |
| 207 | GYTFTDYS | CDR-H1 |
| 208 | INTETREP | CDR-H2 |
| 209 | ALDYSYAMDY | CDR-H3 |
| 210 | ESVTILGSHL | CDR-L1 |
| 211 | LA | CDR-L2 |
| 212 | LQSRTIPRT | CDR-L3 |
| 213 | DIVLTQSPPSLAMSLGKRATISCRASESVTILGSHLIHWYQQKPGQPPTLLIQLA | scFv |
| | SNVQTGVPARFSGSGSRTDFTLTIDPVEEDDVAVYYCLQSRTIPRTFGGGTKLEI | |
| | KGSTSGSGKPGSGEGSTKGQIQLVQSGPELKKPGETVKISCKASGYTFTDYSINW | |
| | VKPAPGKGLKWMGWINTETPEPAYAYDFRGRFAFSLETSASTAYLQINNLKYEDT | |
| | ATYFCALDYSYAMDYTSVTVSS | |
| 214 | atggcactcc ccgtcaccgc ccttctcttg cccctcgccc tgctgctgca | BCMA CAR (nt) |
| | tgctgccagg cccgacattg tgctcactca gtcacctccc agcctggcca | |
| | tgagcctggg aaaaagggcc accatctcct gtagagccag tgagtccgtc | |
| | acaatcttgg ggagccatct tattcactgg tatcagcaga agcccgggca | |
| | gcctccaacc cttcttattc agctcgcgtc aaacgtccag acgggtgtac | |
| | ctgccagatt ttctggtagc gggtcccgca ctgattttac actgaccata | |
| | gatccagtgg aagaagacga tgtggccgtg tattattgtc tgcagagcag | |
| | aacgattcct cgcacatttg gtgggggtac taagctggag attaagggaa | |
| | gcacgtccgg ctcagggaag ccgggctccg gcgagggaag cacgaagggg | |
| | caaattcagc tggtccagag cggacctgag ctgaaaaaac ccggcgagac | |
| | tgttaagatc agttgtaaag catctggcta taccttcacc gactacagca | |
| | taaattgggt gaaacgggcc cctggaaagg gcctcaaatg gatgggttgg | |
| | atcaataccg aaactaggga gcctgcttat gcatatgact tccgcgggag | |
| | attcgccttt tcactcgaga catctgcctc tactgcttac ctccaaataa | |
| | acaacctcaa gtatgaagat acagccactt acttttgcgc cctcgactat | |
| | agttacgcca tggactactg gggacaggga acctccgtta ccgtcagttc | |
| | cgcggccgca accacaacac ctgctccaag gccccccaca cccgctccaa | |
| | ctatagccag ccaaccattg agcctcagac ctgaagcttg caggcccgca | |
| | gcaggaggcg ccgtccatac gcgaggcctg gacttcgcgt gtgatattta | |
| | tatttgggcc cctttggccg gaacatgtgg ggtgttgctt ctctcccttg | |
| | tgatcactct gtattgtaag cgcgggagaa agaagctcct gtacatcttc | |
| | aagcagcctt ttatgcgacc tgtgcaaacc actcaggaag aagatgggtg | |
| | ttcatgccgc ttccccgagg aggaagaagg agggtgtgaa ctgagggtga | |
| | aattttctag aagcgccgat gctcccgcat atcagcaggg tcagaatcag | |
| | ctctacaatg aattgaatct cggcaggcga gaagagtacg atgttctgga | |
| | caagagacgg ggcagggatc ccgagatggg gggaaagccc cggagaaaaa | |
| | atcctcagga ggggttgtac aatgagctgc agaaggacaa gatggctgaa | |
| | gcctatagcg agatcggaat gaaaggcgaa agacgcagag gcaaggggca | |
| | tgacggtctg taccagggtc tctctacagc caccaaggac acttatgatg | |
| | cgttgcatat gcaagccttg ccaccccgct aatga | |
| 215 | MLQMAGQCSQNEYFDSLLHACIPCQLRCSSNTPPLTCQRYCNASVTNSVKGTNAI | Human BCMA |
| | LWTCLGLSLIISLAVFVLMFLLRKINSEPLKDEFKNTGSGLLGMANIDLEKSPTG | |
| | DEIILPRGLEYTVEECTCEDCIKSKPKVDSDHCFPLPAMEEGATILVTTKTNDYC | |
| | KSLPAALSATEIEKSISAR | |
| 216 | EVQLLESGGGLVQPGGSLRLSCAASGFTFSSYAMSWVRQAPGKGLEWVSAISGSG | Anti-BCMA scFv |
| | GSTYYADSVKGPFTISRDNSKNTLYLQMNSLRAEDTAVYYCAPAEMGAVFDIWGQ | |
| | GTMVTVSSGSTSGSGKPGSGEGSTKGEIVLTQSPATLSLSPGERATLSCRASQSV | |
| | SPYLAWYQQKPGQAPRLLIYDASNRATGIPARFSGSGSGTDFTLTISSLEPEDFA | |
| | VYYCQQRISWPFTFGGGTKVEIK | |
| 217 | EIVLTQSPATLSLSPGEPATLSCRASQSVSRYLAWYQQKPGQAPRLLIYDASNRA | Anti-BCMA scFv |
| | TGIPARFSGSGSGTDFTLTISSLEPEDFAVYYCQQRISWPFTFGGGTKVEIKRGS | |
| | TSGSGKPGSGEGSTKGEVQLLESGGGLVQPGGSLRLSCAASGFTFSSYAMSWVRQ | |
| | APGKGLEWVSAISGSGGSTYYADSVKGRFTISRDNSKNTLYLQMNSLPAEDTAVY | |
| | YCARAEMGAVFDIWGQGTMVTVSS | |
| 218 | QVQLVESGGGVVQPGPSLRLSCAASGFTFSSYGMHWVRQAPGKGLEWVAVISYDG | Anti-BCMA scFv |
| | SNKYYADSVKGRFTISRDNSKNTLYLQMNSLPAEDTAVYYCARDGTYLGGLWYFD | |
| | LWGRGTLVTVSSGSTSGSGKPGSGEGSTKGDIVMTQSPLSLPVTPGEPASISCPS | |
| | SQSLLHSNGYNYLDWYLQKPGQSPQLLIYLGSNRASGVPDRFSGSGSGTDFTLKI | |
| | SRVEAEDVGVYYCMQGLGLPLTFGGGTKVEIK | |
| 219 | DIVMTQSPLSLPVTPGEPASISCRSSQSLLHSNGYNYLDWYLQKPGQSPQLLIYL | Anti-BCMA scFv |
| | GSNRASGVPDRFSGSGSGTDFTLKISRVEAEDVGVYYCMQGLGLPLTFGGGTKVE | |
| | IKRGSTSGSGKPGSGEGSTKGQVQLVESGGGVVQPGRSLRLSCAASGFTFSSYGM | |
| | HWVRQAPGKGLEWVAVISYDGSNKYYADSVKGRFTISRDNSKNTLYLQMNSLRAE | |
| | DTAVYYCARDGTYLGGLWYFDLWGRGTLVTVSS | |
| 220 | QVQLVQSGAEVKKPGASVKVSCKASGYTFTSYYMHWVRQAPGQGLEWMGIINPGG | Anti-BCMA scFv |
| | GSTSYAQKFQGRVTMTRDTSTSTVYMELSSLRSEDTAVYYCARESWPMDVWGQGT | |
| | TVTVSSGSTSGSGKPGSGEGSTKGEIVMTQSPATLSVSPGERATLSCRASQSVSS | |
| | NLAWYQQKPGQAPRLLIYGASTRATGIPARFSGSGSGTEFTLTISSLQSEDFAVY | |
| | YCQQYAAYPTFGGGTKVEIK | |
| 221 | EIVMTQSPATLSVSPGERATLSCRASQSVSSNLAWYQQKPGQAPRLLIYGASTRA | Anti-BCMA scFv |
| | TGIPARFSGSGSGTEFTLTISSLQSEDFAVYYCQQYAAYPTFGGGTKVEIKRGST | |
| | SGSGKPGSGEGSTKGQVQLVQSGAEVKKPGASVKVSCKASGYTFTSYYMHWVRQA | |
| | PGQGLEWMGIINPGGGSTSYAQKFQGRVTMTRDTSTSTVYMELSSLRSEDTAVYY | |
| | CARESWPMDVWGQGTTVTVSS | |
| 222 | QLQLQESGPGLVKPSETLSLTCTVSGGSISSSSYYWGWIRQPPGKGLEWIGSISY | Anti-BCMA scFv |
| | SGSTYYNPSLKSRVTISVDTSKNQFSLKLSSVTAADTAVYYCARGRGYATSLAFD | |
| | IWGQGTMVTVSSGSTSGSGKPGSGEGSTKGEIVLTQSPATLSLSPGERATLSCRA | |
| | SQSVSSYLAWYQQKPGQAPRLLIYDASNRATGIPARFSGSGSGTDFTLTISSLEP | |
| | EDFAVYYCQQRHVWPPTFGGGTKVEIK | |
| 223 | EIVLTQSPATLSLSPGERATLSCRASQSVSSYLAWYQQKPGQAPRLLIYDASNRA | Anti-BCMA scFv |
| | TGIPARFSGSGSGTDFTLTISSLEPEDFAVYYCQQRHVWPPTFGGGTKVEIKRGS | |
| | TSGSGKPGSGEGSTKGQLQLQESGPGLVKPSETLSLTCTVSGGSISSSSYYWGWI | |
| | RQPPGKGLEWIGSISYSGSTYYNPSLKSRVTISVDTSKNQFSLKLSSVTAADTAV | |
| | YYCARGRGYATSLAFDIWGQGTMVTVSS | |
| 224 | EVQLVESGGGLVQPGGSLRLSCAASGFTFSSYSMNWVRQAPGKGLEWVSTISSSS | Anti-BCMA scFv |
| | STIYYADSVKGRFTISRDNAKNSLYLQMNSLRAEDTAVYYCARGSQEHLIFDYWG | |
| | QGTLVTVSSGSTSGSGKPGSGEGSTKGEIVLTQSPATLSLSPGERATLSCRASQS | |
| | VSRYLAWYQQKPGQAPRLLIYDASNRATGIPARFSGSGSGTDFTLTISSLEPEDF | |
| | AVYYCQQRFYYPWTFGGGTKVEIK | |
| 225 | EIVLTQSPATLSLSPGERATLSCRASQSVSRYLAWYQQKPGQAPRLLIYDASNRA | Anti-BCMA scFv |
| | TGIPARFSGSGSGTDFTLTISSLEPEDFAVYYCQQRFYYPWTFGGGTKVEIKRGS | |
| | TSGSGKPGSGEGSTKGEVQLVESGGGLVQPGGSLRLSCAASGFTFSSYSMNWVRQ | |
| | APGKGLEWVSTISSSSSTIYYADSVKGRFTISRDNAKNSLYLQMNSLRAEDTAVY | |
| | YCARGSQEHLIFDYWGQGTLVTVSS | |
| 226 | QVQLVESGGGVVQPGRSLRLSCAASGFTFSSYGMHWVRQAPGKGLEWVAVISYDG | Anti-BCMA scFv |
| | SNKYYADSVKGRFTISRDNSKNTLYLQMNSLRAEDTAVYYCARTDFWSGSPPGLD | |
| | YWGQGTLVTVSSGSTSGSGKPGSGEGSTKGDIQLTQSPSSVSASVGDRVTITCRA | |
| | SQGISSWLAWYQQKPGKAPKLLIYGASSLQSGVPSRFSGSGSGTDFTLTISSLQP | |
| | EDFATYYCQQIYTFPFTFGGGTKVEIK | |
| 227 | DIQLTQSPSSVSASVGDRVTITCRASQGISSWLAWYQQKPGKAPKLLIYGASSLQ | Anti-BCMA scFv |
| | SGVPSRFSGSGSGTDFTLTISSLQPEDFATYYCQQIYTFPFTFGGGTKVEIKRGS | |
| | TSGSGKPGSGEGSTKGQVQLVESGGGVVQPGRSLRLSCAASGFTFSSYGMHWVRQ | |
| | APGKGLEWVAVISYDGSNKYYADSVKGRFTISRDNSKNTLYLQMNSLRAEDTAVY | |
| | YCARTDFWSGSPPGLDYWGQGTLVTVSS | |
| 228 | QVQLVQSGAEVKKPGSSVKVSCKASGGTFSSYAISWVRQAPGQGLEWMGGIIPIF | Anti-BCMA scFv |
| | GTANYAQKFQGRVTITADESTSTAYMELSSLRSEDTAVYYCARTPEYSSSIWHYY | |
| | YGMDVWGQGTTVTVSSGSTSGSGKPGSGEGSTKGDIVMTQSPDSLAVSLGERATI | |
| | NCKSSQSVLYSSNNKNYLAWYQQKPGQPPKLLIYWASTRESGVPDRFSGSGSGTD | |
| | FTLTISSLQAEDVAVYYCQQFAHTPFTFGGGTKVEIK | |
| 229 | DIVMTQSPDSLAVSLGERATINCKSSQSVLYSSNNKNYLAWYQQKPGQPPKLLIY | Anti-BCMA scFv |
| | WASTRESGVPDRFSGSGSGTDFTLTISSLQAEDVAVYYCQQFAHTPFTFGGGTKV | |
| | EIKRGSTSGSGKPGSGEGSTKGQVQLVQSGAEVKKPGSSVKVSCKASGGTFSSYA | |
| | ISWVRQAPGQGLEWMGGIIPIFGTANYAQKFQGRVTITADESTSTAYMELSSLRS | |
| | EDTAVYYCARTPEYSSSIWHYYYGMDVWGQGTTVTVSS | |
| 230 | QVQLVESGGGVVQPGRSLRLSCAASGFTFSSYGMHWVRQAPGKGLEWVAVISYDG | Anti-BCMA scFv |
| | SNKYYADSVKGRFTISRDNSKNTLYLQMNSLRAEDTAVYYCVKGPLQEPPYDYGM | |
| | DVWGQGTTVTVSSGSTSGSGKPGSGEGSTKGEIVMTQSPATLSVSPGERATLSCR | |
| | ASQSVSSNLAWYQQKPGQAPRLLIYSASTRATGIPARFSGSGSGTEFTLTISSLQ | |
| | SEDFAVYYCQQHHVWPLTFGGGTKVEIK | |
| 231 | EIVMTQSPATLSVSPGERATLSCRASQSVSSNLAWYQQKPGQAPRLLIYSASTRA | Anti-BCMA scFv |
| | TGIPARFSGSGSGTEFTLTISSLQSEDFAVYYCQQHHVWPLTFGGGTKVEIKRGS | |
| | TSGSGKPGSGEGSTKGQVQLVESGGGVVQPGRSLRLSCAASGFTFSSYGMHWVRQ | |
| | APGKGLEWVAVISYDGSNKYYADSVKGRFTISRDNSKNTLYLQMNSLRAEDTAVY | |
| | YCVKGPLQEPPYDYGMDVWGQGTTVTVSS | |
| 232 | DIVLTQSPASLAVSLGERATINCRASESVSVIGAHLIHWYQQKPGQPPKLLIYLA | Anti-BCMA scFv |
| | SNLETGVPARFSGSGSGTDFTLTISSLQAEDAAIYYCLQSRIFPRTFGQGTKLEI | |
| | KGSTSGSGKPGSGEGSTKGQVQLVQSGSELKKPGASVKVSCKASGYTFTDYSINW | |
| | VRQAPGQGLEWMGWINTETREPAYAYDFRGRFVFSLDTSVSTAYLQISSLKAEDT | |
| | AVYYCARDYSYAMDYWGQGTLVTVSS | |
| 233 | DIVLTQSPASLAVSLGERATINCRASESVSVIGAHLIHWYQQKPGQPPKLLIYLA | Anti-BCMA scFv |
| | SNLETGVPARFSGSGSGTDFTLTISSLQAEDAAIYYCLQSRIFPRTFGQGTKLEI | |
| | KGSTSGSGKPGSGEGSTKGQVQLVQSGSELKKPGASVKVSCKASGYTFTDYSINW | |
| | VRQAPGQGLEWMGWINTETREPAYAYDFRGRFVFSLDTSVSTAYLQISSLKAEDT | |
| | AVYYCARDYSYAMDYWGQGTLVTVSS | |
| 234 | DIVLTQSPASLAVSLGERATINCRASESVSVIGAHLIHWYQQKPGQPPKLLIYLA | Anti-BCMA scFv |
| | SNLETGVPARFSGSGSGTDFTLTISSLQAEDAAIYYCLQSRIFPRTFGQGTKLEI | |
| | KGSTSGSGKPGSGEGSTKGQVQLVQSGSELKKPGASVKVSCKASGYTFTDYSINW | |
| | VRQAPGQGLEWMGWINTETREPAYAYDFRGRFVFSLDTSVSTAYLQISSLKAEDT | |
| | AVYYCARDYSYAMDYWGQGTLVTVSS | |
| 235 | EVQLVESGGGLVQPGGSLRLSCAVSGFALSNHGMSWVRRAPGKGLEWVSGIVYSG | Anti-BCMA scFv |
| | STYYAASVKGRFTISRDNSRNTLYLQMNSLRPEDTAIYYCSAHGGESDVWGQGTT | |
| | VTVSSASGGGGSGGRASGGGGSDIQLTQSPSSLSASVGDRVTITCRASQSISSYL | |
| | NWYQQKPGKAPKLLIYAASSLQSGVPSRFSGSGSGTDFTLTISSLQPEDFATYYC | |
| | QQSYSTPYTFGQGTKVEIK | |
| 236 | QVQLVESGGGLVQPGRSLRLSCAASGFTFSNYAMSWVRQAPGKGLGWVSGISRSG | Anti-BCMA scFv |
| | ENTYYADSVKGRFTISRDNSKNTLYLQMNSLRDEDTAVYYCARSPAHYYGGMDVW | |
| | GQGTTVTVSSASGGGGSGGRASGGGGSDIVLTQSPGTLSLSPGERATLSCRASQS | |
| | ISSSFLAWYQQKPGQAPRLLIYGASRRATGIPDRFSGSGSGTDFTLTISRLEPED | |
| | SAVYYCQQYHSSPSWTFGQGTKLEIK | |
| 237 | QVQLVESGGGLVQPGGSLRLSCAVSGFALSNHGMSWVRRAPGKGLEWVSGIVYSG | Anti-BCMA scFv |
| | STYYAASVKGRFTISRDNSRNTLYLQMNSLRPEDTAIYYCSAHGGESDVWGQGTT | |
| | VTVSSASGGGGSGGRASGGGGSDIRLTQSPSPLSASVGDRVTITCQASEDINKFL | |
| | NWYHQTPGKAPKLLIYDASTLQTGVPSRFSGSGSGTDFTLTINSLQPEDIGTYYC | |
| | QQYESLPLTFGGGTKVEIK | |
| 238 | EVQLVESGGGLVQPGGSLRLSCAVSGFALSNHGMSWVRRAPGKGLEWVSGIVYSG | Anti-BCMA scFv |
| | STYYAASVKGRFTISRDNSRNTLYLQMNSLRPEDTAIYYCSAHGGESDVWGQGTT | |
| | VTVSSASGGGGSGGRASGGGGSEIVLTQSPGTLSLSPGERATLSCRASQSIGSSS | |
| | LAWYQQKPGQAPRLLMYGASSRASGIPDRFSGSGSGTDFTLTISRLEPEDFAVYY | |
| | CQQYAGSPPFTFGQGTKVEIK | |
| 239 | QIQLVQSGPELKKPGETVKISCKASGYTFTDYSINWVKRAPGKGLKWMGWINTET | Anti-BCMA scFv |
| | REPAYAYDFRGRFAFSLETSASTAYLQINNLKYEDTATYFCALDYSYAMDYWGQG | |
| | TSVTVSSGGGGSGGGGSGGGGSDIVLTQSPASLAMSLGKRATISCRASESVSVIG | |
| | AHLIHWYQQKPGQPPKLLIYLASNLETGVPARFSGSGSGTDFTLTIDPVEEDDVA | |
| | IYSCLQSRIFPRTFGGGTKLEIK | |
| 240 | QIQLVQSGPELKKPGETVKISCKASGYTFRHYSMNWVKQAPGKGLKWMGRINTES | Anti-BCMA scFv |
| | GVPIYADDFKGRFAFSVETSASTAYLVINNLKDEDTASYFCSNDYLYSLDFWGQG | |
| | TALTVSSGGGGSGGGGSGGGGSDIVLTQSPPSLAMSLGKRATISCRASESVTILG | |
| | SHLIYWYQQKPGQPPTLLIQLASNVQTGVPARFSGSGSRTDFTLTIDPVEEDDVA | |
| | VYYCLQSRTIPRTFGGGTKLEIK | |
| 241 | QIQLVQSGPELKKPGETVKISCKASGYTFTHYSMNWVKQAPGKGLKWMGRINTET | Anti-BCMA scFv |
| | GEPLYADDFKGRFAFSLETSASTAYLVINNLKNEDTATFFCSNDYLYSCDYWGQG | |
| | TTLTVSSGGGGSGGGGSGGGGSDIVLTQSPPSLAMSLGKRATISCRASESVTILG | |
| | SHLIYWYQQKPGQPPTLLIQLASNVQTGVPARFSGSGSRTDFTLTIDPVEEDDVA | |
| | VYYCLQSRTIPRTFGGGTKLEIK | |
| 242 | QVQLVQSGAEVKKPGASVKVSCKASGYSFPDYYINWVRQAPGQGLEWMGWIYFAS | Anti-BCMA scFv |
| | GNSEYNQKFTGRVTMTRDTSINTAYMELSSLTSEDTAVYFCASLYDYDWYFDVWG | |
| | QGTMVTVSSGGGGSGGGGSGGGGSDIVMTQTPLSLSVTPGQPASISCKSSQSLVH | |
| | SNGNTYLHWYLQKPGQSPQLLIYKVSNRFSGVPDRFSGSGSGTDFTLKISRVEAE | |
| | DVGIYYCSQSSIYPWTFGQGTKLEIK | |
| 243 | QVQLVQSGAEVKKPGASVKVSCKASGYSFPDYYINWVRQAPGQGLEWMGWIYFAS | Anti-BCMA scFv |
| | GNSEYNQKFTGRVTMTRDTSINTAYMELSSLTSEDTAVYFCASLYDYDWYFDVWG | |
| | QGTMVTVSSGGGGSGGGGSGGGGSDIVMTQTPLSLSVTPGQPASISCKSSQSLVH | |
| | SNGNTYLHWYLQKPGQSPQLLIYKVSNRFSGVPDRFSGSGSGTDFTLKISRVEAE | |
| | DVGIYYCSQSSIYPWTFGQGTKLEIK | |
| 244 | QVQLVQSGAEVKKPGASVKVSCKASGYSFPDYYINWVRQAPGQGLEWMGWIYFAS | Anti-BCMA scFv |
| | GNSEYNQKFTGRVTMTRDTSINTAYMELSSLTSEDTAVYFCASLYDYDWYFDVWG | |
| | QGTMVTVSSGGGGSGGGGSGGGGSDIVMTQTPLSLSVTPGQPASISCKSSQSLVH | |
| | SNGNTYLHWYLQKPGQSPQLLIYKVSNRFSGVPDRFSGSGSGTDFTLKISRVEAE | |
| | DVGIYYCSQSSIYPWTFGQGTKLEIK | |
| 245 | QVQLVQSGAEVKKPGASVKVSCKASGYSFPDYYINWVRQAPGQGLEWMGWIYFAS | Anti-BCMA scFv |
| | GNSEYNQKFTGRVTMTRDTSSSTAYMELSSLRSEDTAVYFCASLYDYDWYFDVWG | |
| | QGTMVTVSSGGGGSGGGGSGGGGSDIVMTQTPLSLSVTPGEPASISCKSSQSLVH | |
| | SNGNTYLHWYLQKPGQSPQLLIYKVSNRFSGVPDRFSGSGSGADFTLKISRVEAE | |
| | DVGVYYCAETSHVPWTFGQGTKLEIK | |
| 246 | QVQLVQSGAEVKKPGASVKVSCKASGYSFPDYYINWVRQAPGQGLEWMGWIYFAS | Anti-BCMA scFv |
| | GNSEYNQKFTGRVTMTRDTSSSTAYMELSSLRSEDTAVYFCASLYDYDWYFDVWG | |
| | QGTMVTVSSGGGGSGGGGSGGGGSDIVMTQTPLSLSVTPGEPASISCKSSQSLVH | |
| | SNGNTYLHWYLQKPGQSPQLLIYKVSNRFSGVPDRFSGSGSGADFTLKISRVEAE | |
| | DVGVYYCAETSHVPWTFGQGTKLEIK | |
| 247 | QVQLVQSGAEVKKPGASVKVSCKASGYSFPDYYINWVRQAPGQGLEWMGWIYFAS | Anti-BCMA scFv |
| | GNSEYNQKFTGRVTMTRDTSSSTAYMELSSLRSEDTAVYFCASLYDYDWYFDVWG | |
| | QGTMVTVSSGGGGSGGGGSGGGGSDIVMTQTPLSLSVTPGEPASISCKSSQSLVH | |
| | SNGNTYLHWYLQKPGQSPQLLIYKVSNRFSGVPDRFSGSGSGADFTLKISRVEAE | |
| | DVGVYYCAETSHVPWTFGQGTKLEIK | |

1. A method of treating a subject having a multiple myeloma (MM), comprising:
(a) determining that a subject has:
(i) a serum soluble B cell maturation antigen (sBCMA) level lower than about 600 ng/ml; and/or
(ii) an absence of IgG heavy chain disease (HCD); and
(b) administering to the subject a T cell therapy comprising a dose of genetically engineered T cells expressing a chimeric antigen receptor (CAR) that binds to human BCMA.

2. A method of treating a subject having a multiple myeloma (MM), comprising administering to the subject a T cell therapy comprising a dose of genetically engineered T cells expressing a chimeric antigen receptor (CAR) that binds to human B cell maturation antigen (BCMA), wherein the subject was previously determined to have:
(i) a serum soluble B cell maturation antigen (sBCMA) level lower than about 600 ng/ml; and/or
(ii) an absence of IgG heavy chain disease (HCD).

3. A method of treating a subject having a multiple myeloma (MM), comprising:
(a) selecting a subject for treatment with a T cell therapy comprising a dose of genetically engineered T cells expressing a chimeric antigen receptor (CAR) that binds to human BCMA, wherein the subject was previously determined to have:
(i) a serum soluble B cell maturation antigen (sBCMA) level lower than about 600 ng/ml; and/or
(ii) an absence of IgG heavy chain disease (HCD); and
(b) administering the T cell therapy to the subject.

4. A method of selecting a subject having a multiple myeloma (MM) for treatment with a T cell therapy comprising a dose of genetically engineering T cells, comprising determining that a subject has:
(i) a serum soluble B cell maturation antigen (sBCMA) level lower than about 600 ng/ml; and/or
(ii) an absence of IgG heavy chain disease (HCD),
wherein, if the subject is determined to have (i) and/or (ii), the subject is selected for administration with a T cell therapy comprising a dose of genetically engineered T cells expressing a chimeric antigen receptor (CAR) that binds to human BCMA.

5. A method of predicting the response of a subject having a multiple myeloma (MM) to treatment with a T cell therapy comprising a dose of genetically engineered T cells expressing a chimeric antigen receptor (CAR) that binds to human B cell maturation antigen (BCMA), comprising determining that a subject has:
(i) a serum soluble B cell maturation antigen (sBCMA) level lower than about 600 ng/ml; and/or
(ii) an absence of IgG heavy chain disease (HCD),
wherein, if the subject has (i) and/or (ii), the subject is predicted to achieve a complete response (CR) or a stringent complete response (sCR),
and wherein the treatment comprises administration of the dose of genetically engineered T cells to the subject.

6. A method of treating a subject having a multiple myeloma (MM), comprising:
(a) determining that the subject has:
(i) a serum soluble B cell maturation antigen (sBCMA) level higher than about 600 ng/ml; and/or
(ii) a presence of IgG heavy chain disease (HCD);
(b) debulking the MM; and
(c) administering to the subject a T cell therapy comprising a dose of genetically engineered T cells expressing a chimeric antigen receptor (CAR) that binds to human BCMA.

7. A method of treating a subject having a multiple myeloma (MM), comprising administering to a subject a T cell therapy comprising a dose of genetically engineered T cells expressing a chimeric antigen receptor (CAR) that binds to human B cell maturation antigen (BCMA), wherein:
(a) the subject was previously determined to have:
(i) a serum soluble B cell maturation antigen (sBCMA) level higher than about 600 ng/ml; and/or
(ii) a presence of IgG heavy chain disease (HCD); and
(b) the MM was debulked at a time between: (1) the subject being determined to have (i) and/or (ii); and (2) the subject being administered the T cell therapy.

8. A method of selecting a subject having a multiple myeloma (MM) for de-bulking, comprising determining that a subject has:
(i) a serum soluble B cell maturation antigen (sBCMA) level higher than about 600 ng/ml; and/or
(ii) a presence of IgG heavy chain disease (HCD),
wherein, if the subject is determined to have (i) and/or (ii), the subject is selected for debulking the MM prior to administration to the subject of a T cell therapy comprising a dose of genetically engineered T cells expressing a chimeric antigen receptor (CAR) that binds to human BCMA.

9. A method of predicting the response of a subject having a multiple myeloma (MM) to treatment with a T cell therapy comprising a dose of genetically engineered cells expressing a chimeric antigen receptor (CAR) that binds to human B cell maturation antigen (BCMA), comprising determining that a subject has:
(i) a serum soluble B cell maturation antigen (sBCMA) level higher than about 600 ng/ml; and/or
(ii) a presence of IgG heavy chain disease (HCD),
wherein, if the subject has (i) and/or (ii), the subject is predicted not to achieve a complete response (CR) or stringent complete response (sCR)),
and wherein the treatment comprises administration of the dose of genetically engineered T cells to the subject.

10. A method of treating a subject having a multiple myeloma (MM), comprising:
(a) determining that a subject has a first serum soluble B cell maturation antigen (sBCMA) level higher than about 600 ng/ml;
(b) debulking the MM;
(c) determining that the subject has a post-debulking serum sBCMA level lower than about 600 ng/ml; and
(d) administering to the subject a T cell therapy comprising a dose of genetically engineered T cells expressing a chimeric antigen receptor (CAR) that binds to human BCMA.

11. The method of claim 2, 3, or 7, further comprising determining that the subject has:
(i) a serum sBCMA level or a post-debulking serum sBCMA level higher or lower than about 600 ng/ml; and/or
(ii) a presence or absence of IgG HCD.

12. The method of any one of claims 1-11, wherein the method comprises determining that the subject has a serum sBCMA level or a post-debulking serum sBCMA level higher or lower than about 600 ng/ml.

13. The method of any one of claims 1-12, wherein:
the method comprises determining that the subject has a serum sBCMA level higher or lower than about 590 ng/ml, about 580 ng/ml, about 570 ng/ml, about 566 ng/ml, about 560 ng/ml, about 550 ng/ml, about 540 ng/ml, about 530 ng/ml, about 520 ng/ml, about 510 ng/ml, or about 500 ng/ml; and/or
the subject is determined to have a serum sBCMA level higher or lower than about 590 ng/ml, about 580 ng/ml, about 570 ng/ml, about 566 ng/ml, about 560 ng/ml, about 550 ng/ml, about 540 ng/ml, about 530 ng/ml, about 520 ng/ml, about 510 ng/ml, or about 500 ng/ml.

14. The method of any one of claims 1-9 and 11-13, wherein the method comprises determining that the subject has a presence or absence of IgG HCD.

15. The method of any one of claims 1-9 and 11-14, wherein determining the subject has a presence of IgG HCD comprises detecting IgG in serum and/or urine of the subject.

16. The method of any one of claims 1-15, wherein the determination of the serum sBCMA level is carried out about three months before, about two months before, about one month before, about three weeks before, about two weeks before, or about one week before: (i) administration of the T cell therapy to the subject; or (ii) the cells of the dose of genetically engineered CAR T cells are obtained from the subject.

17. The method of any one of claims 1-9 and 11-16, wherein the determination that the subject has a presence or absence of IgG HCD is carried out between about three months before, about two months before, about one month before, about three weeks before, about two weeks before, or about one week before: (i) administration of the T cell therapy to the subject; or (ii) the cells of the dose of genetically engineered CAR T cells are obtained from the subject.

18. The method of any one of claims 4, 5, 8, 9, and 12-17, further comprising administering the dose of genetically engineered cells to the subject.

19. The method of any one of claims 5, 9, and 12-18, wherein:
(i) if the subject is predicted to achieve a CR or a sCR, the method further comprises administering to the subject a T cell therapy comprising a dose of genetically engineered T cells expressing a chimeric antigen receptor (CAR) that binds to BCMA; or
(ii) if the subject is predicted not to achieve a CR or a sCR, the method further comprises selecting the subject for debulking the MM prior to administration to the subject of a dose of genetically engineered T cells expressing a chimeric antigen receptor (CAR) that binds to BCMA.

20. The method of any one of claims 1-19, wherein, following administration of the dose of genetically engineered T cells to the subject, the subject achieves a complete response (CR) or a stringent complete response (sCR).

21. The method of any one of claims 1-20, wherein, following administration of the dose of genetically engineered T cells to the subject, the subject achieves a CR.

22. The method of any one of claims 1-20, wherein, following administration of the dose of genetically engineered T cells to the subject, the subject achieves a sCR.

23. The method of any one of claims 6-8, 10, and 12-22, wherein the debulking comprises administering chemotherapy, radiation, and/or an immunomodulatory agent to the subject.

24. The method of claim 23 wherein the chemotherapy comprises melphalan, doxorubicin, or cyclophosphamide chemotherapy.

25. The method of claim 23 or claim 24 wherein the immunomodulatory agent is thalidomide, lenalidomide, or pomalidomide.

26. The method of any one of claims 23-25, wherein the immunomodulatory agent is a checkpoint inhibitor.

27. The method of any one of claims 6-8, 10, and 12-26, wherein the debulking is carried out within about three months before, within about two months before, within about one month before, within about three weeks before, within about two weeks before, or within about one week before administration of the T cell therapy to the subject.

28. The method of any one of claims 1-27, wherein prior to the administration of the dose of genetically engineered T cells to the subject, the subject has received a lymphodepleting therapy comprising the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

29. The method of any one of claims 1-28, wherein prior to the administration of the dose of genetically engineered T cells to the subject, the subject has received a lymphodepleting therapy comprising the administration of fludarabine at or about 30 mg/m² body surface area of the subject, daily, and cyclophosphamide at or about 300 mg/m² body surface area of the subject, daily, for 3 days.

30. The method of claim 28 or claim 29 wherein the debulking is carried out prior to the lymphodepleting therapy.

31. The method of any of claims 28-30, wherein the debulking is carried out after the lymphodepleting therapy.

32. The method of any one of claims 1-31, wherein the MM is a high-risk MM and/or a relapsed and/or refractory (r/r) MM.

33. The method of any one of claims 1-32, wherein the MM is a high-risk MM.

34. The method of any one of claims 1-33, wherein the MM is a relapsed and/or refractory (r/r) MM.

35. The method of any one of claims 1-34, wherein the subject is 18 year of age or older.

36. The method of any one of claims 1-35, wherein the subject has previously received three or more prior lines of therapy for the MM.

37. The method of claim 36, wherein each of the three or more prior lines of therapy comprises two consecutive cycles, unless progressive disease (PD; progression within 60 days after last dose) was the best response to the line of therapy.

38. The method of claim 36 or claim 37, wherein the three or more prior lines of therapy comprise a proteasome inhibitor, an immunomodulatory agent, and an anti-CD38 antibody.

39. The method of any one of claims 36-38, wherein the subject is refractory to the last of the three or more prior lines of therapy.

40. The method of any one of claims 1-39, wherein the subject has an Eastern Cooperative Oncology Group (ECOG) performance status of 0 or 1.

41. The method of any one of claims 1-40, wherein the subject has measurable disease at the time of the administration of the dose of genetically engineered T cells.

42. The method claim 41, wherein measurable disease comprises:
(i) serum M-protein greater or equal to 1.0 g/dL;
(ii) urine M-protein greater or equal to 200 mg/24 h; and/or
(iii) involved serum free light chain (FLC) level greater or equal to 10 mg/dL if serum FLC ratio is abnormal.

43. The method of any one of claims 1-42, wherein the subject does not have:
(i) central nervous system (CNS) involvement; and/or
(ii) a history or presence of clinically relevant CNS pathology.

44. The method of any one of claims 1-43, wherein the subject does not have active or a history of plasma cell leukemia (PCL).

45. The method of any one of claims 1-44, wherein the CAR comprises an extracellular antigen-binding domain that binds to BCMA, a transmembrane domain, and an intracellular signaling region.

46. The method of claim 45, wherein the extracellular antigen-binding domain comprises a variable heavy chain (V_{H}) region and, optionally, a variable light chain (V_{L}) region.

47. The method of claim 46, wherein:
the V_{H} region comprises a CDR-H1, a CDR-H2, and a CDR-H3 comprising the amino acid sequences set forth in SEQ ID NOS: 189, 190, and 191, respectively; and the V_{L} region comprises a CDR-L1, a CDR-L2, and a CDR-L3 comprising the amino acid sequences set forth in SEQ ID NOS: 192, 193, and 194, respectively; or
the V_{H} region comprises a CDR-H1, a CDR-H2, and a CDR-H3 comprising the amino acid sequences set forth in SEQ ID NOS: 173, 174 and 175, respectively; and the V_{L} region comprises a CDR-L1, a CDR-L2, and a CDR-L3 comprising the amino acid sequences set forth in SEQ ID NOS: 183, 184 and 185, respectively.

48. The method of claim 46 or claim 47, wherein:
the V_{H} region comprises an amino acid sequence set forth in SEQ ID NO: 18 and the V_{L} region comprises the amino acid sequence set forth in SEQ ID NO: 19;
or the V_{H} region comprises an amino acid sequence set forth in SEQ ID NO: 24, and the V_{L} region comprises the amino acid sequence set forth in SEQ ID NO: 25.

49. The method of any one of claims 45-48, wherein the extracellular antigen-binding domain is a single chain variable fragment (scFv).

50. The method of claim 49, wherein the scFv comprises the amino acid sequence set forth in SEQ ID NO: 213 or SEQ ID NO: 188.

51. The method of any one of claims 45-50, wherein the intracellular signaling region further comprises a costimulatory signaling domain.

52. The method of claim 51, wherein the costimulatory signaling domain comprises an intracellular signaling domain of CD28, 4-1BB, or ICOS, or a signaling portion thereof.

53. The method of claim 51 or claim 52, wherein the costimulatory signaling domain is between the transmembrane domain and the cytoplasmic signaling domain of a CD3-zeta (CD3ζ) chain.

54. The method of any one of claims 45-53, wherein the transmembrane domain is or comprises a transmembrane domain from CD28 or CD8, optionally human CD28 or CD8.

55. The method of any one of claims 45-54, wherein the CAR further comprises an extracellular spacer between the antigen binding domain and the transmembrane domain.

56. The method of claim 55, wherein the spacer is from CD8, optionally wherein the spacer is a CD8a hinge.

57. The method of claim 55 or claim 56, wherein the transmembrane domain and the spacer are from CD8.

58. The method of any one of claims 45-57, wherein the CAR comprises the amino acid sequence set forth in SEQ ID NO: 116 or SEQ ID NO: 124.

59. The method of any one of claims 45-58, wherein the CAR is encoded by the polynucleotide sequence set forth in SEQ ID NO: 214.

60. The method of any one of claims 1-59, wherein the CAR is ABECMA®.

61. The method of any one of claims 1-60, wherein the dose of genetically engineered T cells comprises: idecabtagene vicleucel cells: bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells: PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells, or GC012F cells.

62. The method of any one of claims 1-61, wherein the dose of genetically engineered T cells comprises idecabtagene vicleucel cells.

63. The method of any of claims 1-62, wherein the dose of genetically engineered T cells comprises CD3⁺ CAR-expressing T cells.

64. The method of any of claims 1-63, wherein the dose of genetically engineered T cells comprises a combination of CD4⁺ T cells and CD8⁺ T cells and/or a combination of CD4⁺ CAR-expressing T cells and CD8⁺ CAR-expressing T cells.

65. The method of claim 64, wherein the ratio of CD4⁺ CAR-expressing T cells to CD8⁺ CAR-expressing T cells and/or of CD4⁺ T cells to CD8⁺ T cells, is or is approximately 1:1 or is between at or approximately 1:3 and at or approximately 3:1.

66. The method of any of claims 1-65, wherein:
the percentage of naive-like T cells and/or central memory T cells is greater than or greater than about 60% of the total genetically engineered T cells in the dose, optionally greater than or greater than about 65%, 70%, 80%, 90% or 95%;
the percentage of naive-like T cells and/or central memory T cells is greater than or greater than about 40% of the total CD4+ genetically engineered T cells in the dose, optionally greater than or greater than about 50%, 60%, 70%, 80%, 90% or 95%; or
the percentage of naive-like T cells and/or central memory T cells is greater than or greater than about 40% of the total CD8+ genetically engineered T cells in the dose, optionally greater than or greater than about 50%, 60%, 70%, 80%, 90% or 95%.

67. The method of claim 66, wherein the naive-like T cells are CCR7+CD45RA+, CD27+CCR7+, or CD62L-CCR7+.

68. The method of any one of claims 1-67, wherein the dose of genetically engineered T cells comprises between about 0.5×10⁶ and about 600×10⁶ CAR-positive T cells.

69. The method of any one of claims 1-68, wherein the dose of genetically engineered T cells comprises between about 100×10⁶ and about 600×10⁶ CAR-positive T cells.

70. The method of any one of claims 1-69, wherein the dose of genetically engineered T cells comprises between about 150×10⁶ and about 450×10⁶ CAR-positive T cells.

71. The method of any one of claims 1-70, wherein the dose of genetically engineered T cells comprises about 150×10⁶, 300×10⁶, or about 450×10⁶ CAR-positive T cells.

72. The method of any one of embodiments 1-68, wherein the dose of genetically engineered T cells comprises between about 0.5×10⁶ and about 10×10⁶ CAR-positive T cells.

73. The method of any one of claims 1-72, wherein the cells of the dose of genetically engineered CAR T cells were obtained from the subject.

74. The method of any one of claims 1-73, wherein the dose of genetically engineered T cells are autologous to the subject.

75. The method of any one of claims 1-72, wherein the dose of genetically engineered T cells are allogeneic to the subject.


SEQUENCE LISTING (247 sequences)

SEQ ID NO: 1 - protein, 12 aa, Homo sapiens
  REGION 1..12: note = spacer (IgG4hinge)
ESKYGPPCPPCP

SEQ ID NO: 2 - protein, 36 aa, Homo sapiens
  REGION 1..36: note = spacer (IgG4hinge)
GAATCTAAGTACGGACCGCCCTGCCCCCCTTGCCCT

SEQ ID NO: 3 - protein, 119 aa, Homo sapiens
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, synthetic construct
  REGION 1..24: note = T2A
LEGGGEGRGSLLTCGDVEENPGPR

SEQ ID NO: 7 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  REGION 1..27: note = CD28
FWVLVVVGGVLACYSLLVTVAFIIFWV

SEQ ID NO: 9 - protein, 66 aa, Homo sapiens
  REGION 1..66: note = CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  REGION 1..41: note = CD28
RSKRSRLLHSDYMNMTPRRPGPTRKHYQPYAPPRDFAAYRS

SEQ ID NO: 11 - protein, 41 aa, Homo sapiens
  REGION 1..41: note = CD28
RSKRSRGGHSDYMNMTPRRPGPTRKHYQPYAPPRDFAAYRS

SEQ ID NO: 12 - protein, 42 aa, Homo sapiens
  REGION 1..42: note = 4-1BB
KRGRKKLLYIFKQPFMRPVQTTQEEDGCSCRFPEEEEGGCEL

SEQ ID NO: 13 - protein, 112 aa, Homo sapiens
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 10 aa, synthetic construct
  REGION 1..10: note = linker
  REPEAT 5..9: note = SGGGG is repeated 5 times
PGGGSGGGGP

SEQ ID NO: 17 - protein, 17 aa, synthetic construct
  REGION 1..17: note = linker
GSADDAKKDAAKKDGKS

SEQ ID NO: 18 - protein, 117 aa, synthetic construct
  REGION 1..117: note = Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 19 - protein, 111 aa, synthetic construct
  REGION 1..111: note = Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 20 - protein, 122 aa, synthetic construct
  REGION 1..122: note = Variable heavy (VH) Anti-BCMA
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 21 - protein, 107 aa, synthetic construct
  REGION 1..107: note = Variable light (VL) Anti-BCMA
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 22 - protein, 116 aa, synthetic construct
  REGION 1..116: note = Variable heavy (VH) Anti-BCMA
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 23 - protein, 111 aa, synthetic construct
  REGION 1..111: note = Variable light (VL) Anti-BCMA
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 24 - protein, 118 aa, synthetic construct
  REGION 1..118: note = Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 25 - protein, 105 aa, synthetic construct
  REGION 1..105: note = Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 26 - protein, 5 aa, synthetic construct
  REGION 1..5: note = Linker
GGGGS

SEQ ID NO: 27 - protein, 4 aa, synthetic construct
  REGION 1..4: note = Linker
GGGS

SEQ ID NO: 28 - protein, 15 aa, synthetic construct
  REGION 1..15: note = Linker
GGGGSGGGGSGGGGS

SEQ ID NO: 29 - protein, 18 aa, synthetic construct
  REGION 1..18: note = Linker
GSTSGSGKPGSGEGSTKG

SEQ ID NO: 30 - protein, 21 aa, synthetic construct
  REGION 1..21: note = Linker
SRGGGGSGGGGSGGGGSLEMA

SEQ ID NO: 31 - protein, 228 aa, Homo sapiens
  REGION 1..228: note = Hinge-CH2-CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 122 aa, synthetic construct
  REGION 1..122: note = Variable heavy (VH) Anti-BCMA
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 33 - protein, 111 aa, synthetic construct
  REGION 1..111: note = Variable light (VL) Anti-BCMA
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 34 - protein, 120 aa, synthetic construct
  REGION 1..120: note = Variable heavy (VH) Anti-BCMA
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 35 - protein, 112 aa, synthetic construct
  REGION 1..112: note = Variable light (VL) Anti-BCMA
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 36 - protein, 118 aa, synthetic construct
  REGION 1..118: note = Variable heavy (VH) Anti-BCMA
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 37 - protein, 112 aa, synthetic construct
  REGION 1..112: note = Variable light (VL) Anti-BCMA
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 38 - protein, 17 aa, synthetic construct
  REGION 1..17: note = Linker
ASGGGGSGGRASGGGGS

SEQ ID NO: 39 - protein, 21 aa, synthetic construct
  REGION 1..21: note = CD8a signal peptide
MALPVTALLLPLALLLHAARP

SEQ ID NO: 40 - protein, 20 aa, synthetic construct
  REGION 1..20: note = signal peptide
METDTLLLWVLLLWVPGSTG

SEQ ID NO: 41 - protein, 118 aa, synthetic construct
  REGION 1..118: note = Variable heavy (VH) Anti-BCMA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 42 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 43 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 44 - protein, 112 aa, synthetic construct
  REGION 1..112: note = Variable light (VL) Anti-BCMA
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 45 - protein, 116 aa, synthetic construct
  REGION 1..116: note = Variable heavy (VH) Anti-BCMA
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 46 - protein, 106 aa, synthetic construct
  REGION 1..106: note = Variable light (VL) Anti-BCMA
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 47 - protein, 122 aa, synthetic construct
  REGION 1..122: note = Variable heavy (VH) Anti-BCMA
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 48 - protein, 107 aa, synthetic construct
  REGION 1..107: note = Variable light (VL) Anti-BCMA
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 49 - protein, 119 aa, synthetic construct
  REGION 1..119: note = Variable heavy (VH) Anti-BCMA
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 50 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 51 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 52 - protein, 107 aa, synthetic construct
  REGION 1..107: note = Variable light (VL) Anti-BCMA
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 53 - protein, 126 aa, synthetic construct
  REGION 1..126: note = Variable heavy (VH) Anti-BCMA
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 54 - protein, 113 aa, synthetic construct
  REGION 1..113: note = Variable light (VL) Anti-BCMA
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 55 - protein, 123 aa, synthetic construct
  REGION 1..123: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 56 - protein, 107 aa, synthetic construct
  REGION 1..107: note = Variable light (VL) Anti-BCMA
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 57 - protein, 121 aa, synthetic construct
  REGION 1..121: note = Variable heavy (VH) Anti-BCMA
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 58 - protein, 112 aa, synthetic construct
  REGION 1..112: note = Variable light (VL) Anti-BCMA
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 59 - protein, 117 aa, synthetic construct
  REGION 1..117: note = Variable heavy (VH) Anti-BCMA
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 60 - protein, 111 aa, synthetic construct
  REGION 1..111: note = Variable light (VL) Anti-BCMA
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 61 - protein, 115 aa, synthetic construct
  REGION 1..115: note = Variable heavy (VH) Anti-BCMA
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 62 - protein, 107 aa, synthetic construct
  REGION 1..107: note = Variable light (VL) Anti-BCMA
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 63 - protein, 120 aa, synthetic construct
  REGION 1..120: note = Variable heavy (VH) Anti-BCMA
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 64 - protein, 109 aa, synthetic construct
  REGION 1..109: note = Variable light (VL) Anti-BCMA
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 65 - protein, 115 aa, synthetic construct
  REGION 1..115: note = Variable heavy (VH) Anti-BCMA
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 66 - protein, 107 aa, synthetic construct
  REGION 1..107: note = Variable light (VL) Anti-BCMA
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 67 - protein, 115 aa, synthetic construct; identical to SEQ ID NO: 61
  REGION 1..115: note = Variable heavy (VH) Anti-BCMA

SEQ ID NO: 68 - protein, 109 aa, synthetic construct
  REGION 1..109: note = Variable light (VL) Anti-BCMA
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 69 - protein, 117 aa, synthetic construct
  REGION 1..117: note = Variable heavy (VH) Anti-BCMA
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 70 - protein, 111 aa, synthetic construct
  REGION 1..111: note = Variable light (VL) Anti-BCMA
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 71 - protein, 117 aa, synthetic construct
  REGION 1..117: note = Variable heavy (VH) Anti-BCMA
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 72 - protein, 111 aa, synthetic construct
  REGION 1..111: note = Variable light (VL) Anti-BCMA
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 73 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 74 - protein, 112 aa, synthetic construct
  REGION 1..112: note = Variable light (VL) Anti-BCMA
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 75 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 76 - protein, 112 aa, synthetic construct
  REGION 1..112: note = Variable light (VL) Anti-BCMA
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 77 - protein, 122 aa, synthetic construct
  REGION 1..122: note = Anti-BCMA sdAb
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 78 - protein, 39 aa, synthetic construct
  REGION 1..39: note = CD28 spacer
IEVMYPPPYLDNEKSNGTIIHVKGKHLCPSPLFPGPSKP

SEQ ID NO: 79 - protein, 25 aa, synthetic construct
  REGION 1..25: note = CD8a TM
IYIWAPLAGTCGVLLLSLVITLYCN

SEQ ID NO: 80 - protein, 30 aa, synthetic construct
  REGION 1..30: note = CD28 spacer (truncated)
LDNEKSNGTIIHVKGKHLCPSPLFPGPSKP

SEQ ID NO: 81 - protein, 46 aa, synthetic construct
  REGION 1..46: note = CD8a hinge
PTTTPAPRPPTPAPTIASQPLSLRPEACRPAAGGAVHTRGLDFACD

SEQ ID NO: 82 - protein, 45 aa, synthetic construct
  REGION 1..45: note = CD8a hinge
TTTPAPRPPTPAPTIASQPLSLRPEACRPAAGGAVHTRGLDFACD

SEQ ID NO: 83 - protein, 55 aa, synthetic construct
  REGION 1..55: note = CD8a hinge
FVPVFLPAKPTTTPAPRPPTPAPTIASQPLSLRPEACRPAAGGAVHTRGLDFACD

SEQ ID NO: 84 - protein, 39 aa, synthetic construct
  REGION 1..39: note = CTLA4 hinge
DTGLYICKVELMYPPPYYLGIGNGTQIYVIDPEPCPDSD

SEQ ID NO: 85 - protein, 24 aa, synthetic construct
  REGION 1..24: note = CTLA4 TM
FLLWILAAVSSGLFFYSFLLTAVS

SEQ ID NO: 86 - protein, 38 aa, synthetic construct
  REGION 1..38: note = PD-1 hinge
QIKESLRAELRVTERRAEVPTAHPSPSPRPAGQFQTLV

SEQ ID NO: 87 - protein, 21 aa, synthetic construct
  REGION 1..21: note = PD-1 TM
VGVVGGLLGSLVLLVWVLAVI

SEQ ID NO: 88 - protein, 16 aa, synthetic construct
  REGION 1..16: note = Fc(gamma)RIIIa hinge
GLAVSTISSFFPPGYQ

SEQ ID NO: 89 - protein, 231 aa, synthetic construct
  REGION 1..231: note = IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 457 aa, synthetic construct
  REGION 1..457: note = anti-BCMA CAR
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 457 aa, synthetic construct
  REGION 1..457: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 466 aa, synthetic construct
  REGION 1..466: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 466 aa, synthetic construct
  REGION 1..466: note = anti-BCMA CAR
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 454 aa, synthetic construct
  REGION 1..454: note = anti-BCMA CAR
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 454 aa, synthetic construct
  REGION 1..454: note = anti-BCMA CAR
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 461 aa, synthetic construct
  REGION 1..461: note = anti-BCMA CAR
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 461 aa, synthetic construct
  REGION 1..461: note = anti-BCMA CAR
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 458 aa, synthetic construct
  REGION 1..458: note = anti-BCMA CAR
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 458 aa, synthetic construct
  REGION 1..458: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 461 aa, synthetic construct
  REGION 1..461: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 461 aa, synthetic construct
  REGION 1..461: note = anti-BCMA CAR
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 471 aa, synthetic construct
  REGION 1..471: note = anti-BCMA CAR
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 471 aa, synthetic construct
  REGION 1..471: note = anti-BCMA CAR
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 462 aa, synthetic construct
  REGION 1..462: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 462 aa, synthetic construct
  REGION 1..462: note = anti-BCMA CAR
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 466 aa, synthetic construct
  REGION 1..466: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 473 aa, synthetic construct
  REGION 1..473: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 470 aa, synthetic construct
  REGION 1..470: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 476 aa, synthetic construct
  REGION 1..476: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 475 aa, synthetic construct
  REGION 1..475: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 482 aa, synthetic construct
  REGION 1..482: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 476 aa, synthetic construct
  REGION 1..476: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 481 aa, synthetic construct
  REGION 1..481: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 479 aa, synthetic construct
  REGION 1..479: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 472 aa, synthetic construct
  REGION 1..472: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 472 aa, synthetic construct
  REGION 1..472: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 472 aa, synthetic construct
  REGION 1..472: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 466 aa, synthetic construct
  REGION 1..466: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAADTGLYICKVELMYPPPYYLGIGNGTQIYVIDPEPCPDSDFLLWILAAVSSGLFFYSFLLTAVSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 464 aa, synthetic construct
  REGION 1..464: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAAQIKESLRAELRVTERRAEVPTAHPSPSPRPAGQFQTLVVGVVGGLLGSLVLLVWVLAVICSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 653 aa, synthetic construct
  REGION 1..653: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 650 aa, synthetic construct
  REGION 1..650: note = anti-BCMA CAR
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 651 aa, synthetic construct
  REGION 1..651: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 658 aa, synthetic construct
  REGION 1..658: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 654 aa, synthetic construct
  REGION 1..654: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 653 aa, synthetic construct
  REGION 1..653: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 462 aa, synthetic construct
  REGION 1..462: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 469 aa, synthetic construct
  REGION 1..469: note = anti-BCMA CAR
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 462 aa, synthetic construct
  REGION 1..462: note = anti-BCMA CAR
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 464 aa, synthetic construct
  REGION 1..464: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 467 aa, synthetic construct
  REGION 1..467: note = anti-BCMA CAR
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 472 aa, synthetic construct
  REGION 1..472: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 466 aa, synthetic construct
  REGION 1..466: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 466 aa, synthetic construct
  REGION 1..466: note = anti-BCMA CAR
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 466 aa, synthetic construct
  REGION 1..466: note = anti-BCMA CAR
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 482 aa, synthetic construct
  REGION 1..482: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 28 aa, synthetic construct
  REGION 1..28: note = CD8a TM
IYIWAPLAGTCGVLLLSLVITLYCNHRN

SEQ ID NO: 143 - protein, 21 aa, synthetic construct
  REGION 1..21: note = CD8a TM
IYIWAPLAGTCGVLLLSLVIT

SEQ ID NO: 144 - protein, 4 aa, synthetic construct
  REGION 1..4: note = linking peptide
RAAA

SEQ ID NO: 145 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 146 - protein, 110 aa, synthetic construct
  REGION 1..110: note = Variable light (VL) Anti-BCMA
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 147 - protein, 118 aa, synthetic construct
  REGION 1..118: note = Variable heavy (VH) Anti-BCMA
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 148 - protein, 107 aa, synthetic construct
  REGION 1..107: note = Variable light (VL) Anti-BCMA
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 149 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 150 - protein, 108 aa, synthetic construct
  REGION 1..108: note = Variable light (VL) Anti-BCMA
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 151 - protein, 122 aa, synthetic construct; identical to SEQ ID NO: 32
  REGION 1..122: note = Variable heavy (VH) Anti-BCMA

SEQ ID NO: 152 - protein, 111 aa, synthetic construct; identical to SEQ ID NO: 33
  REGION 1..111: note = Variable light (VL) Anti-BCMA

SEQ ID NO: 153 - protein, 16 aa, synthetic construct
  REGION 1..16: note = CD33 Signal peptide
MPLLLLLPLLWAGALA

SEQ ID NO: 154 - protein, 18 aa, synthetic construct
  REGION 1..18: note = CD8 alpha signal peptide
MALPVTALLLPLALLLHA

SEQ ID NO: 155 - DNA, 66 bp, synthetic construct
  misc_feature 1..66: note = GMCSFR alpha chain signal sequence
  source 1..66: mol_type = other DNA
atgcttctcctggtgacaagccttctgctctgtgagttaccacacccagcattcctcctgatccca

SEQ ID NO: 156 - protein, 22 aa, synthetic construct
  REGION 1..22: note = GMCSFR alpha chain signal sequence
MLLLVTSLLLCELPHPAFLLIP

SEQ ID NO: 157 - protein, 14 aa, synthetic construct
  REGION 1..14: note = Exemplary IgG Hinge
EVVVKYGPPCPPCP

SEQ ID NO: 158 - skipped

SEQ ID NO: 159 - protein, 15 aa, synthetic construct
  REGION 1..15: note = Exemplary IgG Hinge
EPKSCDKTHTCPPCP

SEQ ID NO: 160 - protein, 12 aa, synthetic construct
  REGION 1..12: note = Exemplary IgG Hinge
ERKCCVECPPCP

SEQ ID NO: 161 - protein, 61 aa, synthetic construct
  REGION 1..61: note = Exemplary IgG Hinge
ELKTPLGDTHTCPRCPEPKSCDTPPPCPRCPEPKSCDTPPPCPRCPEPKSCDTPPPCPRCP

SEQ ID NO: 162 - protein, 12 aa, synthetic construct
  REGION 1..12: note = Exemplary IgG Hinge
ESKYGPPCPSCP

SEQ ID NO: 163 - protein, 12 aa, synthetic construct
  REGION 1..12: note = Exemplary IgG Hinge
ESKYGPPCPPCP

SEQ ID NO: 164 - protein, 9 aa, synthetic construct
  REGION 1..9: note = Exemplary IgG Hinge
YGPPCPPCP

SEQ ID NO: 165 - protein, 10 aa, synthetic construct
  REGION 1..10: note = Exemplary IgG Hinge
KYGPPCPPCP

SEQ ID NO: 166 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 167 - protein, 18 aa, synthetic construct
  REGION 1..18: note = T2A
EGRGSLLTCGDVEENPGP

SEQ ID NO: 168 - protein, 22 aa, synthetic construct
  REGION 1..22: note = P2A
GSGATNFSLLKQAGDVEENPGP

SEQ ID NO: 169 - protein, 19 aa, synthetic construct
  REGION 1..19: note = P2A
ATNFSLLKQAGDVEENPGP

SEQ ID NO: 170 - protein, 20 aa, synthetic construct
  REGION 1..20: note = E2A
QCTNYALLKLAGDVESNPGP

SEQ ID NO: 171 - protein, 22 aa, synthetic construct
  REGION 1..22: note = F2A
VKQTLNFDLLKLAGDVESNPGP

SEQ ID NO: 172 - protein, 290 aa, synthetic construct
  REGION 1..290: note = BCMA-Fc fusion polypeptide
MLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 5 aa, synthetic construct
  REGION 1..5: note = CDR-H1
DYYVY

SEQ ID NO: 174 - protein, 17 aa, synthetic construct
  REGION 1..17: note = CDR-H2
WINPNSGGTNYAQKFQG

SEQ ID NO: 175 - protein, 9 aa, synthetic construct
  REGION 1..9: note = CDR-H3
SQRDGYMDY

SEQ ID NO: 176 - protein, 7 aa, synthetic construct
  REGION 1..7: note = CDR-H1
GYTFIDY

SEQ ID NO: 177 - protein, 6 aa, synthetic construct
  REGION 1..6: note = CDR-H2
NPNSGG

SEQ ID NO: 178 - protein, 10 aa, synthetic construct
  REGION 1..10: note = CDR-H1
GYTFIDYYVY

SEQ ID NO: 179 - protein, 10 aa, synthetic construct
  REGION 1..10: note = CDR-H2
WINPNSGGTN

SEQ ID NO: 180 - protein, 8 aa, synthetic construct
  REGION 1..8: note = CDR-H1
GYTFIDYY

SEQ ID NO: 181 - protein, 8 aa, synthetic construct
  REGION 1..8: note = CDR-H2
INPNSGGT

SEQ ID NO: 182 - protein, 11 aa, synthetic construct
  REGION 1..11: note = CDR-H3
ARSQRDGYMDY

SEQ ID NO: 183 - protein, 8 aa, synthetic construct
  REGION 1..8: note = CDR-L1
TGTSSDVG

SEQ ID NO: 184 - protein, 7 aa, synthetic construct
  REGION 1..7: note = CDR-L2
EDSKRPS

SEQ ID NO: 185 - protein, 10 aa, synthetic construct
  REGION 1..10: note = CDR-L3
SSNTRSSTLV

SEQ ID NO: 186 - protein, 9 aa, synthetic construct
  REGION 1..9: note = CDR-L1
ISCTGTSSD

SEQ ID NO: 187 - skipped

SEQ ID NO: 188 - protein, 244 aa, synthetic construct
  REGION 1..244: note = anti-BCMA scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 189 - protein, 5 aa, synthetic construct
  REGION 1..5: note = CDR-H1
DYSIN

SEQ ID NO: 190 - protein, 17 aa, synthetic construct
  REGION 1..17: note = CDR-H2
WINTETREPAYAYDFRG

SEQ ID NO: 191 - protein, 8 aa, synthetic construct
  REGION 1..8: note = CDR-H3
DYSYAMDY

SEQ ID NO: 192 - protein, 15 aa, synthetic construct
  REGION 1..15: note = CDR-L1
RASESVTILGSHLIH

SEQ ID NO: 193 - protein, 7 aa, synthetic construct
  REGION 1..7: note = CDR-L2
LASNVQT

SEQ ID NO: 194 - protein, 9 aa, synthetic construct
  REGION 1..9: note = CDR-L3
LQSRTIPRT

SEQ ID NO: 195 - protein, 7 aa, synthetic construct
  REGION 1..7: note = CDR-H1
GYTFTDY

SEQ ID NO: 196 - protein, 6 aa, synthetic construct
  REGION 1..6: note = CDR-H2
NTETRE

SEQ ID NO: 197 - protein, 8 aa, synthetic construct
  REGION 1..8: note = CDR-H3
DYSYAMDY

SEQ ID NO: 198 - protein, 15 aa, synthetic construct
  REGION 1..15: note = CDR-L1
RASESVTILGSHLIH

SEQ ID NO: 199 - protein, 7 aa, synthetic construct
  REGION 1..7: note = CDR-L2
LASNVQT

SEQ ID NO: 200 - protein, 9 aa, synthetic construct
  REGION 1..9: note = CDR-L3
LQSRTIPRT

SEQ ID NO: 201 - protein, 10 aa, synthetic construct
  REGION 1..10: note = CDR-H1
GYTFTDYSIN

SEQ ID NO: 202 - protein, 10 aa, synthetic construct
  REGION 1..10: note = CDR-H2
WINTETREPA

SEQ ID NO: 203 - protein, 8 aa, synthetic construct
  REGION 1..8: note = CDR-H3
DYSYAMDY

SEQ ID NO: 204 - protein, 15 aa, synthetic construct
  REGION 1..15: note = CDR-L1
RASESVTILGSHLIH

SEQ ID NO: 205 - protein, 7 aa, synthetic construct
  REGION 1..7: note = CDR-L2
LASNVQT

SEQ ID NO: 206 - protein, 9 aa, synthetic construct
  REGION 1..9: note = CDR-L3
LQSRTIPRT

SEQ ID NO: 207 - protein, 8 aa, synthetic construct
  REGION 1..8: note = CDR-H1
GYTFTDYS

SEQ ID NO: 208 - protein, 8 aa, synthetic construct
  REGION 1..8: note = CDR-H2
INTETREP

SEQ ID NO: 209 - protein, 10 aa, synthetic construct
  REGION 1..10: note = CDR-H3
ALDYSYAMDY

SEQ ID NO: 210 - protein, 10 aa, synthetic construct
  REGION 1..10: note = CDR-L1
ESVTILGSHL

SEQ ID NO: 211 - skipped

SEQ ID NO: 212 - protein, 9 aa, synthetic construct
  REGION 1..9: note = CDR-L3
LQSRTIPRT

SEQ ID NO: 213 - protein, 246 aa, synthetic construct
  REGION 1..246: note = scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 214 - DNA, 1485 bp, synthetic construct
  misc_feature 1..1485: note = BCMA CAR (nt)
  source 1..1485: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 215 - protein, 184 aa, Homo sapiens
  REGION 1..184: note = Human BCMA
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 216 - protein, 243 aa, synthetic construct
  REGION 1..243: note = Anti-BCMA scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 217 - protein, 244 aa, synthetic construct
  REGION 1..244: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 218 - protein, 252 aa, synthetic construct
  REGION 1..252: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 219 - protein, 253 aa, synthetic construct
  REGION 1..253: note = Anti-BCMA scFv
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 220 - protein, 240 aa, synthetic construct
  REGION 1..240: note = Anti-BCMA scFv
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 221 - protein, 241 aa, synthetic construct
  REGION 1..241: note = Anti-BCMA scFv
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 222 - protein, 247 aa, synthetic construct
  REGION 1..247: note = Anti-BCMA scFv
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 223 - protein, 248 aa, synthetic construct
  REGION 1..248: note = Anti-BCMA scFv
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 224 - protein, 244 aa, synthetic construct
  REGION 1..244: note = Anti-BCMA scFv
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 225 - protein, 245 aa, synthetic construct
  REGION 1..245: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 226 - protein, 247 aa, synthetic construct
  REGION 1..247: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 227 - protein, 248 aa, synthetic construct
  REGION 1..248: note = Anti-BCMA scFv
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 228 - protein, 257 aa, synthetic construct
  REGION 1..257: note = Anti-BCMA scFv
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 229 - protein, 258 aa, synthetic construct
  REGION 1..258: note = Anti-BCMA scFv
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 230 - protein, 248 aa, synthetic construct
  REGION 1..248: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 231 - protein, 249 aa, synthetic construct
  REGION 1..249: note = Anti-BCMA scFv
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 232 - protein, 246 aa, synthetic construct
  REGION 1..246: note = Anti-BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 233 - protein, 246 aa, synthetic construct; identical to SEQ ID NO: 232
  REGION 1..246: note = Anti-BCMA scFv

SEQ ID NO: 234 - protein, 246 aa, synthetic construct; identical to SEQ ID NO: 232
  REGION 1..246: note = Anti-BCMA scFv

SEQ ID NO: 235 - protein, 239 aa, synthetic construct
  REGION 1..239: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 236 - protein, 246 aa, synthetic construct
  REGION 1..246: note = Anti-BCMA scFv
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 237 - protein, 239 aa, synthetic construct
  REGION 1..239: note = Anti-BCMA scFv
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 238 - protein, 241 aa, synthetic construct
  REGION 1..241: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 239 - protein, 243 aa, synthetic construct
  REGION 1..243: note = Anti-BCMA scFv
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 240 - protein, 243 aa, synthetic construct
  REGION 1..243: note = Anti-BCMA scFv
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 241 - protein, 243 aa, synthetic construct
  REGION 1..243: note = Anti-BCMA scFv
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 242 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 243 - protein, 246 aa, synthetic construct; identical to SEQ ID NO: 242
  REGION 1..246: note = Anti-BCMA scFv

SEQ ID NO: 244 - protein, 246 aa, synthetic construct; identical to SEQ ID NO: 242
  REGION 1..246: note = Anti-BCMA scFv

SEQ ID NO: 245 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 246 - protein, 246 aa, synthetic construct; identical to SEQ ID NO: 245
  REGION 1..246: note = Anti-BCMA scFv

SEQ ID NO: 247 - protein, 246 aa, synthetic construct; identical to SEQ ID NO: 245
  REGION 1..246: note = Anti-BCMA scFv